(19) United States
(12) Patent Application Publication
(10) Pub. No.: US 2011/0218156 A1
(43) Pub. Date: Sep. 8, 2011

(54) MOLECULAR ENTITIES FOR BINDING, STABILIZATION AND CELLULAR DELIVERY OF NEGATIVELY CHARGED MOLECULES

(76) Inventors: Alexander Chucholowski, San Diego, CA (US); Alisher Khasanov, San Diego, CA (US); Tingmin Wang, San Diego, CA (US); Tong Zhu, San Diego, CA (US)

(21) Appl. No.: 13/016,720

(22) Filed: Jan. 28, 2011

Related U.S. Application Data
(60) Provisional application No. 61/301,556, filed on Feb. 4, 2010.

(51) Int. Cl. A61K 38/14; C08B 37/16; C12N 5/07; C07K 9/00; A61K 31/7088; A61K 31/713

(52) U.S. Cl. 514/20.9; 536/103; 435/375; 530/322; 514/44 R; 514/44 A; 428/402

(57) ABSTRACT

In accordance with the present invention, it has been discovered that the uptake of negatively charged entities into cells can be enhanced by noncovalently associating such charged entities with molecular entities comprising an amphiphilic core with positively charged arms, wherein a plurality of lipophilic (e.g., bile acid) moieties are covalently attached to the positively charged arms. The molecular entities form well defined stoichiometric complexes with negatively charged entities. Various compositions and methods for stabilizing anionic charged entities and for enhancing the cellular uptake of any anionic charged entities, e.g. double-stranded or hairpin nucleic acid, are provided.

### RELATED APPLICATIONS

This application claims priority from U.S. Provisional Application No. 61/301,556, filed Feb. 4, 2010, the entire contents of which are hereby incorporated by reference herein.

### SEQUENCE LISTING

The instant application contains a Sequence Listing which has been submitted in ASCII format via EFS-Web and is hereby incorporated by reference in its entirety. Said ASCII copy, created on Apr. 21, 2011, is named RGO11401.txt and is 47,585 bytes in size.

### TECHNICAL FIELD

This invention relates to molecular entities for binding, stabilization and cellular delivery of negatively charged molecules and for therapeutic treatment of diseases using same.

### BACKGROUND

The potential use of negatively charged molecules such as polynucleotides as therapeutic agents has attracted great attention as a novel approach for treating severe and chronic diseases. However, polynucleotides have poor bioavailability and uptake into cells because polynucleotides do not readily permeate the cellular membrane due to the charge repulsion between the negatively charged membrane and the high negative charge on the polynucleotide. In addition, polynucleotides are also highly susceptible to rapid nuclease degradation both inside and outside the cytoplasm; see examples from Geary et al, J. Pharmacol. Exp. Ther. 296:890-897 (2001).

One strategy to improve the structural stability of polynucleotides in vivo is to modify the phosphodiester backbone structure of the polynucleotides in efforts to reduce enzymatic susceptibility. Other strategies for addressing stability and delivery of polynucleotides include condensation of cationic molecules (such as viral vectors) with polynucleotides and cationic delivery system (such as lipid vesicles, lipid nanoparticles, polyethyleneimines and cyclodextrin-based polymers). However, concerns with intracellular vehicle fate and toxicity remain high. There is an ongoing need for improved compositions and methods for binding, stabilization and cellular delivery of negatively charged molecules and for therapeutic treatment of diseases using same.

### SUMMARY OF INVENTION

In accordance with the present invention, it has been discovered that the uptake of charged molecules (especially negatively charged entities) into cells can be enhanced by noncovalently associating such molecules with molecular entities comprising an amphiphilic core with oppositely charged (especially positively charged) arms, wherein a plurality of lipophilic (e.g., bile acid) moieties are covalently attached to the oppositely charged arms. For example, anionic charged molecules can be delivered employing molecular entities comprising cationic charged arms. The molecular entities form well defined stoichiometric complexes with charged molecules. Various compositions and methods for stabilizing charged molecules and for enhancing the cellular uptake of any charged molecules, e.g. anionic charged molecules such as double-stranded or hairpin nucleic acids, are provided.

### BRIEF DESCRIPTION OF THE FIGURES

FIG. 1 presents the structure of compound 6a from Example 5.

FIG. 2 presents the structure of compound 6b from Example 5.

FIG. 3 presents the structure of compound 6c from Example 5.

FIG. 4 presents the structure of compound 6d from Example 5.

FIG. 5 presents the structure of compound 6e from Example 5.

FIG. 6 presents the structure of compound 6f from Example 5.

FIG. 7 presents the structure of compound 6g from Example 5.

FIG. 8 presents the structure of compound 6h from Example 5.

FIG. 9 presents the structure of compound 6i from Example 5.

FIG. 10 presents the structure of compound 6j from Example 5.

FIG. 11 presents the structure of compound 6k from Example 5.

FIG. 12 presents the structure of compound 6l from Example 5.

FIG. 13 presents the structure of compound 6m from Example 5.

FIG. 14 presents the structure of compound 6n from Example 5.

FIG. 15 presents the structure of compound 6o from Example 5.

FIG. 16 presents the structure of compound 7b from Example 6.

FIG. 17 presents the structure of compound 7c from Example 6.

FIG. 18 presents the structure of compound 7d from Example 6.

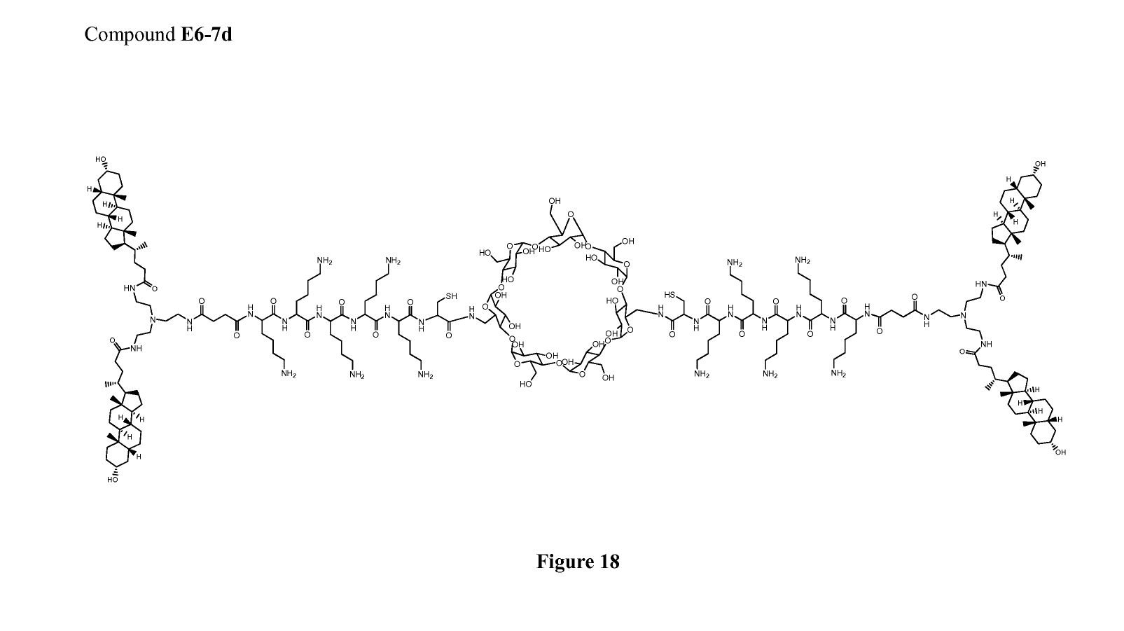

FIG. 19 presents the structure of compound 7e from Example 6.

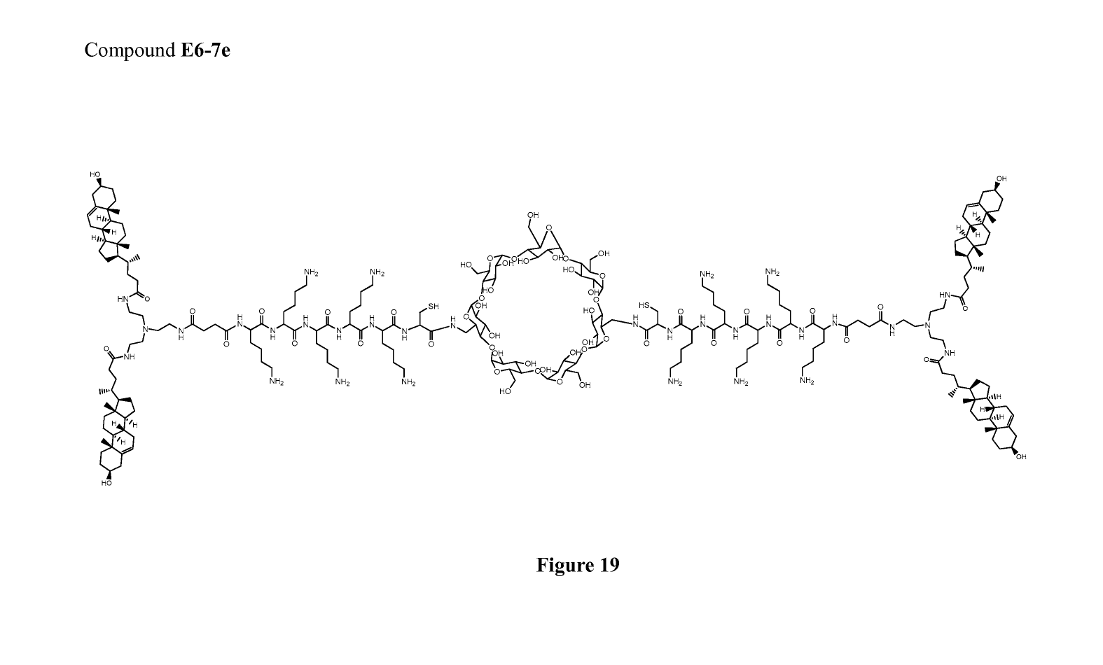

FIG. 20 presents the structure of compound 8a from Example 7.

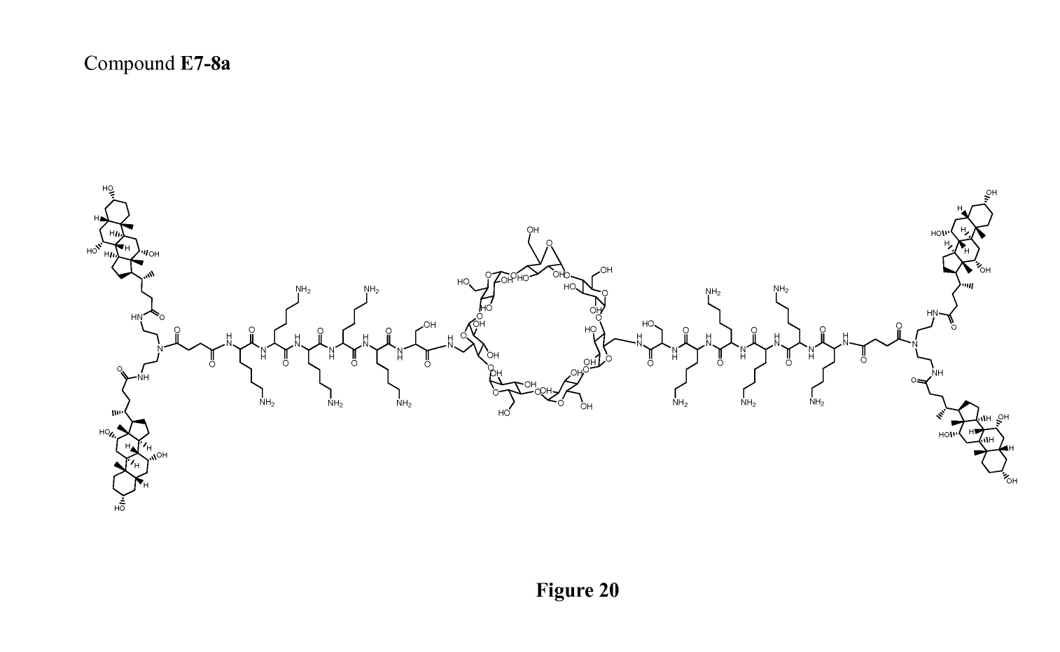

FIG. 21 presents the structure of compound 8b from Example 8.

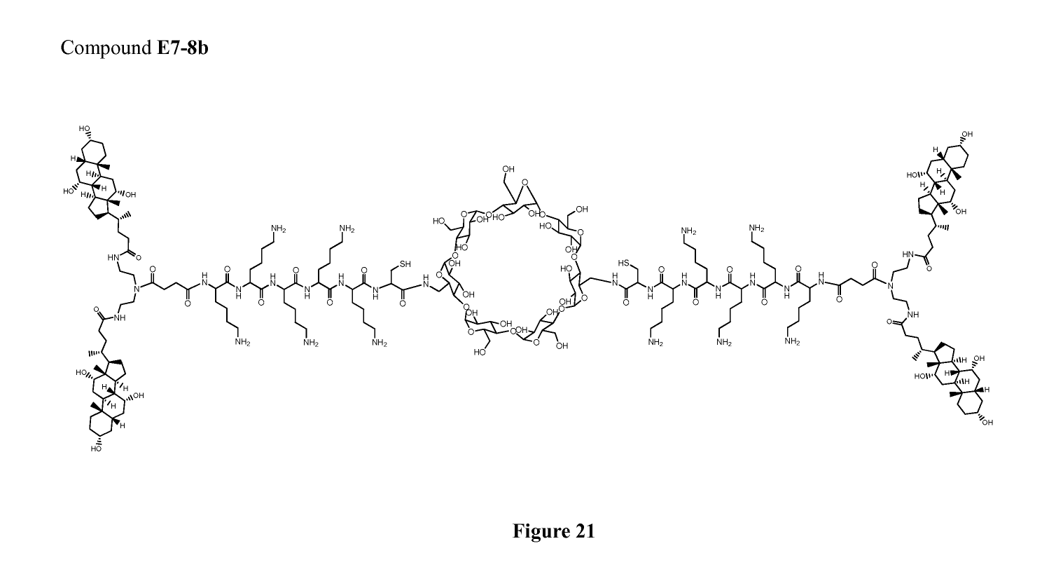

FIG. 22 presents the structure of compound 10a from Example 8.

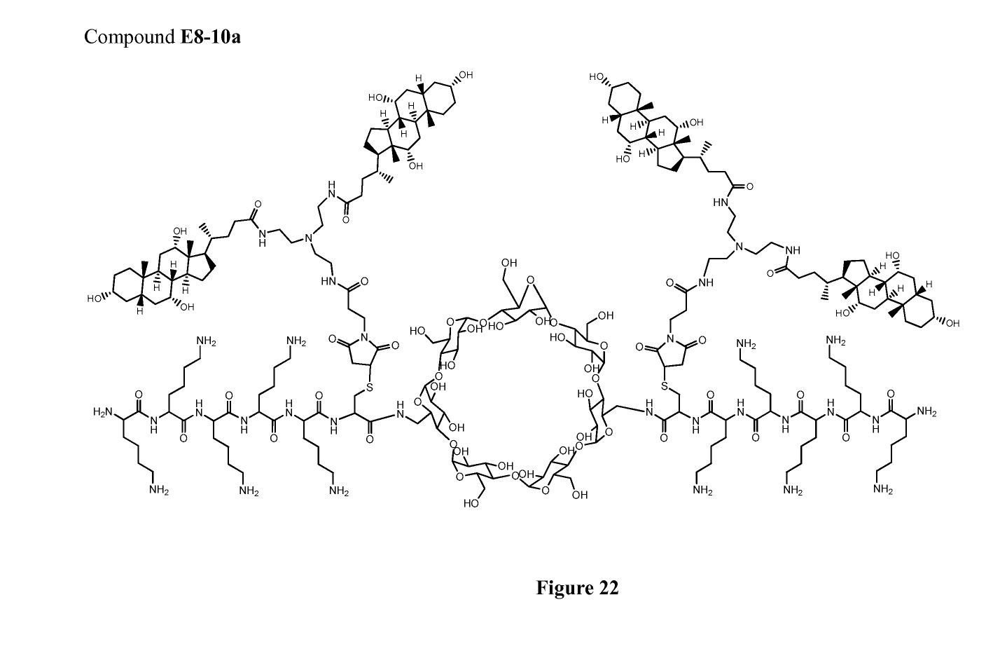

FIG. 23 presents the structure of compound 10b from Example 8.

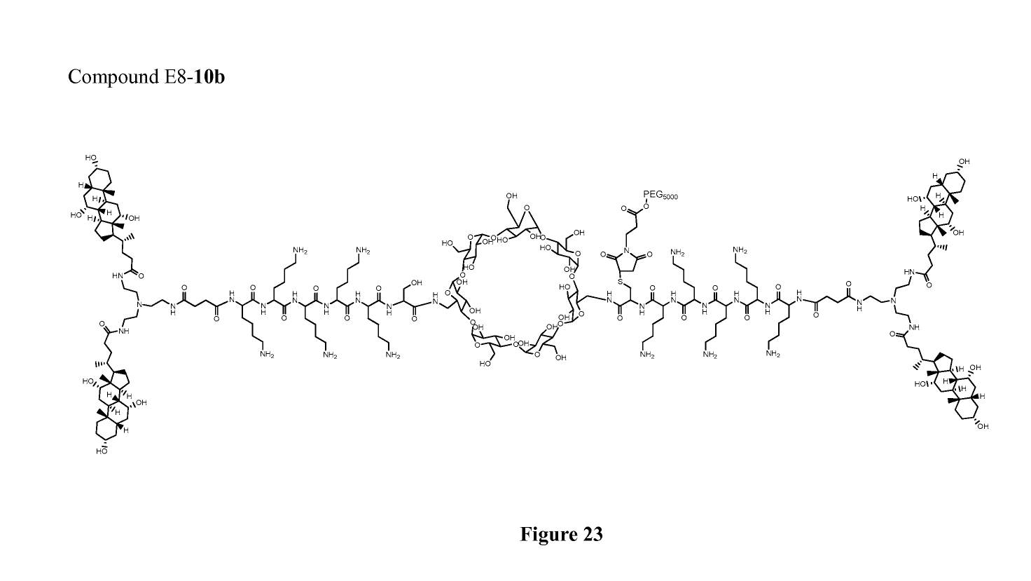

FIG. 24 presents the structure of compound 10c from Example 8.

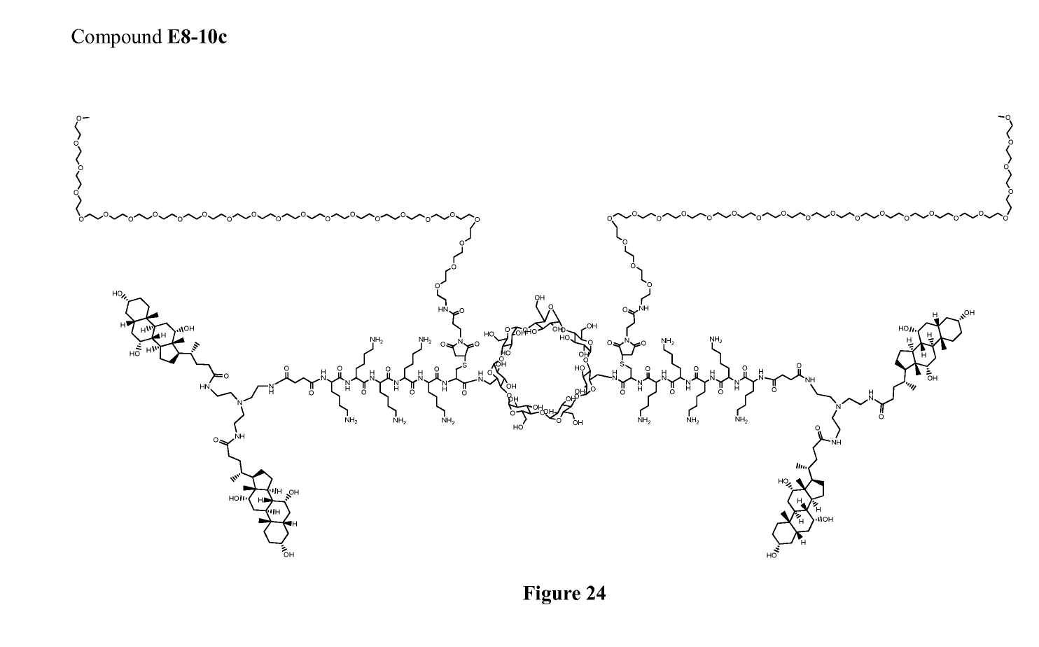

FIG. 25 presents the structure of compound 10d from Example 8.

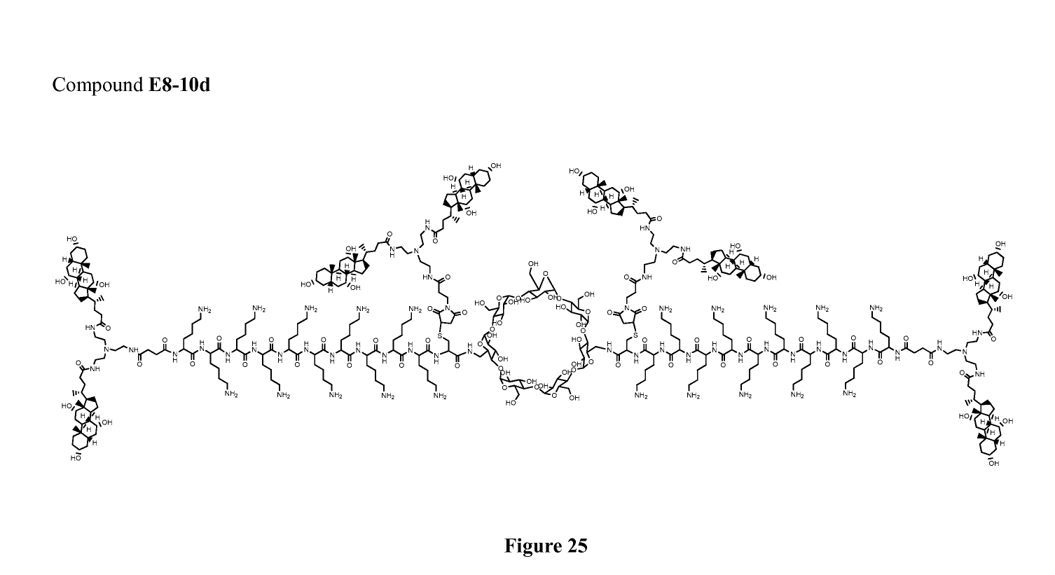

FIG. 26 presents the structure of compound 11 from Example 8.

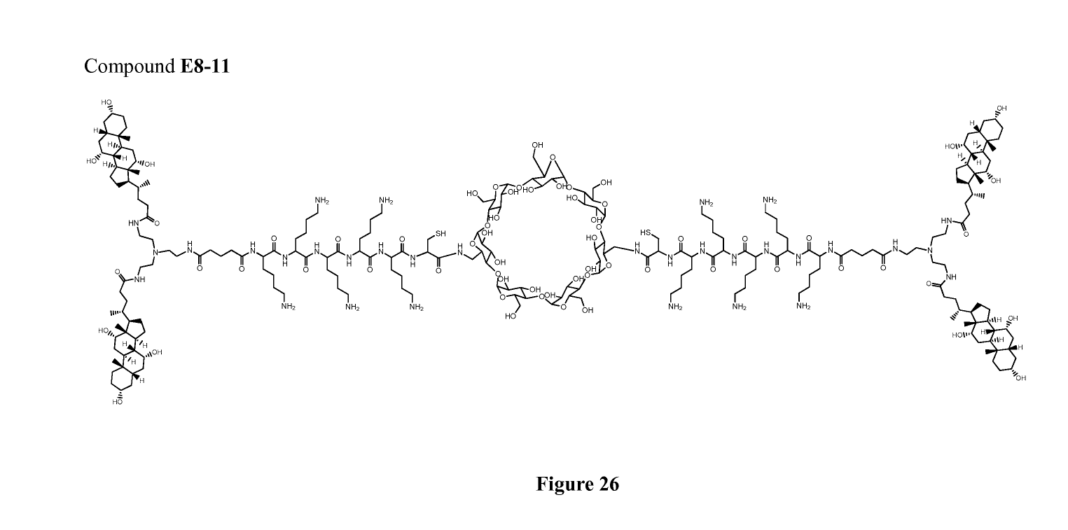

FIG. 27 presents the structure of compound 12 from Example 8.

FIG. 28 presents the structure of compound 13 from Example 8.

FIG. 29 presents the structure of compound 14 from Example 8.

FIG. 30 presents the structure of compound 15 from Example 8.

FIG. 31 presents the structure of compound 16 from Example 8.

FIG. 32 presents the structure of compound 17 from Example 8.

FIG. 33 presents the structure of compound 18 from Example 8.

FIG. 34 presents the structure of compound 19 from Example 8.

FIG. 35 presents the structure of compound 20 from Example 8.

FIG. 36 presents the structure of compound 21 from Example 8.

FIG. 37 presents the structure of compound 22 from Example 8.

FIG. 38 presents the structure of compound 23 from Example 8.

FIG. 39 presents the structure of compound 24 from Example 8.

FIG. 40 presents the structure of compound 25 from Example 8.

FIG. 41 presents the structure of compound 26 from Example 8.

FIG. 42 presents the structure of compound 27 from Example 8.

FIG. 43 presents the structure of compound 28 from Example 8.

FIG. 44 presents the structure of compound 29 from Example 8.

FIG. 45 presents the structure of compound 30 from Example 8.

FIG. 46 presents the structure of compound 31 from Example 8.

FIG. 47 presents the structure of compound 32 from Example 8.

FIG. 48 presents the structure of compound 33 from Example 8.

FIG. 49 presents the structure of compound 34 from Example 8.

FIG. 50 presents the structure of compound 35 from Example 8.

FIG. 51 presents the structure of compound 36 from Example 8.

FIG. 52 presents the structure of compound 37 from Example 8.

FIG. 53 presents the structure of compound 38 from Example 8.

FIG. 54 presents the structure of compound 39 from Example 8.

FIG. 55 presents the structure of compound 40 from Example 8.

FIG. 56 presents the structure of compound 41 from Example 8.

FIG. 57 presents the structure of compound 42 from Example 8.

FIG. 58 presents the structure of compound 43 from Example 8.

FIG. 59 presents the structure of compound 44 from Example 8.

FIG. 60 presents the structure of compound 45 from Example 8.

FIG. 61 presents the structure of compound 46 from Example 8.

FIG. 62 presents the structure of compound 47 from Example 8.

FIG. 63 presents the structure of compound 48 from Example 8.

FIG. 64 presents the structure of compound 49 from Example 8.

FIG. 65 presents the structure of compound 50 from Example 8.

FIG. 66 presents the structure of compound 51 from Example 8.

FIG. 67 presents the structure of compound 52 from Example 8.

FIG. 68 presents the structure of compound 53 from Example 8.

FIG. 69 presents the structure of compound 54 from Example 8.

FIG. 70 presents the structure of compound 55 from Example 8.

FIG. 71 presents the structure of compound 56 from Example 8.

FIG. 72 presents the structure of compound 57 from Example 8.

FIG. 73 presents the structure of compound 58 from Example 8.

FIG. 74 presents the structure of compound 59 from Example 8.

FIG. 75 presents the structure of compound 60 from Example 8.

FIG. 76 presents the structure of compound 61 from Example 8.

FIG. 77 presents the structure of compound 62 from Example 8.

FIG. 78 presents the structure of compound 63 from Example 8.

FIG. 79 presents the structure of compound 64 from Example 8.

FIG. 80 presents the structure of compound 65 from Example 8.

FIG. 81 presents the structure of compound 11 from Example 10.

FIG. 82 presents the structure of compound 12 from Example 10.

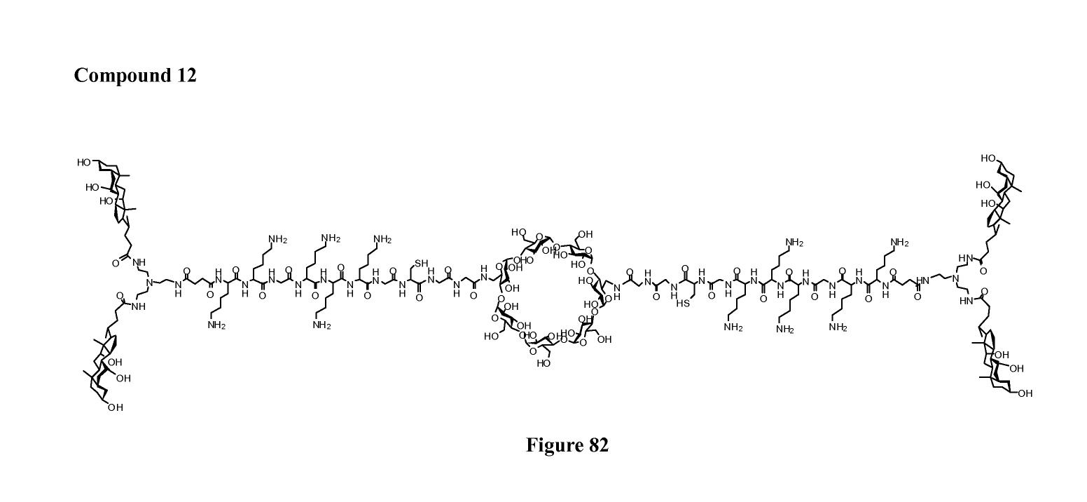

FIG. 83 presents the structure of compound 13 from Example 10.

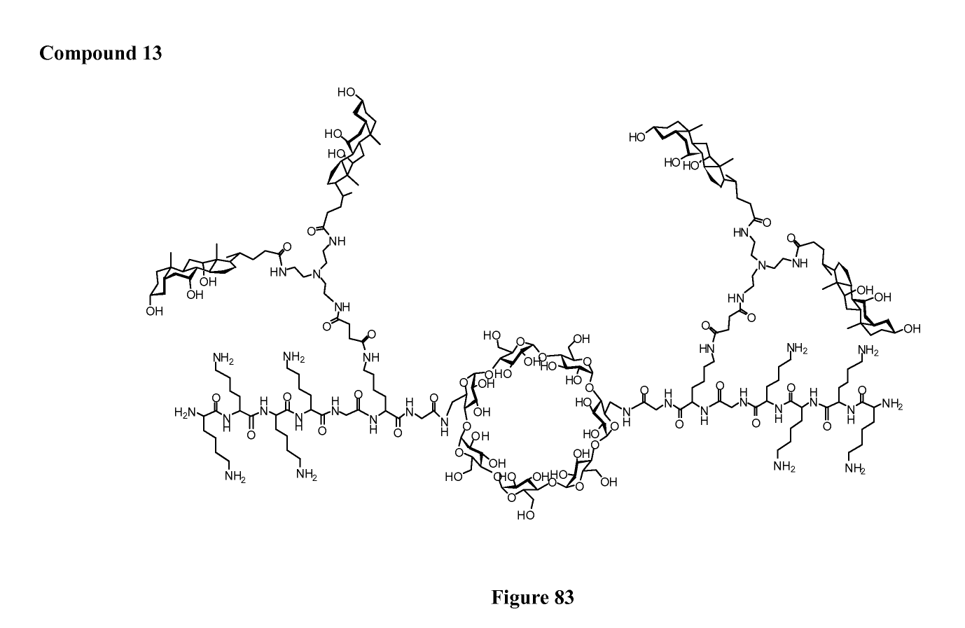

FIG. 84 presents the structure of compound 14 from Example 10.

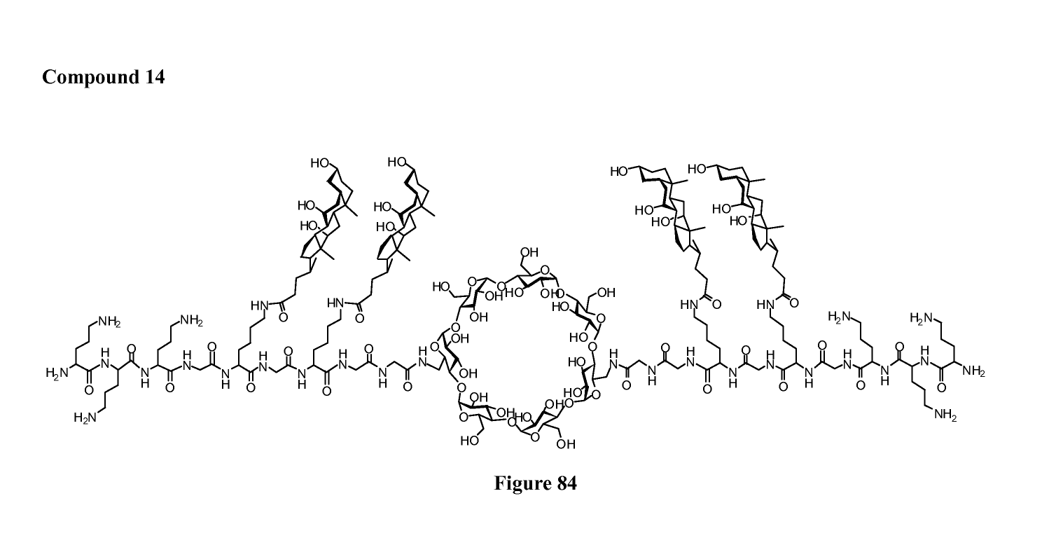

FIG. 85 presents the structure of compound 15 from Example 10.

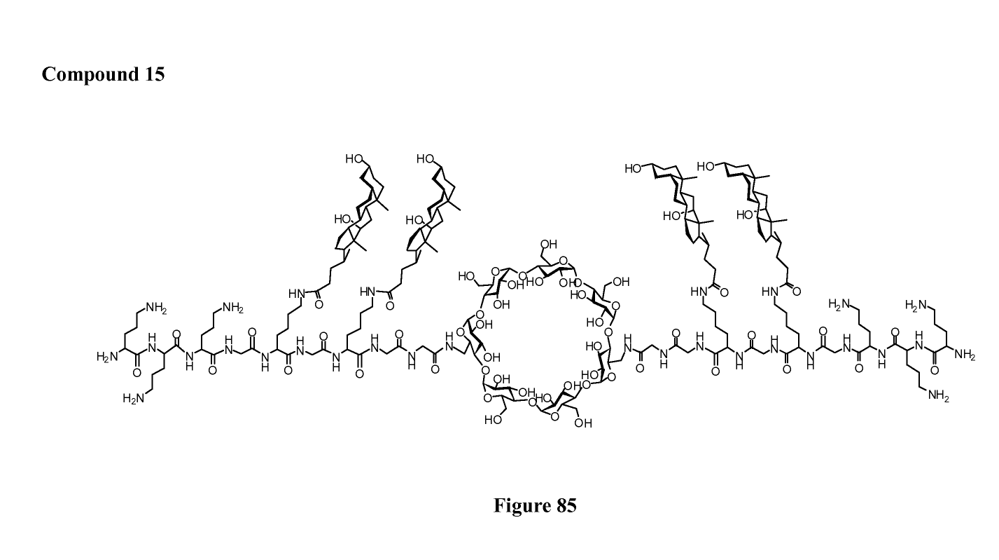

FIG. 86 presents the structure of compound 16 from Example 10.

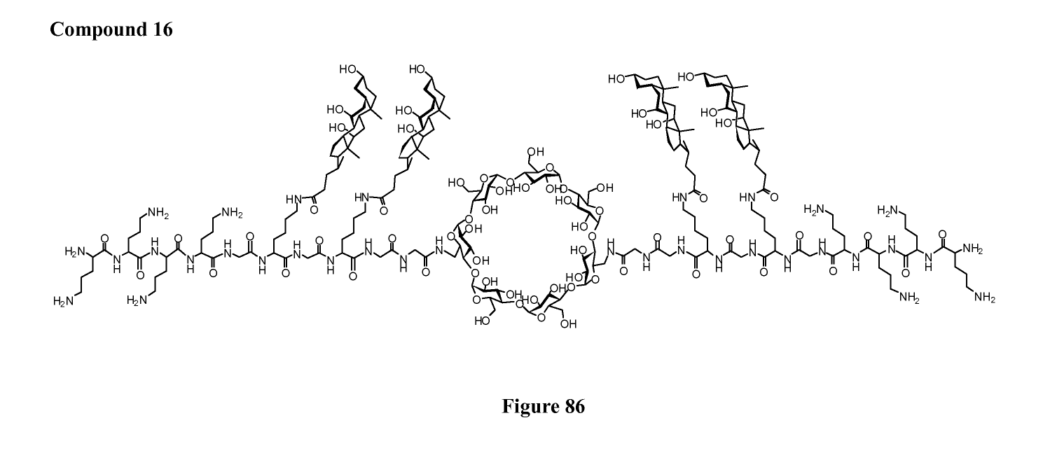

FIG. 87 presents the structure of compound 17 from Example 10.

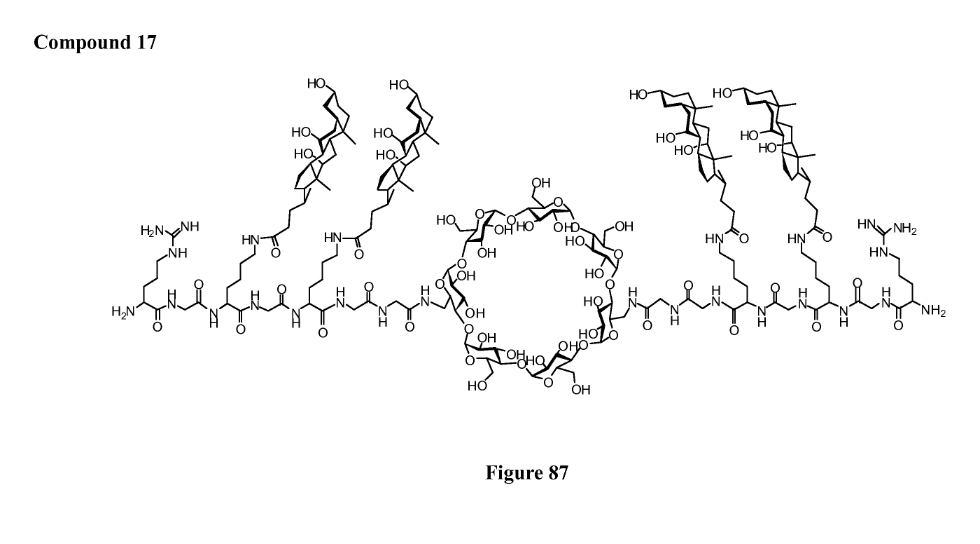

FIG. 88 presents the structure of compound 18 from Example 10.

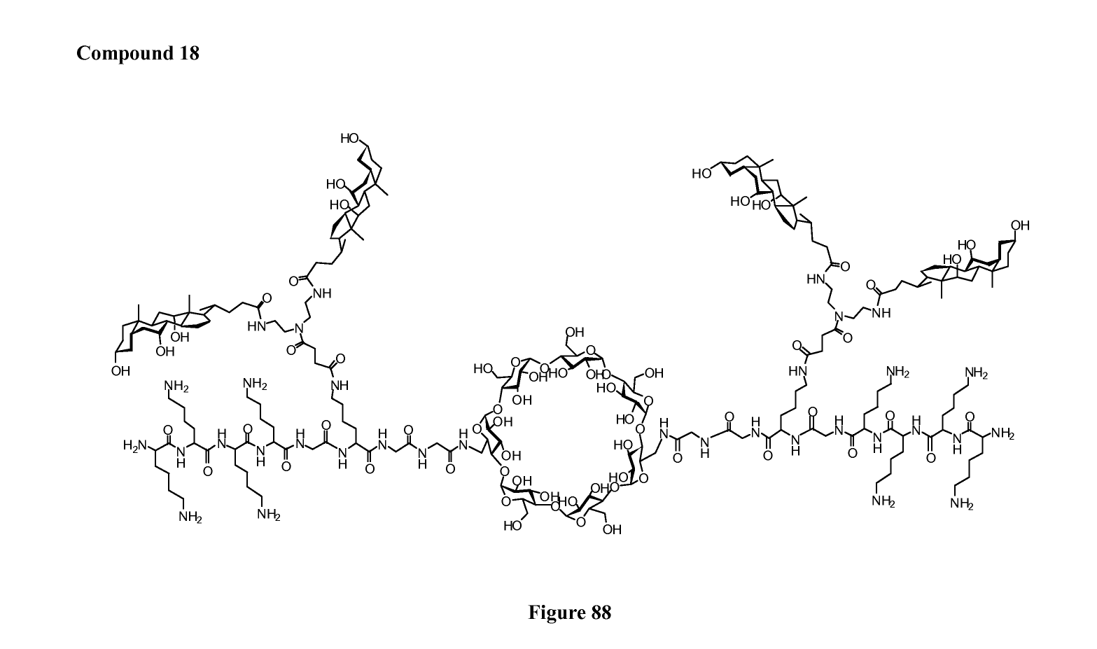

FIG. 89 presents the structure of compound 19 from Example 10.

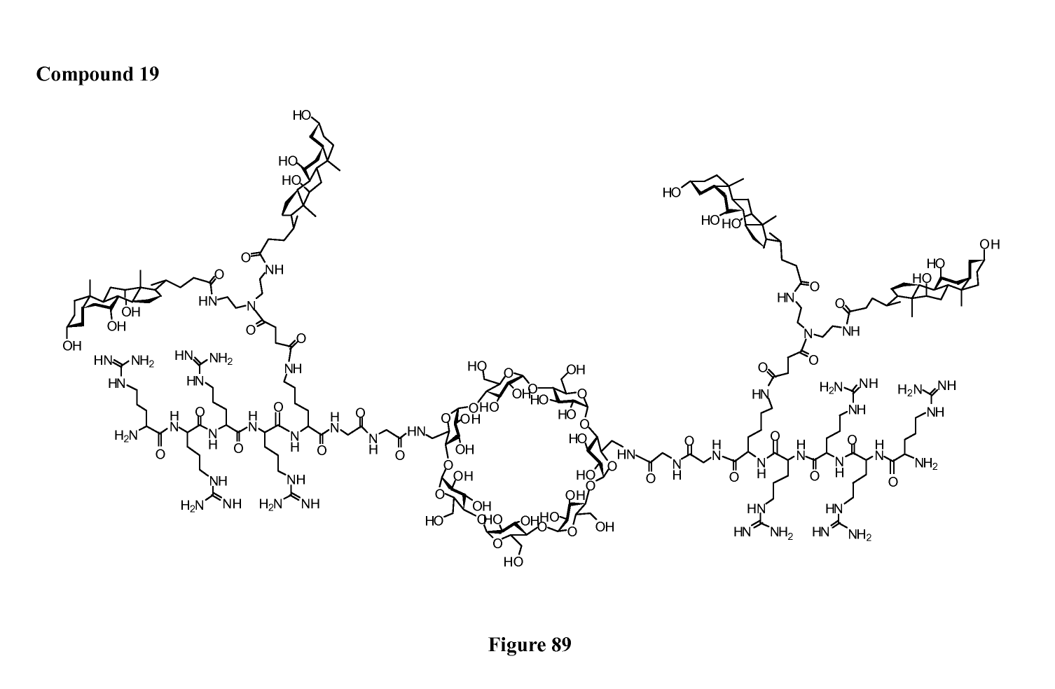

FIG. 90 presents the structure of compound 20 from Example 10.

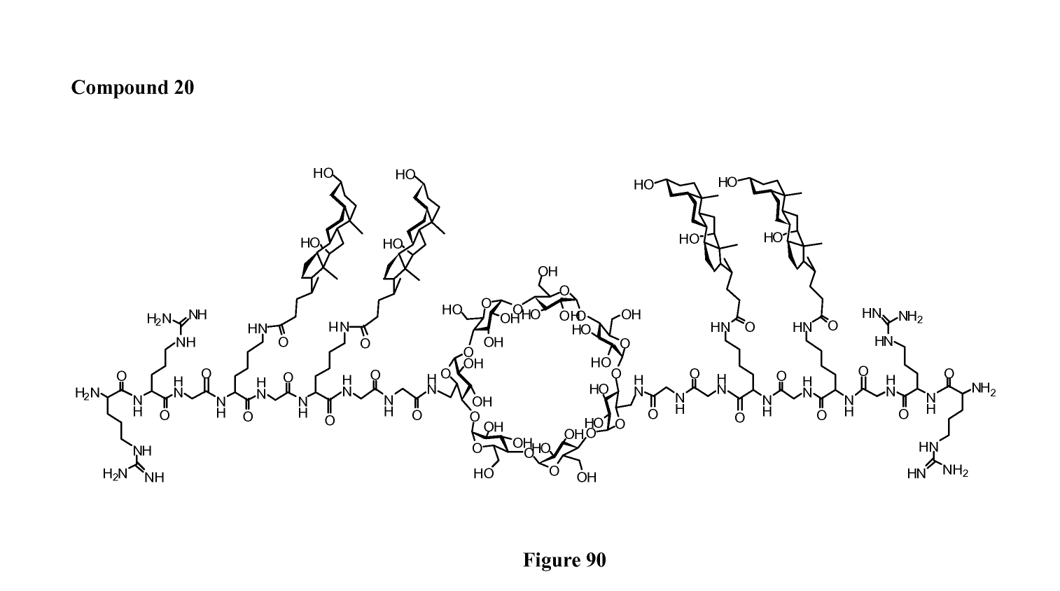

FIG. 91 presents the structure of compound 21 from Example 10.

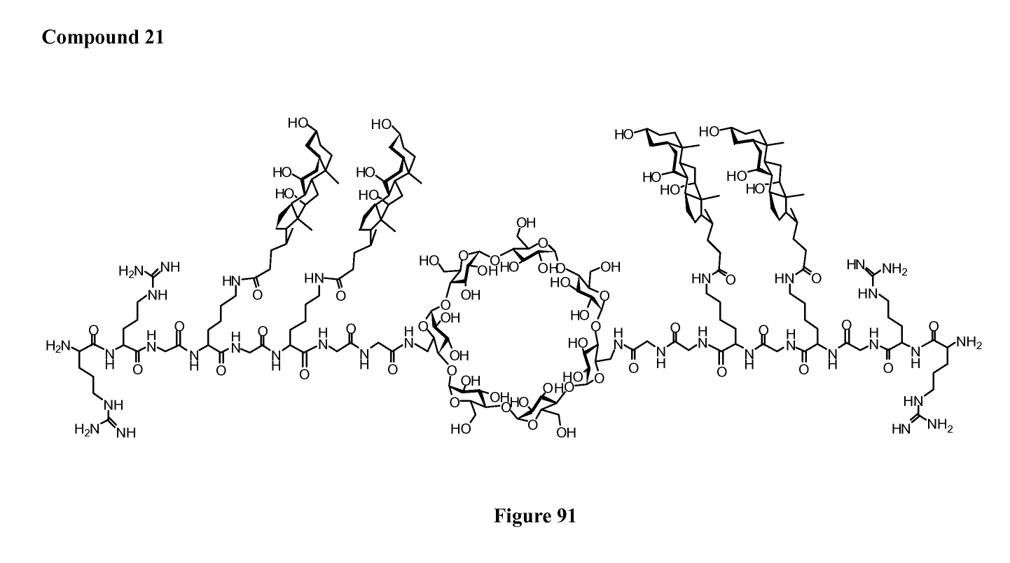

FIG. 92 presents the structure of compound 22 from Example 10.

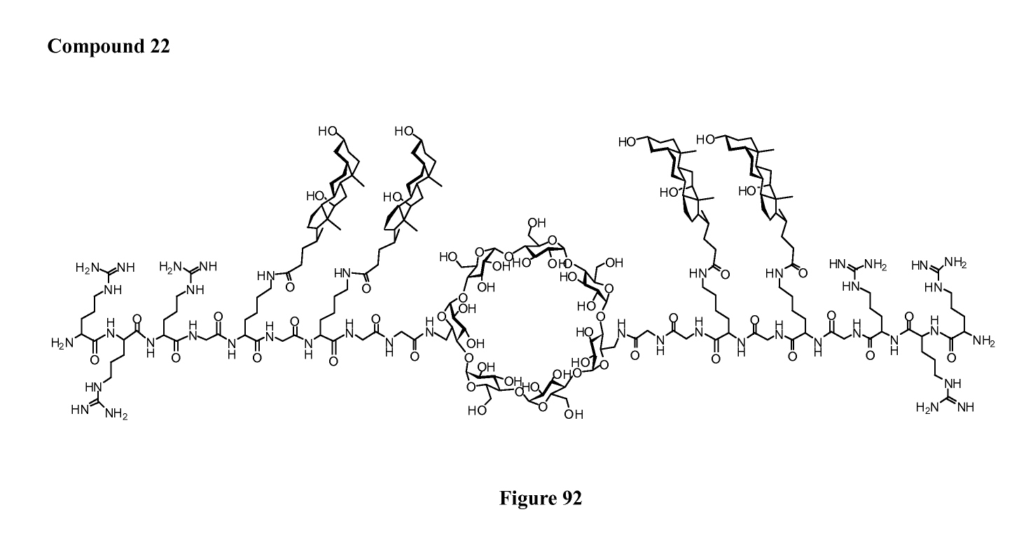

FIG. 93 presents the structure of compound 23 from Example 10.

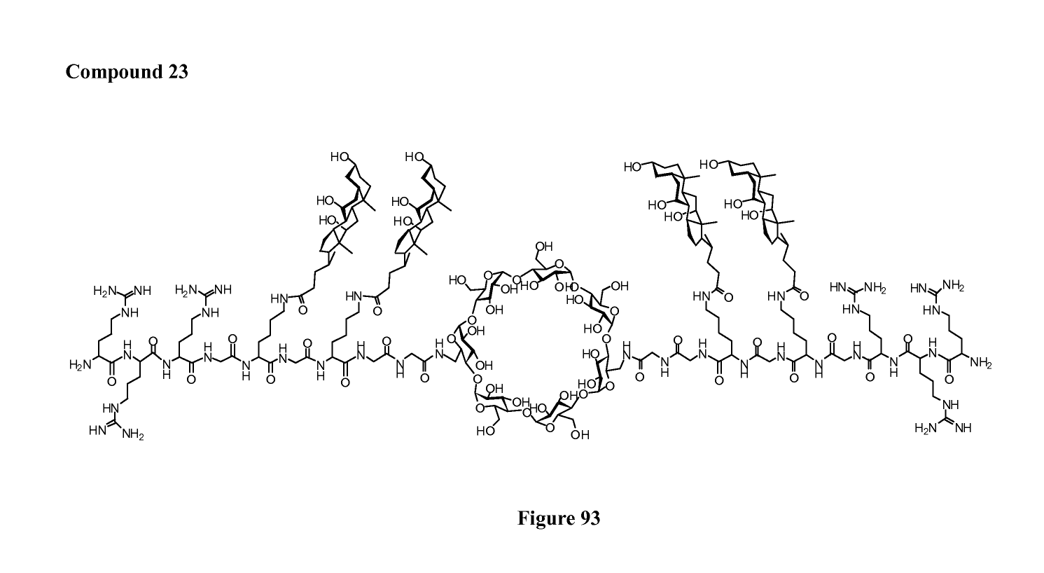

FIG. 94 presents the structure of compound 24 from Example 10.

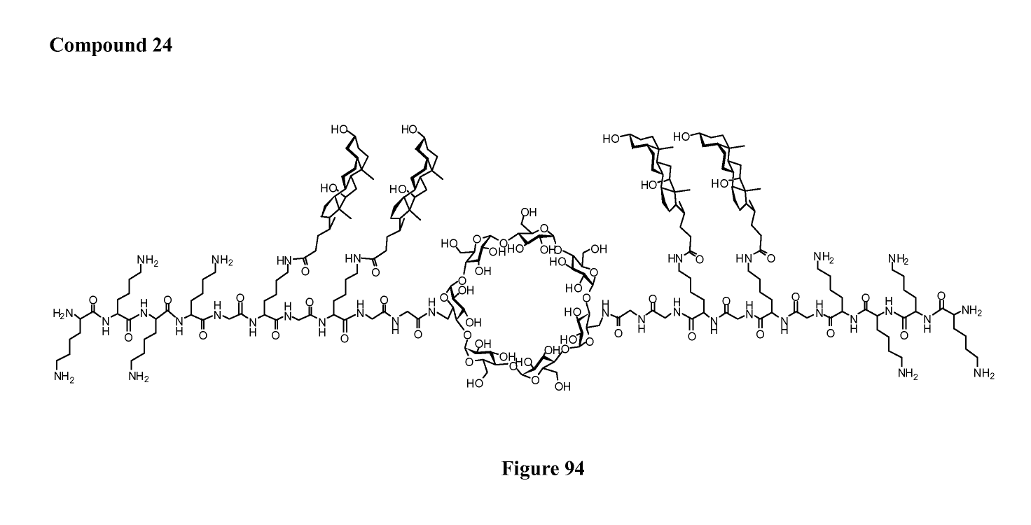

FIG. 95 presents the structure of compound 25 from Example 10.

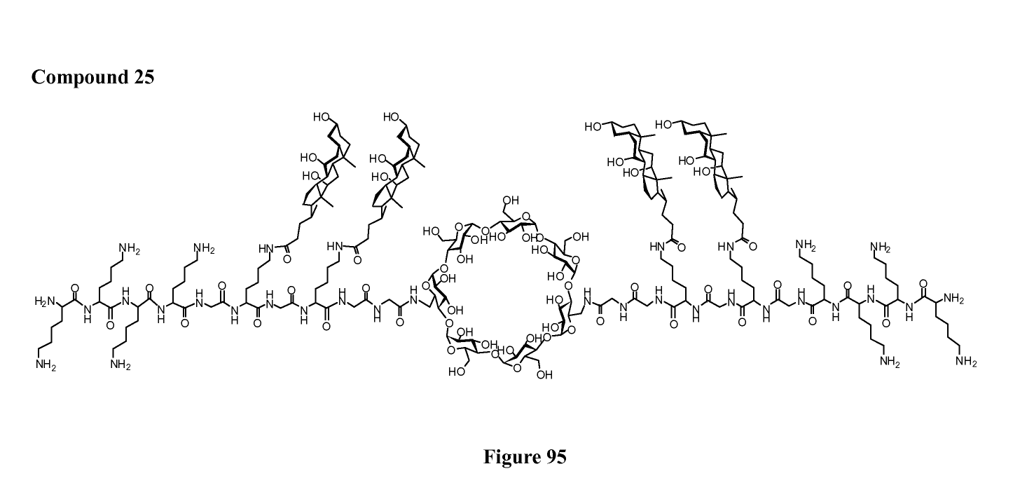

FIG. 96 presents the structure of compound 26 from Example 10.

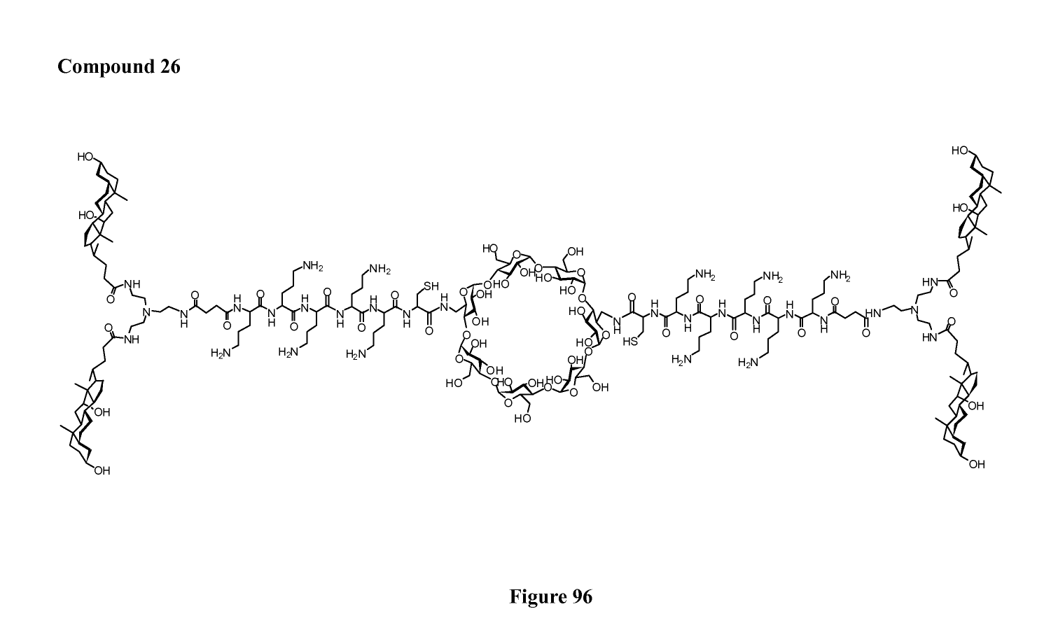

FIG. 97 presents the structure of compound 27 from Example 10.

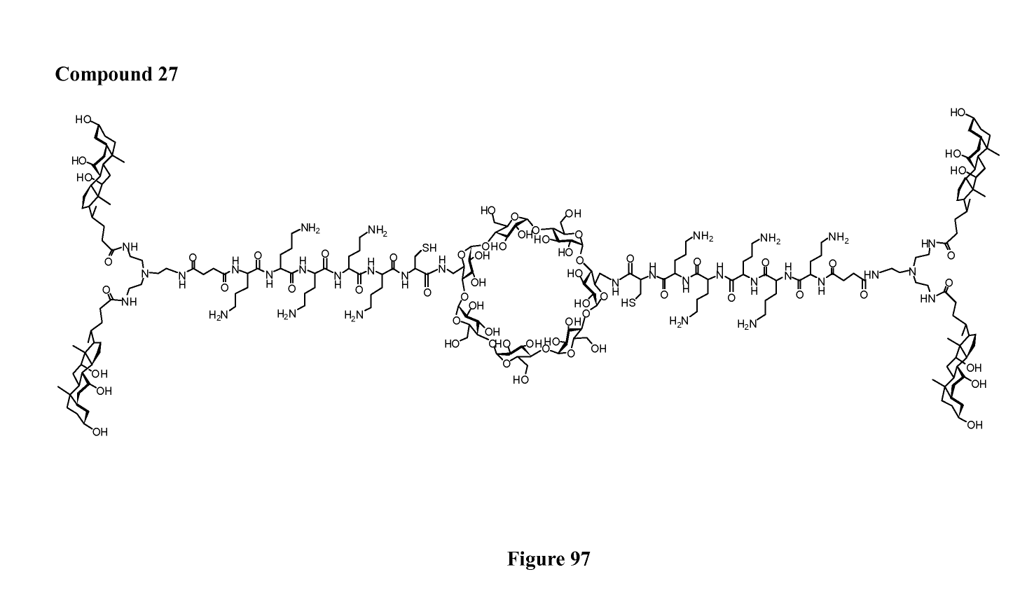

FIG. 98 presents the structure of compound 28 from Example 10.

FIG. 99 presents the structure of compound 29 from Example 10.

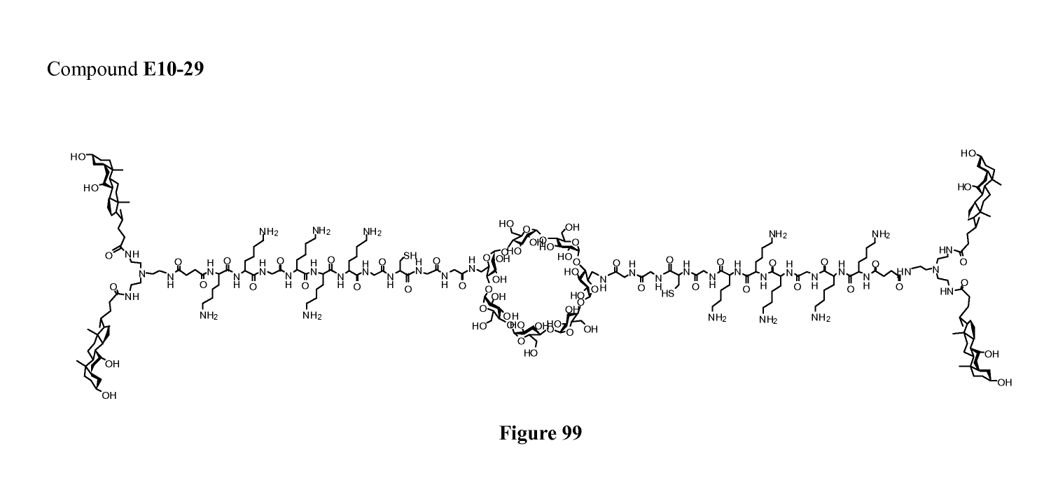

FIG. 100 presents the structure of compound 30 from Example 10.

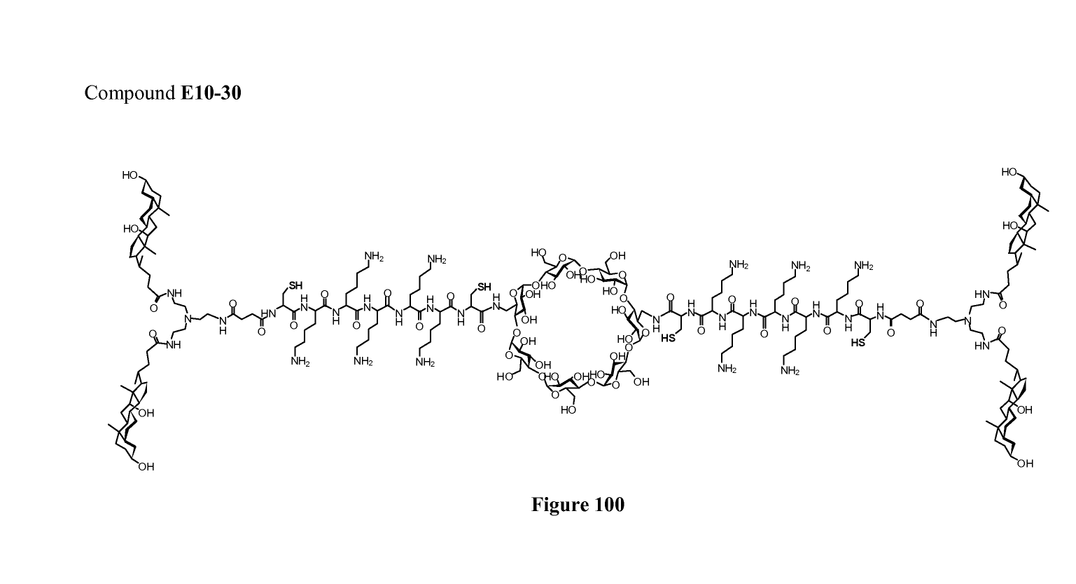

FIG. 101 presents the structure of compound 31 from Example 10.

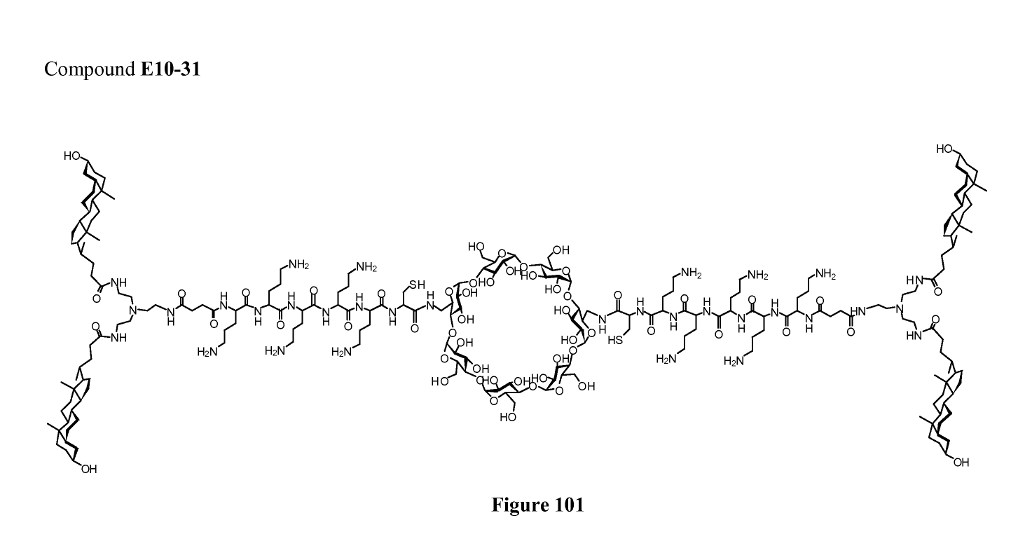

FIG. 102 presents the structure of compound 32 from Example 10.

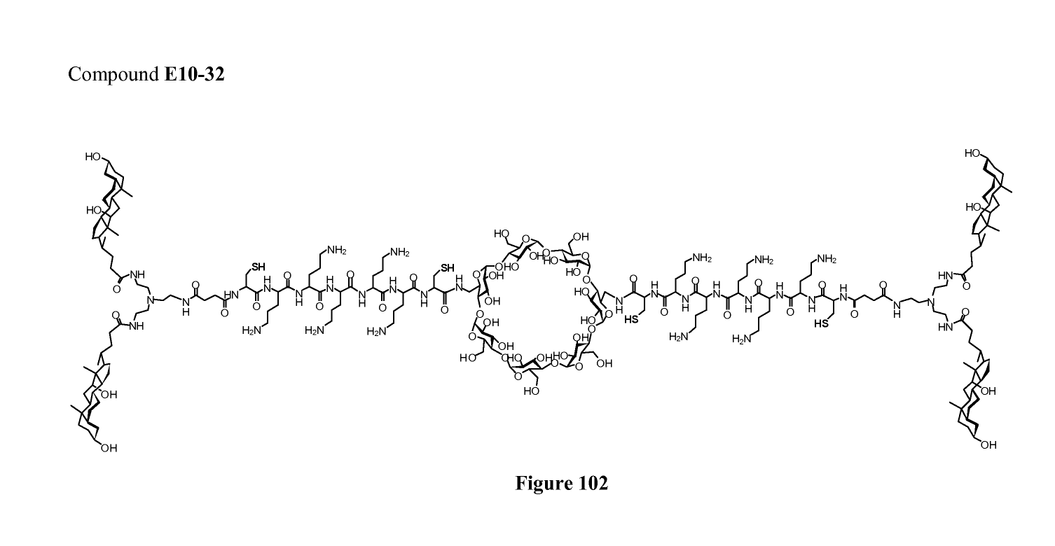

FIG. 103 presents the structure of compound 33 from Example 10.

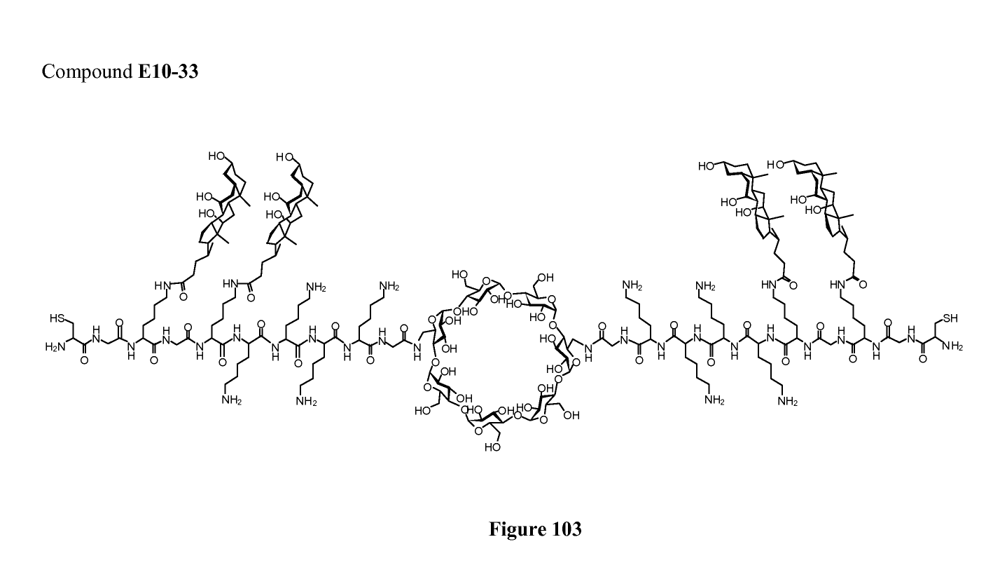

FIG. 104 presents the structure of compound 34 from Example 10.

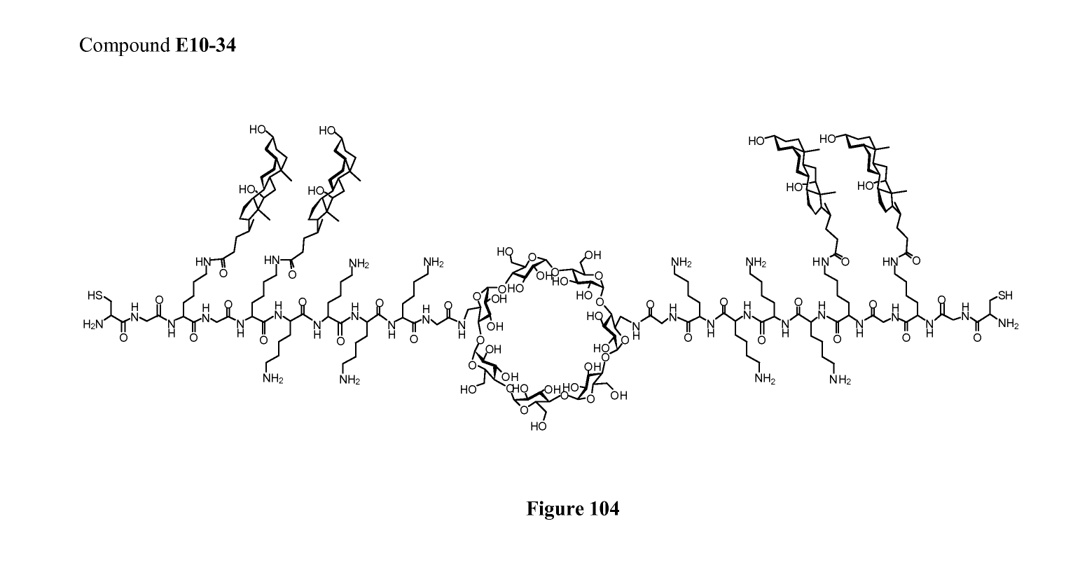

FIG. 105 presents the structure of compound 36 from Example 10.

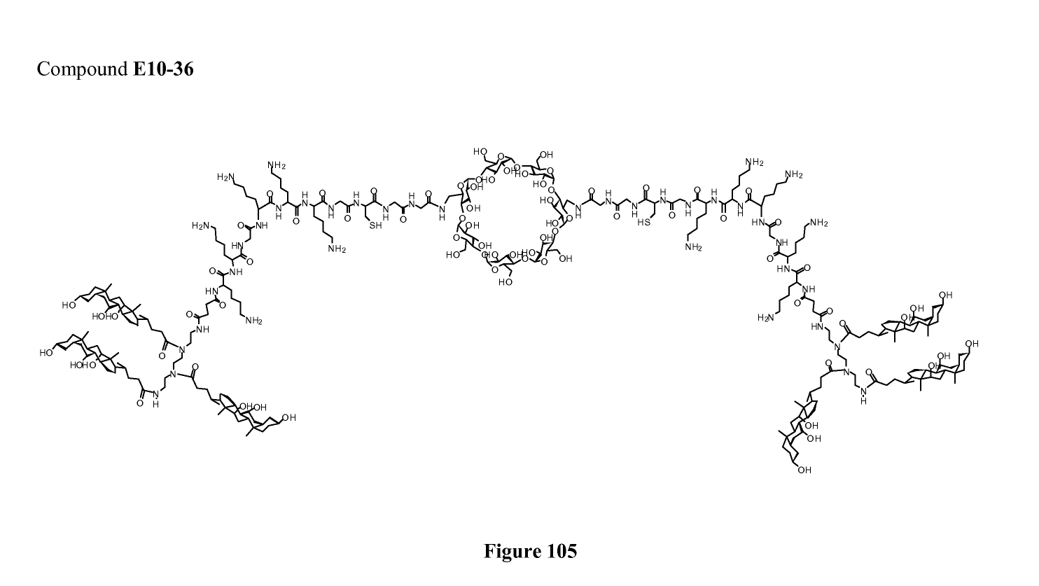

FIG. 106 presents the structure of compound 37 from Example 10.

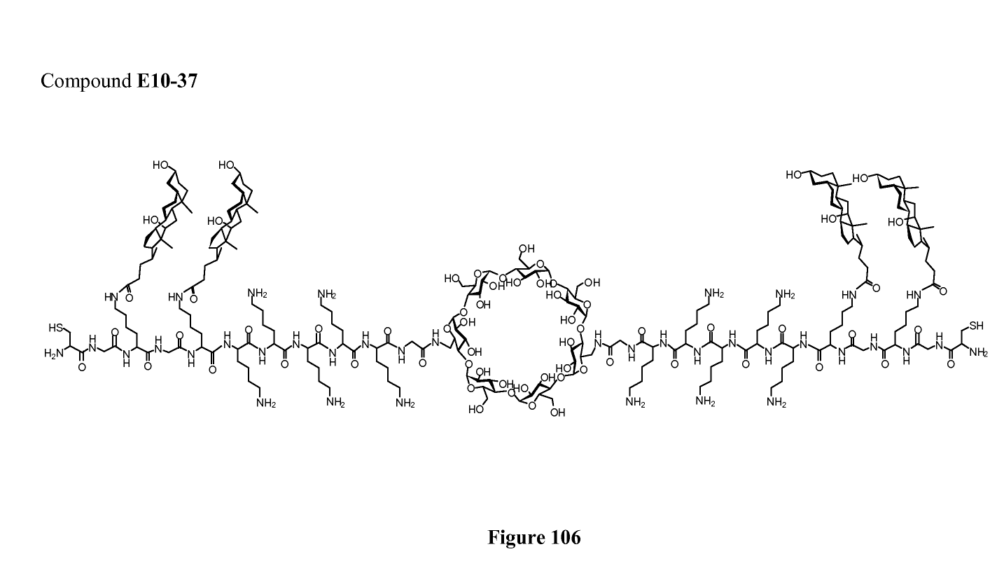

FIG. 107 presents the structure of compound 38 from Example 10.

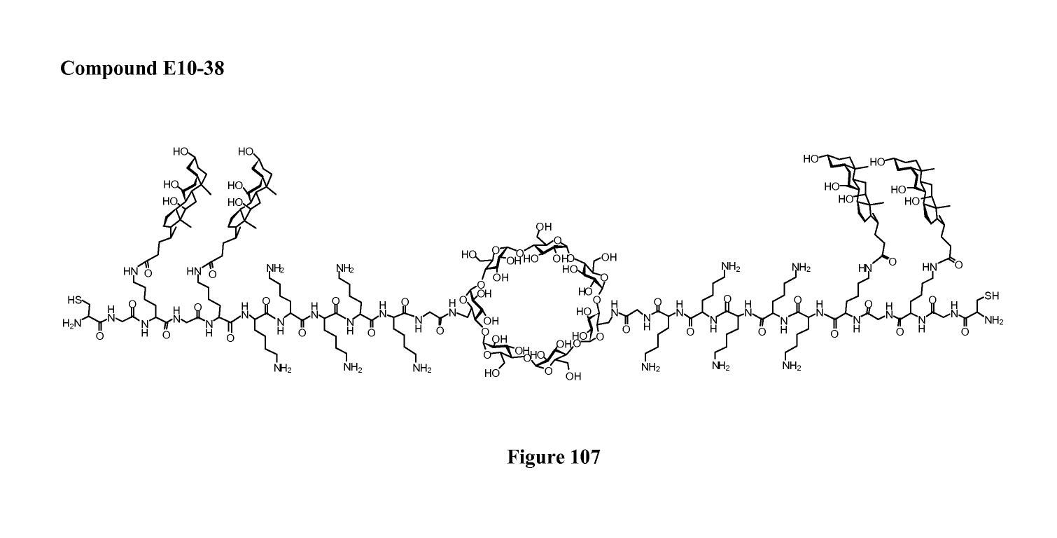

FIG. 108 presents the structure of compound 39 from Example 10.

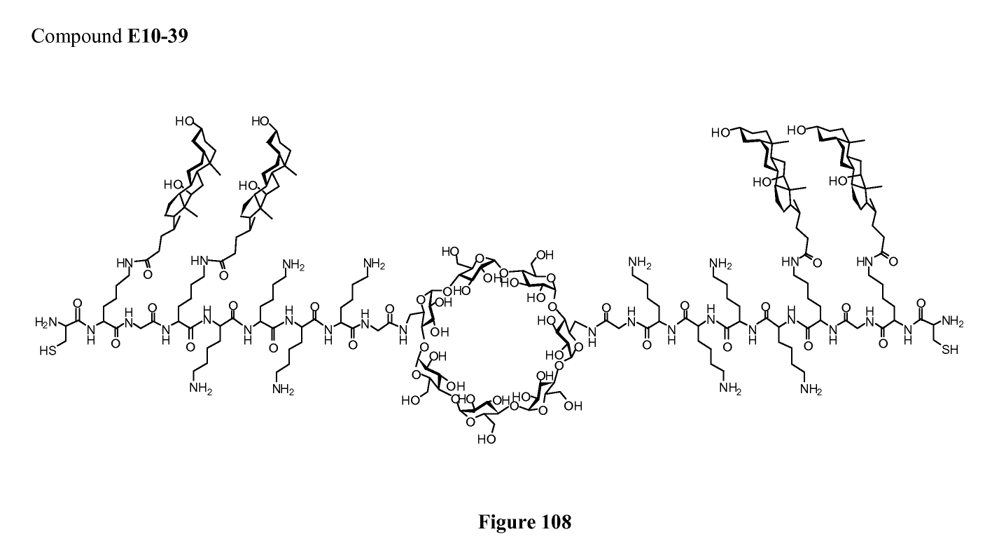

FIG. 109 presents the structure of compound 40 from Example 10.

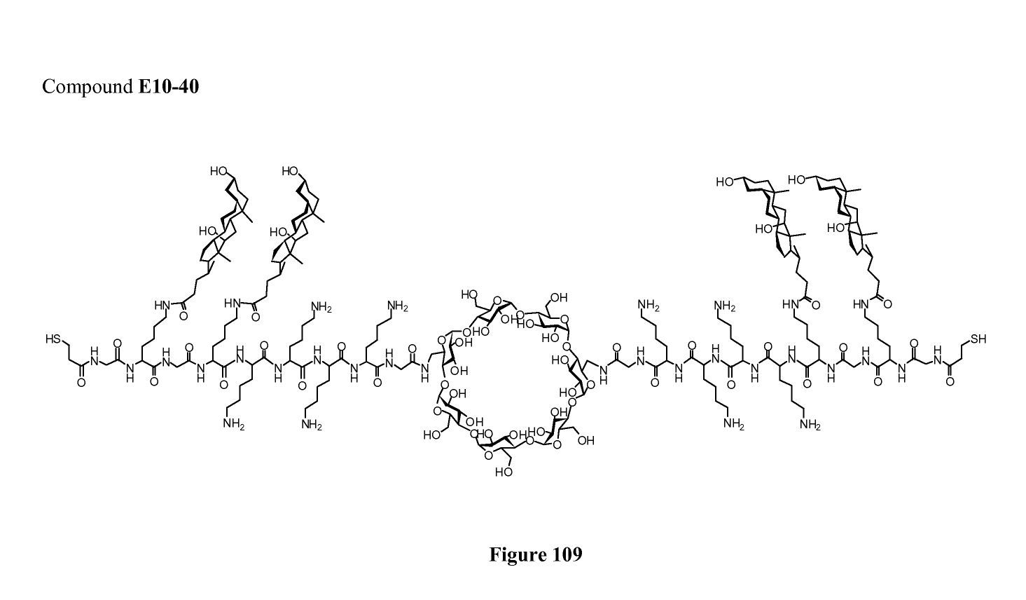

FIG. 110 presents the structure of compound 41 from Example 10.

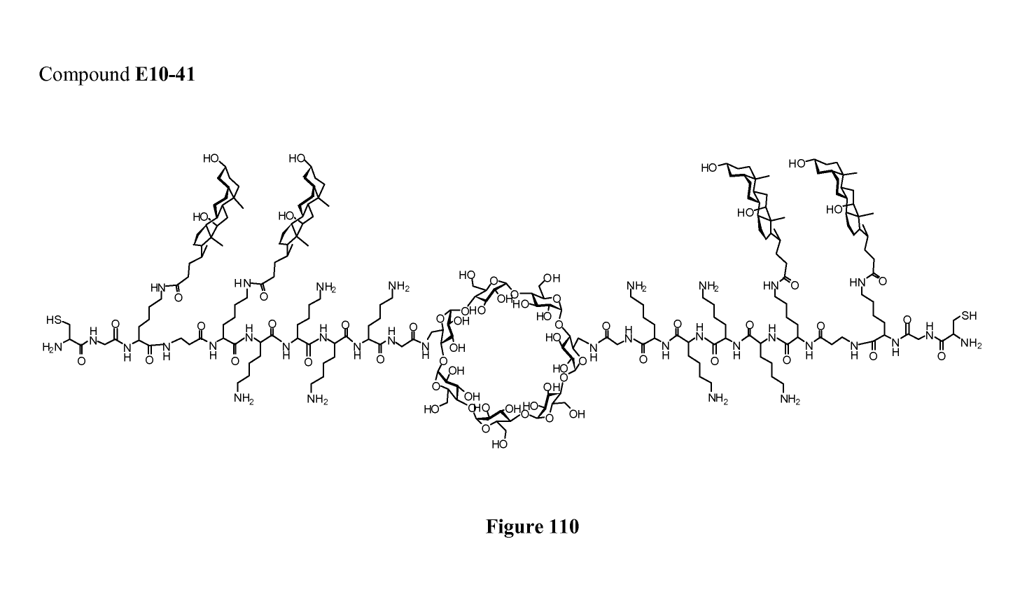

FIG. 111 presents the structure of compound 42 from Example 10.

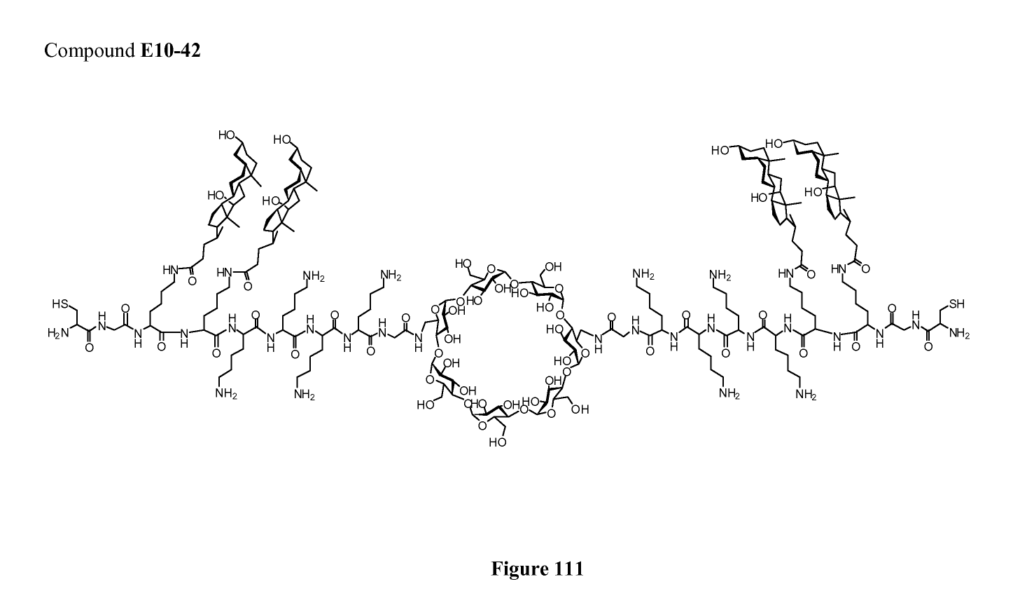

FIG. 112 presents the structure of compound 43 from Example 10.

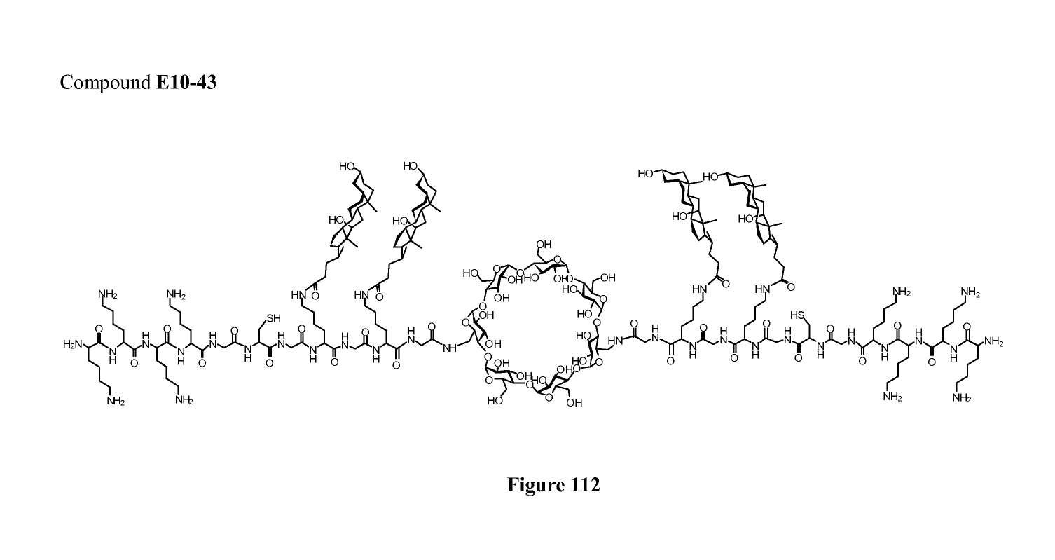

FIG. 113 presents the structure of compound 44 from Example 10.

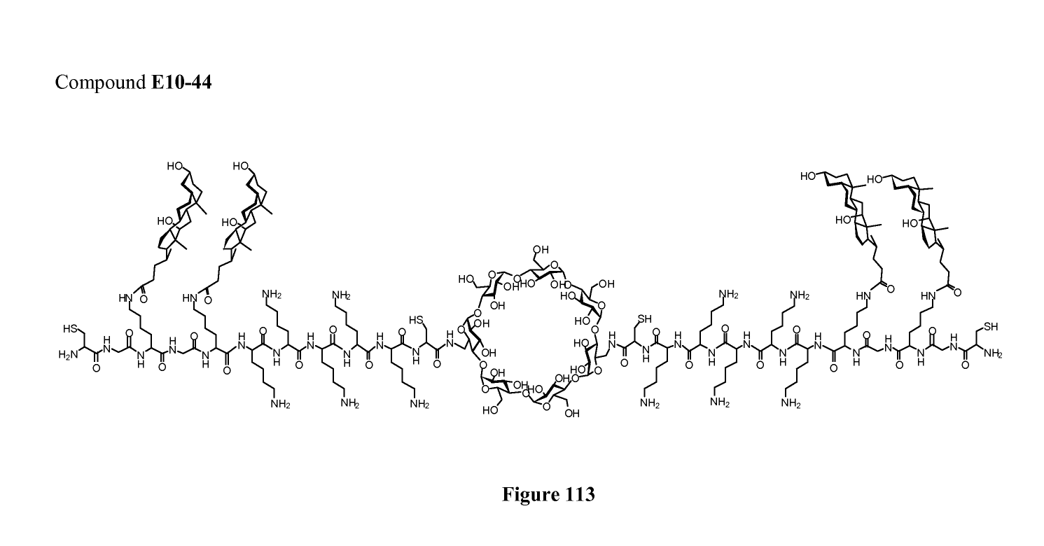

FIG. 114 presents the structure of compound 45 from Example 10.

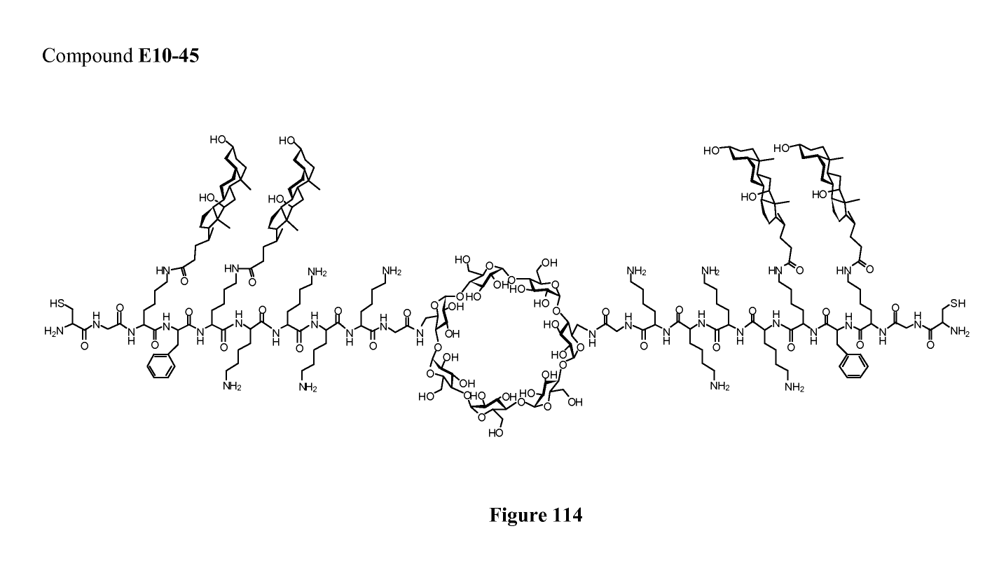

FIG. 115 presents the structure of compound 46 from Example 10.

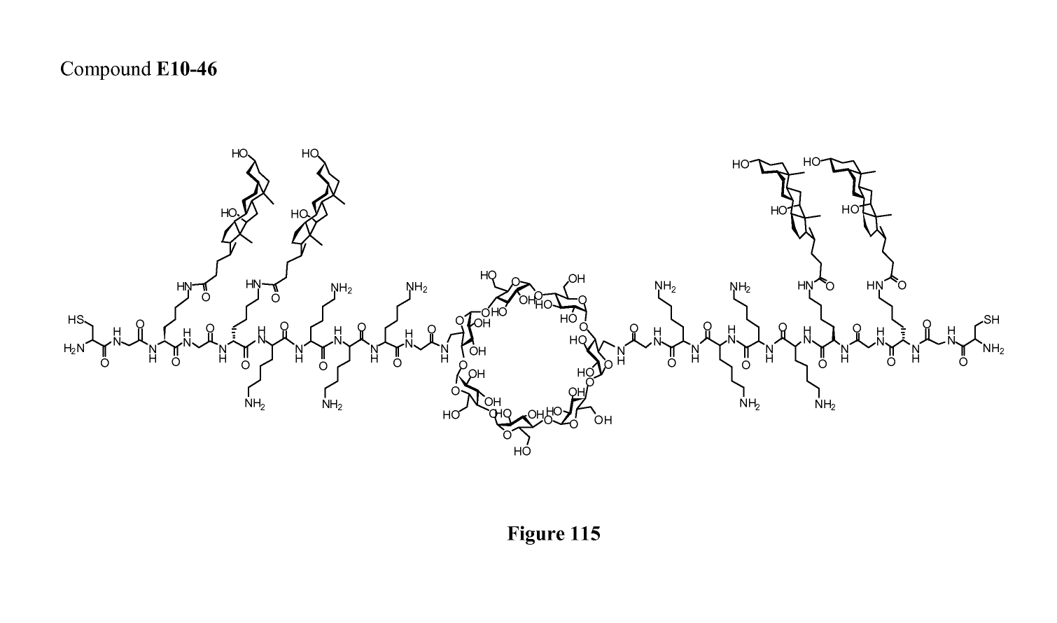

FIG. 116 presents the structure of compound 47 from Example 10.

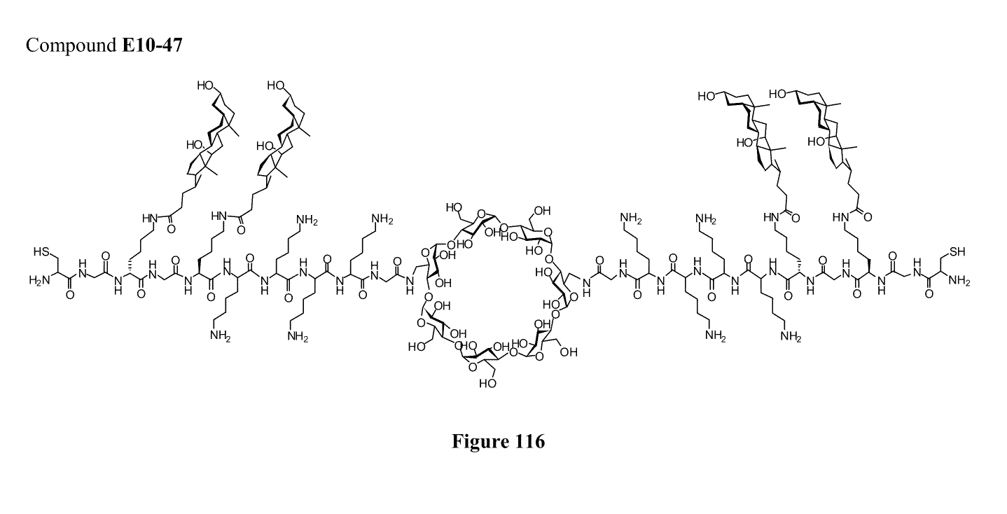

FIG. 117 presents the structure of compound 48 from Example 10.

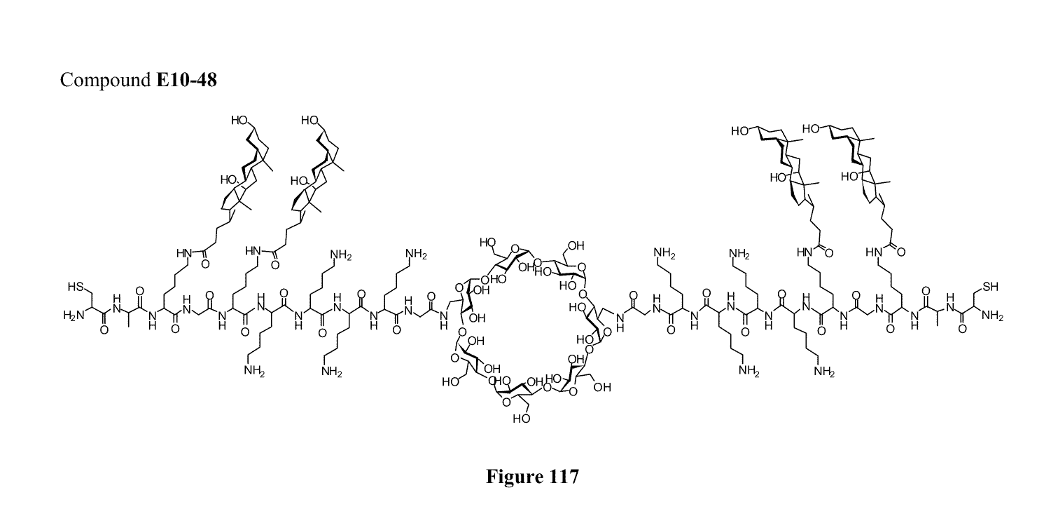

FIG. 118 presents the structure of compound 49 from Example 10.

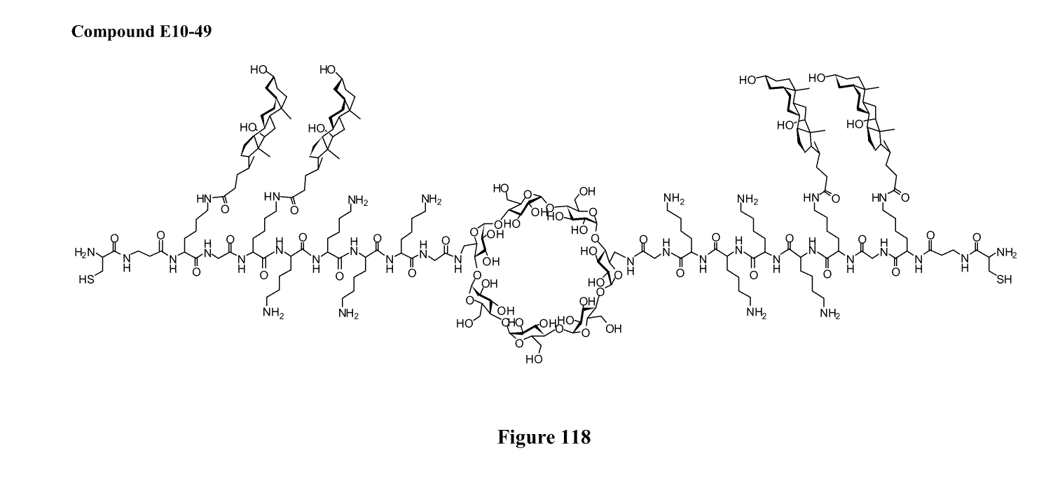

FIG. 119 presents the structure of compound 50 from Example 10.

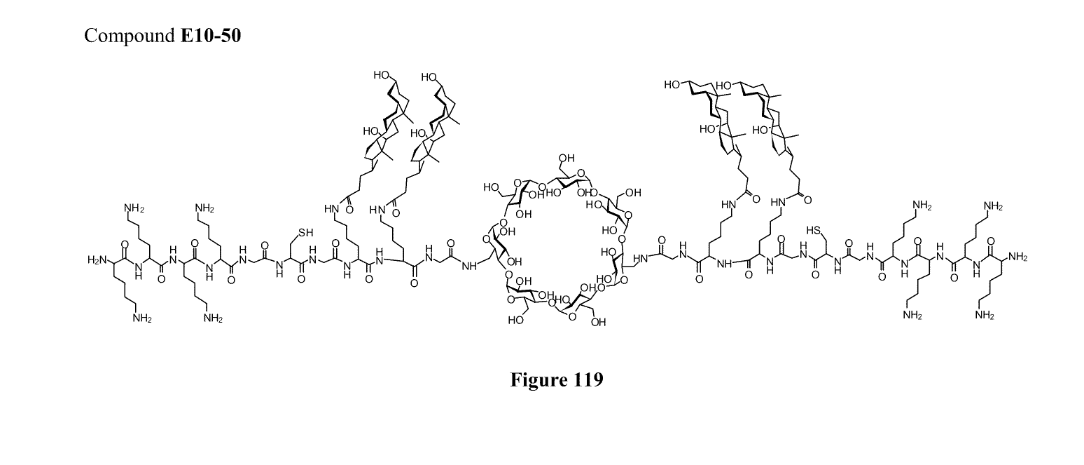

FIG. 120 presents the structure of compound 51 from Example 10.

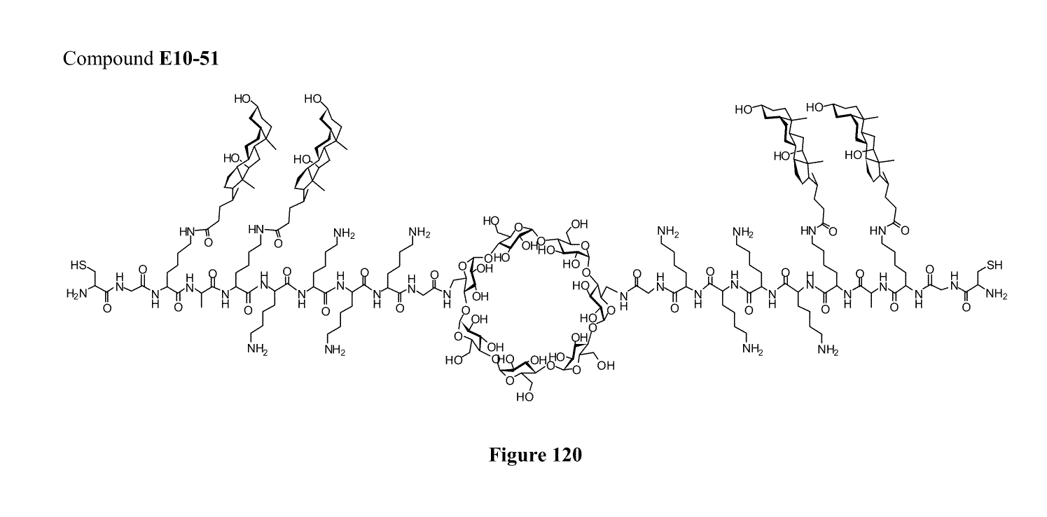

FIG. 121 presents the structure of compound 52 from Example 10.

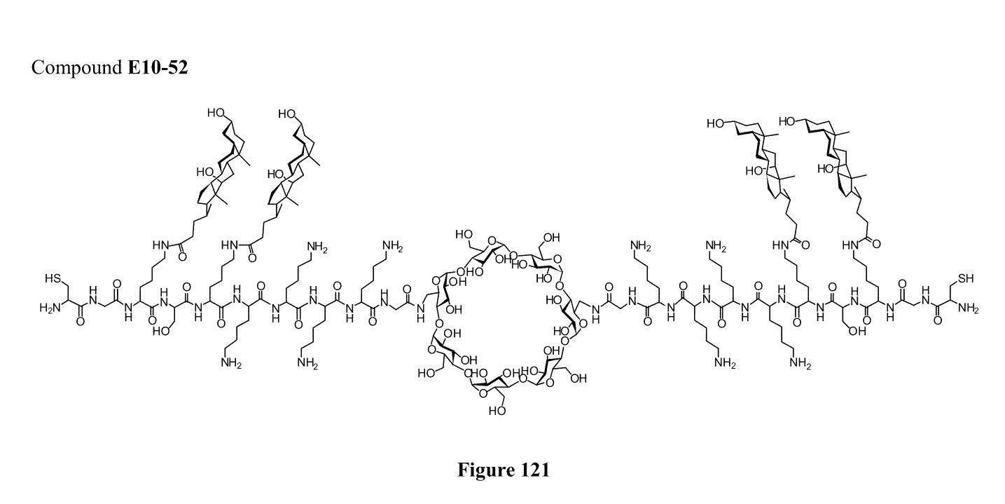

FIG. 122 presents the structure of compound 53 from Example 10.

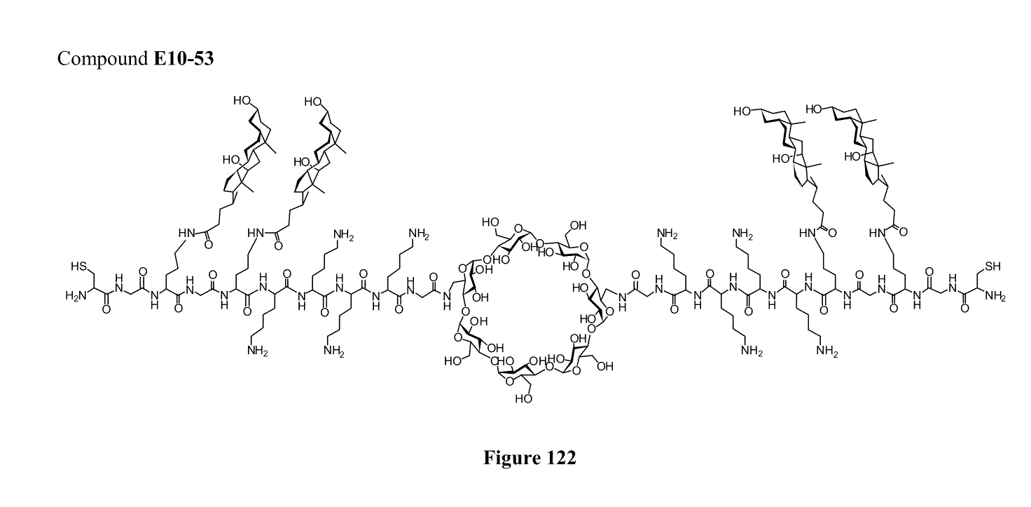

FIG. 123 presents the structure of compound 54 from Example 10.

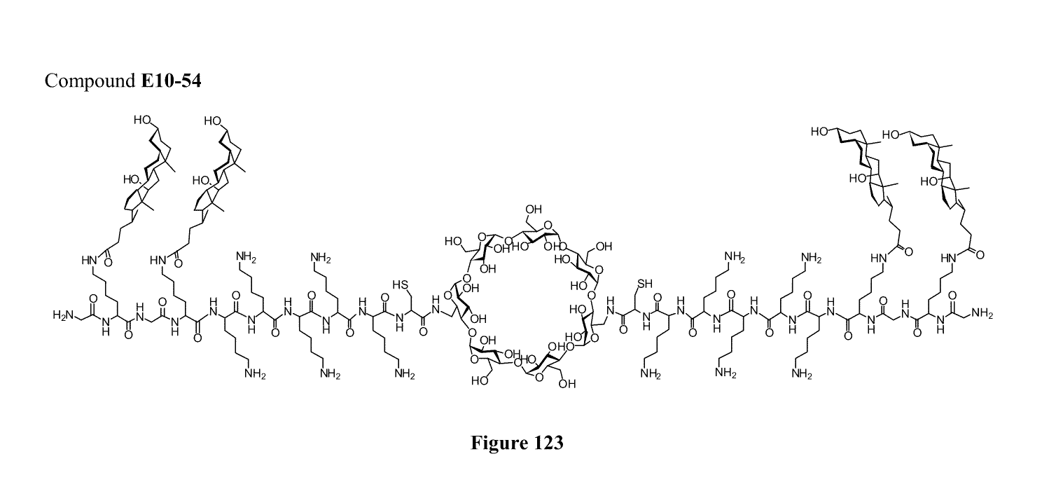

FIG. 124 presents the structure of compound 55 from Example 10.

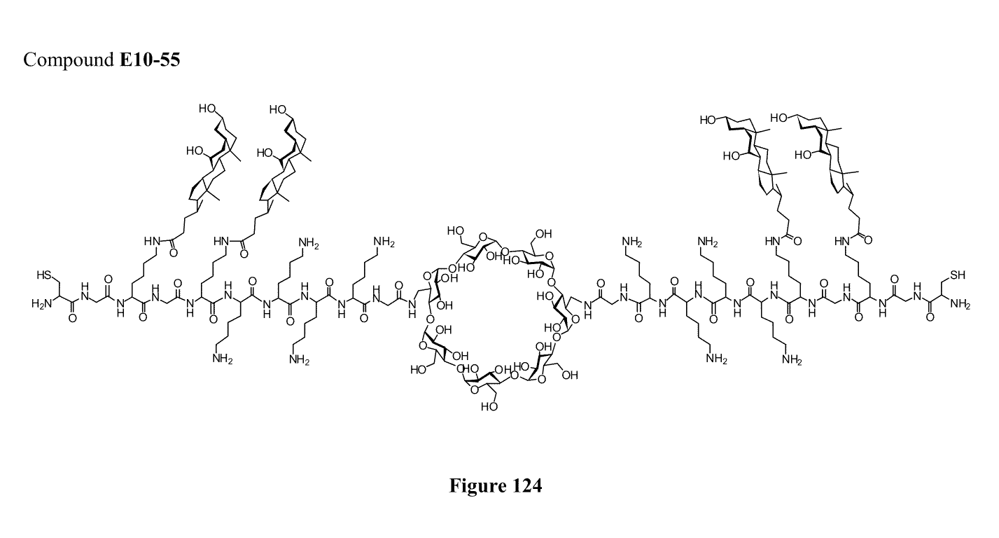

FIG. 125 presents the structure of compound 56 from Example 10.

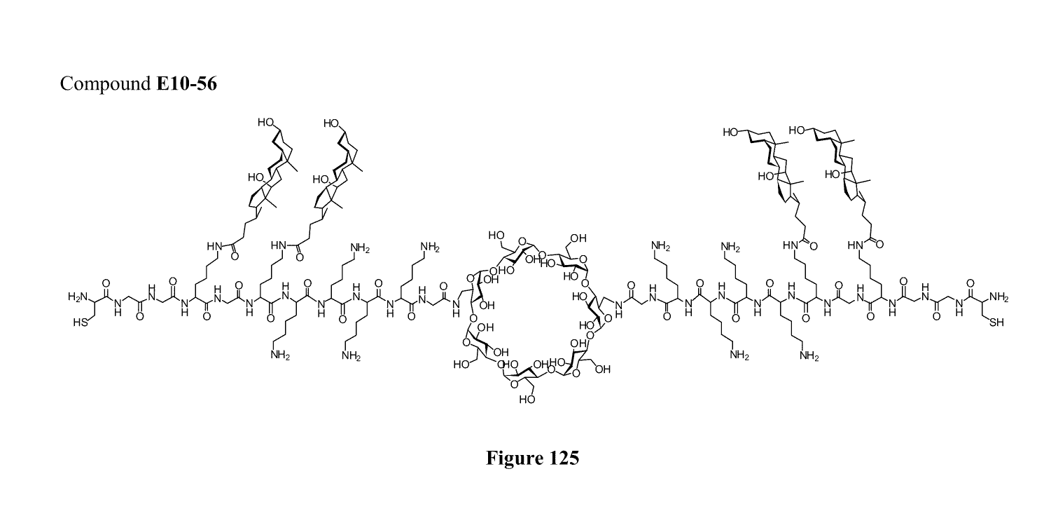

FIG. 126 presents the structure of compound 57 from Example 10.

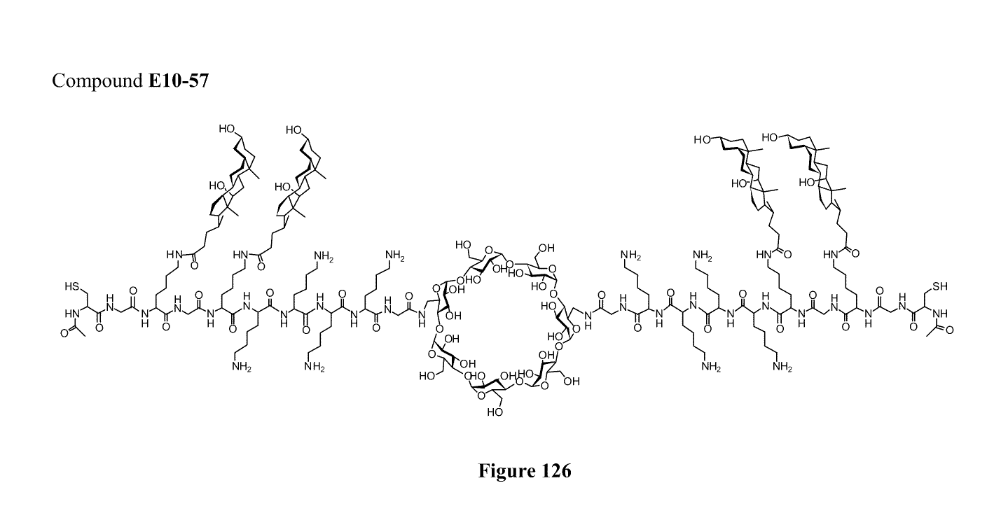

FIG. 127 presents the structure of compound 58 from Example 10.

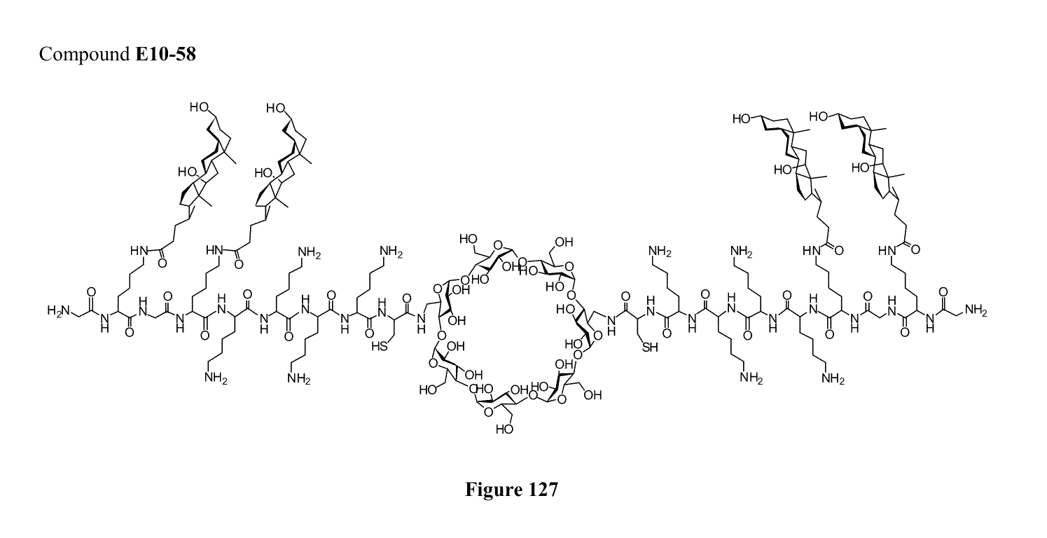

FIG. 128 presents the structure of compound 59 from Example 10.

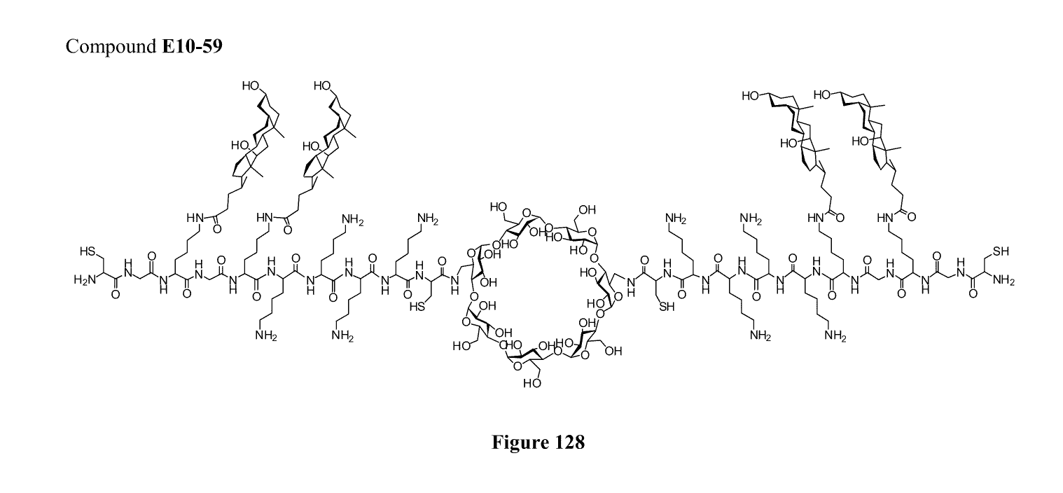

FIG. 129 presents the structure of compound 60 from Example 10.

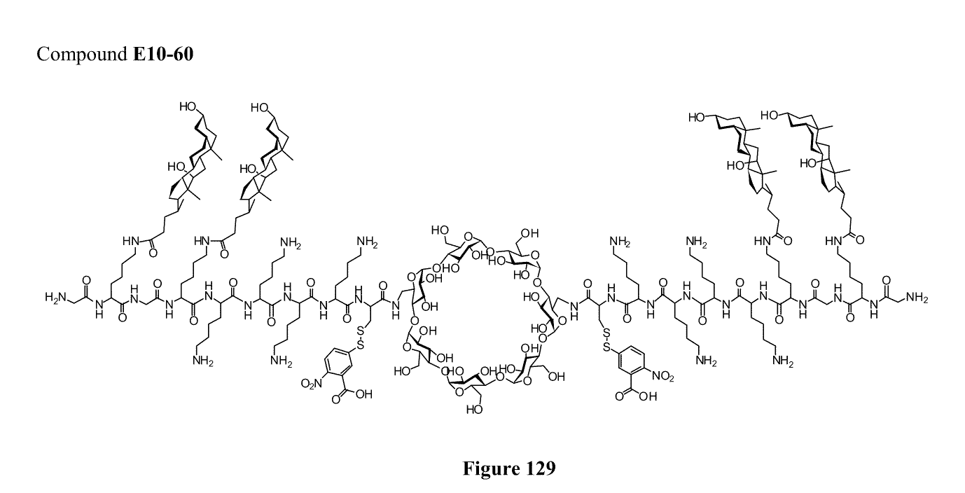

FIG. 130 presents the structure of compound 61 from Example 10.

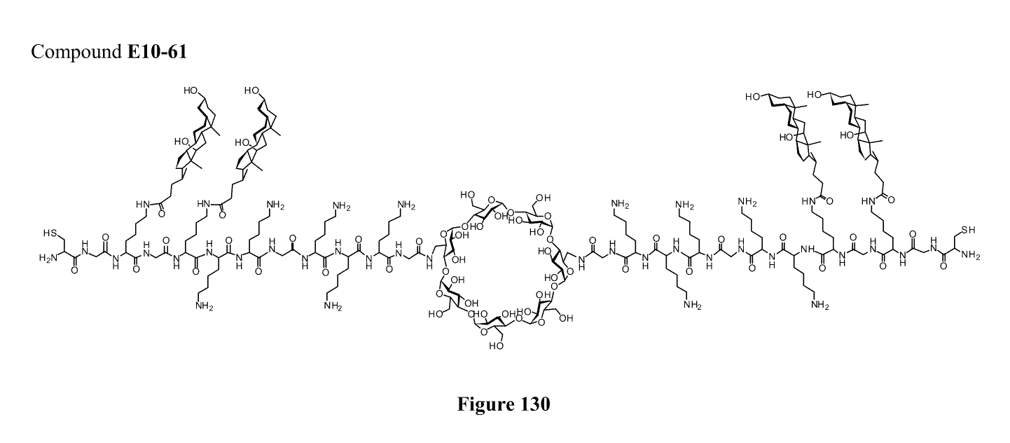

FIG. 131 presents the structure of compound 62 from Example 10.

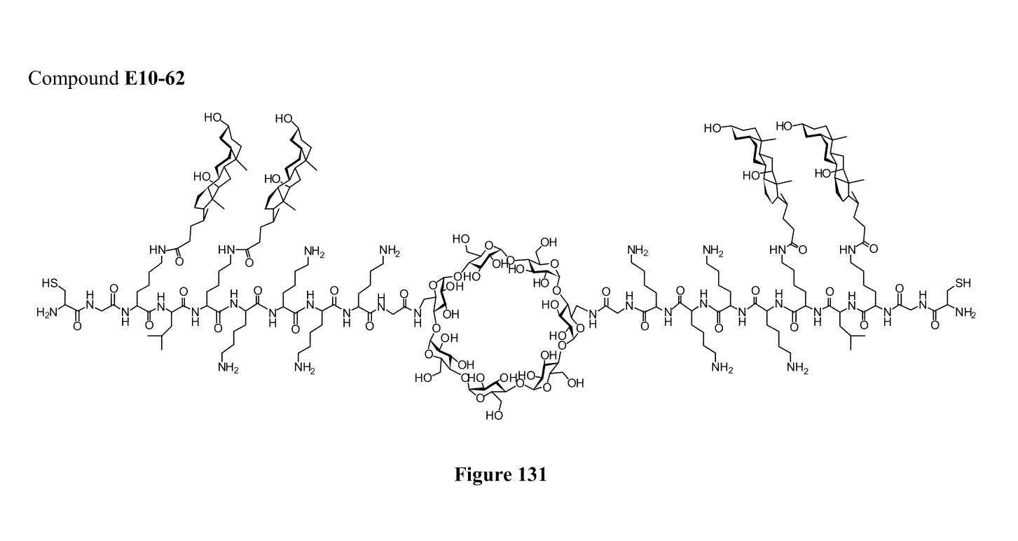

FIG. 132 presents the structure of compound 63 from Example 10.

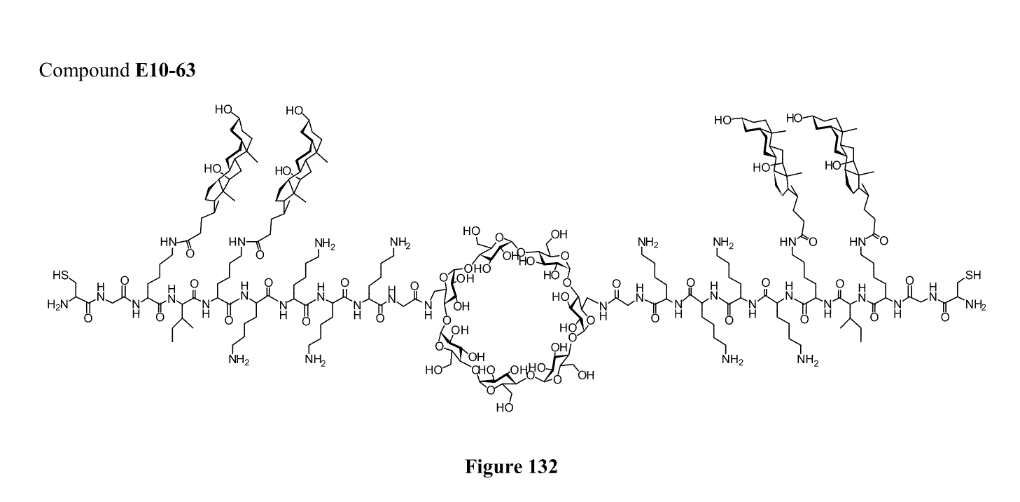

FIG. 133 presents the structure of compound 64 from Example 10.

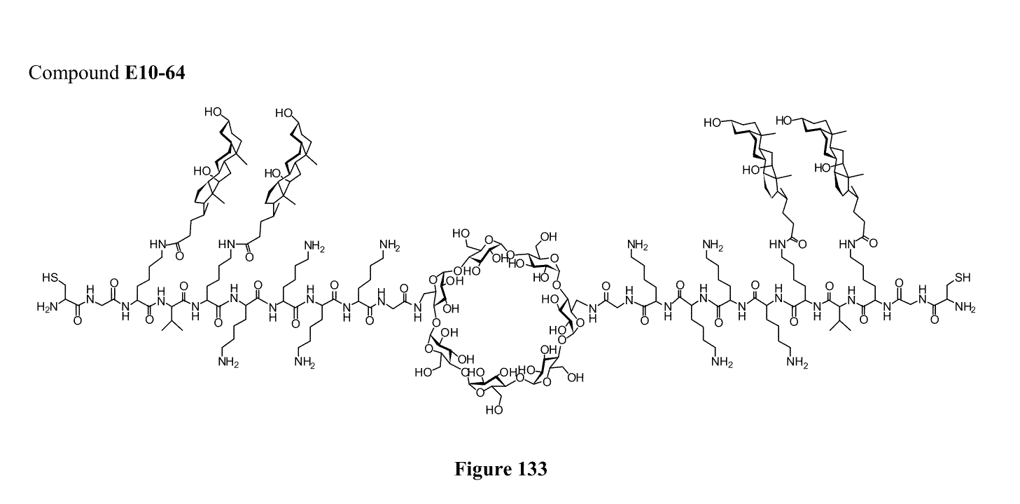

FIG. 134 presents the structure of compound 65 from Example 10.

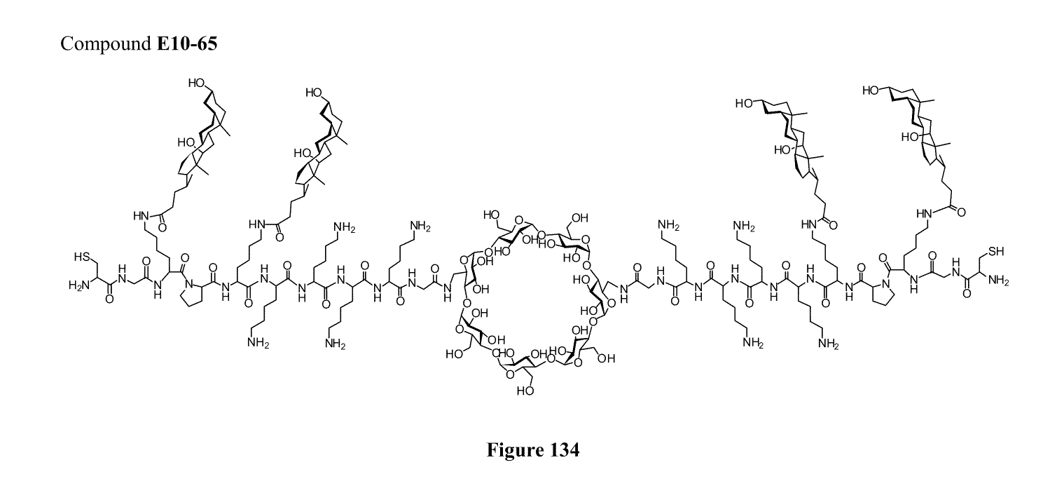

FIG. 135 presents the structure of compound 66 from Example 10.

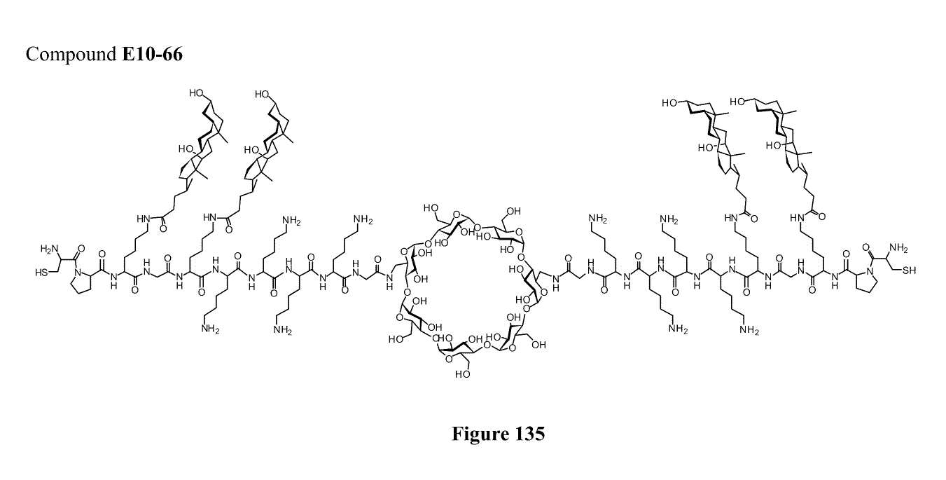

FIG. 136 presents the structure of compound 67 from Example 10.

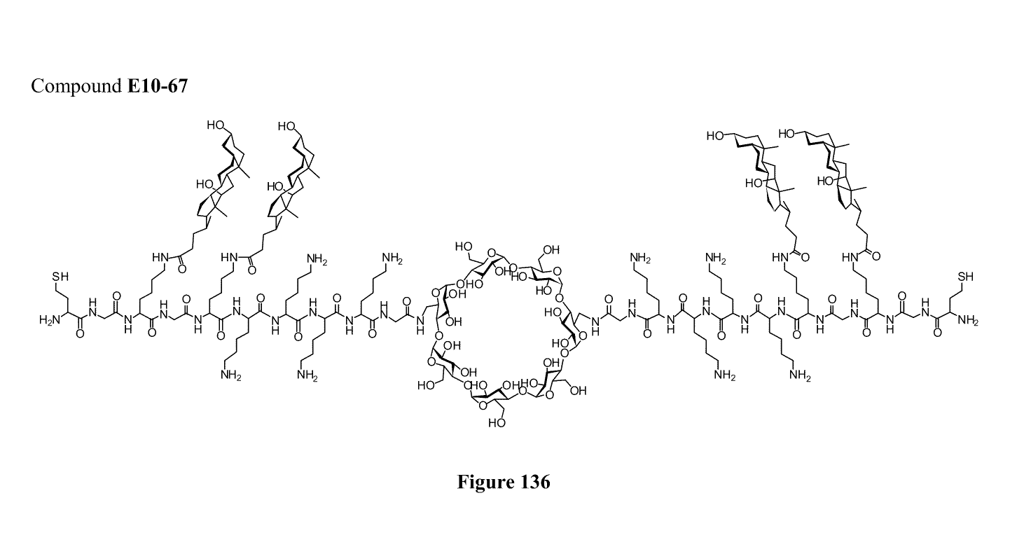

FIG. 137 presents the structure of compound 68 from Example 10.

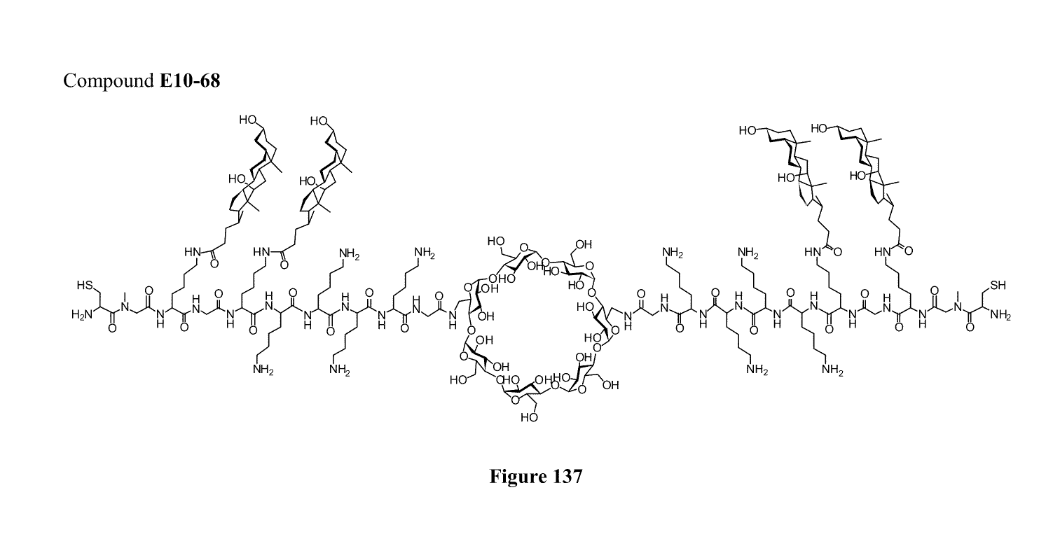

FIG. 138 presents the structure of compound 69 from Example 10.

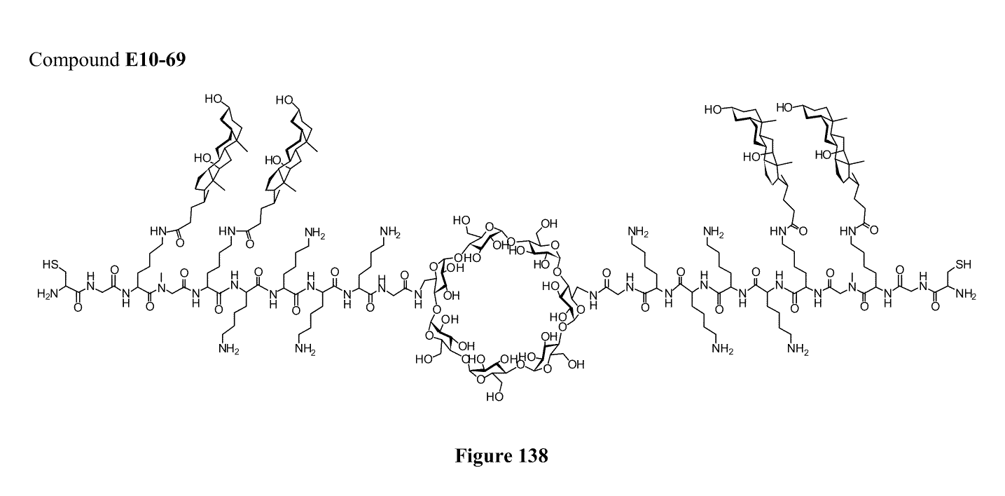

FIG. 139 presents the structure of compound 70 from Example 10.

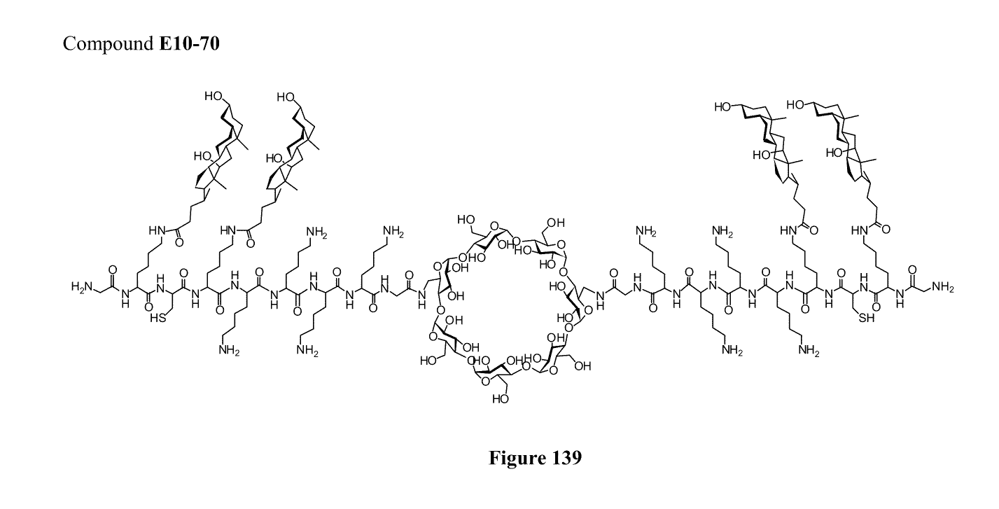

FIG. 140 presents the structure of compound 71 from Example 10.

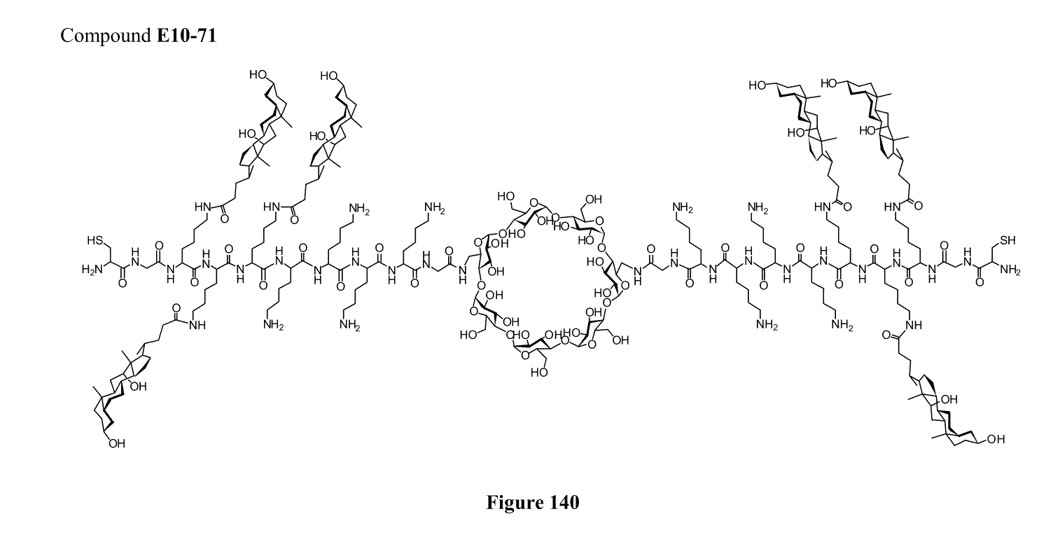

FIG. 141 presents the structure of compound 72 from Example 10.

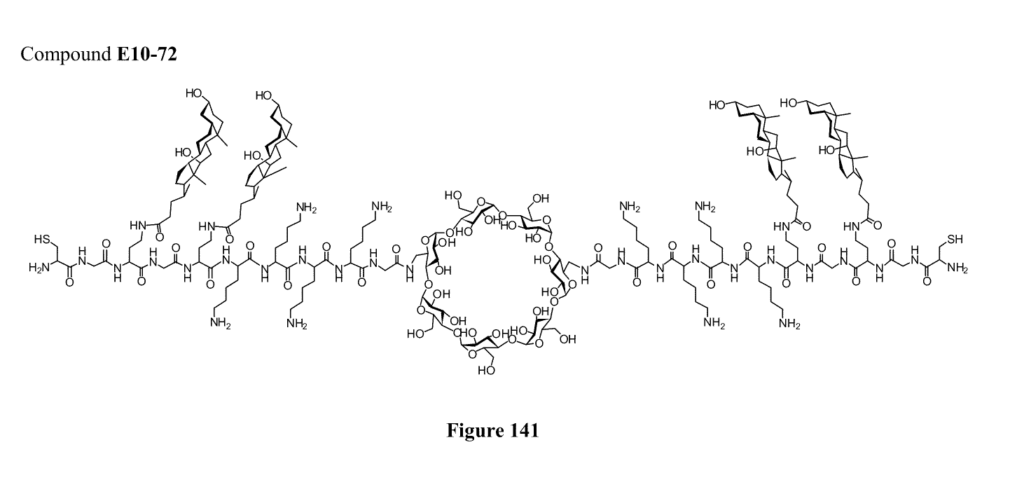

FIG. 142 presents the structure of compound 73 from Example 10.

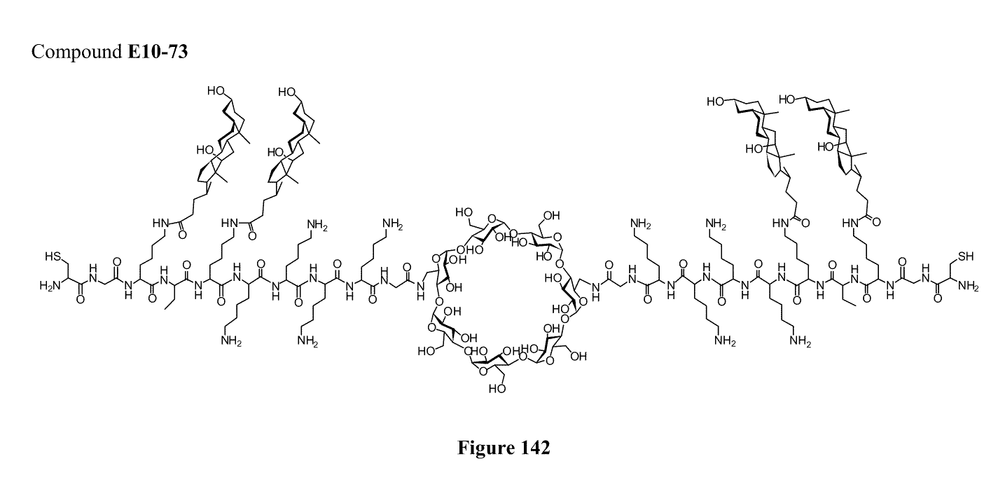

FIG. 143 presents the structure of compound 74 from Example 10.

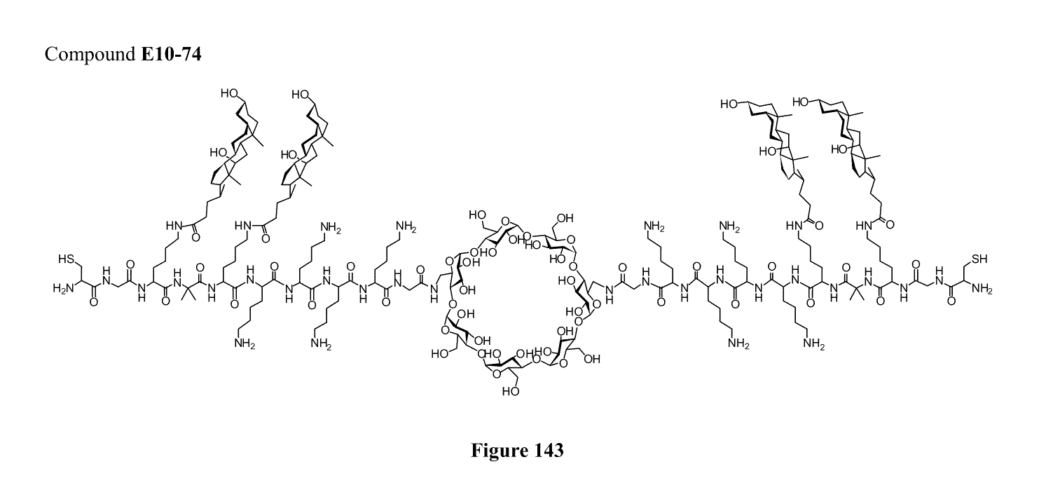

FIG. 144 presents the structure of compound 75 from Example 10.

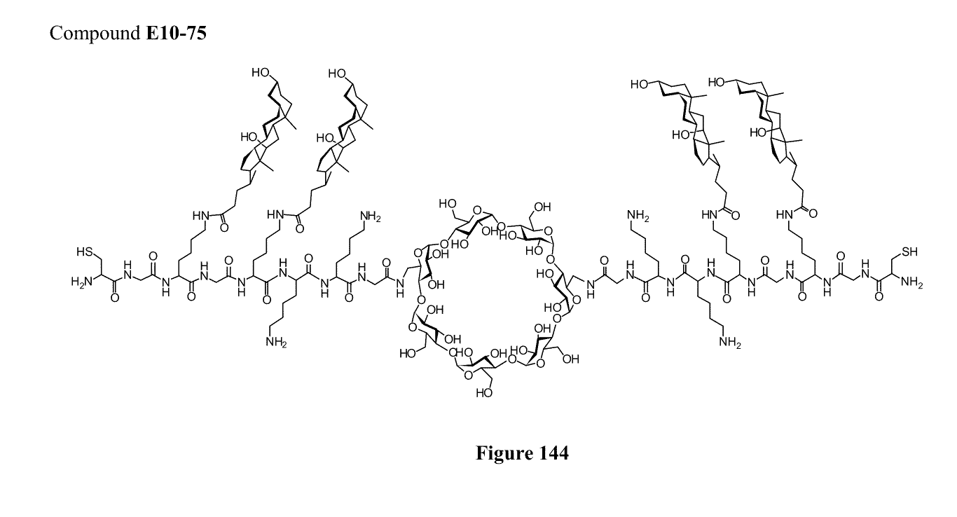

FIG. 145 presents the structure of compound 76 from Example 10.

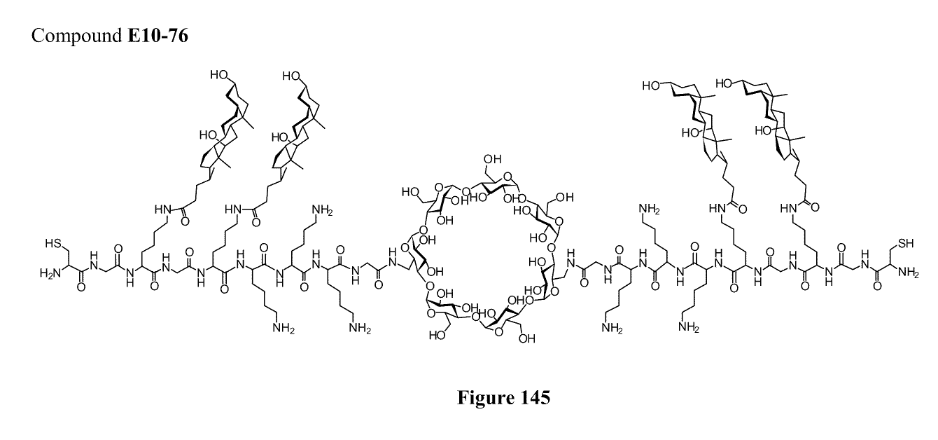

FIG. 146 presents the structure of compound 77 from Example 10.

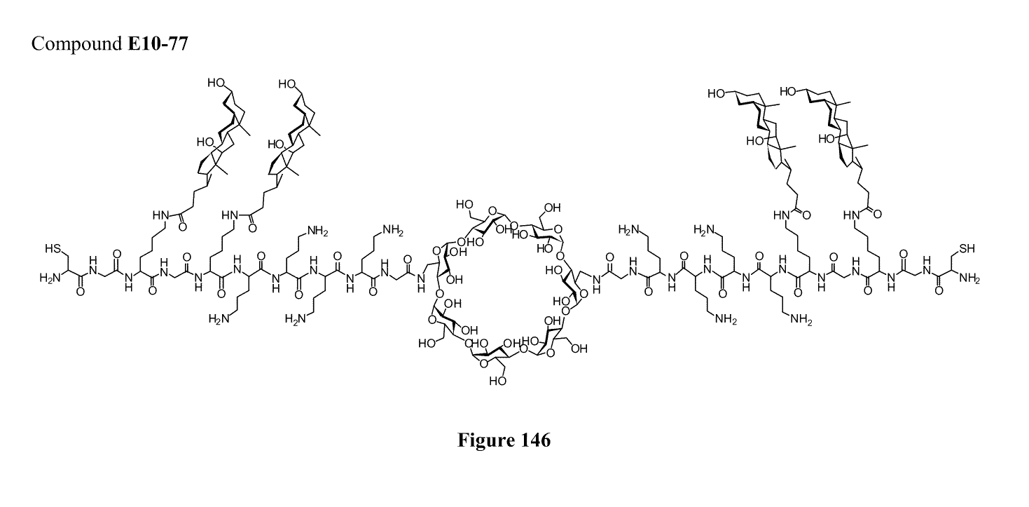

FIG. 147 presents the structure of compound 78 from Example 10.

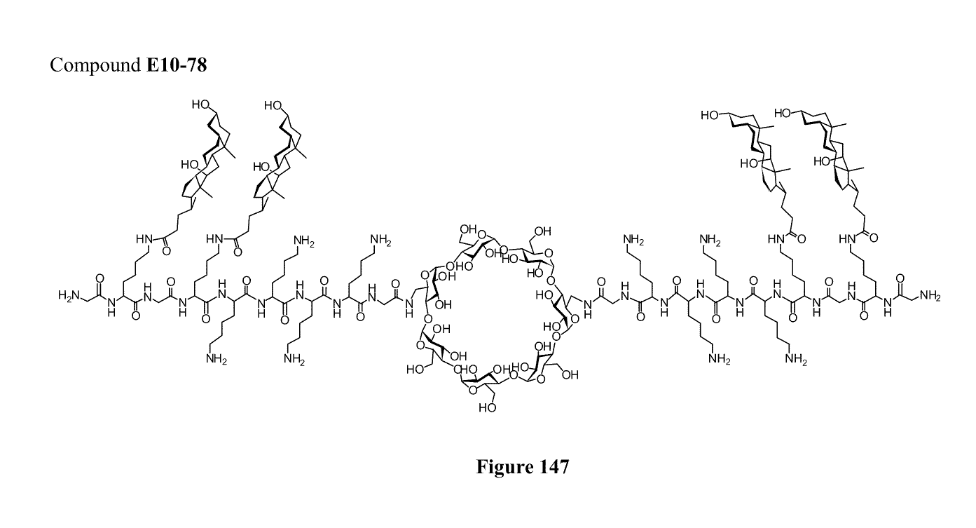

FIG. 148 presents the structure of compound 79 from Example 10.

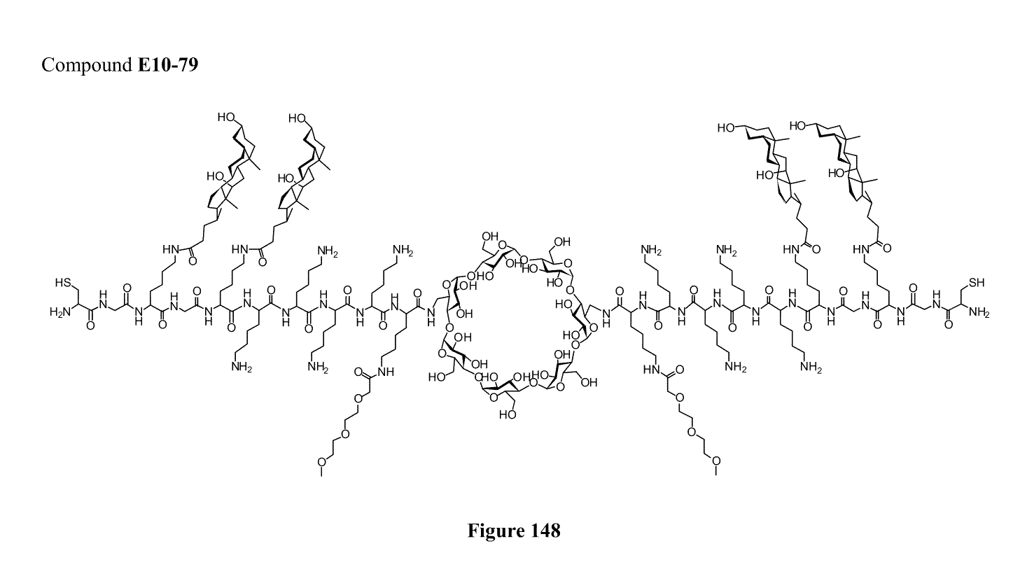

FIG. 149 presents the structure of compound 80 from Example 10.

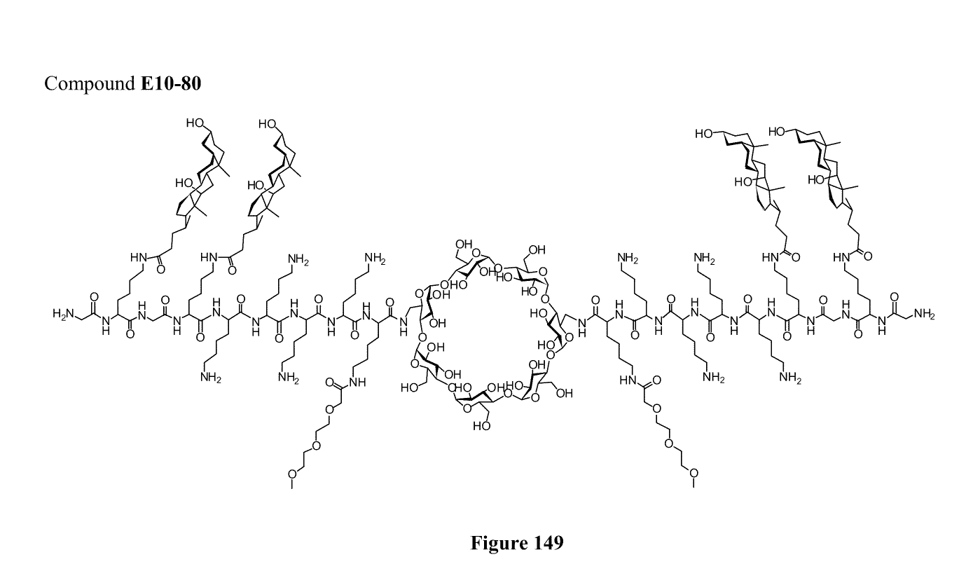

FIG. 150 presents the structure of compound 81 from Example 10.

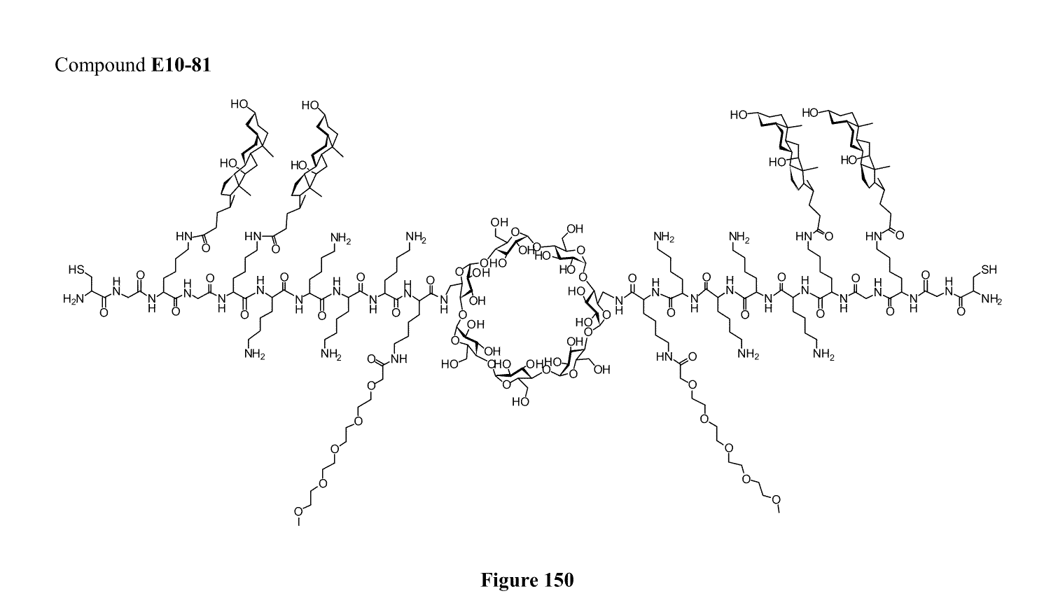

FIG. 151 presents the structure of compound 82 from Example 10.

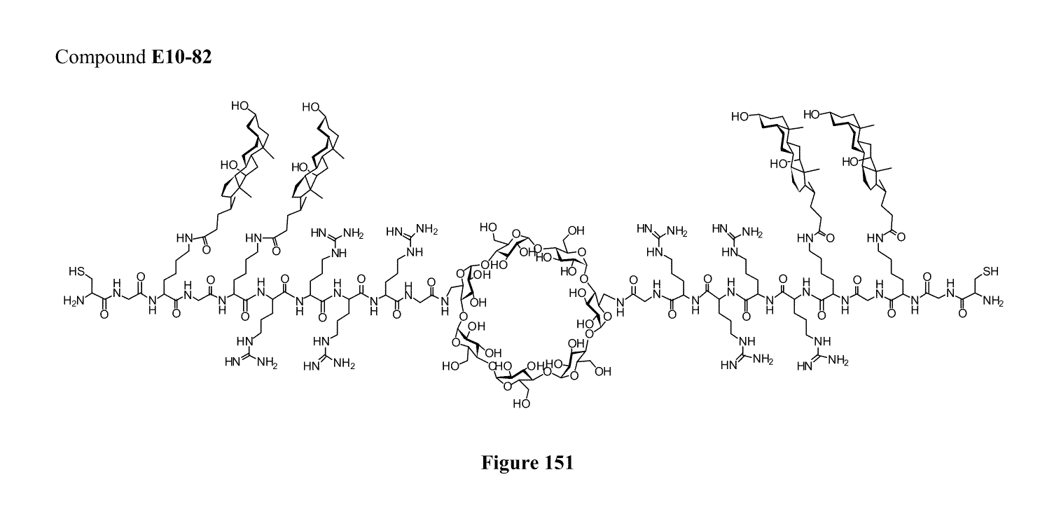

FIG. 152 presents the structure of compound 83 from Example 10.

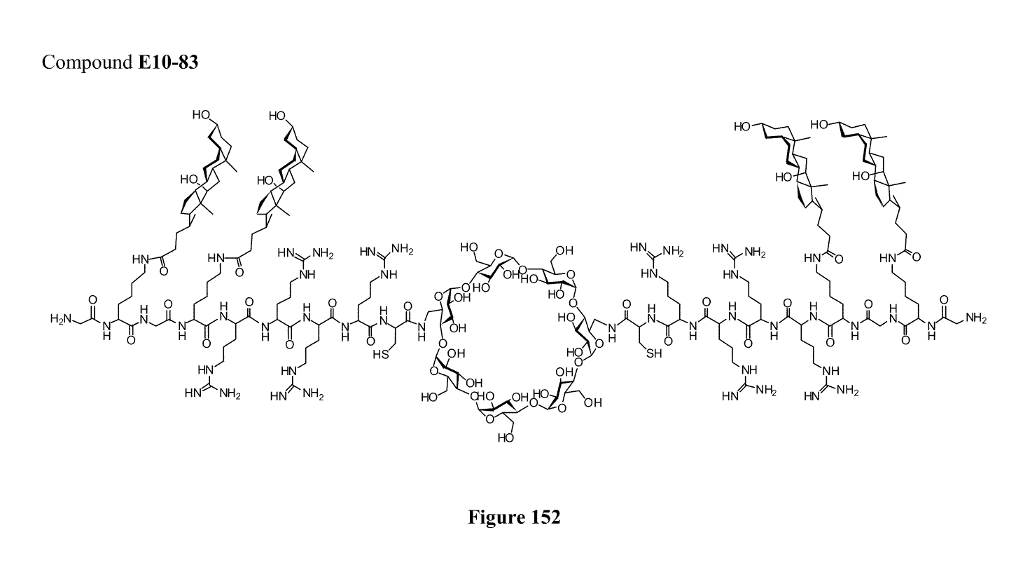

FIG. 153 presents the structure of compound 84 from Example 10.

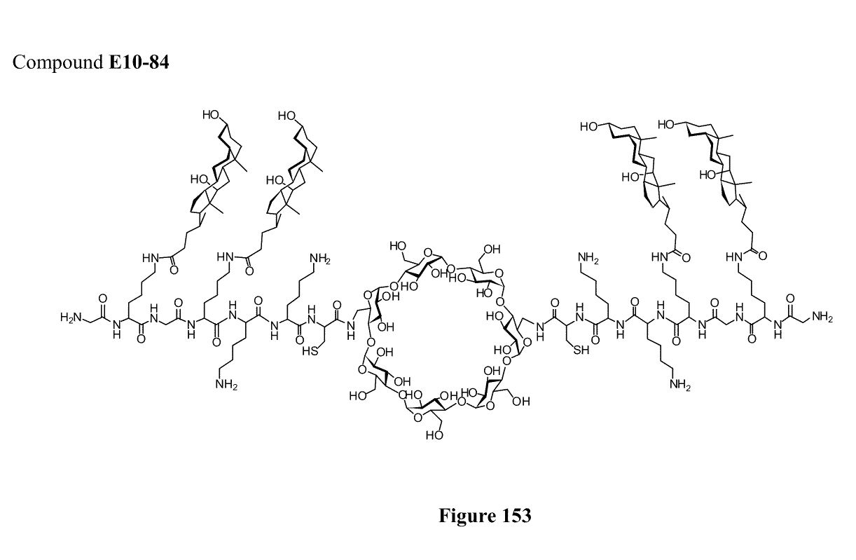

FIG. 154 presents the structure of compound 85 from Example 10.

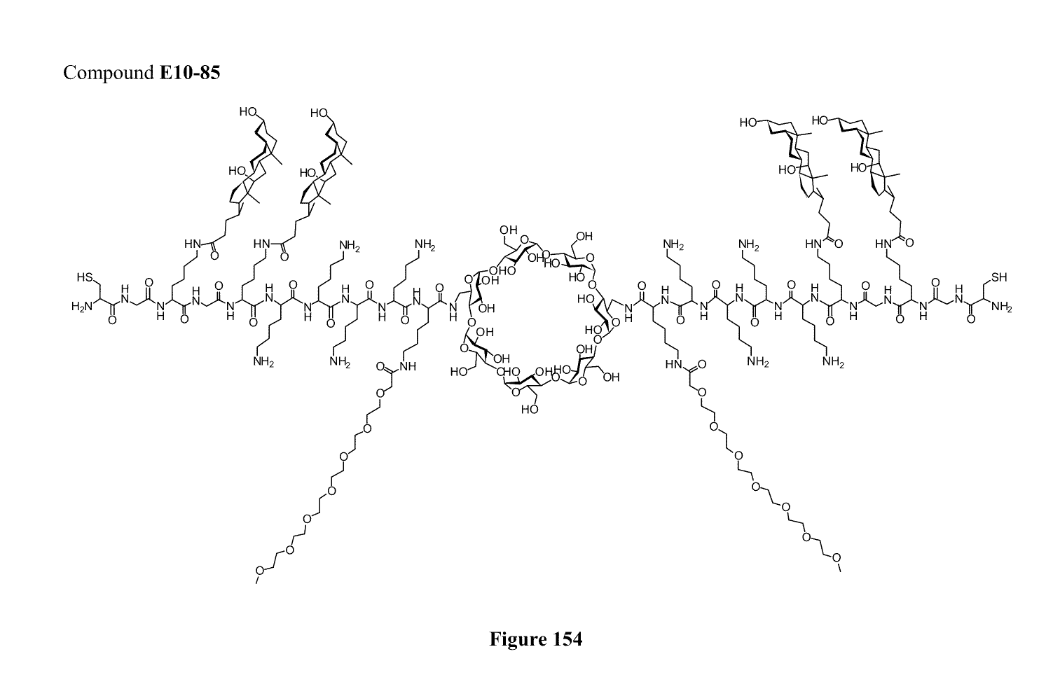

FIG. 155 presents the structure of compound 86 from Example 10.

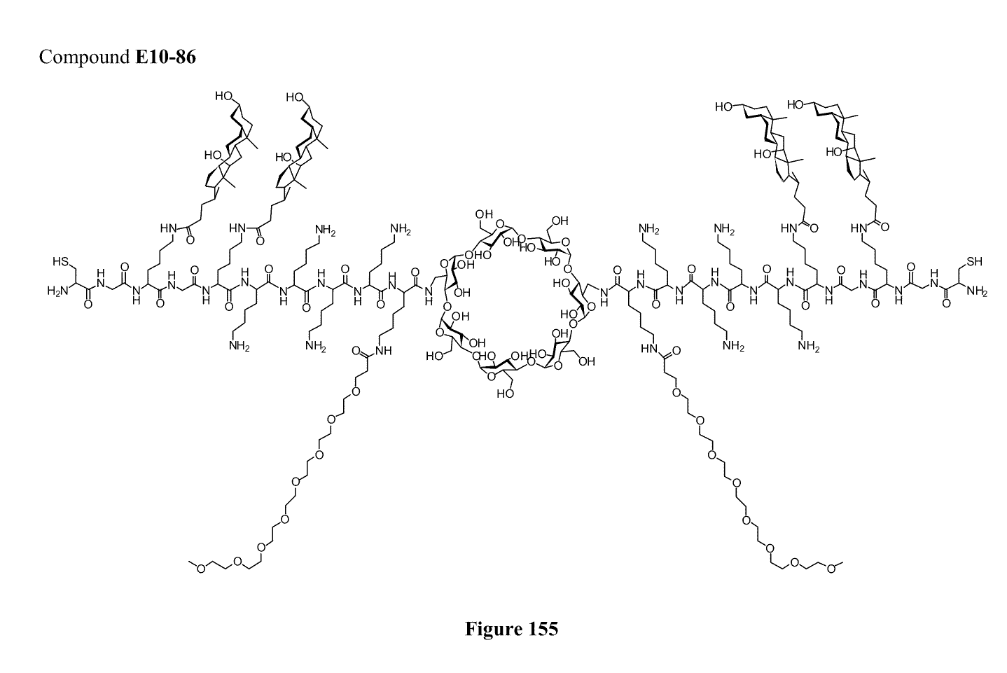

FIG. 156 presents the structure of compound 87 from Example 10.

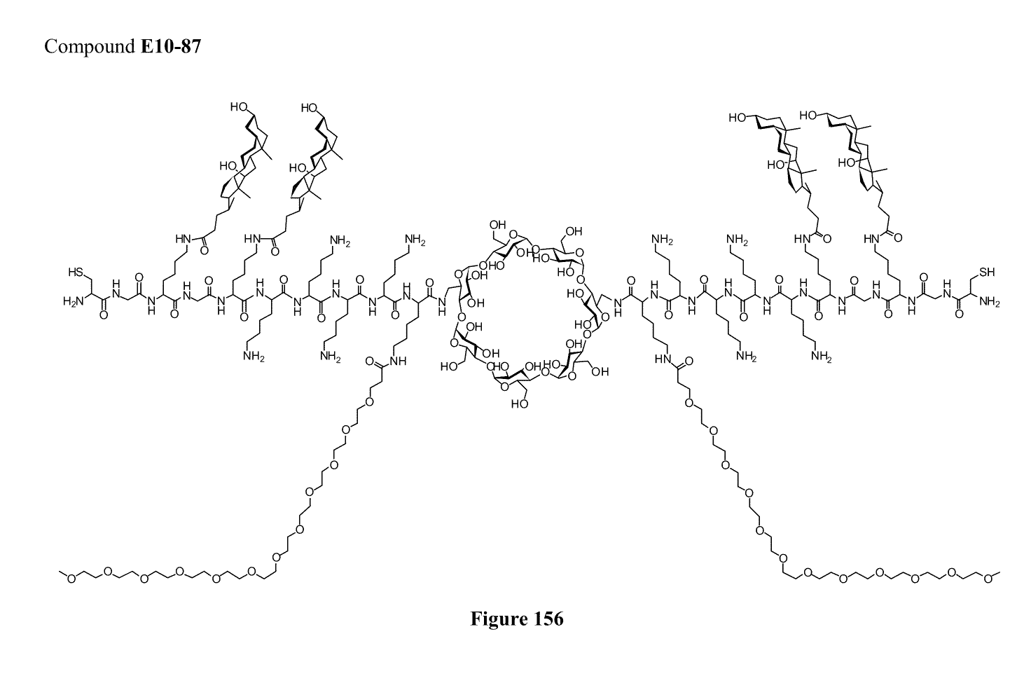

FIG. 157 presents the structure of compound 88 from Example 10.

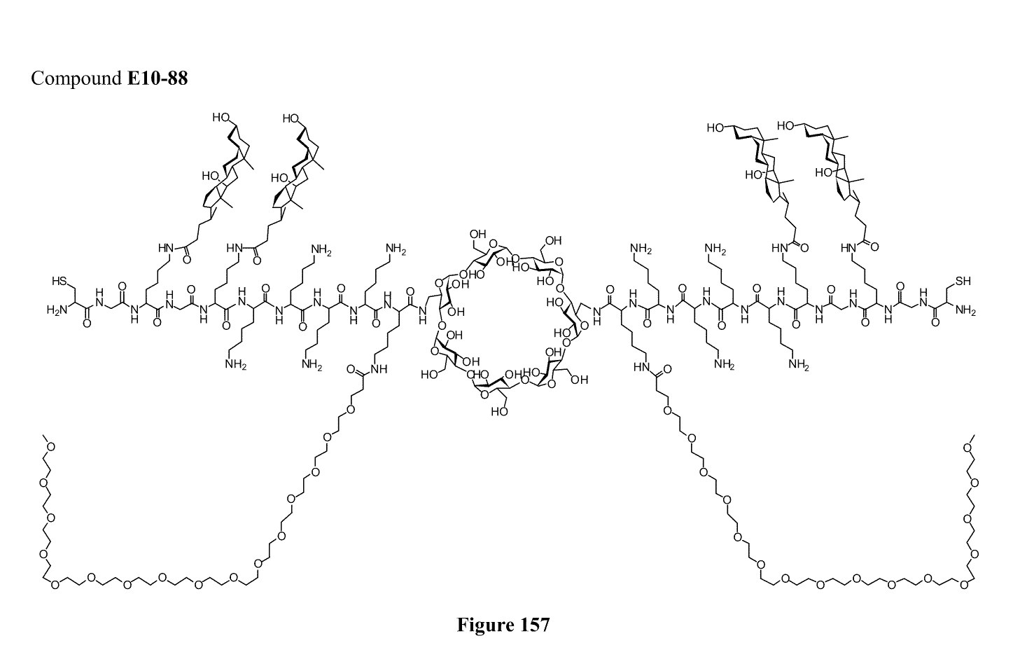

FIG. 158 presents the structure of compound 89 from Example 10.

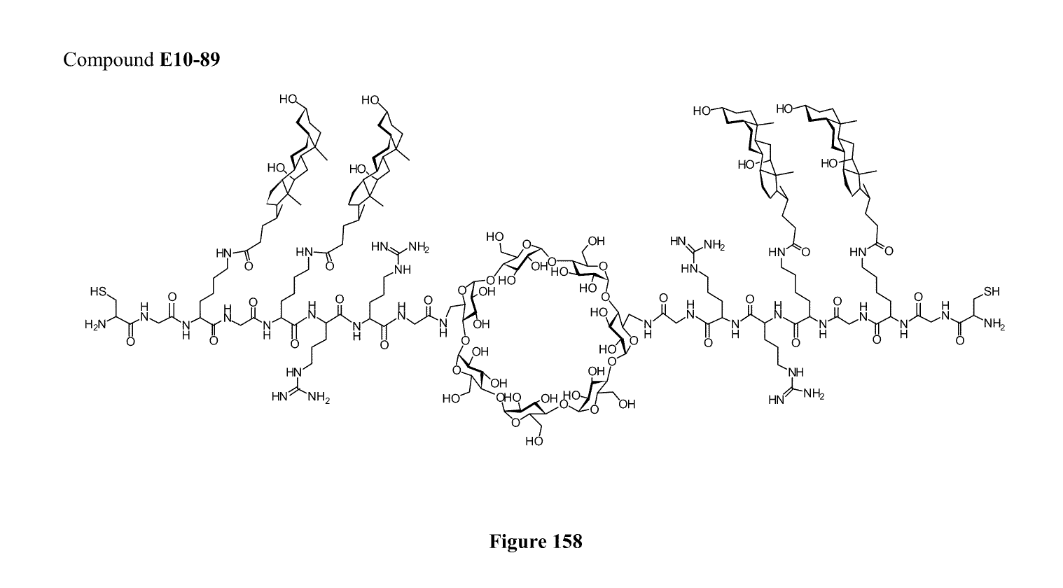

FIG. 159 presents the structure of compound 90 from Example 10.

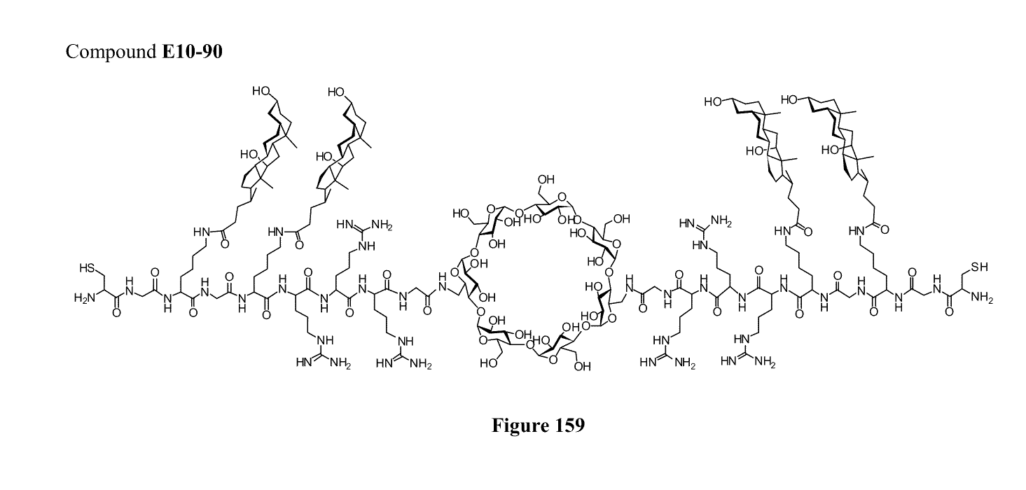

FIG. 160 presents the structure of compound 91 from Example 10.

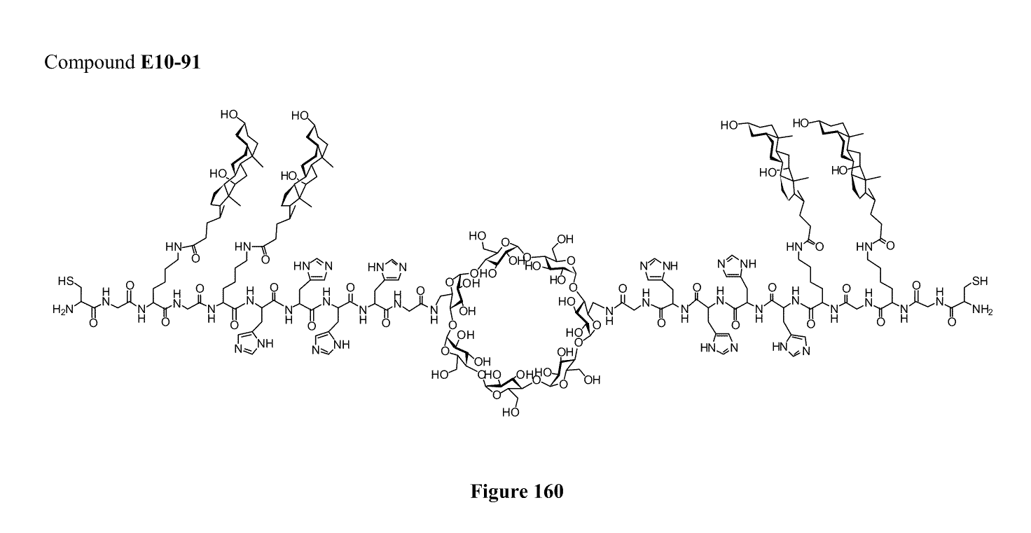

FIG. 161 presents the structure of compound 92 from Example 10.

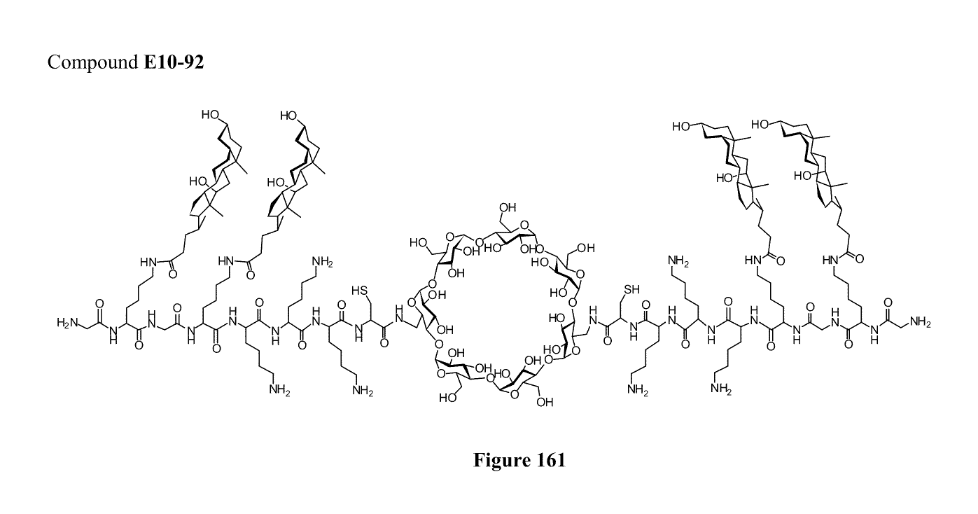

FIG. 162 presents the structure of compound 93 from Example 10.

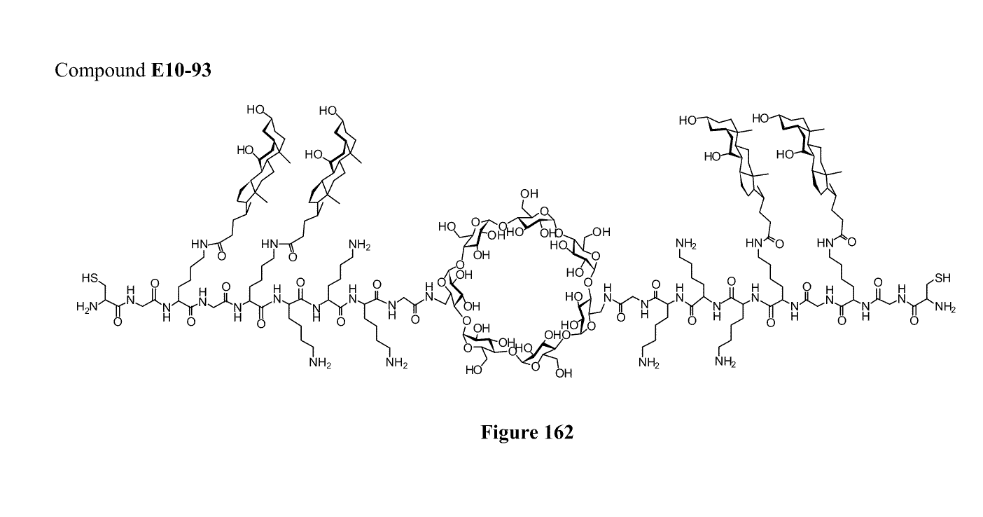

FIG. 163 presents the structure of compound 94 from Example 10.

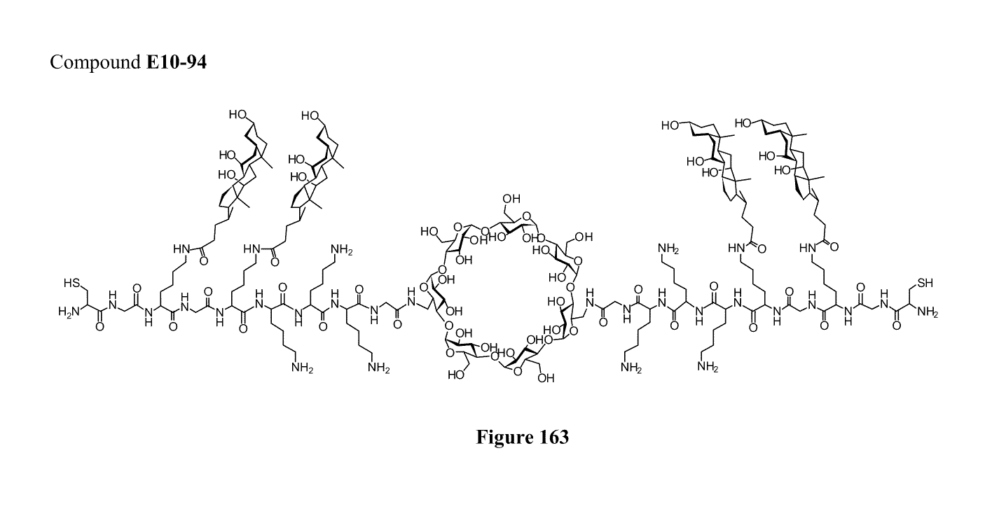

FIG. 164 presents the structure of compound 95 from Example 10.

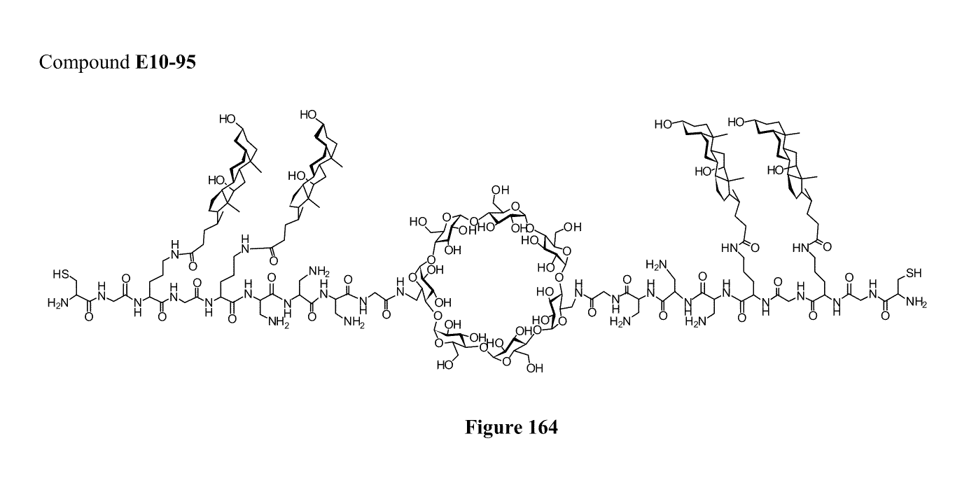

FIG. 165 presents the structure of compound 96 from Example 10.

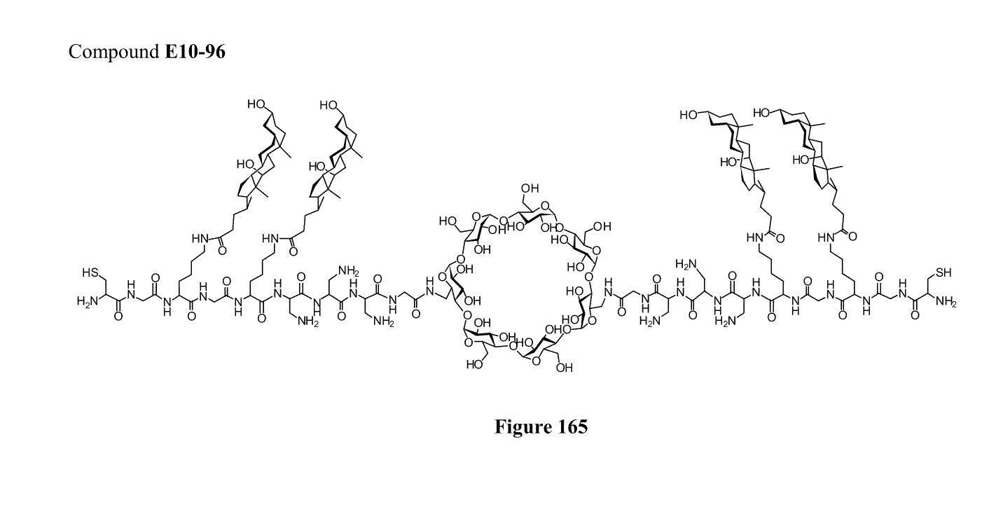

FIG. 166 presents the structure of compound 97 from Example 10.

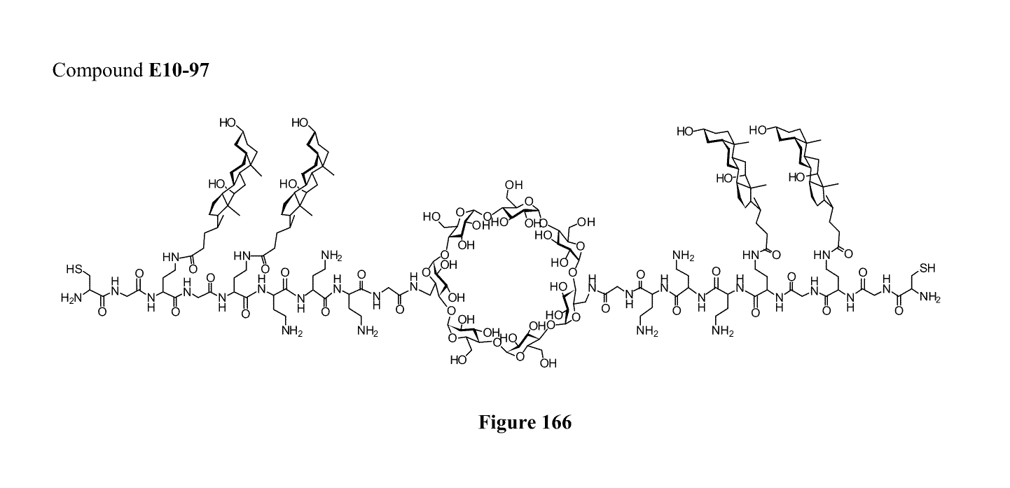

FIG. 167 presents the structure of compound 98 from Example 10.

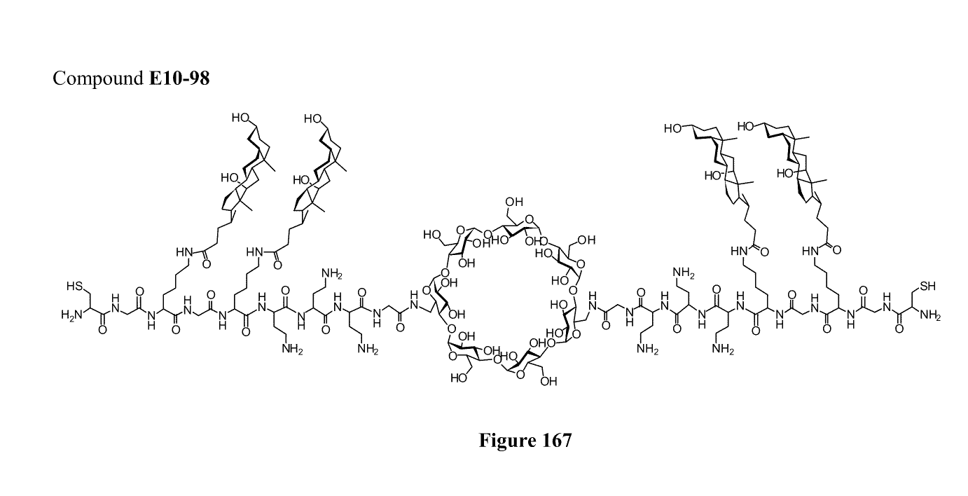

FIG. 168 presents the structure of compound 99 from Example 10.

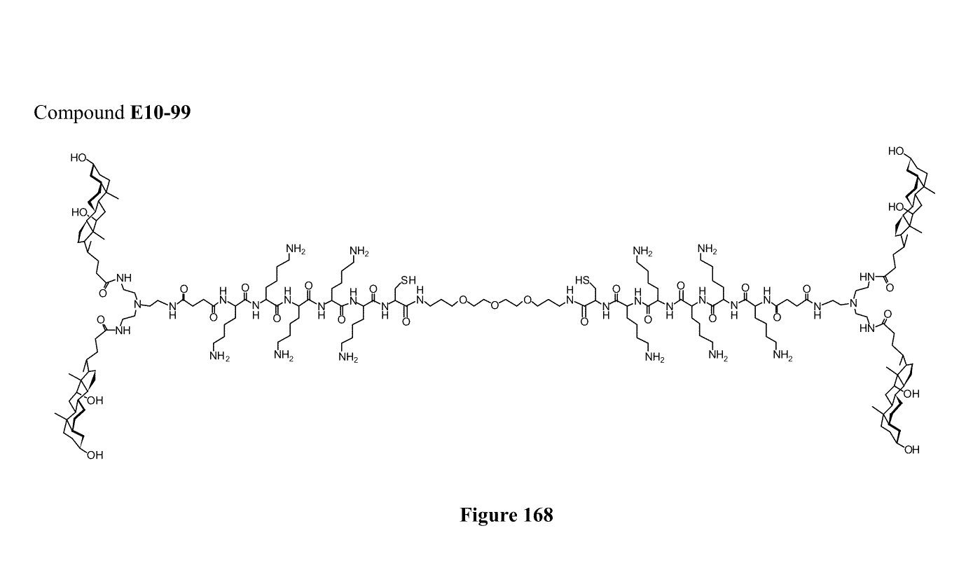

FIG. 169 presents the structure of compound 100 from Example 10.

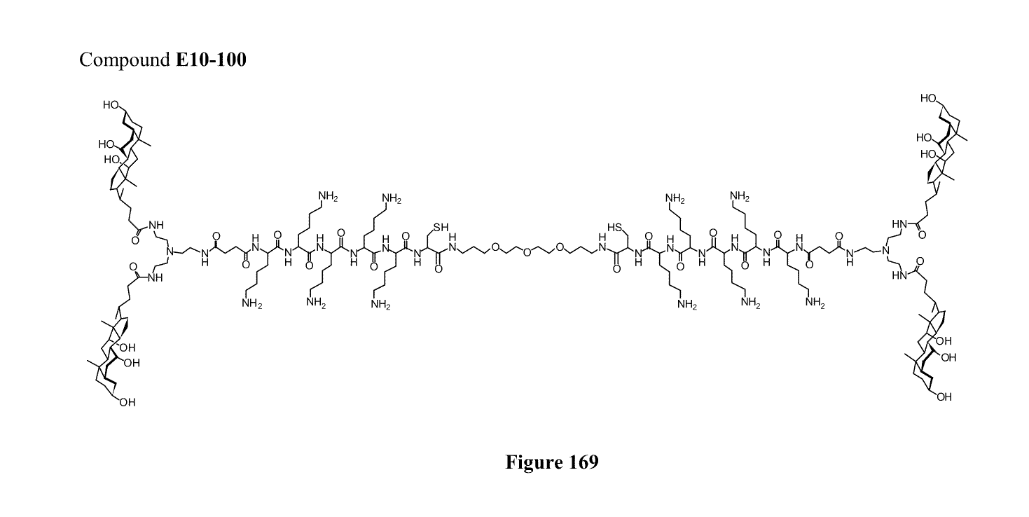

FIG. 170 presents the structure of compound 101 from Example 10.

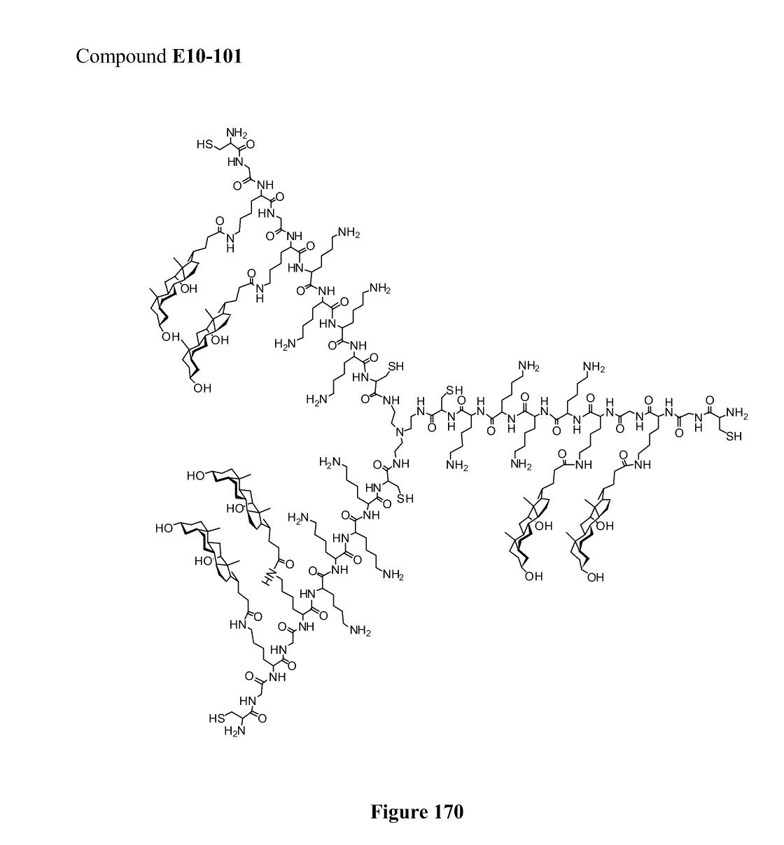

FIG. 171 presents the structure of compound 102 from Example 10.

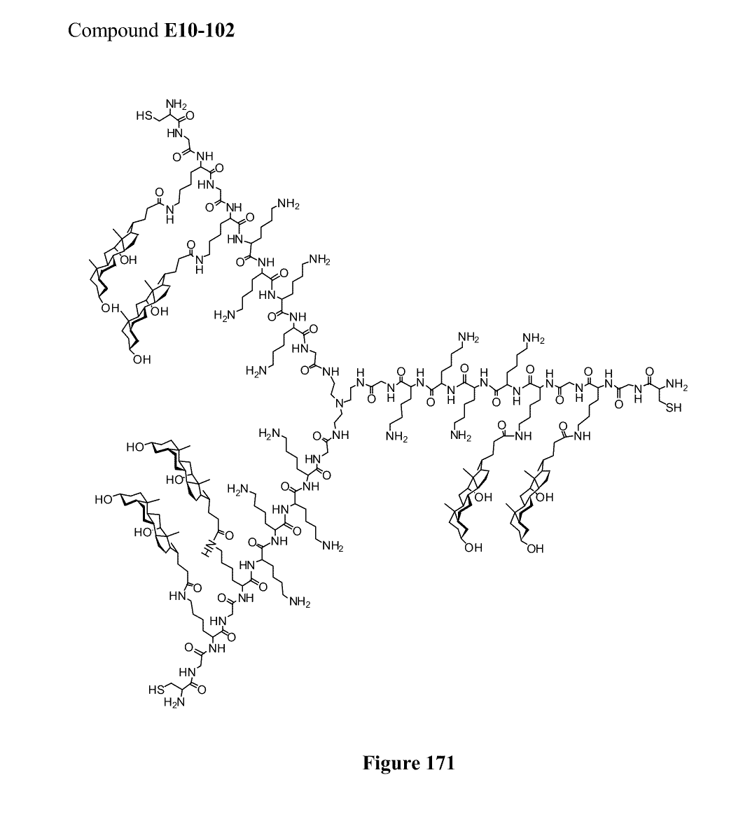

FIG. 172 presents the structure of compound 103 from Example 10.

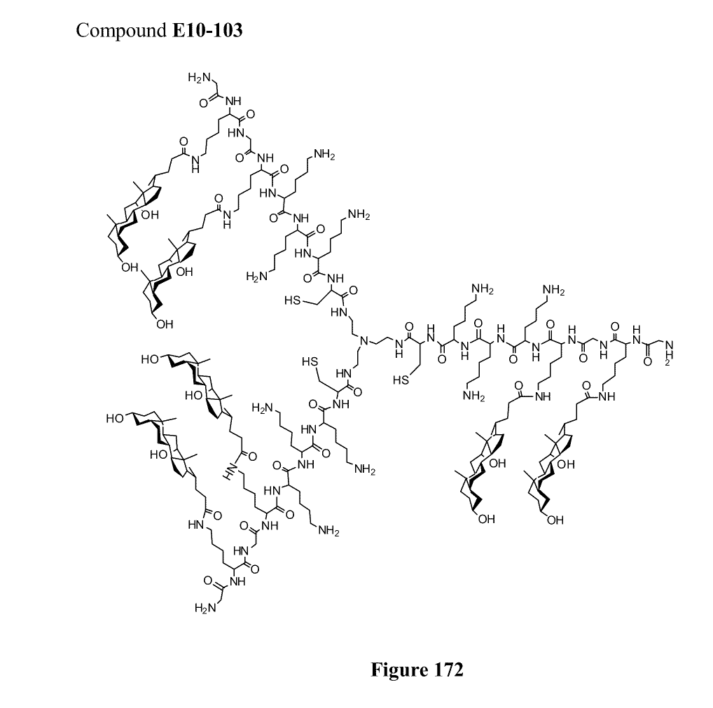

FIG. 173 presents the structure of compound 104 from Example 10.

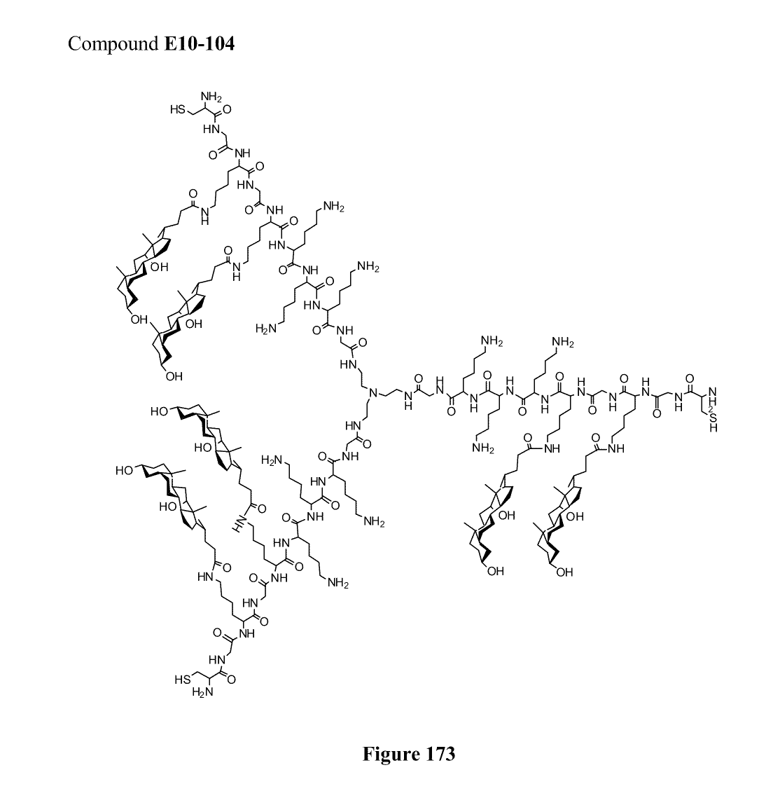

FIG. 174 presents the structure of compound 105 from Example 10.

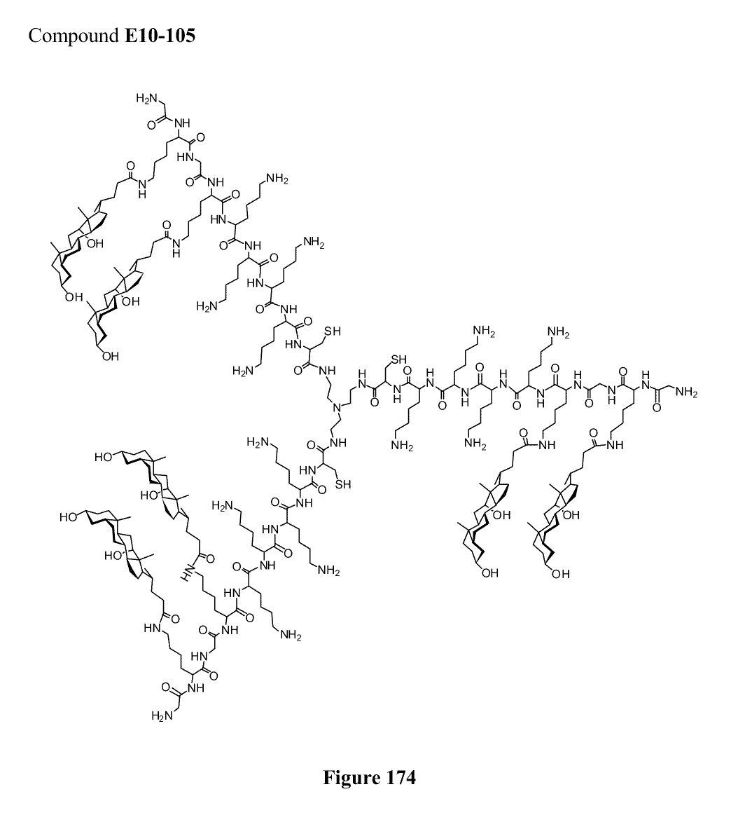

FIG. 175 presents the structure of compound 106 from Example 10.

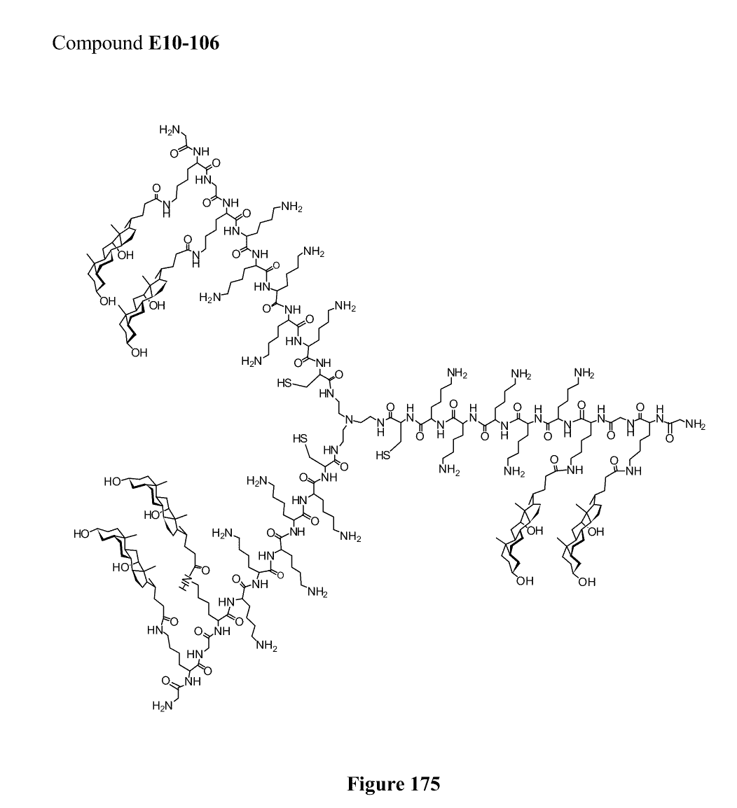

FIG. 176 presents the structure of compound 107 from Example 10.

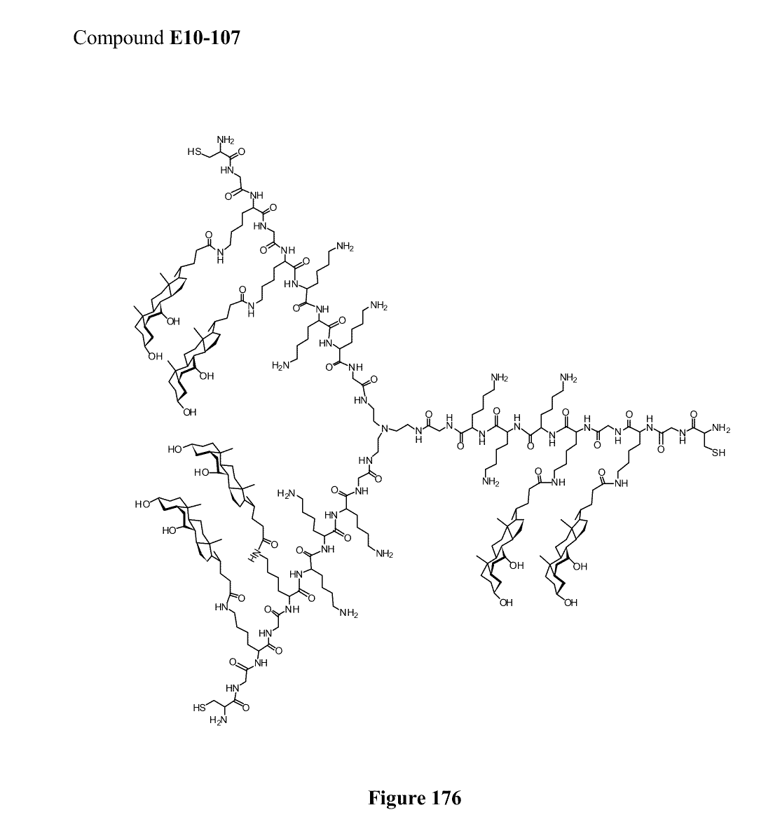

FIG. 177 presents the structure of compound 108 from Example 10.

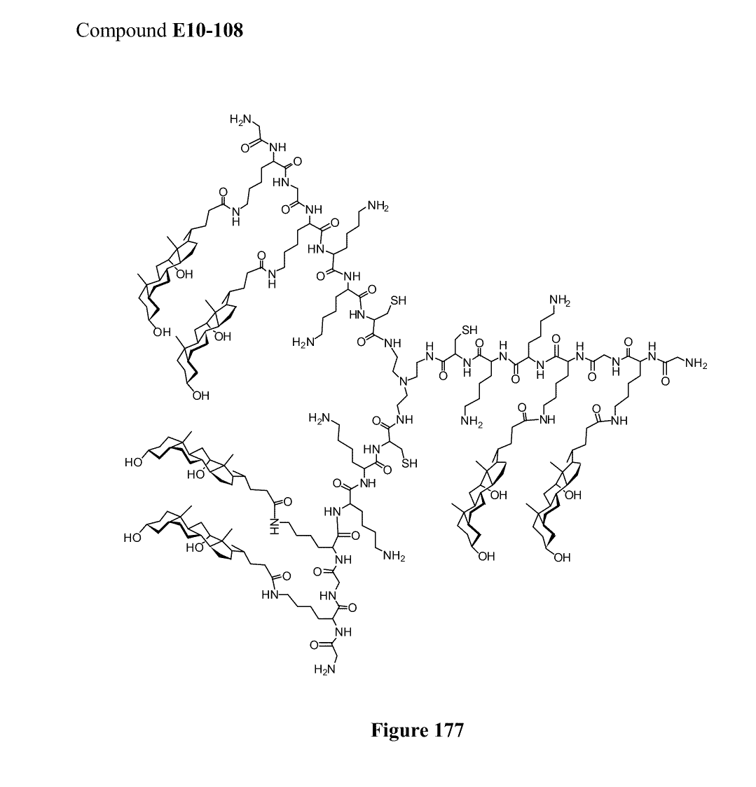

FIG. 178 presents the structure of compound 109 from Example 10.

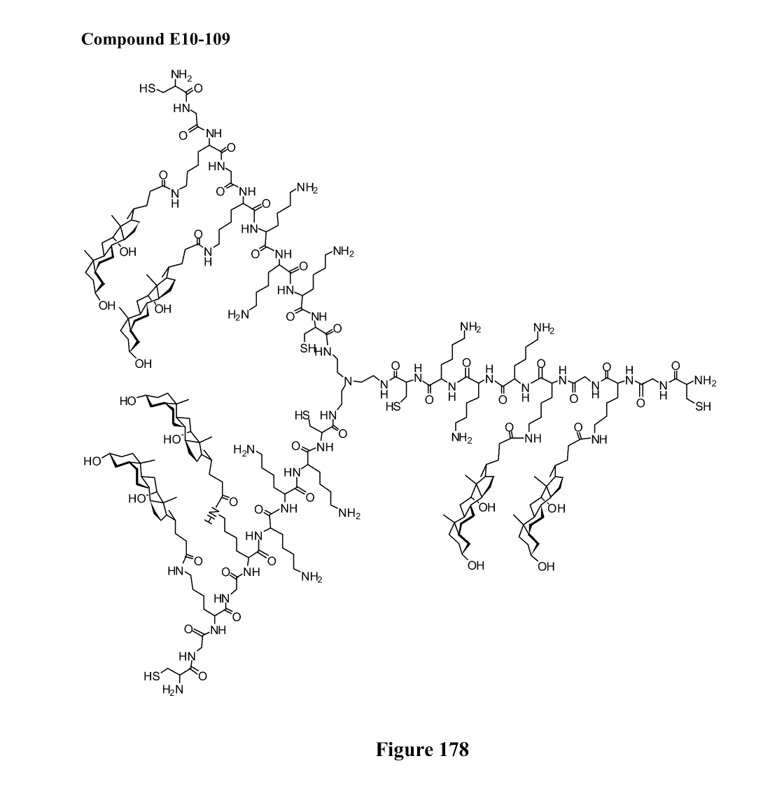

FIG. 179 presents the structure of compound 110 from Example 10.

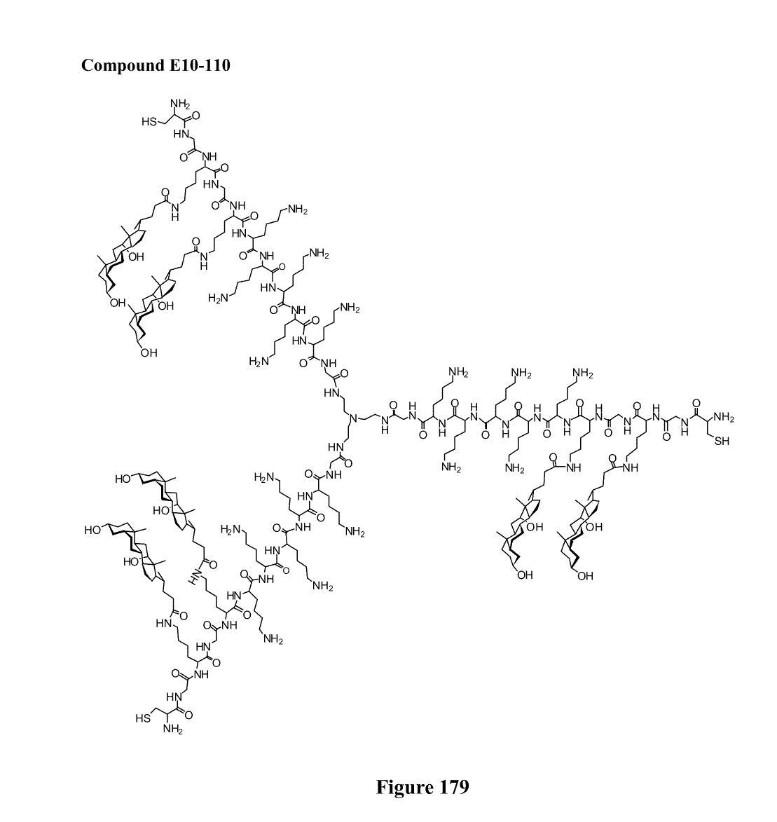

FIG. 180 presents the structure of compound 111 from Example 10.

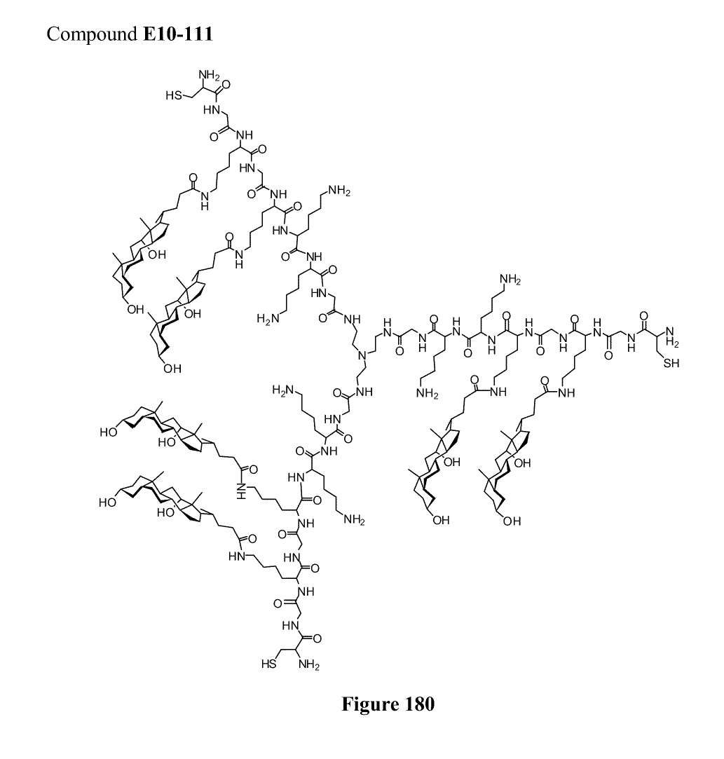

FIG. 181 presents the structure of compound 112 from Example 10.

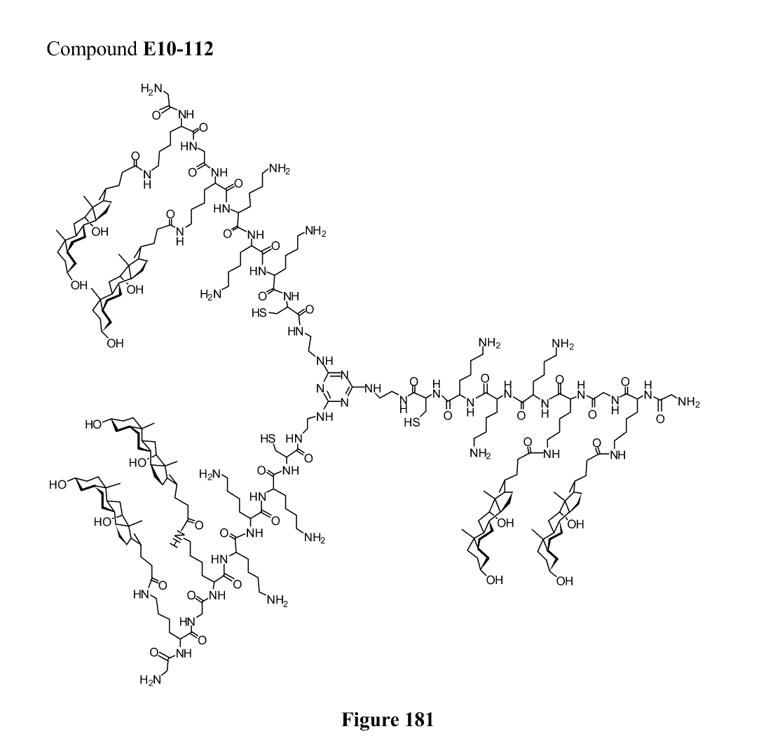

FIG. 182 presents the structure of compound 113 from Example 10.

FIG. 183 presents the structure of compound 2 from Example 11.

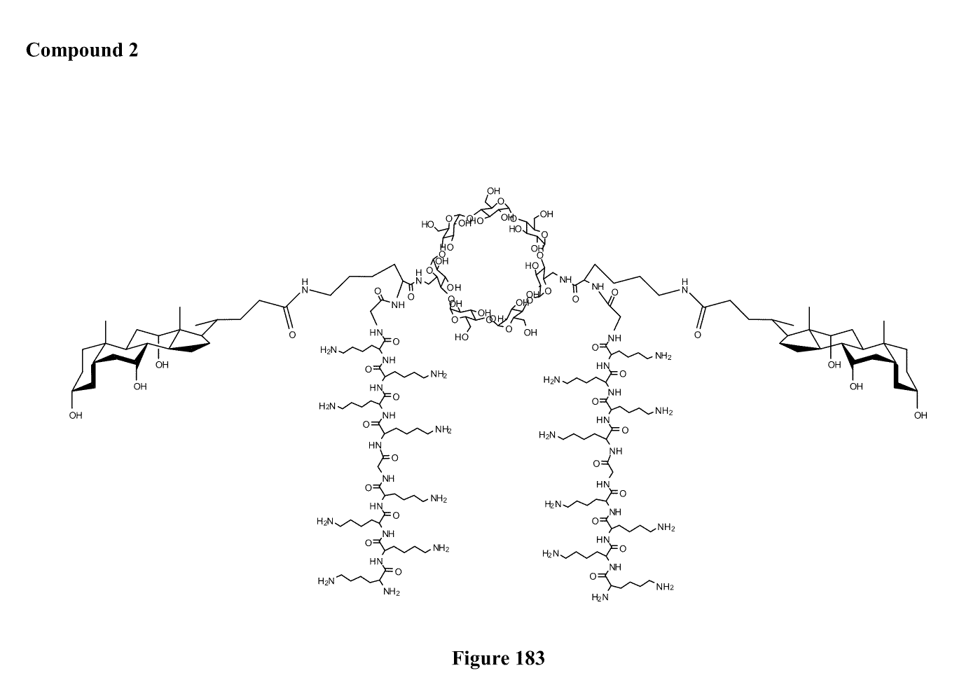

FIG. 184 presents the structure of compound 3 from Example 11.

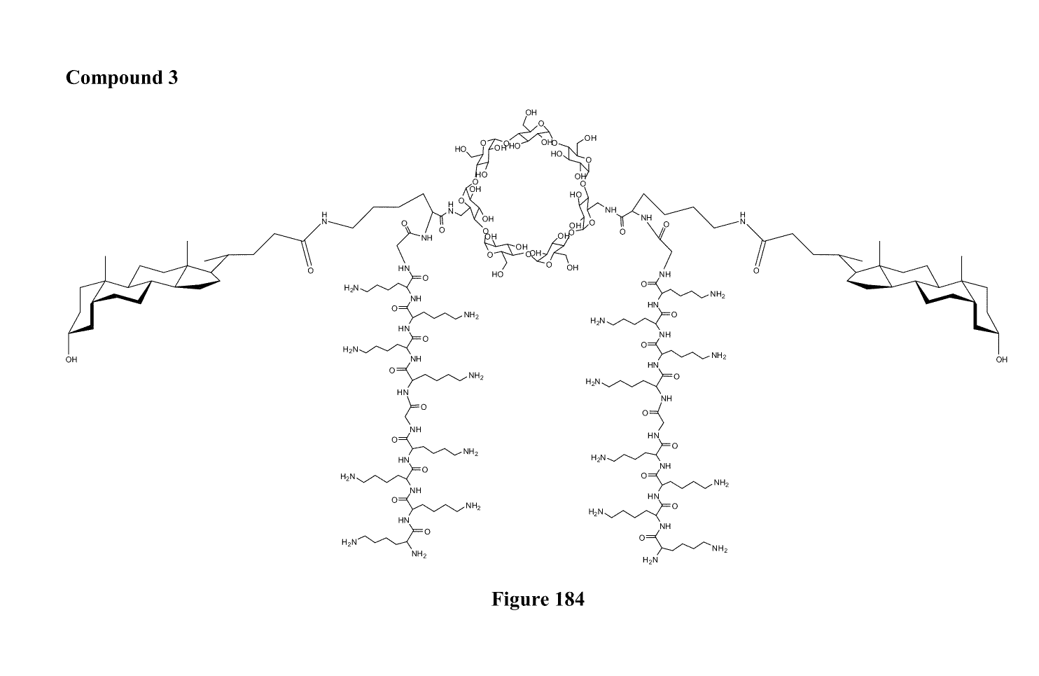

FIG. 185 presents the structure of compound 4 from Example 11.

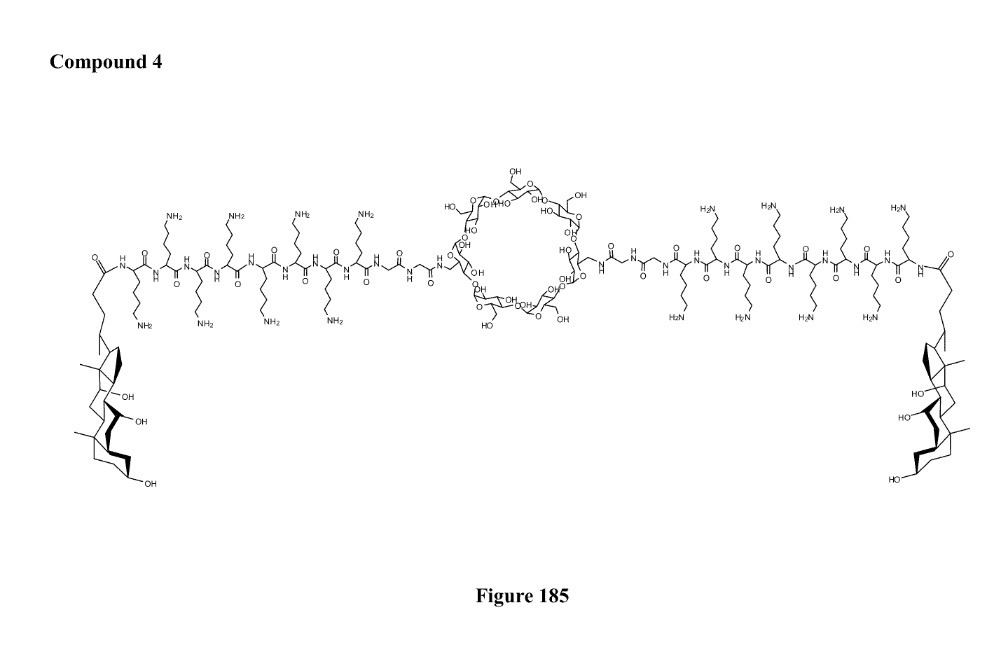

FIG. 186 presents the structure of compound 5 from Example 11.

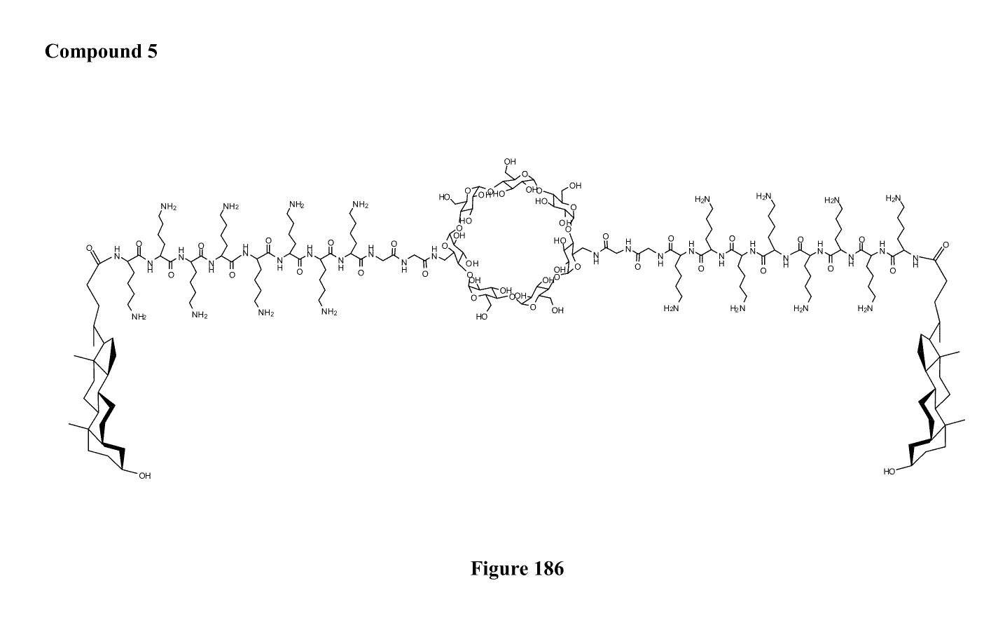

FIG. 187 presents the structure of compound 6 from Example 11.

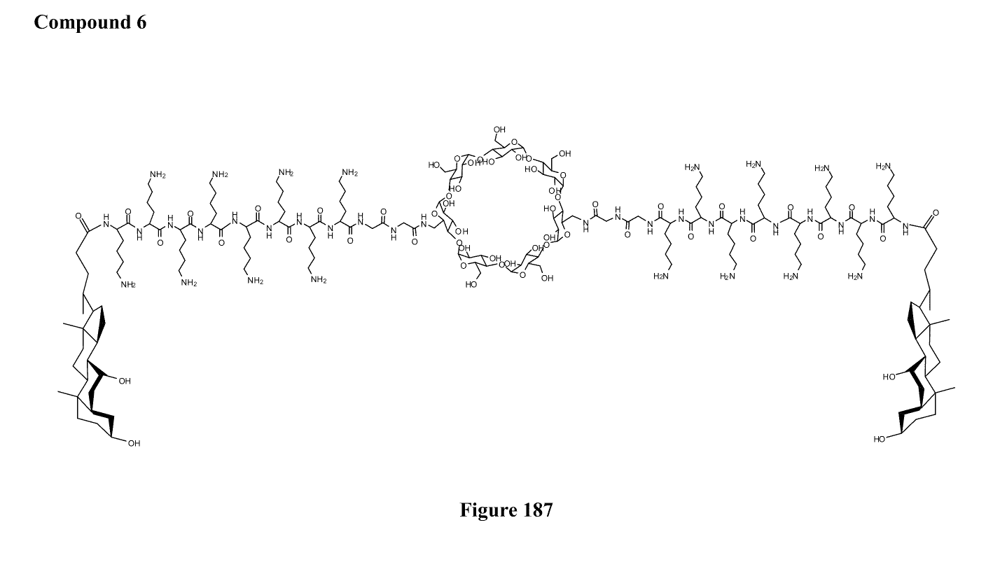

FIG. 188 presents the structure of compound 7 from Example 11.

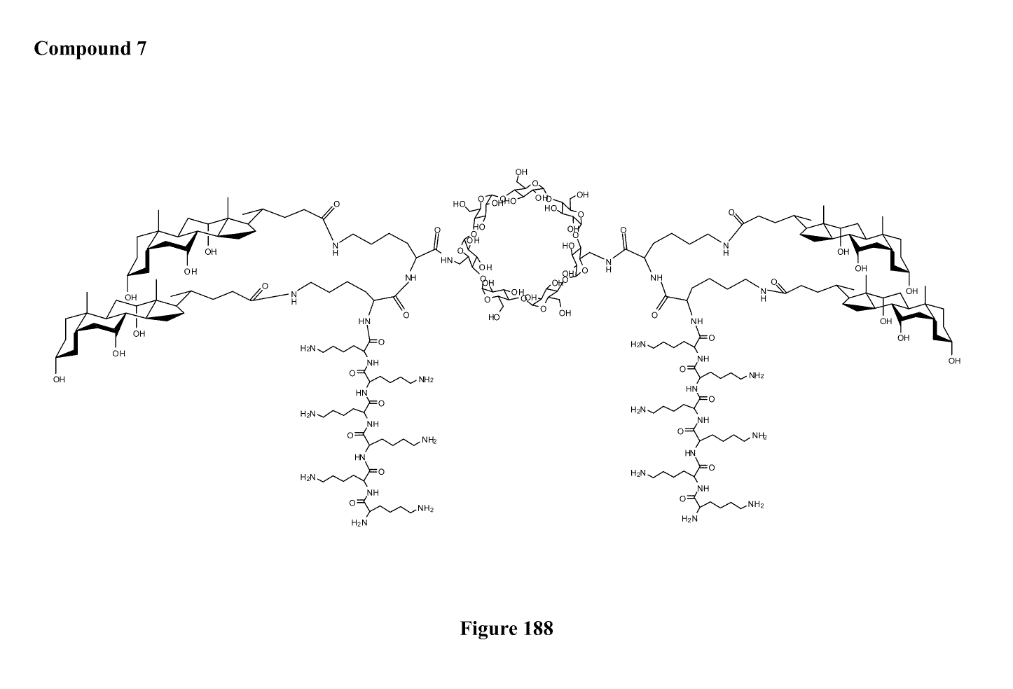

FIG. 189 presents the structure of compound 8 from Example 11.

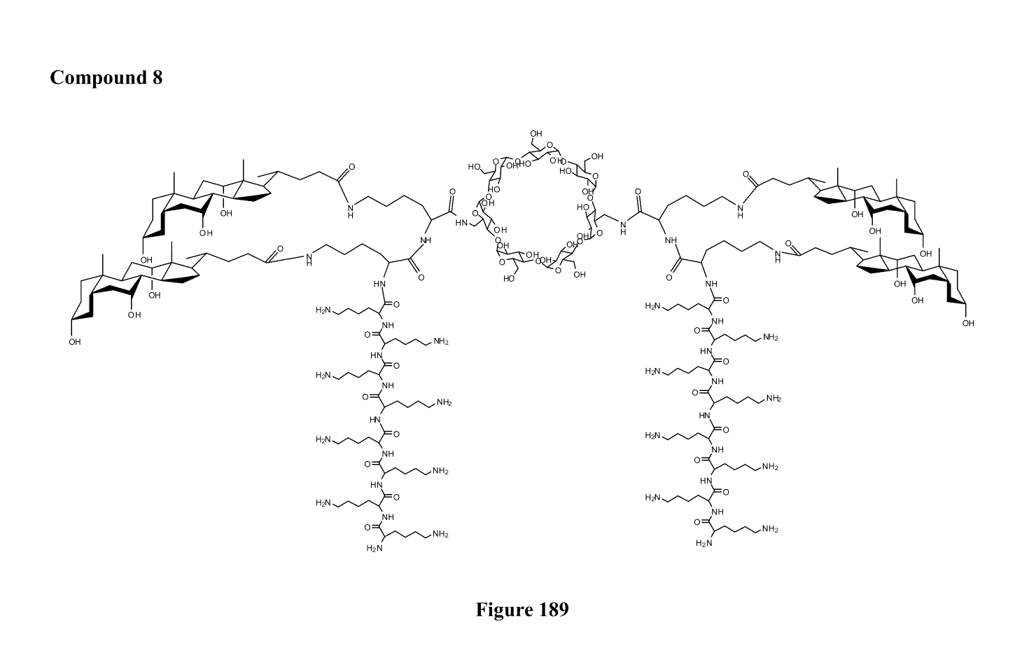

FIG. 190 presents the structure of compound 9 from Example 11.

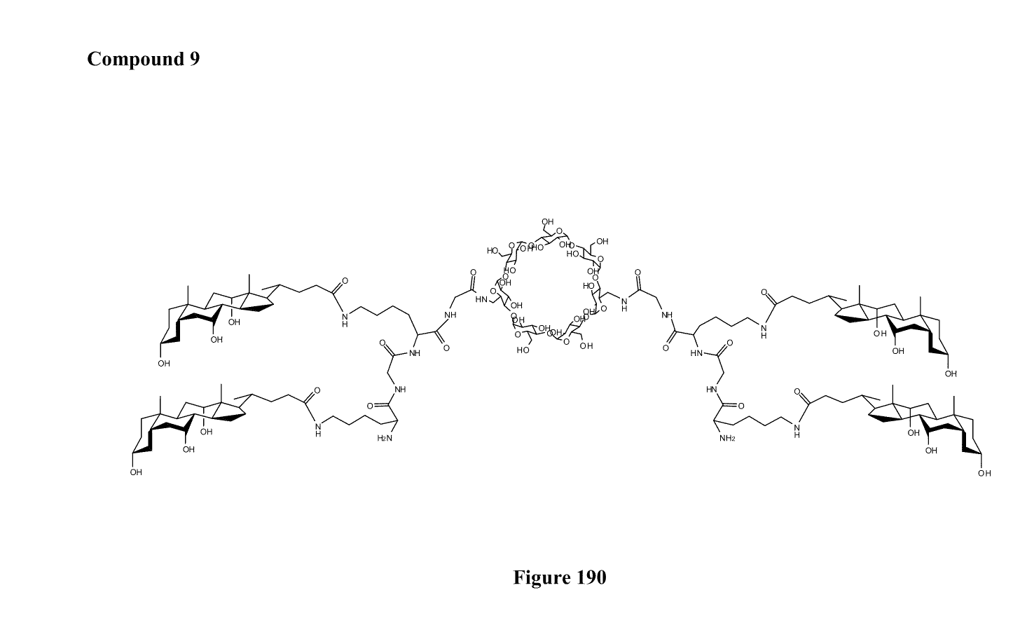

FIG. 191 presents the structure of compound 10 from Example 11.

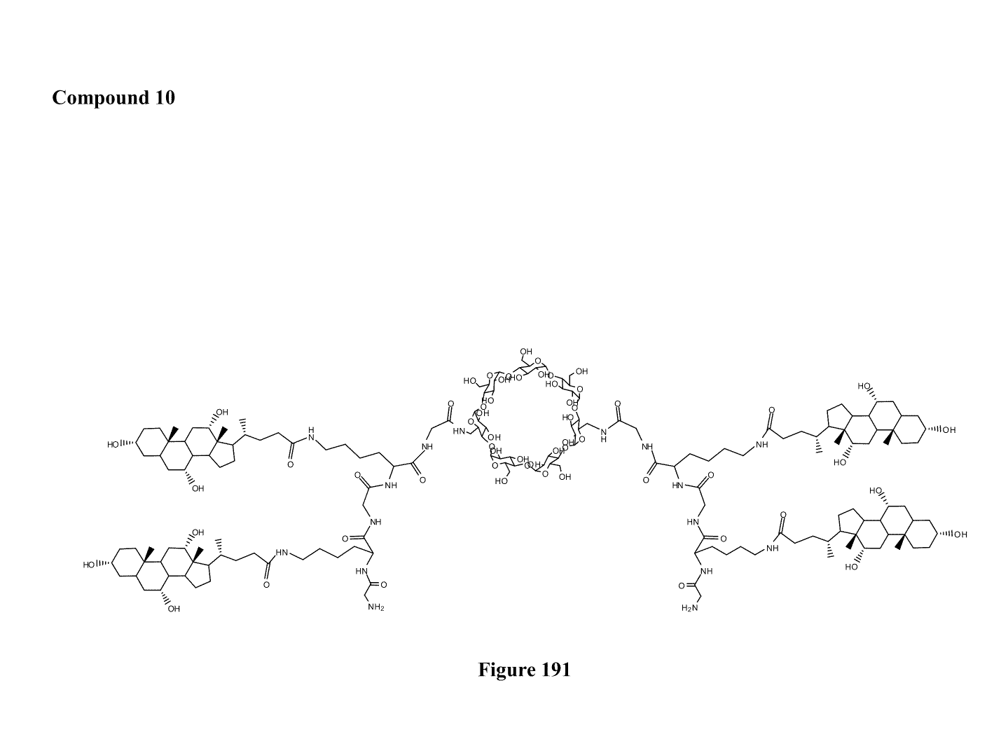

FIG. 192 presents the structure of compound 11 from Example 11.

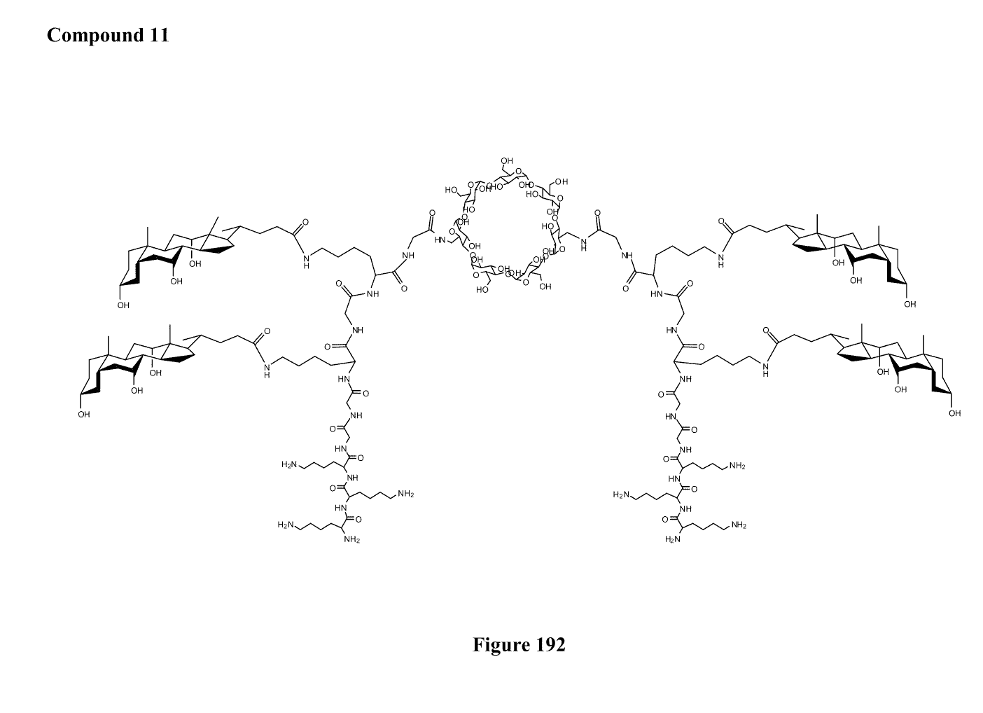

FIG. 193 presents the structure of compound 12 from Example 11.

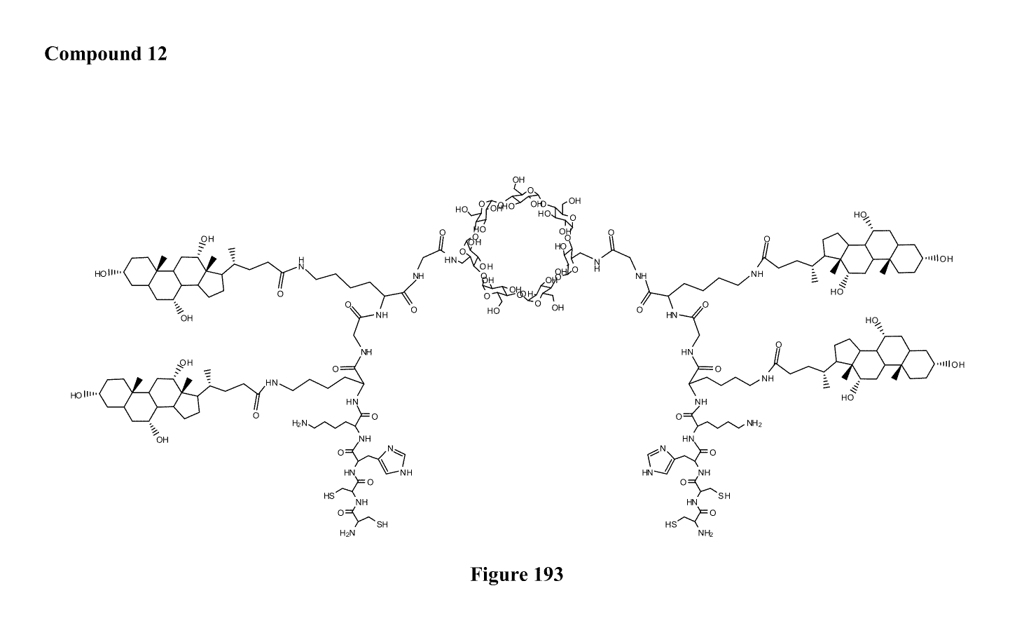

FIG. 194 presents the structure of compound 13 from Example 11.

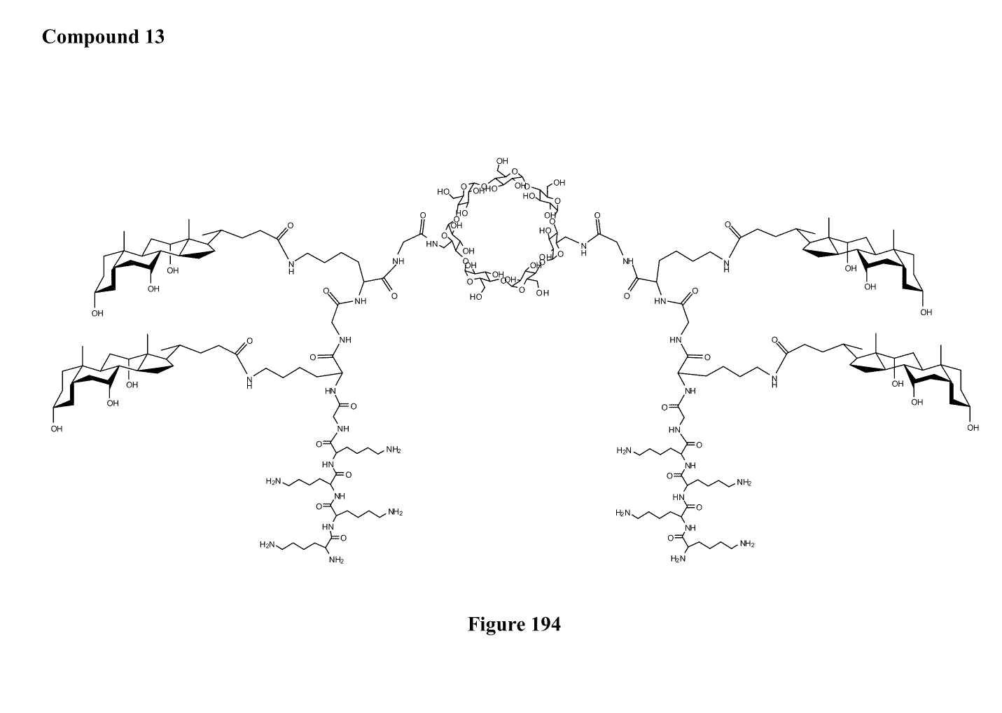

FIG. 195 presents the structure of compound 14 from Example 11.

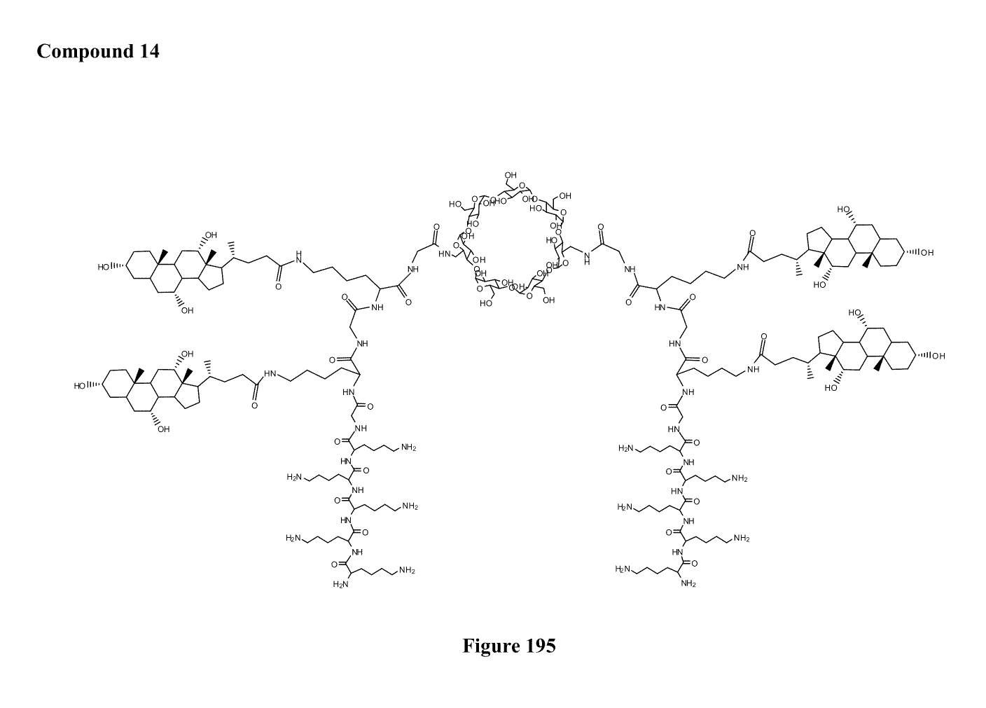

FIG. 196 presents the structure of compound 15 from Example 11.

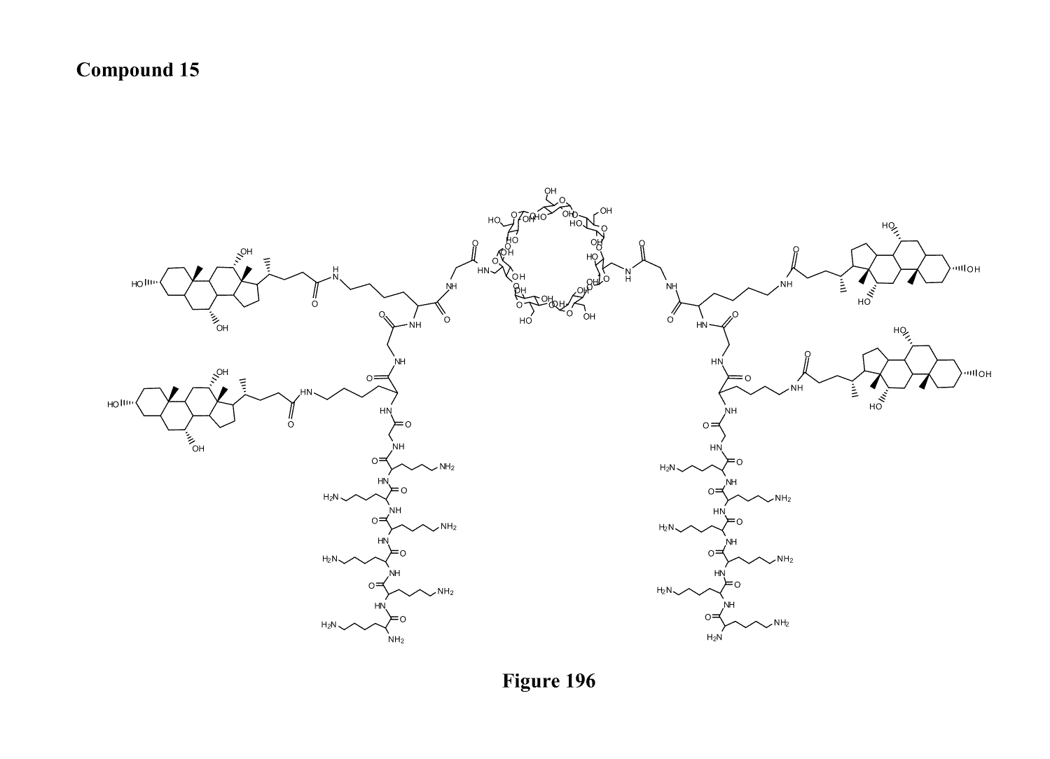

FIG. 197 presents the structure of compound 16 from Example 11.

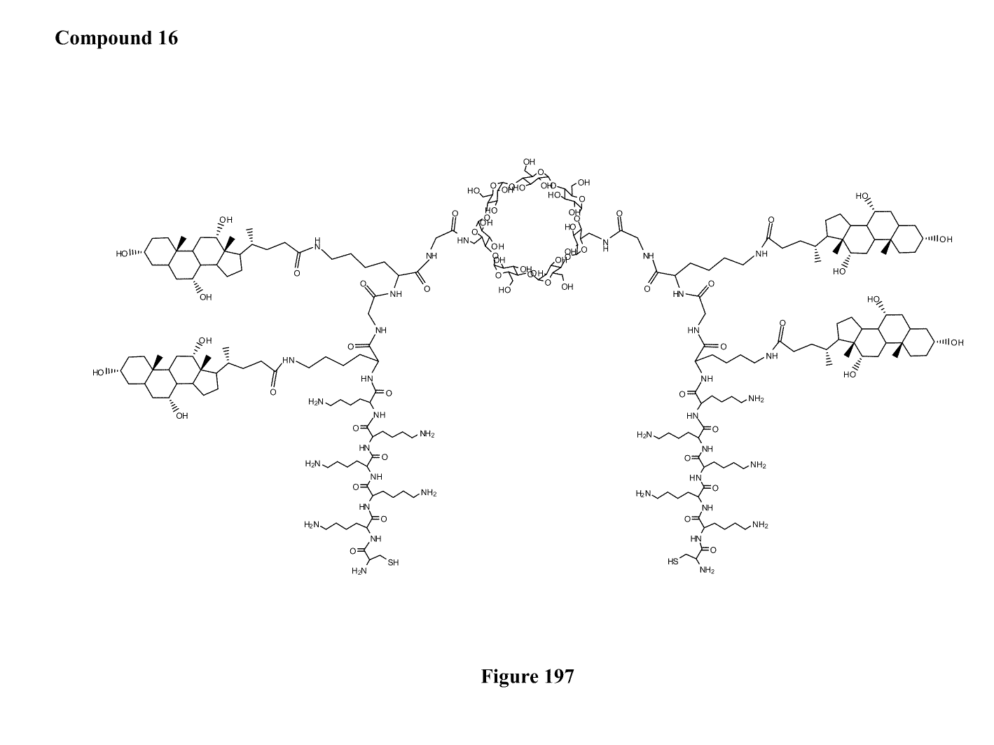

FIG. 198 presents the structure of compound 17 from Example 11.

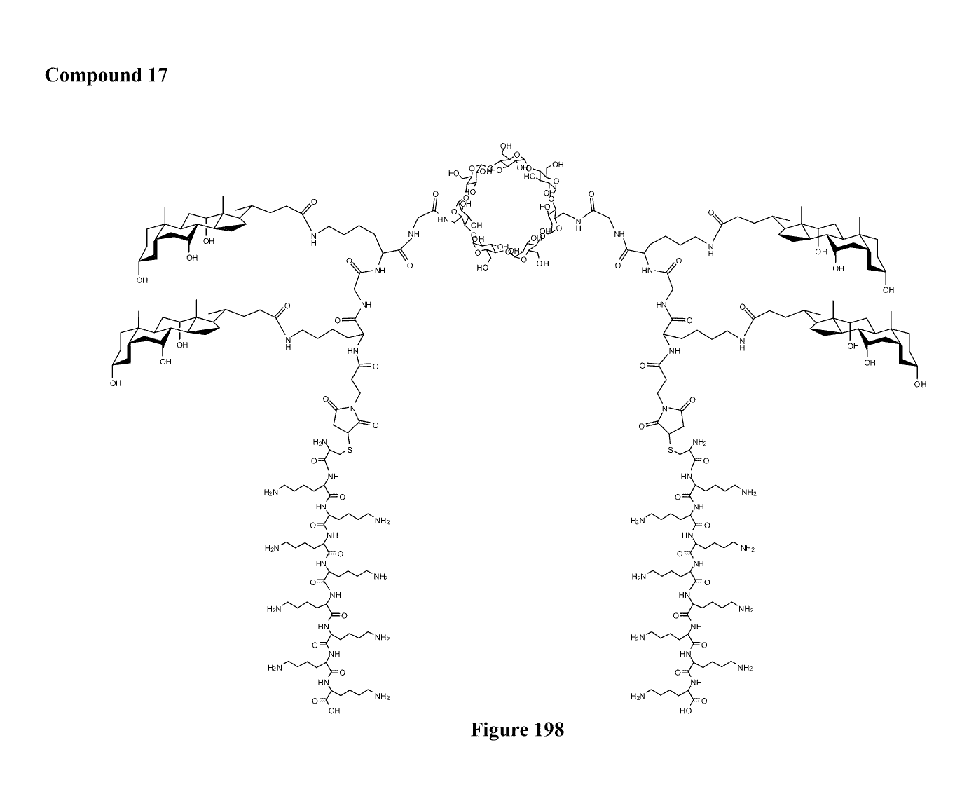

FIG. 199 presents the structure of compound 18 from Example 11.

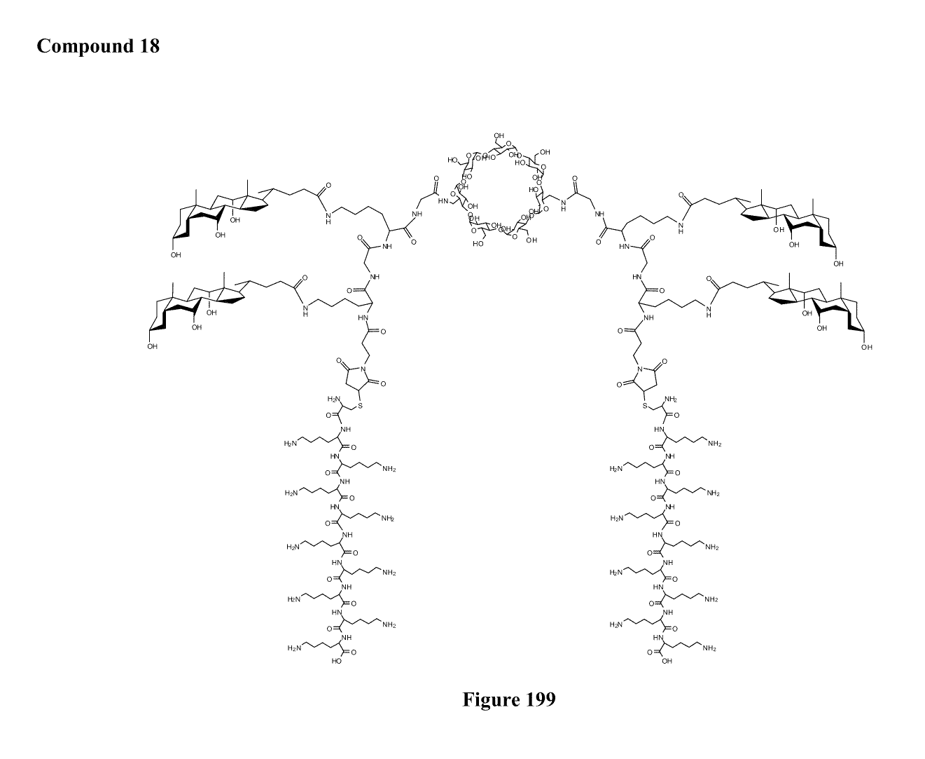

FIG. 200 presents the structure of compound 19 from Example 11.

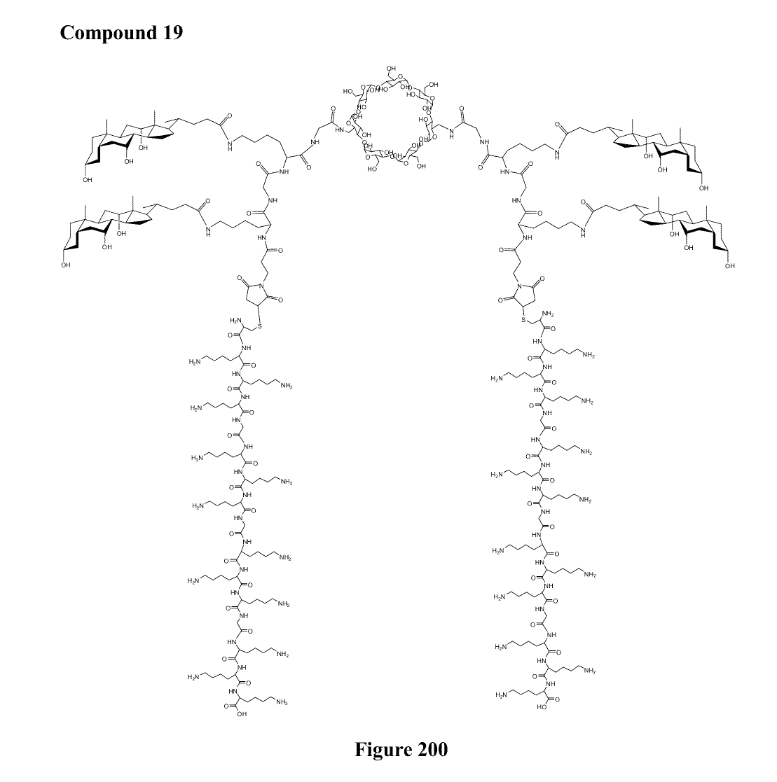

FIG. 201 presents the structure of compound 20 from Example 11.

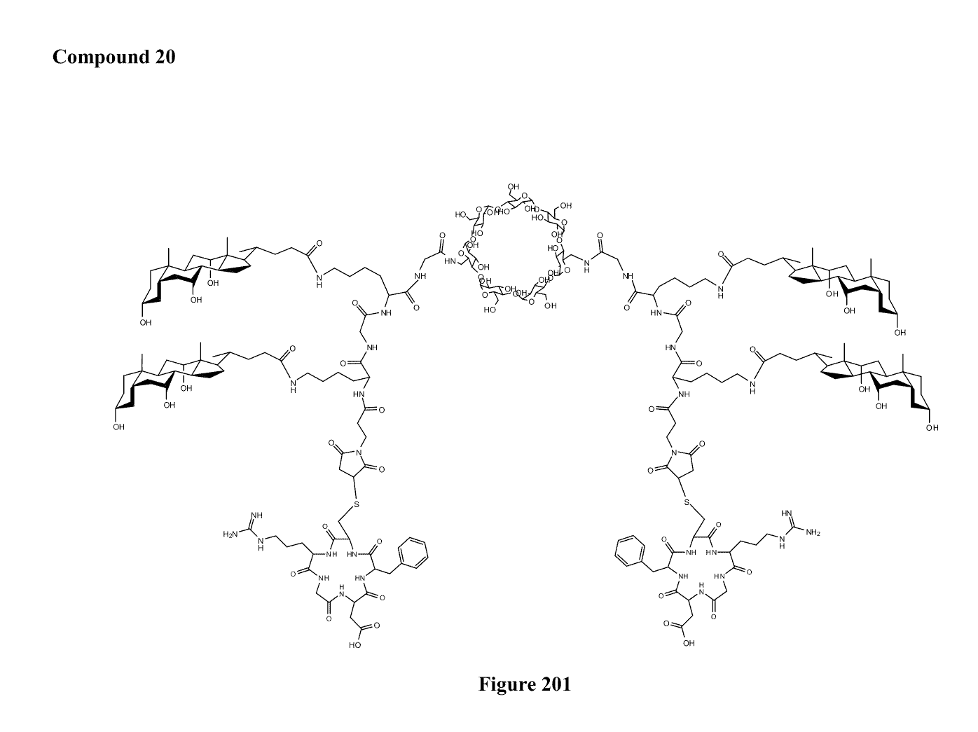

FIG. 202 presents the structure of compound 21 from Example 11.

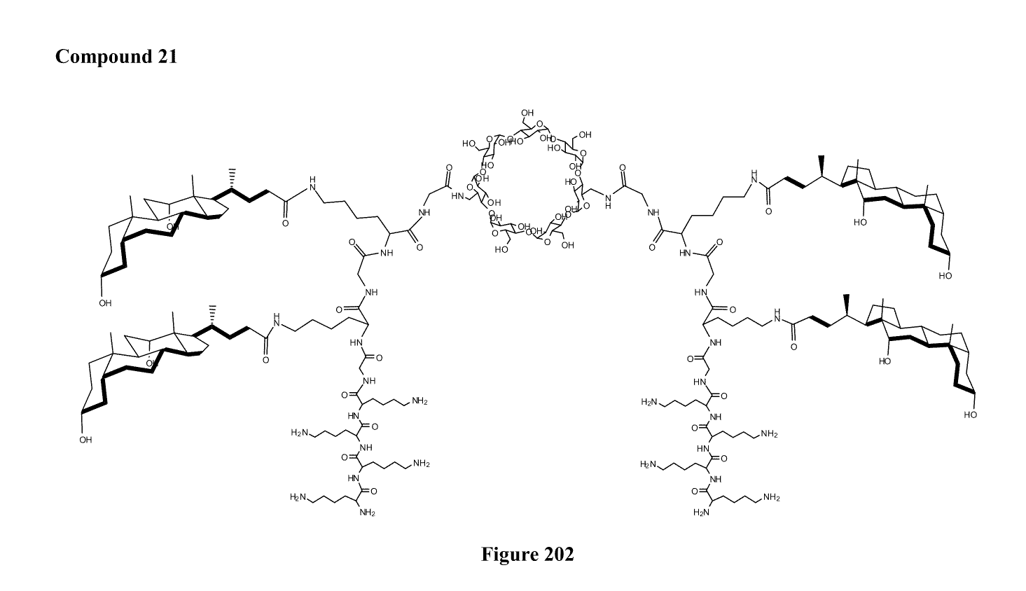

FIG. 203 presents the structure of compound 22 from Example 11.

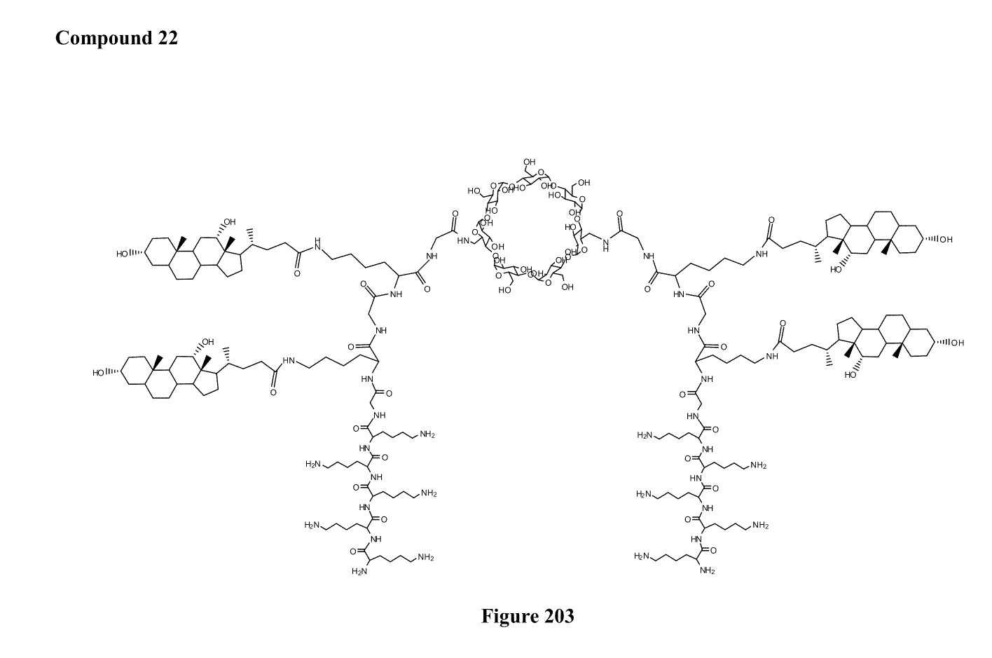

FIG. 204 presents the structure of compound 23 from Example 11.

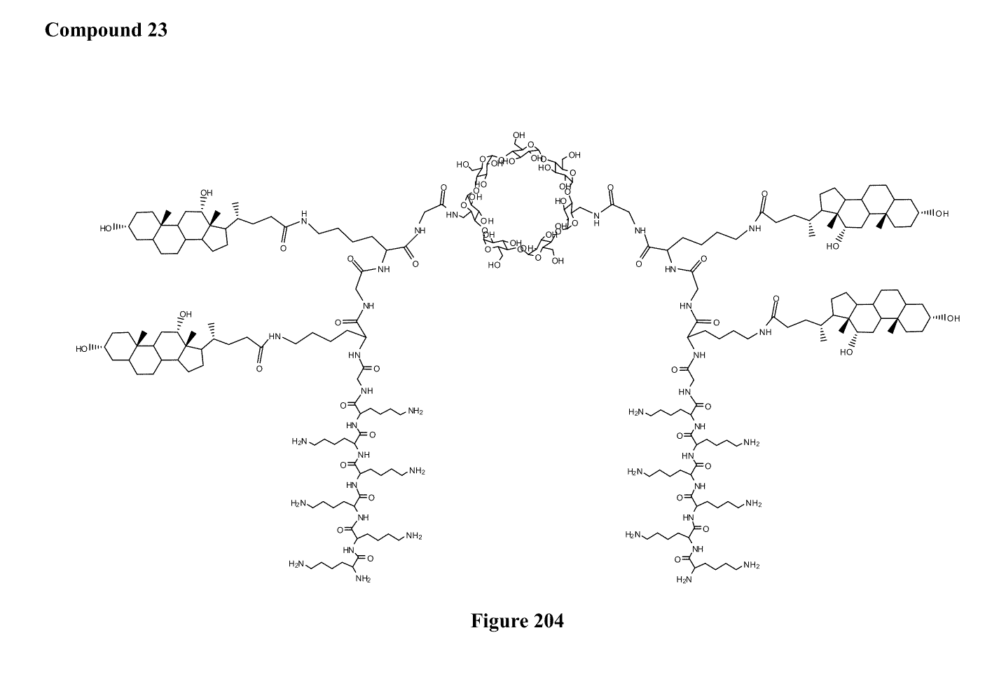

FIG. 205 presents the structure of compound 24 from Example 11.

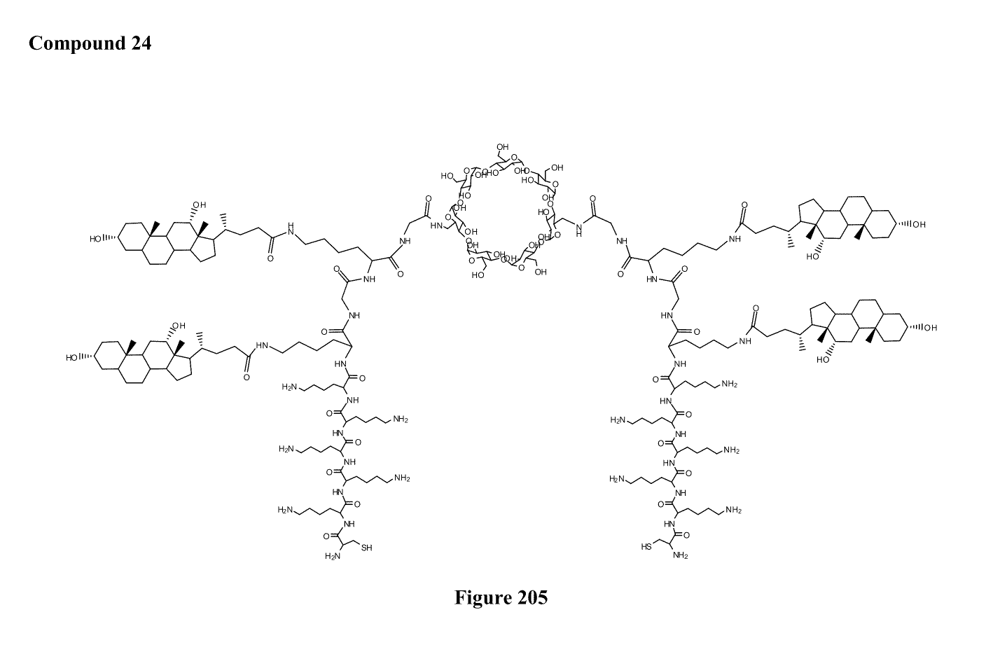

FIG. 206 presents the structure of compound 25 from Example 11.

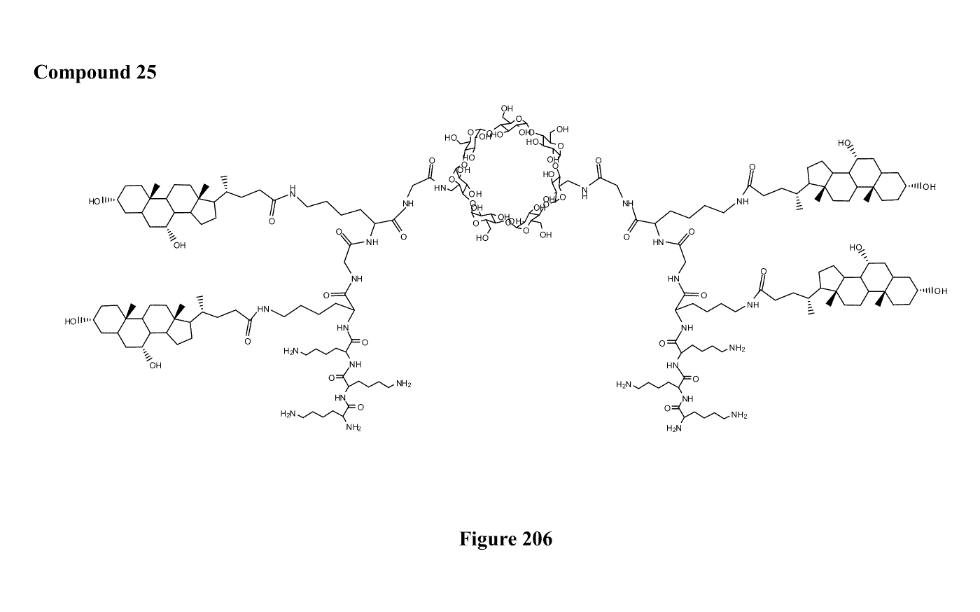

FIG. 207 presents the structure of compound 26 from Example 11.

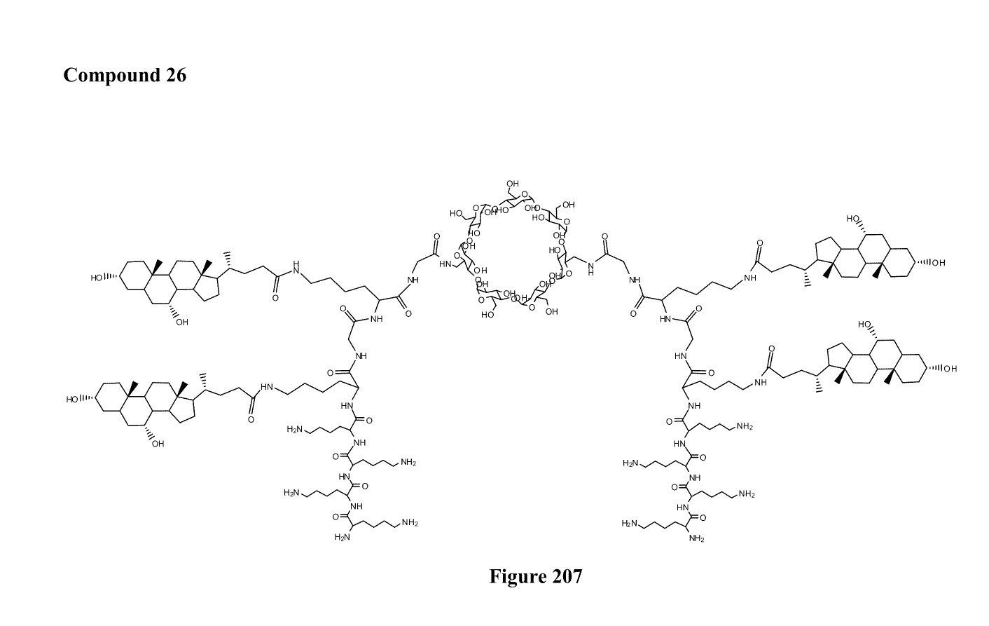

FIG. 208 presents the structure of compound 27 from Example 11.

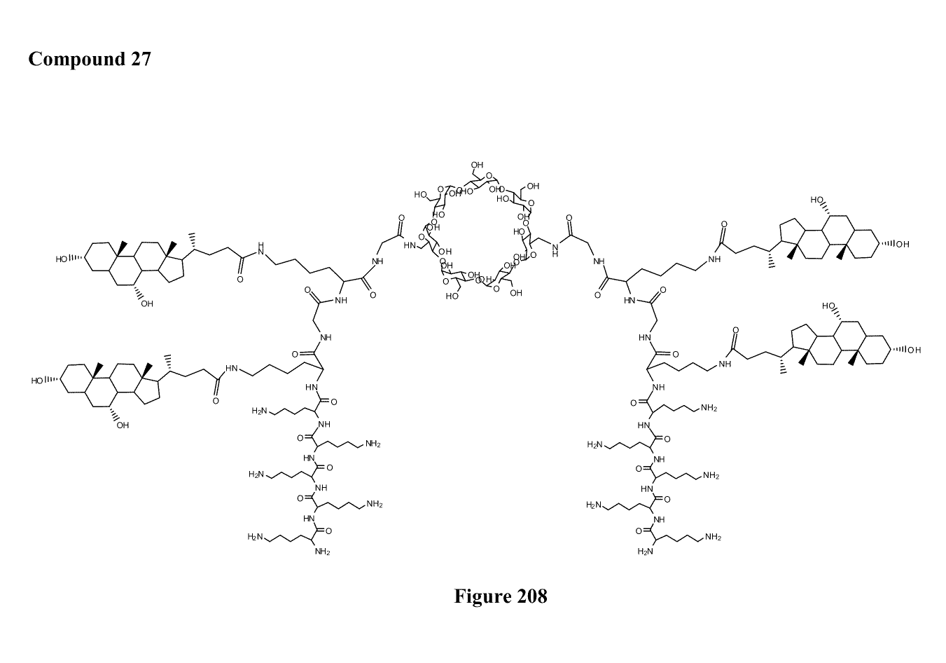

FIG. 209 presents the structure of compound 28 from Example 11.

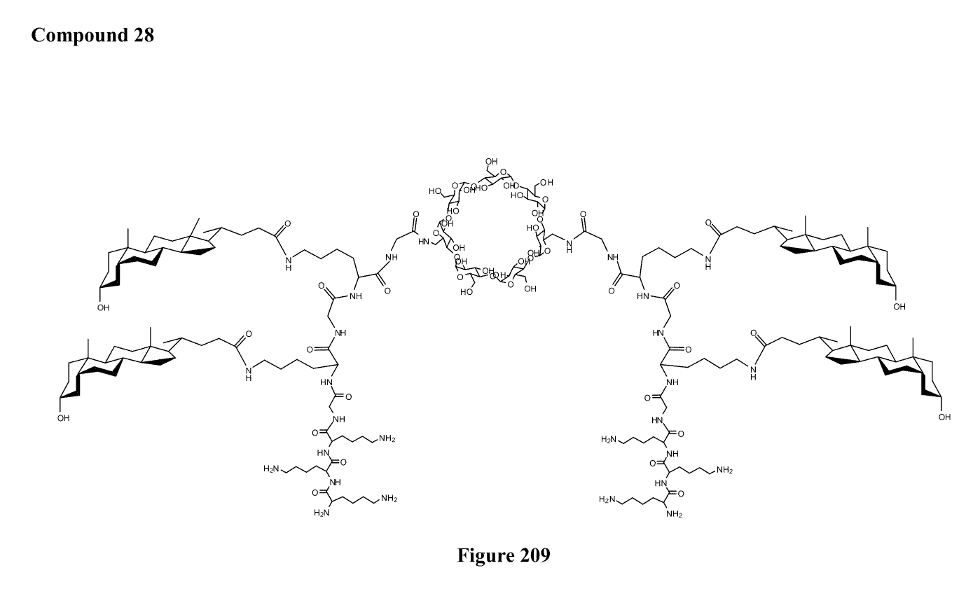

FIG. 210 presents the structure of compound 29 from Example 11.

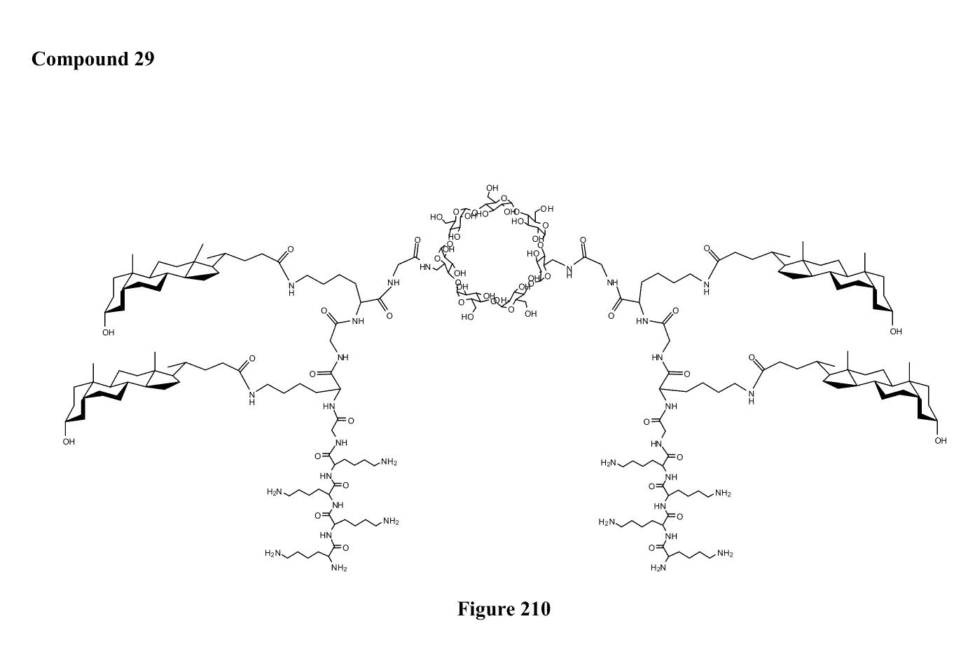

FIG. 211 presents the structure of compound 30 from Example 11.

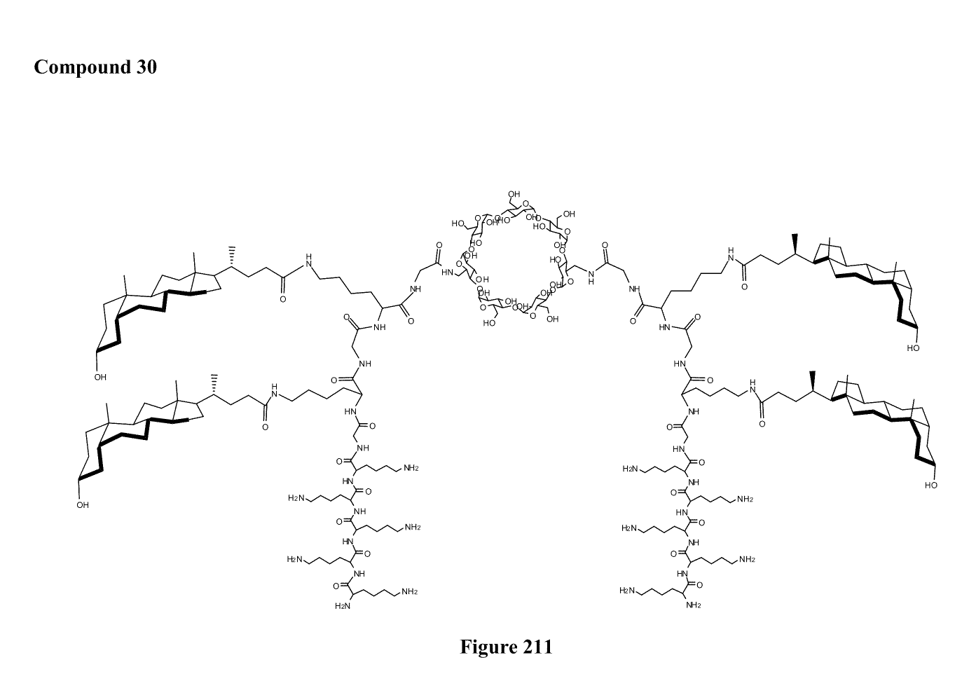

FIG. 212 presents the structure of compound 31 from Example 11.

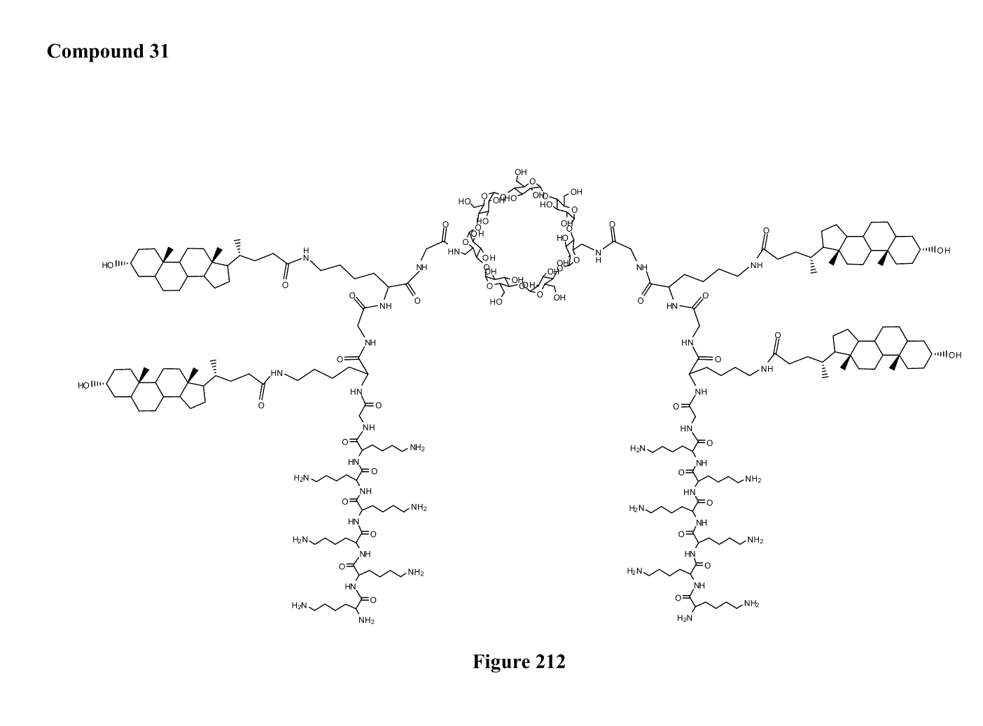

FIG. 213 presents the structure of compound 32 from Example 11.

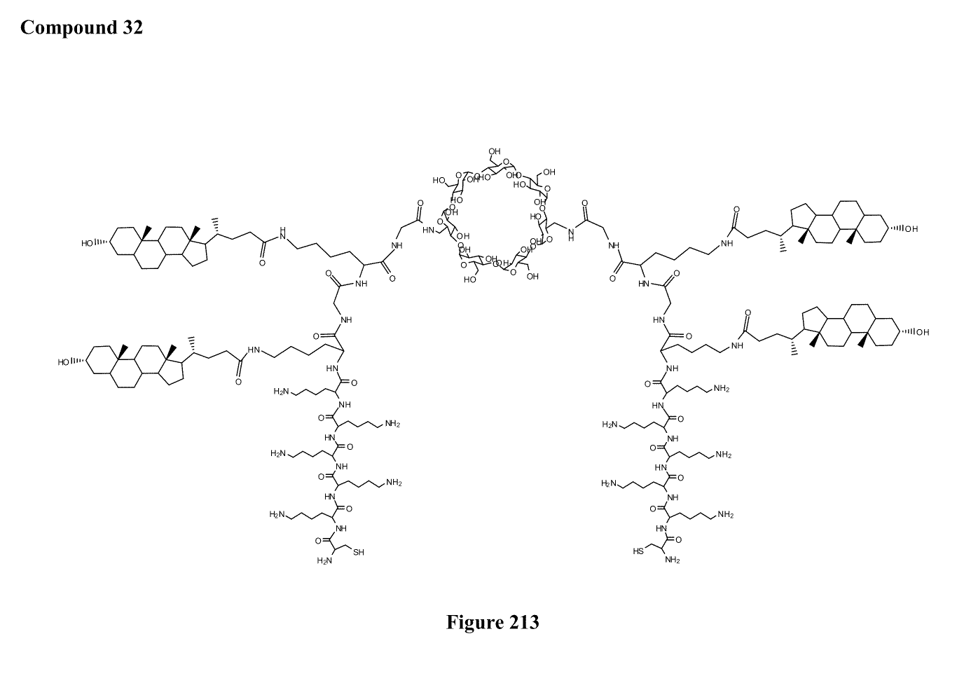

FIG. 214 presents the structure of compound 33 from Example 11.

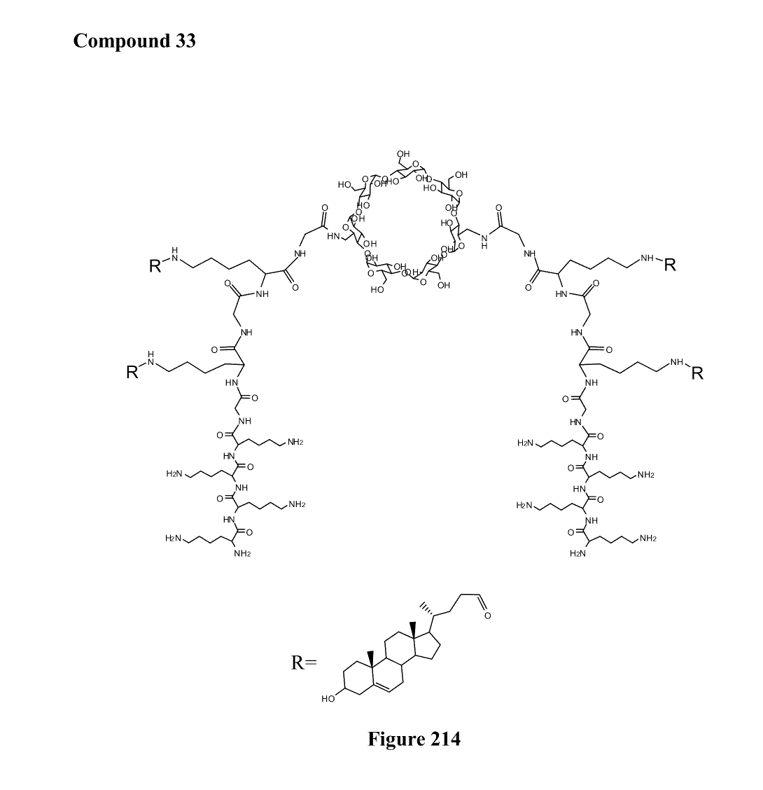

FIG. 215 presents the structure of compound 34 from Example 11.

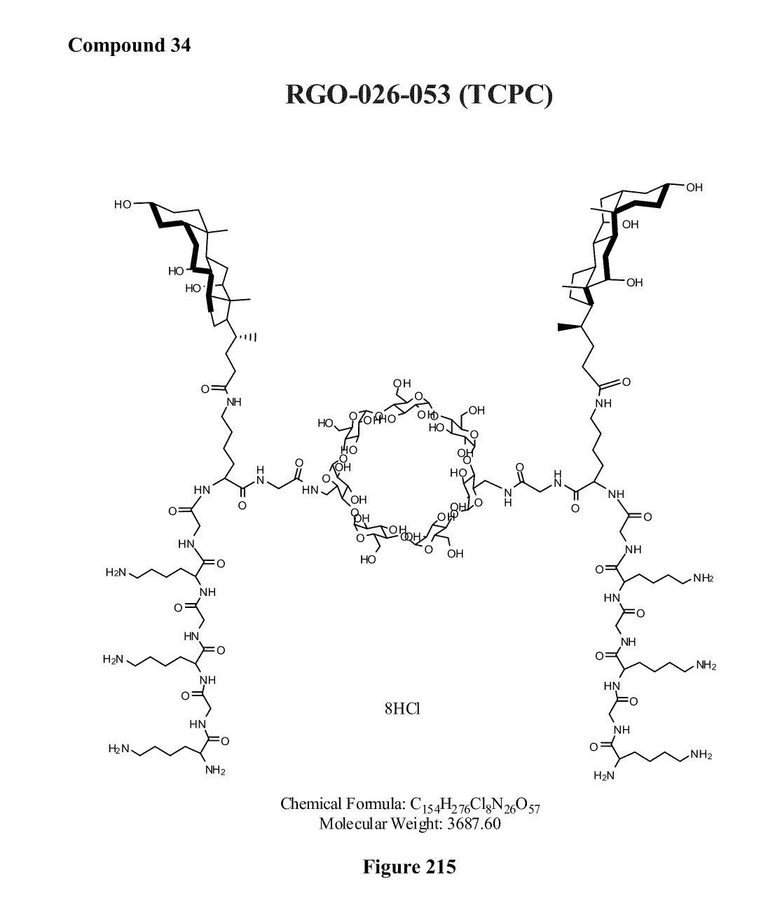

FIG. 216 presents the structure of compound 35 from Example 11.

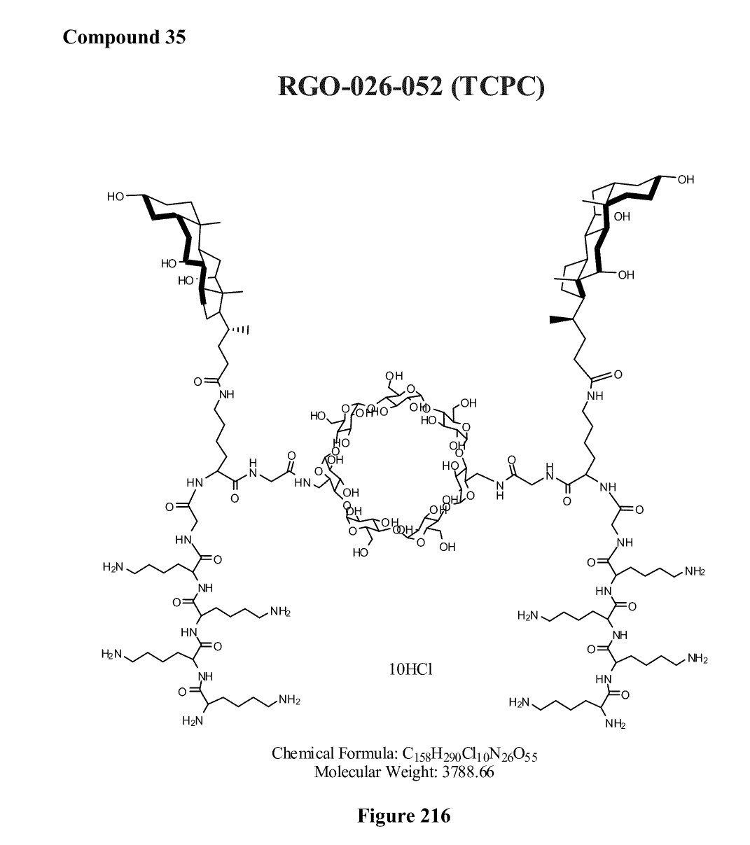

FIG. 217 presents the structure of compound 36 from Example 11.

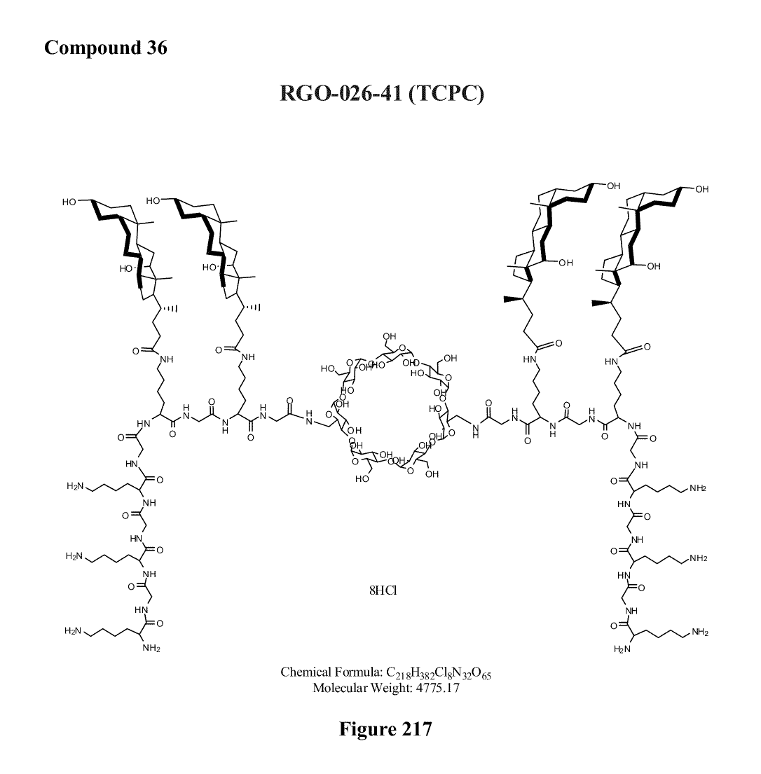

FIG. 218 presents the structure of compound 37 from Example 11.

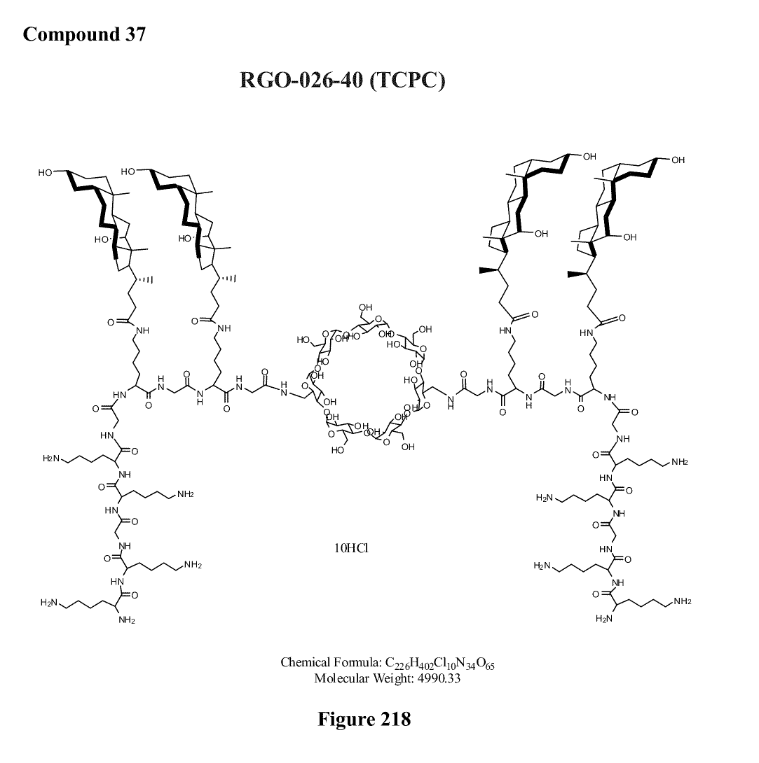

FIG. 219 presents the structure of compound 38 from Example 11.

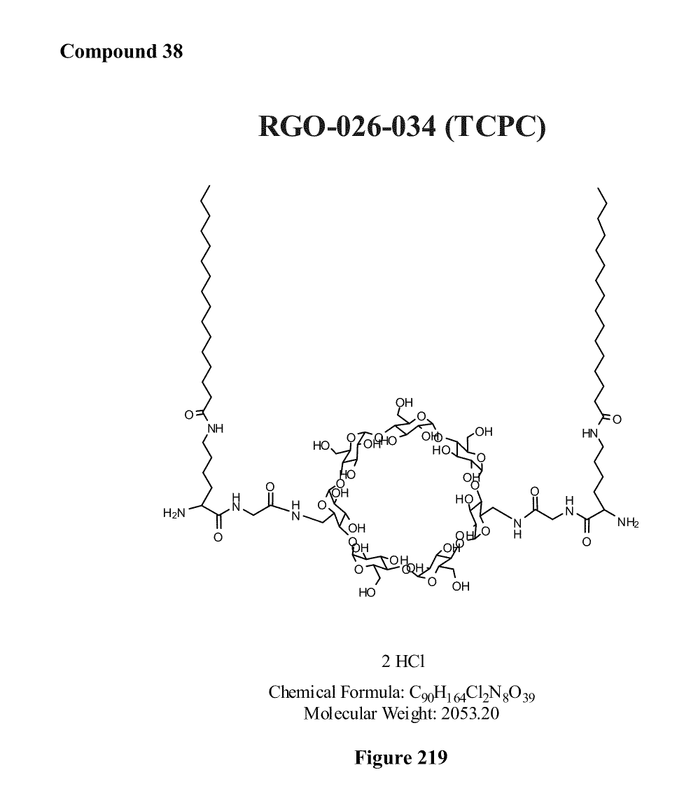

FIG. 220 presents the structure of compound 39 from Example 11.

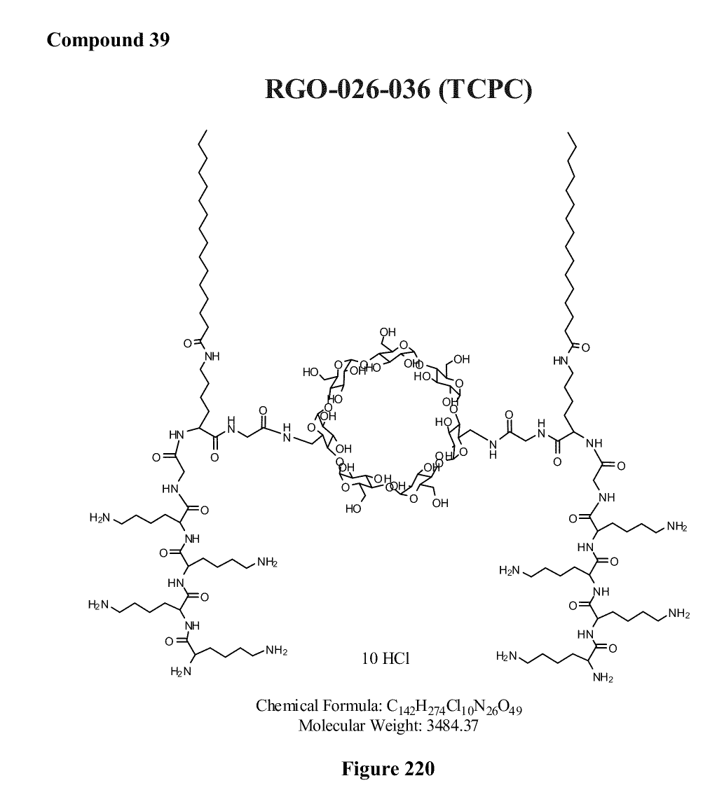

FIG. 221 presents the structure of compound 40 from Example 11.

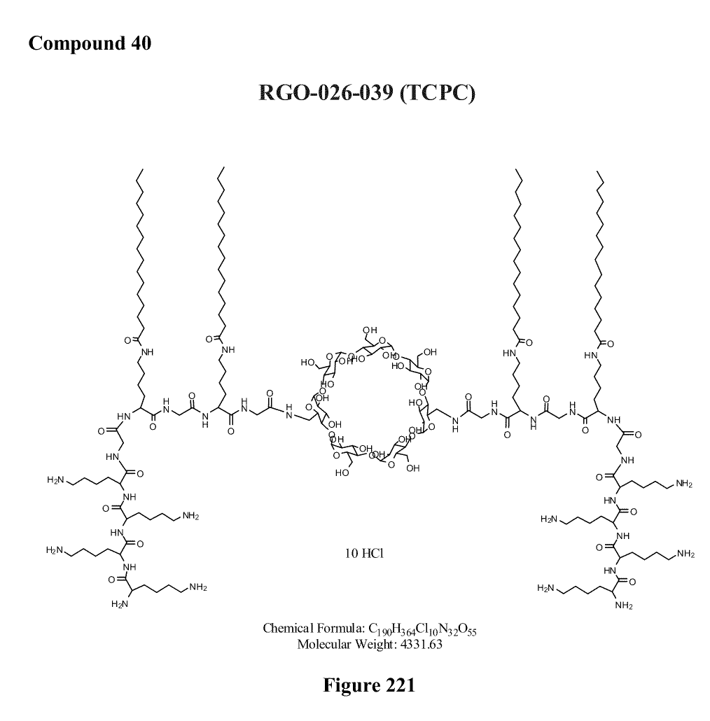

FIG. 222 presents the structure of compound 41 from Example 11.

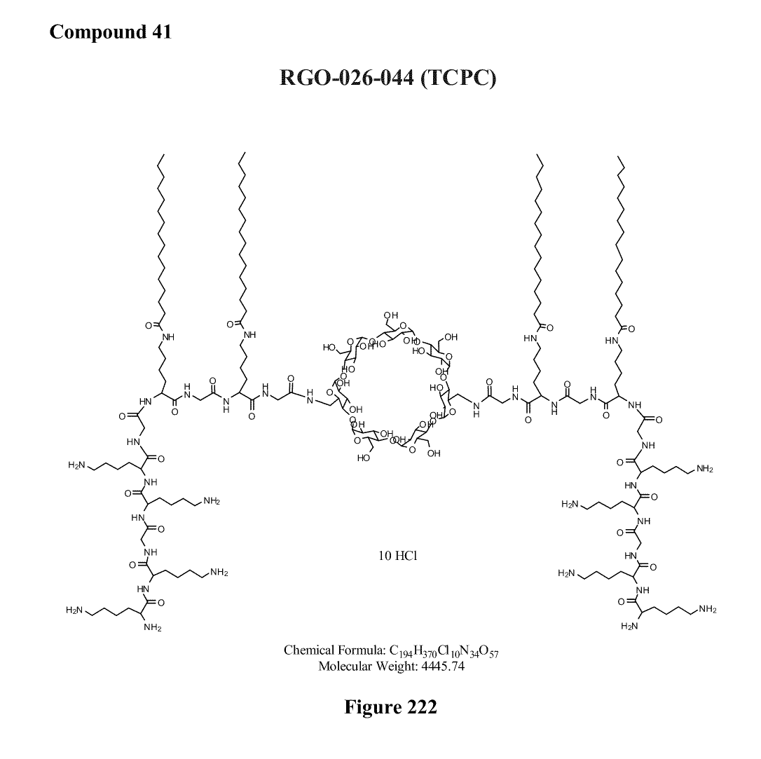

FIG. 223 presents the structure of compound 42 from Example 11.

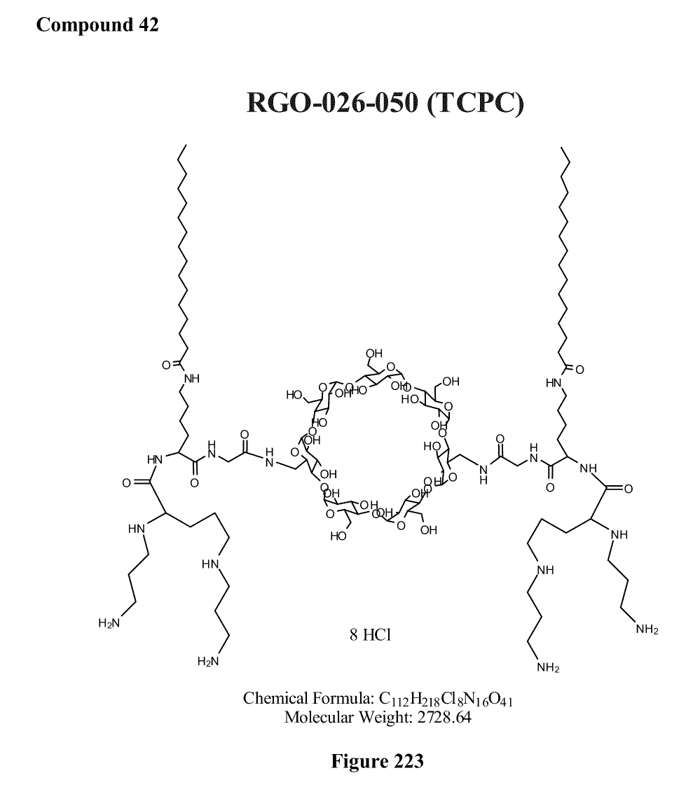

FIG. 224 presents the structure of compound 43 from Example 11.

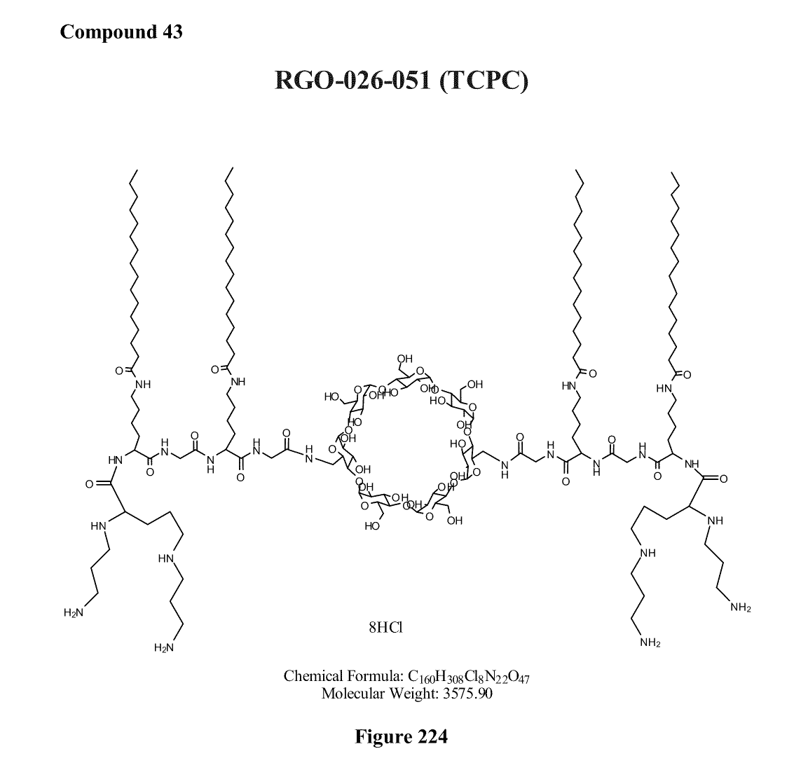

FIG. 225 presents the structure of compound 45 from Example 11.

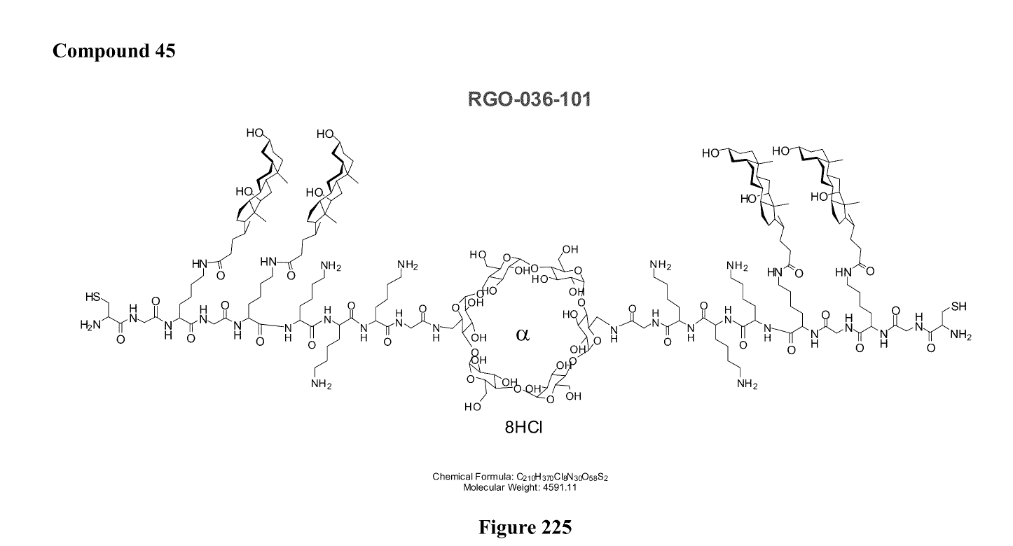

FIG. 226 presents the structure of compound 46 from Example 11.

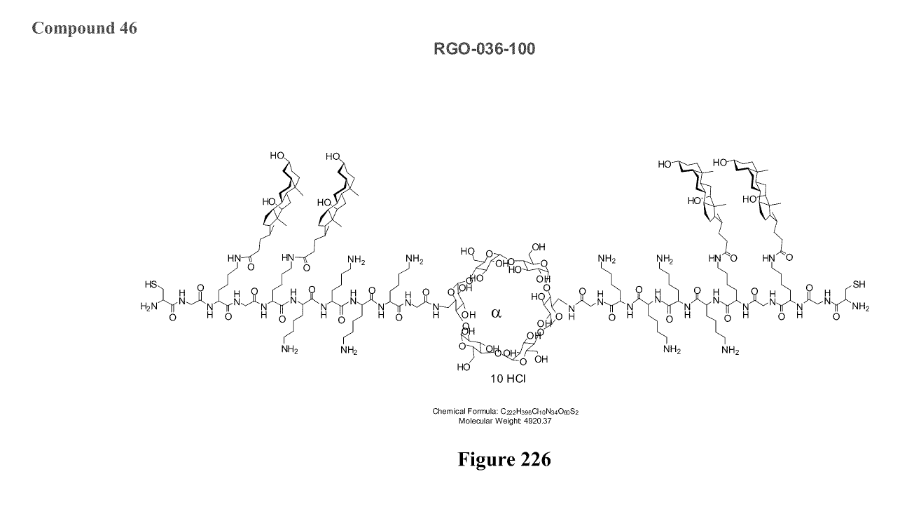

FIG. 227 presents the structure of compound 47 from Example 11.

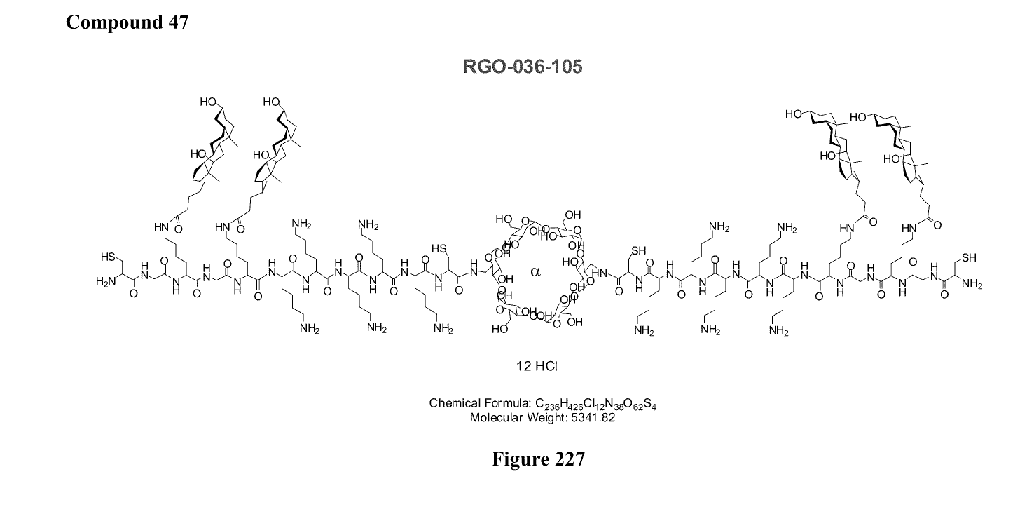

FIG. 228 presents the structure of compound 48 from Example 11.

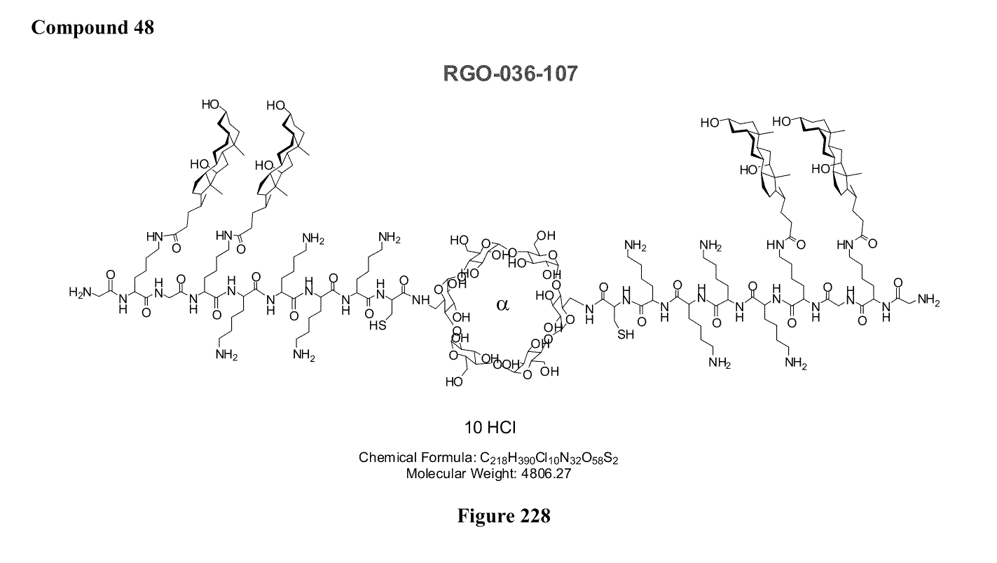

FIG. 229 presents the structure of compound 49 from Example 11.

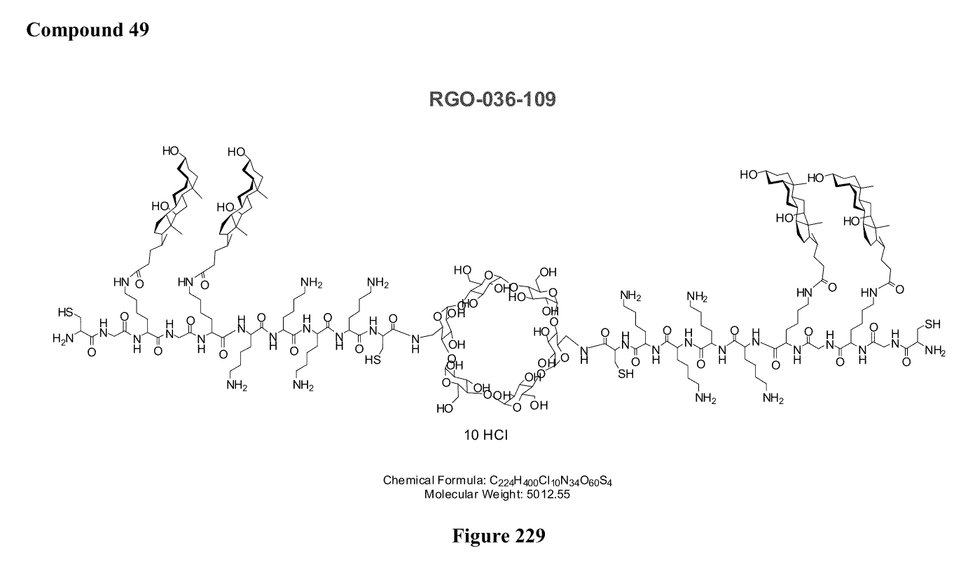

FIG. 230 presents the structure of compound 50 from Example 11.

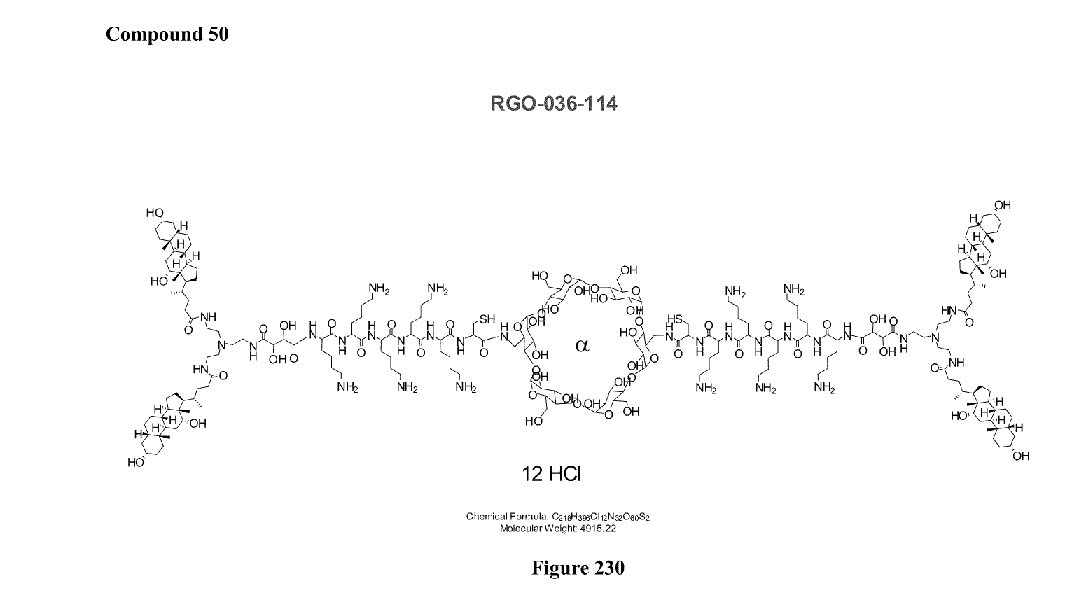

### DETAILED DESCRIPTION OF INVENTION

In accordance with the present invention, there are provided molecular entities comprising:
an amphiphilic core having
at least two positively charged arms covalently attached thereto, and
a plurality of lipophilic (e.g., bile acid) moieties covalently attached to said positively charged arms,
   wherein said positively charged arms are optionally symmetrically substituted with said plurality of lipophilic (e.g., bile acid) moieties. Preferably, each of said positively charged arms is substituted with two lipophilic (e.g., bile acid) moieties.

In certain embodiments of the present invention, the lipophilic (e.g., bile acid) moieties of the molecular entity are capable of forming a stable complex with said amphiphilic core. To facilitate such interaction, in certain embodiments of the present invention it is presently preferred that the charged arms are of sufficient length and flexibility so as to allow said lipophilic (e.g., bile acid) moieties to form a stable complex with said amphiphilic core.

Invention molecular entities are useful for a variety of applications, e.g., such entities bind, stabilize and/or facilitate cellular delivery of opposite charged molecules. In some embodiments, the molecular entities bind, stabilize and/or facilitate cellular delivery of anionic charged molecules when positively charged arms are used.

Exemplary molecular entities according to the present invention have the Formula I, as follows:

A(-B)ₙ

wherein:
A is an amphiphilic core,
each B is independently a positively charged arm having a plurality of positive charges thereon, and a plurality of bile acid moieties covalently attached thereto, and
n is an integer from 2 to 7.
   In certain embodiments of the invention, it is presently preferred that the number of positively charged arm fall in the range of 2 to 4 (i.e., n is an integer from 2 to 4).

In accordance with the present invention, the amphiphilic cores employed herein can be any amphiphilic molecules that have at least two attachment sites typically separated by a distance in the range of about 5-35 Angstroms for linkage of said arms to said core.

The amphiphilic core may be an atom, a linearly extended structure, a branched structure, a cyclic structure, a macrocyclic structure or a cyclic peptide, wherein said core provides at least two attachment points for the charged arms. In some embodiments, the amphiphilic core provides at least three attachment points for the charged arms. In some embodiments, the amphiphilic core provides at least four attachment points for the charged arms. In some embodiments, the amphiphilic core provides at least five attachment points for the charged arms. In some embodiments, the amphiphilic core provides at least six attachment points for the charged arms. In some embodiments, the amphiphilic core provides at least seven attachment points for the charged arms.

In certain embodiments, the amphiphilic core may also interact with charged molecules, e.g., nucleic acid base pairs. The amphiphilic core is selected so as to interact with at least a portion of target molecules, e.g., solvent-exposed bases (purine and pyrimidine heterocycles) in nucleic acids, specifically such bases that are involved in base-pairing via hydrogen bonding. The amphiphilic core may be, on one side, a substantially flat or minimally convex surface which has relatively lower polarity (lower hydrophilicity) than the opposite side of the core, which has relatively higher polarity (higher hydrophilicity). This characteristic facilitates interaction with at least a portion of certain target molecules, e.g., solvent-exposed bases (e.g. purine or pyrimidine heterocycles) in nucleic acids, specifically such bases that are involved in base-pairing via hydrogen bonding. By interacting in said manner, the core surface of lower hydrophilicity shields the hydrophobic surface of the target molecules from interaction with other portions of the target molecules, and from unfavorable interactions with the solvent, which both potentially lead to aggregation and precipitation of the target molecules. Favorable interactions with the solvent, which might improve solubility of the complex, are achieved via the core surface of higher hydrophilicity, opposite to the surface of lower hydrophilicity.

Examples of linear core systems contemplated for use in the practice of the present invention, i.e., linear core systems with at least two attachment sites separated by a distance in the range of about 5-35 Angstroms, include substituted biphenyls (10 Angstrom distance between anchor points (A,B) at the para-positions), substituted biphenyl ethers (10 Angstrom distance between anchor points at the para-positions), bilirubin (15 Angstrom distance between anchor points for arms) and octaphenyl (35 Angstrom distance between anchor points for arms) as illustrated below.

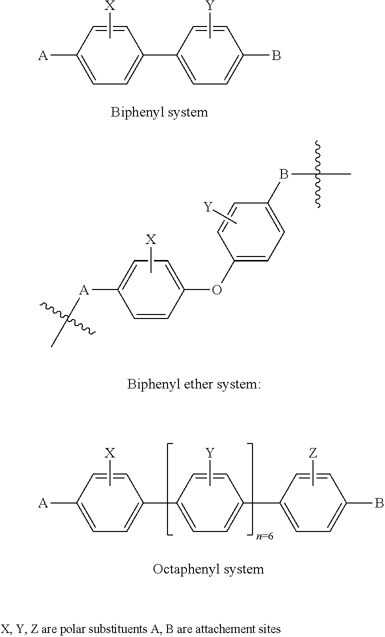

Additional linear core systems contemplated for use in the practice of the present invention include polyalkylene oxides having the structure —(CH₂CH₂—O)₁₋₅—CH₂CH₂—, and the like.

Exemplary macrocyclic molecules contemplated for use in the practice of the present invention as the amphiphilic core include cyclic peptides, cyclic oligosaccharides (e.g. cyclodextrins), cyclic oligoethyleneglycols, substituted porphyrins, substituted corrins, substituted corroles, or the like (see examples illustrated below).

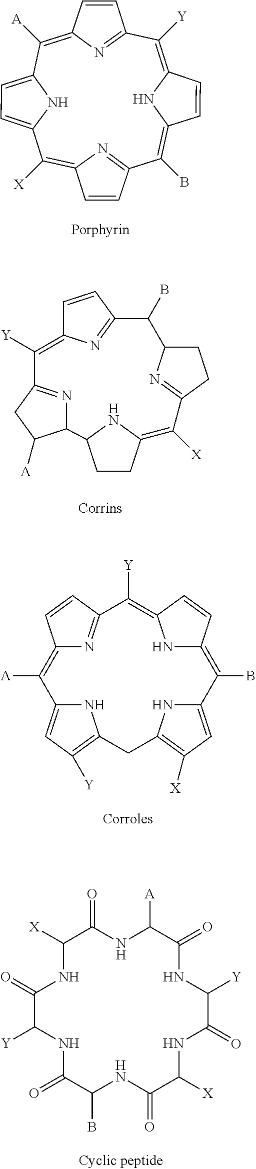

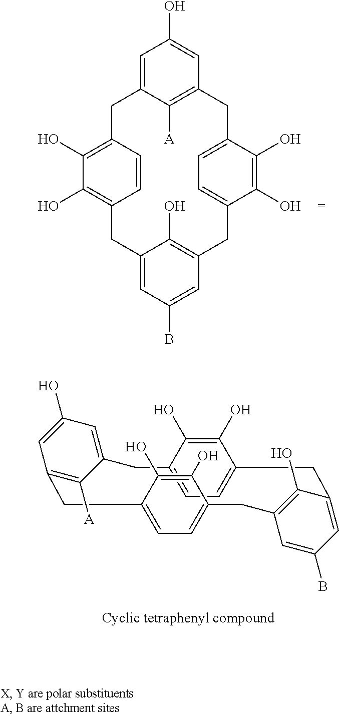

In preferred embodiments, the macrocyclic molecules may be cyclodextrins, e.g. α-cyclodextrin, β-cyclodextrin or γ-cyclodextrin.

Cyclodextrins (CDs) are a group of cyclic polysaccharides comprising six to eight naturally occurring D(+)-glucopyranose units in alpha-(1,4) linkage. The numbering of the carbon atoms of D(+)-glucopyranose units is illustrated below.

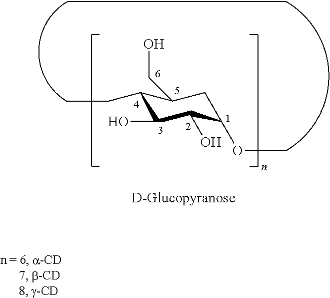

CDs are classified by the number of glucose units they contain: α-cyclodextrin has six glucose units; β-cyclodextrin has seven; and γ-cyclodextrin has eight. Each glucose unit is referred to as ring A, ring B, etc., as exemplified below for β-CD. The diameter of β-CD is measured to be around 15 Angstroms. In accordance with the present invention, the charged arms may be attached via the 6 positions of the A,C-, A,D- or A,E-rings of cyclodextrins.

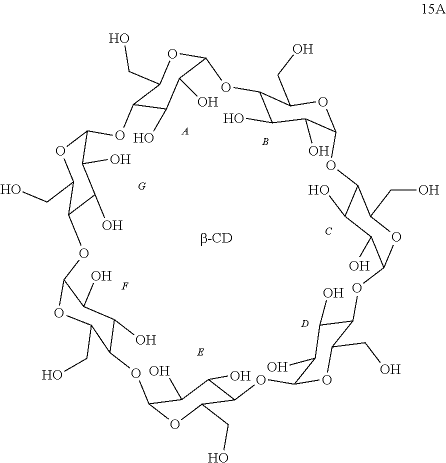

The three-dimensional architecture of CDs consists of cup-like shapes with relatively polar exteriors and nonpolar interiors. The resulting amphiphilic structure is thought to be able to imbibe hydrophobic compounds to form host-guest complexes. According to both in vitro and in vivo studies, CDs, especially alkylated CD derivatives, may have enhancer activity on transport through cell membranes. For example, Agrawal et al. (U.S. Pat. No. 5,691,316) describes a composition including an oligonucleotide complexed with a CD to achieve enhancing cellular uptake of oligonucleotide.

Additional core structures contemplated for use in the practice of the present invention include the following:

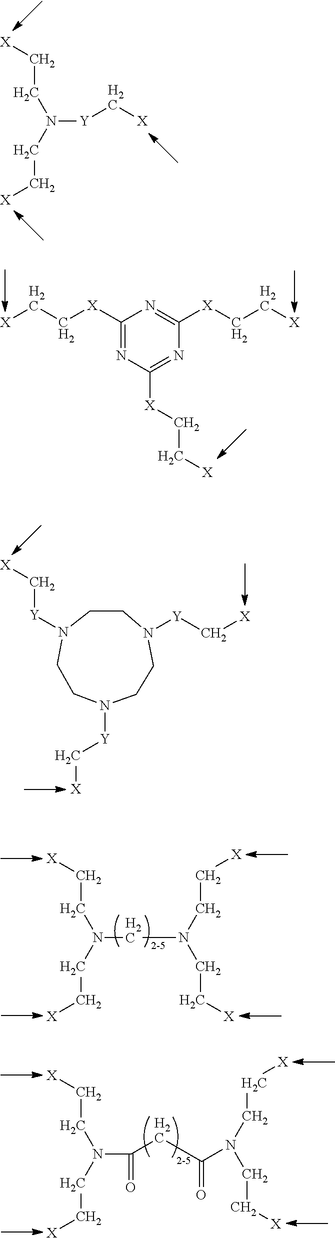

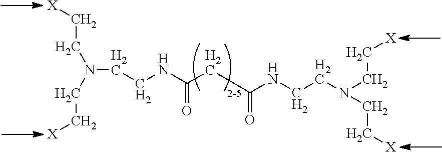

wherein:
→ identifies the atom through which the positively charged arm is attached to the amphiphilic core;
X═NH, O, S, CH₂NH, C(═O), SO₂, SO₂NH or NHC(═O); and
Y═CH₂, O, or C(═O).

In some embodiments, the positively charged arms comprise a plurality of residues selected from amines, guanidines, amidines, N-containing heterocycles, or combinations thereof. In related embodiments, one or both of the positively charged arms further comprises neutral and/or polar functional groups. In related embodiments, each positively charged arm may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. In preferred embodiments, each positively charged arm may be an oligomer selected from the group consisting of oligopeptide, oligoamide, cationically functionalized oligoether, cationically functionalized oligosaccharide, oligoamine, oligoethyleneimine, and combinations thereof. The oligomers may be oligopeptides where all the amino acid residues of the oligopeptide are capable of forming positive charges. Yet in other embodiments, the length of the contiguous backbone of each positively charged arm is about 12 to 200 Angstroms. For example, the positively charged arms may be oligopeptides comprising 3 to 15 amino acids (approximately 12 to 80 Angstroms); preferably 3 to 10 amino acids (approximately 12 to 55 Angstroms).

As used herein, the term “amino acids” include the (D) and (L) stereoisomers of such amino acids when the structure of the amino acid admits stereoisomeric forms. The configuration of the amino acids and amino acid residues herein are designated by the appropriate symbols (D), (L) or (DL), furthermore when the configuration is not designated the amino acid or residue can have the configuration (D), (L) or (DL).

As used herein, the term “cationically functional monosaccharides” may include any amine-containing monosaccharide such as glucosamine, galactosamine and 2-amino-sialic acid. It may also include any natural or unnatural derivatized monosaccharides containing one or more functional groups that can form positive charge, e.g. amine and phosphorus containing groups.

As used herein, the term “cationically functionalized oligosaccharide” is an oligosaccharide comprising one or more “cationically functionalized monosaccharides.”

As used herein, the term “cationically functionalized ethylene glycols” may include any substituted ethylene glycols where the substituents comprise functional groups that can form positive charge, e.g. amine and phosphorus containing groups.

As used herein, the term “cationically functionalized oligoether” may include any substituted oligoether where the substituents comprise functional groups that can form positive charge, e.g. amine and phosphorus containing groups.

In certain embodiments of the present invention, the positively charged arms of the molecular entities have the Formula (II) as follows:

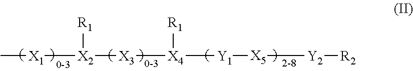

wherein:
each occurrence of X₁, X₂, X₃ and X₄ is independently a monomer unit;
X₅ is a monomer unit having at least one positive charge;
Y₁ is a short spacer;
Y₂ is an extended spacer;
each R₁ is independently selected from the group consisting of bile acids; and
R₂ is H, an amine or a polyethyleneglycol polymer (PEG) optionally linked to a fusogenic moiety, a targeting moiety, or a cell membrane active moiety.

As readily understood by those of skill in the art, each occurrence of X₁, X₂, X₃, X₄ and/or X₅ can be the same or different, i.e., when more than one X₁ is present, each repeat thereof can be the same or different. The same is true for each of the other monomer units, X₂, X₃, X₄ and X₅.

As used herein, the term “short spacer” refers to a bond, a monomer unit having the general Formula (VI) (with the proviso that said monomer unit does not have a cationically charged side chain thereon), and the like, as well as combinations of any two or more thereof.

As used herein, the term “extended spacer” refers to a bond, a monomer unit having the general Formula (VI) (with the proviso that said monomer unit does not have a cationically charged side chain thereon), an hydroxylated alkyl chain, a multi-hydroxylated alkyl chain (i.e., an open-chain carbohydrate), a polyethylene glycol (PEG), and the like, as well as combinations of any two or more thereof.

In certain embodiments of the present invention, the positively charged arms of the molecular entities have the Formula (IIa):

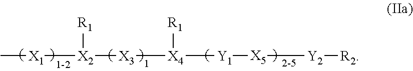

In another embodiment of the present invention, the positively charged arms of the molecular entities have the Formula (III) as follows:

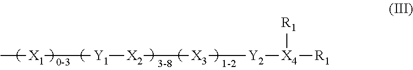

wherein:
each occurrence of X₁ and X₃ is independently a monomer unit;
X₂ is a monomer unit having at least one positive charge;
X₄ is a monomer unit having two sites of attachment;
Y₁ is a short spacer;
Y₂ is an extended spacer; and
each R₁ is independently a bile acid.

In certain embodiments of the present invention, the positively charged arms of the molecular entities have the Formula (IIIa):

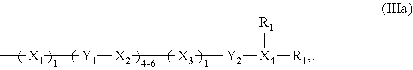

In still another embodiment of the present invention, the positively charged arms of the molecular entities have the Formula (IV) as follows:

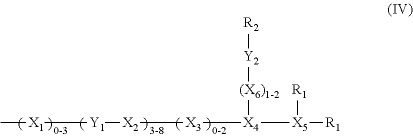

wherein:
each occurrence of X₁, X₃, and X₆ is independently a monomer unit;
X₂ is a monomer unit having at least one positive charge;
X₄ is a monomer having a site for attachment;
X₅ is a monomer unit having two sites of attachment;
Y₁ is a short spacer;
Y₂ is an extended spacer;
each R₁ is independently a bile acid; and
each R₂ is independently selected from the group consisting of H, an amine and a polyethyleneglycol polymer (PEG) optionally linked to a fusogenic moiety, a targeting moiety, or a cell membrane active moiety.

In certain embodiments of the present invention, the positively charged arms of the molecular entities have the Formula (IVa):

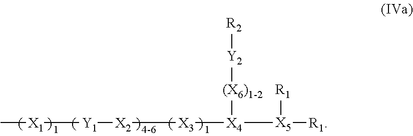

In accordance with still further embodiments of the present invention, the positively charged arms of the molecular entities have the Formula (V) as follows:

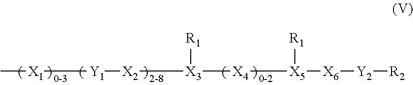

wherein:
each occurrence of X₁, X₃, X₄, X₅ and X₆ is independently a monomer unit;
X₂ is a monomer unit having at least one positive charge;
Y₁ is a short spacer;
Y₂ is an extended spacer;
each R₁ is independently a bile acid; and
R₂ is H, an amine, or a polyethyleneglycol polymer (PEG) optionally linked to a fusogenic moiety, a targeting moiety, or a cell membrane active subunit.

In certain embodiments of the present invention, the positively charged arms of the molecular entities have the Formula (Va):

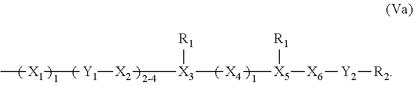

In accordance with the present invention, each occurrence of monomer units X₁, X₂, X₃, X₄, X₅ and X₆ is independently represented by compounds having the general Formula (VI) as follows:

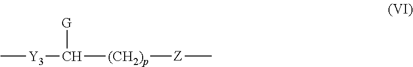

wherein:
G is hydrogen, lower alkyl, or functionalized lower alkyl having any alpha-amino acid side chain, or a cationically or an anionically functionalized side chain thereon;
Y₃ is a covalent bond, O, NR¹, C(O), S or SO₂,
Z is a covalent bond, O, NR¹R², C(O), NR¹C(O), C(O)NR¹, S or SO₂,
R¹ and R² are independently a bond, hydrogen, lower alkyl or heteroatom-substituted lower alkyl; and
p is 0, 1, 2, 3, 4, 5 or 6.

In presently preferred embodiments, G is a cationically functionalized side chain with a length of about 2 to 12 Angstroms comprising functional groups that form one or more positive charges, e.g. amine or phosphorus-containing functional groups. G may be —CH₂—(CH₂)ₙ—W; wherein W is amino, amidino, guanidinyl, imidazolyl or phosphorus containing group. Examples of such side chain may include lysine side chain, arginine side chain, histidine side chain, ornithine side chain, and the like. The skilled artisan would readily realize when n=1,—CH₂—(CH₂)ₙ—W is about 3 Angstroms in length and when n=10, —CH₂—(CH₂)ₙ—W, is about 12 Angstroms in length. The skilled artisan could also readily identify other side chains suitable for use in the practice of the present invention.

In accordance with certain aspects of the present invention, the length of the contiguous backbone of the charged arms is selected so as to correspond to the specific oppositely charged entities which are intended to interact with the molecular entities. In some embodiments, the length of the contiguous backbone of each of the charged arms is 12 to 200 Angstroms; preferably 12 to 160 Angstroms; more preferably 12 to 120 Angstroms; most preferably 12 to 80 Angstroms. For example, when the amphiphilic core provides an anchor for one end of a charged molecule (such as a nucleic acid strand), and assuming that the closest distance between two stacked nucleotides is around 2.5 Angstroms, the lower limit of 12 Angstroms for the arm length corresponds to a nucleic acid of about 5 nucleotides while the upper limit of 200 Angstroms corresponds to a nucleic acid of about 80 nucleotides.

In some embodiments, the anionic charged entity may be a double-stranded or hairpin nucleic acid. In other embodiments, the anionic charged entities may be selected from the group consisting of single-stranded DNA, double-stranded DNA, single-stranded RNA, double-stranded RNA, and oligonucleotide comprising non-natural monomers including 2′-methoxy or 2′-fluoro-modified nucleotides with ribo- or arabino- stereochemistry at the 2′-position, or thio-substituted phosphate groups or the like. The single-stranded RNA may be mRNA or miRNA. The double-stranded RNA may be siRNA. Exemplary siRNA contemplated for use herein is an siRNA that causes destruction of messenger RNA corresponding thereto in cells.

As used herein, the term “nucleic acids” are oligonucleotides consisting of deoxyribonucleic acid (DNA) or ribonucleic acid (RNA), or chimeric oligonucleotides, containing DNA and RNA, or oligonucleotide strands containing non-natural monomers, including but not limited to 2′-methoxy or 2′-fluoro-modified nucleotides with ribo- or arabino-stereochemistry at the 2′-position, or thio-substituted phosphate groups. Nucleic acids contemplated for use in the practice of the present invention may also include conjugated nucleic acids where nucleic acids conjugate to protein, polypeptide or any organic molecules.

As used herein, “double-stranded nucleic acids (hybrids)” are formed from two individual oligonucleotide strands of substantially identical length and complete or near-complete sequence complementarity (“blunt end hybrids”) or offset sequence complementarity (“symmetrical overhang hybrids”, not necessarily implying sequence identity of the overhanging monomers), or from strands of different lengths and complete or offset sequence complementarity (“overhang hybrids”). In symmetrical overhang hybrids, preferred number of the non-hybridized overhang nucleotides is between 1-10; more preferred is between 1-4; most preferred is between 1-2.

As used herein, “sequence complementarity” is defined as the ability of monomers in two oligonucleotides to form base pairs between one nucleotide in one strand and another nucleotide in the second strand by formation of one or more hydrogen bonds between the monomers in the base pair.

As used herein, “complete sequence complementarity” means that each residue in a consecutive stretch of monomers in two oligonucleotides participates in base pair formation.

As used herein, “near-complete sequence complementarity” means that a consecutive stretch of base pairs is disrupted by no greater than one unpaired nucleotide per 3 consecutive monomers involved in base pairing. Preferably, base pairing refers to base pairs between monomers that follow the Watson-Crick rule (adenine-thymine, A-T; adenine-uracil, A-U; guanine-cytosine, G-C) or form a wobble pair (guanine-uracil, G-U).

As used herein, “hairpin nucleic acids” are formed from a single oligonucleotide strand that has complete or near-complete sequence complementarity or offset sequence complementarity between stretches of monomers within the 5′ and 3′ region such that, upon formation of intra-oligonucleotide base pairs, a hairpin structure is formed that consists of a double-stranded (hybridized) domain and a loop domain which contains nucleotides that do not participate in pairing according to the Watson-Crick rule. Preferred length of hairpin oligonucleotides is between 15-70 monomers (nucleotides); more preferred length is between 18-55 monomers; even more preferred length is between 20-35 monomers; most preferred length is between 21-23 monomers. A skilled artisan will realize nucleotides at the extreme 5′ and 3′ termini of the hairpin may but do not have to participate in base pairing.

The terms “polynucleotide” and “nucleic acid molecule” are used broadly herein to refer to a sequence of two or more deoxyribonucleotides, ribonucleotides or analogs thereof that are linked together by a phosphodiester bond or other known linkages. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. The terms also are used herein to include naturally occurring nucleic acid molecules, which can be isolated from a cell using recombinant DNA methods, as well as synthetic molecules, which can be prepared, for example, by methods of chemical synthesis or by enzymatic methods such as by PCR. The term “recombinant” is used herein to refer to a nucleic acid molecule that is manipulated outside of a cell, including, for example, a polynucleotide encoding an siRNA specific for a histone H4 gene operatively linked to a promoter. Preferred length of oligonucleotides in double-stranded nucleic acids is between 15-60 monomers; more preferred length is between 15-45 monomers; even more preferred length is between 19-30 monomers; most preferred length is between 21-27 monomers.

The charged arms may be directly linked to the amphiphilic core via procedures known in the art. For example, oligopeptide arms may be directly attached to the 6 hydroxyl groups of beta-cyclodextrin via an ester linkage. On the other hand, the arms may be indirectly linked to the amphiphilic core via other suitable linkers. In some embodiments, each linker of the entities is independently selected from the group consisting of a disulfide linkage, a protected disulfide linkage, an ether linkage, a thioether linkage, a sulfoxide linkage, a sulfonate linkage, an amine linkage, a hydrazone linkage, a sulfonamide linkage, an urea linkage, an ester linkage, an amide linkage, a carbamate linkage, a dithiocarbamate linkage, and the like, as well as combinations thereof

Linkers with more than one orientation for attachment to the amphiphilic core can be employed in all possible orientations for attachment. For example, an ester linkage may be orientated as —O—C(O)— or —C(O)O—; a sulfonate linkage may be orientated —OS(O)₂— or —S(O)₂O—; a thiocarbamate linkage may be orientated —OC(S)NH— or —NHC(S)O—. A skilled artisan will readily recognize other suitable linkers for attachment of each charged arm.

Lipophilic moieties contemplated for use herein include optionally substituted C₁₄-C₃₀ polycycloalkyl carboxylic acids such as, for example, steroid acids, cholesterol and derivatives thereof. Especially preferred lipophilic moieties contemplated for use herein are bile acids and derivatives thereof.

Bile acids are steroid acids found predominantly in the bile of mammals. Bile salts are bile acids conjugated to glycine or taurine. In humans, taurocholic acid and glycocholic acid (derivatives of cholic acid) represent approximately eighty percent of all bile salts. The two major bile acids are cholic acid, and chenodeoxycholic acid. Bile acids, glycine and taurine conjugates, and 7-alpha-dehydroxylated derivatives (deoxycholic acid and lithocholic acid) are all found in human intestinal bile.

Bile salts constitute a large family of molecules, composed of a steroid structure with four rings, a five or eight carbon side-chain terminating in a carboxylic acid, and the presence and orientation of different numbers of hydroxyl groups. The four rings are labeled from left to right (as commonly drawn) A, B, C, and D, with the D-ring being smaller by one carbon than the other three. The hydroxyl groups have a choice of being in 2 positions, either up (or out) termed beta (often drawn by convention as a solid line), or down, termed alpha (seen as a dashed line in drawings). All bile acids have a hydroxyl group on position 3, which was derived from the parent molecule, cholesterol. In cholesterol, the 4 steroid rings are flat and the position of the 3-hydroxyl is beta.

In many species, the initial step in the formation of a bile acid is the addition of a 7-alpha hydroxyl group. Subsequently, in the conversion from cholesterol to a bile acid, the junction between the first two steroid rings (A and B) is altered, making the molecule bent, and in this process, the 3-hydroxyl is converted to the alpha orientation. Thus, the default simplest bile acid (of 24 carbons) has two hydroxyl groups at positions 3-alpha and 7-alpha. The chemical name for this compound is 3-alpha,7-alpha-dihydroxy-5-beta-cholan-24-oic acid, or as it is commonly known, chenodeoxycholic acid.

Another bile acid, cholic acid (with 3 hydroxyl groups) had already been described, so the discovery of chenodeoxcholic acid (with 2 hydroxyl groups) made the new bile acid a “deoxycholic acid” in that it had one less hydroxyl group than cholic acid. The 5-beta portion of the name denotes the orientation of the junction between rings A and B of the steroid nucleus (in this case, they are bent). The term “cholan” denotes a particular steroid structure of 24 carbons, and the “24-oic acid” indicates that the carboxylic acid is found at position 24, which happens to be at the end of the side-chain. Chenodeoxycholic acid is made by many species, and is quite a functional bile acid. Its chief drawback lies in the ability of intestinal bacteria to remove the 7-alpha hydroxyl group, a process termed dehydroxylation. The resulting bile acid has only a 3-alpha hydroxyl group and is termed lithocholic acid (litho=stone). It is poorly water-soluble and rather toxic to cells. Bile acids formed by synthesis in the liver are termed “primary” bile acids, and those made by bacteria are termed “secondary” bile acids. As a result, chenodeoxycholic acid is a primary bile acid, and lithocholic acid is a secondary bile acid.

The structures of the principal bile acids are as follows:

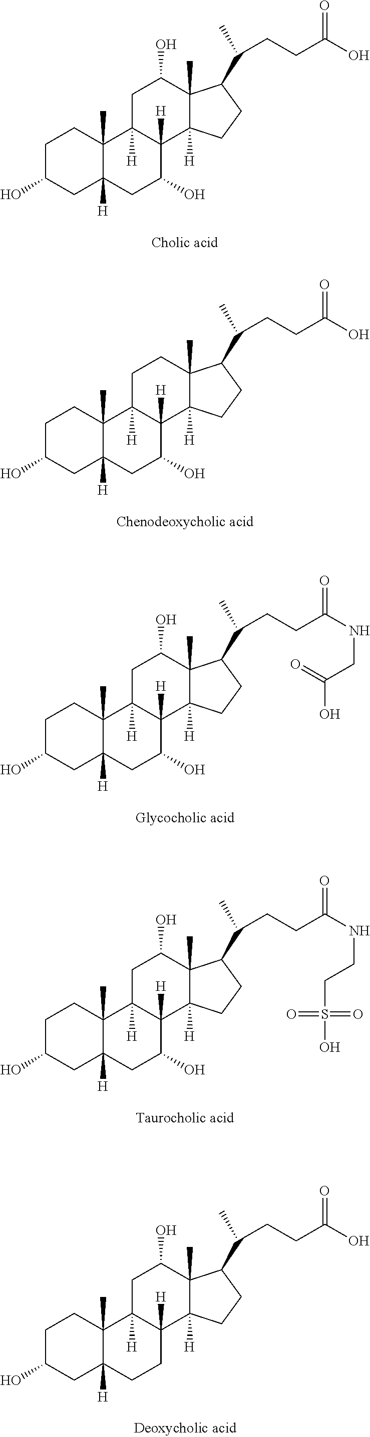

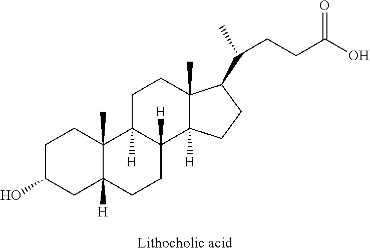

Presently preferred bile acids contemplated for use herein include cholic acid, chenodeoxycholic acid, glycocholic acid, taurocholic acid, deoxycholic acid, lithocholic acid, and the like.

As readily recognized by those of skill in the art, the plurality of lipophilic moieties can be covalently attached to said charged arms via a variety of linkages, e.g., ether, thioether, disulfide, amine, imine, amidine, keto, ester, amide, imide, carboxamide, urea, and the like, linkages. In certain aspects of the invention, it is preferred that the plurality of lipophilic moieties are covalently attached to said charged arms via an ester linkage. In other aspects of the present invention, it is preferred that the plurality of lipophilic moieties are covalently attached to said charged arms via an amide linkage.

In accordance with yet another aspect of the present invention, molecular entities as described herein may further be pegylated. In one aspect, such molecular entities may be partially pegylated. In other aspects, such molecular entities may be substantially completely pegylated. In yet other aspects, such molecular entities may comprise a mixture of non-pegylated, partially pegylated and/or substantially completely pegylated material.

A wide variety of PEG's can be employed in the practice of the present invention, including branched or linear PEG's, and PEG's having a wide range of molecular weights; with molecular weights in the range of about 500 up to about 25,000 being presently preferred.

In certain aspects of the present invention, the PEG employed for preparation of pegylated molecular entities may optionally contain one or more peptide segments which are susceptible to enzymatic cleavage.

When invention molecular entities are pegylated, PEG can be incorporated into the molecular entity in a variety of ways, e.g., via a disulfide linkage, a thioether linkage, an ester linkage, an amide linkage, a maleimide linkage, a thio-maleimide linkage, a sulfone linkage, a carbamate linkage, an urea linkage, and the like.

In some embodiments, invention entities further comprise a bio-recognition molecule. In certain aspects, the bio-recognition molecule could be covalently linked or non-covalently linked to the construct. A wide variety of bio-recognition molecules are contemplated for incorporation into invention molecular entities, e.g., oligopeptides or oligosaccharides that are involved in a large range of biological processes that promote binding or recognition of such oligopeptides or oligosaccharides (examples of such peptidyl-cyclodextrins can be found in Pean et al. J. Chem. Soc. Perkin Trans. 2, 2000, 853-863), cell targeting motifs, cell penetrating motifs, membrane active peptides (e.g., fusogenic peptide sequences, endosomolytic peptide sequences, and the like), and the like. Such bio-recognition molecules can be attached to either the charged arms themselves, or the PEG appendages thereon (if present).

As used herein, the term “cell targeting motifs” embraces a peptide sequence, an epitope on a peptide, or a chemical subunit which has affinitiy to a specific site, location, or recognition site on the surface of a cell without necessarily causing internalization (see, for example, Biochemical Society Transaction (2007) Vlo. 35, 780-783).

As used herein, the term “cell penetrating motifs” embraces a peptide sequence, an epitope on a peptide, or a chemical subunit that translocates the cell membrane and facilitates the transport of various molecular cargo across the cell membrane.

As used herein, the term “membrane active peptides” embraces peptides capable of interacting with and/or destabilizing membrane bilayers. Examples of such peptides include fusogenic peptides, endosomolytic peptides, and the like.

In certain aspects of the present invention, where molecular entities as described herein have one or more thiol groups (SH) thereon, such entities can be converted into dimeric, oligomeric or polymeric forms thereof by interacting the thiol group(s) of a plurality of such molecular entities with one another under conditions suitable to form stable disulfide bonds.

In certain aspects of the present invention, there are also provided compositions comprising an aggregation of a plurality of any of the molecular entities described herein, wherein said aggregation comprises particles of between about 10 nanometers up to about 500 nanometers in size.

In other embodiments, the present invention provides methods for delivering a negatively charged entity to a cell, said method comprising:
a) binding non-covalently a molecular entity as described herein (or oligomer thereof, or aggregate thereof as described herein) to said negatively charged entity, thereby forming a complex; and
b) contacting said cell with said complex; wherein said negatively charged entity is taken up by said cell.
   In related embodiments, the present invention provides methods for delivering a negatively charged entity to a cell, said method comprising contacting said cell with a complex prepared by binding non-covalently a molecular entity as described herein (or oligomer thereof, or aggregate thereof as described herein) to said negatively charged entity, wherein said charged molecule is taken up by said cell.

In yet other embodiments, the present invention provides methods for stabilizing a negatively charged entity in vivo. The methods comprise contacting the negatively charged entity with a molecular entity as described herein (or oligomer thereof, or aggregate thereof as described herein).

In yet other embodiments, the present invention provides methods for reducing the susceptibility of a double-stranded or hairpin nucleic acid to digestion by enzymatic nuclease, said method comprising contacting said nucleic acid with a molecular entity as described herein (or oligomer thereof, or aggregate thereof as described herein). In preferred embodiments, the nuclease is exonuclease.

In yet other embodiments, the present invention provides methods for reducing the susceptibility of a double-stranded or hairpin nucleic acid to hydrolysis of the phosphodiester backbone, said method comprising contacting said nucleic acid with a molecular entity as described herein (or oligomer thereof, or aggregate thereof as described herein).

In yet other embodiments, the present invention provides methods for reducing the susceptibility of charged entities to self-aggregation, said method comprising contacting said charged entity with a molecular entity as described herein (or oligomer thereof, or aggregate thereof as described herein).

In some embodiments, the present invention provides compositions comprising a pharmaceutical excipient, a charged entity and a molecular entity as described herein (or oligomer thereof, or aggregate thereof as described herein), or a pharmaceutically acceptable ester, salt, or hydrate thereof.

As used herein, the term “ pharmaceutical excipient” refers to an inert substance added to a pharmacological composition to further facilitate administration of molecular entities. Examples of pharmaceutical excipients include but are not limited to, calcium carbonate, calcium phosphate, various sugars and types of starch, cellulose derivatives, gelatin, vegetable oils and polyethylene glycols excipient.

As used herein, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutical acceptable ester” within the context of the present invention represents an ester of a construct of the invention having a carboxy group, preferably a carboxylic acid prodrug ester that may be convertible under physiological conditions to the corresponding free carboxylic acid.

As used herein, the term “pharmaceutically acceptable salt” includes salts of acidic or basic groups that may be present in molecular entities used in the present compositions. Molecular entities included in the present compositions that are basic in nature are capable of forming a wide variety of salts with various inorganic and organic acids. The acids that may be used to prepare pharmaceutically acceptable acid addition salts of such basic molecular entities are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Molecular entities included in the present compositions that include an amino moiety may form pharmaceutically acceptable salts with various amino acids, in addition to the acids mentioned above. Molecular entities, included in the present compositions, which are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compositions according to the present invention may be administered to humans and other animals for therapy as either a single dose or in multiple doses. The compositions of the present invention may be administered either as individual therapeutic agents or in combination with other therapeutic agents. The treatments of the present invention may be combined with conventional therapies, which may be administered sequentially or simultaneously. In some embodiments, routes of administration include those selected from the group consisting of oral, intravesically, intravenous, intraarterial, intraperitoneal, local administration, and the like. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection, infusion, and the like.

The phrases “systemic administration,” “administered systemically,” “peripheral administration” and “administered peripherally” as used herein mean the administration of a molecular entity, drug or other material other than directly into the central nervous system, such that it enters the patient's system and, thus, is subject to metabolism and other like processes, for example, subcutaneous administration.

Actual dosage levels of the active ingredients in the compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular molecular entities of the present invention employed, or the ester, salt or amide thereof, the route of administration, the time of administration, the rate of excretion of the particular compositions being employed, the duration of the treatment, other drugs, compounds and/or materials used in combination with the particular motor protein therapeutic employed, the age, sex, weight, condition, general health and prior medical history of the patient being treated, and like factors well known in the medical arts.

In general, a suitable daily dose of a compound of the invention will be that amount of the molecular entities which is the lowest dose effective to produce a therapeutic effect. Such an effective dose will generally depend upon the factors described above. Generally, intravenous, intracerebroventricular and subcutaneous doses of the compositions of the present invention for a patient will range from about 0.0001 to about 100 mg per kilogram of body weight per day.

In other embodiments, the present invention provides complexes comprising an anionic charged entity associated with a molecular entity as described herein (or oligomer thereof, or aggregate thereof as described herein). In preferred embodiments, the amphiphilic core is α, β or γ cyclodextrin. The charge ratio of the molecular entity to said anionic charged molecule ranges from 1:12 to 12:1; preferably ranges from 1:1 to 8:1.

In yet another embodiment of the present invention, there are provided compositions comprising a pharmaceutical excipient, an anionic charged entity and a molecular entity as described herein (or oligomer thereof, or aggregate thereof as described herein). The charge ratio of the entity to the anionic charged entity in the composition may range from 1:12 to 12:1; preferably from 1:1 to 8:1. The anionic charged entity may be double-stranded or hairpin nucleic acid(s). The anionic charged entity may be selected from the group consisting of single-stranded DNA, double-stranded DNA, single-stranded RNA, double-stranded RNA, oligonucleotide comprising non-natural monomers, and the like. The single-stranded DNA, double-stranded DNA, single-stranded RNA and double-stranded RNA may include nucleotides bound to small molecules. In related embodiments, the single-stranded RNAs may be mRNA or miRNA and double-stranded RNA may be siRNA.

In still another embodiment of the present invention, there are provided compositions comprising a pharmaceutical excipient, a cationic charged molecule and a molecular entity as described herein (or oligomer thereof, or aggregate thereof as described herein). The charge ratio of the entity to the cationic charged molecule in the composition may range from 1:12 to 12:1; preferably from 1:1 to 8:1.

### EXAMPLES

The invention now being generally described, it will be more readily understood by reference to the following examples, which are included merely for purposes of illustration of certain aspects and embodiments of the present invention, and are not intended to limit the invention.

Abbreviations used throughout the following examples have the meaning set forth herein unless indicated to the contrary elsewhere:

| | |
|---|---|
| AcOH | Acetic acid, |
| Alloc | Allyloxycarbonyl, |
| Boc | tert-Butoxycarbonyl, |
| DIC | N,N′-Diisopropylcarbodiimide, |
| DIEA | Diisopropylethylamine, |
| DMF | Dimethylformamide, |
| EDT | Ethanedithiol, |
| EDTA | Ethylenediaminetetraacetic acid, |
| Fmoc | 9-Fluorenylmethoxycarbonyl, |
| HATU | 2-(1H-9-Azabenzotrizole-1-yl)-1,1,3,3-tetramethyl-aminium |
| | hexafluorophosphate, |
| HBTU | 2-(1H-Benzotriazol-1-yl)-1,1,3,3-tetramethyl-aminium |
| | hexafluorophosphate, |
| HOBt | N-Hydroxybenzotriazole, |
| TBTU | 2-(1H-Benzotriazol-1-yl)-1,1,3,3-tetramethyl-aminium |
| | tetrafluoroborate, |
| TCEP | tris-(2-Carboxyethyl)phosphine, |
| TES | Triethylsilane, |
| TFA | Trifluoroacetic acid, and |
| Trt | Trityl. |

### Example 1

### General Synthetic Procedures

I . . . for Coupling of Acids with Diaminocyclodextrins (A):

To a solution of carboxylic acid (2.2 eq) in DMF was added TBTU (2.2 eq.), HOBt (2.2 eq), and DIEA (4.4 eq). The mixture was stirred for 2-5 min and added to a solution of diamino functionalized cyclodextrin (1.0 eq) in DMF. The mixture was stirred for 24 h and then evaporated. The residue was suspended in water and the solid was collected by filtration and dried to give cyclodextrin conjugate.

### II . . . for Deprotection of Fmoc Protected Amino Group (B):

Fmoc protected amino compound was dissolved in DMF/piperidine (7:3) mixture and stirred for 1-3 h until Fmoc group is completely removed (monitored by HPLC). The solvent was evaporated and the residue was suspended in diethyl ether. The precipitate was collected and dried to give amino compound.

### III . . . for Removal Acid Sensitive Boc and Trt Groups (C):

Boc and/or Trt protected compound was dissolved in TFA/Et₃SiH/H₂O/ethylenedithiol (90:5:2.5:2.5) mixture and stirred for 1 h. The solvent was evaporated and the residue was dissolved in water and purified by HPLC.

### IV . . . for Removal Alloc Group (D):

To a solution of Alloc protected amino compound in DMF/AcOH/DIEA (10:3:2) mixture was added Pd(PPh₃)₄ (0.1 eq). The mixture was purged with nitrogen and stirred under nitrogen for 12-24 h until all Alloc groups are removed (monitored by HPLC). The solvent was evaporated and the residue was suspended in water. The precipitate was collected, washed with EtOAc and dried to give desired amino compound.

### V . . . for Removal Acid Sensitive Boc and Trt Groups for Compounds Containing Cholic Acids and Thiols (E):

Boc and/or Trt protected compound was dissolved in TFA/Et₃SiH/H₂O/ethylenedithiol (90:5:2.5:2.5) mixture and stirred for 1 h. The solvent was evaporated and the residue was dissolved in 1 mL of 2:1 MeOH/water. Then the vial was flushed with nitrogen and 0.3mL of ammonium hydroxide (50%) was added and stirred under nitrogen for 10-30 min. Then most of the solvent was removed by nitrogen stream and the mixture was immediately acidified by HCl to pH2 and purified by HPLC.

VI . . . for Coupling of Acids with Polyamines (F):

To a solution of carboxylic acid (1.2 eq per amino group) in DMF was added HOBt (1.2 eq), DIEA (2.4 eq), and DIC (4 eq). The mixture was stirred for 2-5 min and added to a solution of polyamino functionalized compound (1.0 eq) in DMF. The mixture was stirred for 24 h and then evaporated. The residue was suspended in water and the solid was collected by filtration and dried to give conjugate with protected side chains.

Boc and/or Trt protected compound was dissolved in TFA/Et₃SiH/H2O (90:5:2.5) mixture and stirred for 20min. The solvent was evaporated and the residue was dissolved in 2:1 MeOH/conc.HCl. Then the vial was flushed with nitrogen and stirred for 10-30 min. Then most of the solvent was removed by nitrogen stream and the mixture was purified by HPLC.

### Example 2

### Preparation of Compounds 2a-2i (2f-2i Peptide Sequences Disclosed as SEQ ID NOS 1-4, Respectively)

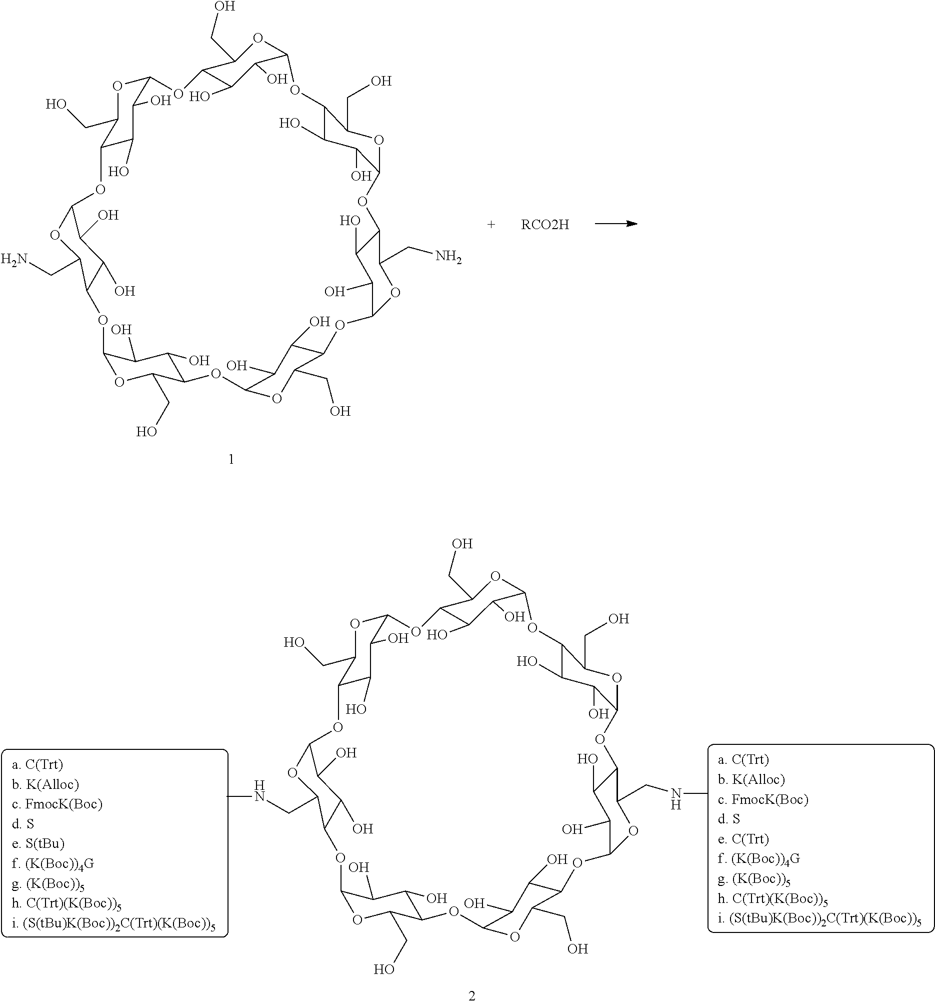

### Preparation of Compound 2a:

Compound 2a was prepared by using general procedure A between diaminocyclodextrin 1 and Fmoc-C(Trt)-OH. Fmoc group was removed using general procedure B to give compound 2a in 83% yield. MS m/z calcd. for C₈₆H₁₁₀N₄O₃₅S₂ 1823, found 1824 (M+H). ¹H-NMR (300 MHz, DMSO-d₆): δ 7.20-7.4 (m, 30H), 5.5-6.0 (m, 12H), 4.83 (s, 7H), 3.0-4.0 (m, 46H), 2.34 (m, 2H), 2.10 (m, 2H).

### Preparation of Compound 2b:

Compound 2b was prepared by using general procedure A between diaminocyclodextrin 1 and Fmoc-K(Alloc)-OH. Fmoc group was removed using general procedure B to give compound 2b. MS m/z calcd. for C₆₂H₁₀₄N₆O₃₉ 1557, found 1558 (M+H). ¹H-NMR (300 MHz, D₂O): δ 5.84 (m, 2H), 5.10-5.25 (m, 4H), 4.95 (s, 7H), 4.45 (m, 4H), 3.0-4.0 (m, 48H), 1.78 (m, 4H), 1.44 (m, 4H), 1.28 (m, 4H).

### Preparation of Compound 2c:

Compound 2c was prepared by using general procedure A between diaminocyclodextrin 1 and Fmoc-K(Boc)-OH to give compound 2c. MS m/z calcd. for C₉₄H₁₃₂N₆O₄₃ 2033, found 2034 (M+H). ¹H-NMR (300 MHz, DMSO-d₆): δ 7.20-7.95 (m, 16H), 5.5-6.0 (m, 12H), 4.83 (s, 7H), 3.0-4.0 (m, 52H), 1.2-1.7 (m, 12H), 1.36 (m, 18H).

### Preparation of Compound 2d:

Compound 2d was prepared by using general procedure A between diaminocyclodextrin 1 and Fmoc-S—OH. Fmoc group was removed using general procedure B to give compound 2b. MS m/z calcd. for C₄₈H₈₂N₄O₃₇ 1306, found 1307 (M+H).

### Preparation of Compound 2e:

To a solution of Fmoc-C(Trt)-OH (1.0 eq) in DMF was added TBTU (1.0 eq.), HOBt (1.0 eq), and DIEA (2.2 eq). The mixture was stirred for 2-5 min and added to a solution of diaminocyclodextrin 1 (1.0 eq) in DMF. The mixture was stirred for 16 h and then evaporated. The residue was purified by HPLC to give monoamino-mono-Fmoc-C(Trt) functionalized cyclodextrin. MS m/z calcd. for C₇₉H₁₀₁N₃O₃₆S 1700, found 1701 (M+H).

To a solution of Fmoc-S(tBu)-OH (1.0 eq) in DMF was added TBTU (1.0 eq.), HOBt (1.0 eq), and DIEA (2.2 eq). The mixture was stirred for 2-5 min and added to a solution of monoamino-mono-Fmoc-C(Trt) functionalized cyclodextrin (1.0 eq) in DMF. The mixture was stirred for 24 h and then evaporated. The residue was suspended in water and the solid was collected by filtration and dried to give mono-Fmoc-S(tBu)-mono-Fmoc-C(Trt) functionalized cyclodextrin. Fmoc group was removed using general procedure B to give compound 2e as a mixture of two isomers in 46% yield. MS m/z calcd. for C₇₁H₁₀₄N₄O₃₆S 1620, found 1621 (M+H). ¹H-NMR (300 MHz, D₂O): δ 7.26-7.47 (m, 15H), 4.90-5.10 (m, 7H), 3.0-4.0 (m, 48H), 1.10-1.25 (two s, 9H).

### Preparation of Compound 2f:

Compound 2f was prepared by using general procedure A between diaminocyclodextrin 1 and Fmoc-(K(Boc))₄G-OH (SEQ ID NO: 1). Fmoc group was removed using general procedure B to give compound 2f. MS m/z calcd. for C₁₃₄H₂₃₈N₂₀O₅₉ 3072, found 1537 ((M+2H)/2).

### Preparation of Compound 2g:

Compound 2g was prepared by using general procedure A between diaminocyclodextrin 1 and Fmoc-(K(Boc))₅-OH (SEQ ID NO: 2). Fmoc group was removed using general procedure B to give compound 2g. MS m/z calcd. for C₁₅₂H₂₇₂N₂₂O₆₃ 3414, found 1708 ((M+2H)/2).

### Preparation of Compound 2 h:

Compound 2h was prepared by using general procedure A between diaminocyclodextrin 1 and Fmoc-C(Trt)(K(Boc))₅-OH (SEQ ID NO: 3). Fmoc group was removed using general procedure B to give compound 2h. MS m/z calcd. for C₁₉₆H₃₁₀N₂₄O₆₅S₂ 4104, found 2053 ((M+2H)/2).

### Preparation of Compound 2i:

Compound 2i was prepared by using general procedure A between cyclodextrin 2h and Fmoc-(S(tBu)K(Boc))₂-OH (SEQ ID NO: 5). Fmoc group was removed using general procedure B to give compound 2i. MS m/z calcd. for C₁₉₆H₃₁₀N₂₄O₆₅S₂ 4104, found 2053 ((M+2H)/2).

### Example 3

### Preparation of Compounds 3b, 3d, 3e, 3j and 3k (Left-Hand Peptide Sequences 3b, 3d, 3e, 3j, and 3k Disclosed as SEQ ID NOS 6-10, Respectively. Right-Hand Peptide Sequences 3b, 3d, 3e, 3j, and 3k Disclosed as SEQ ID NOS 6-7, 12, & 9-10, Respectively)

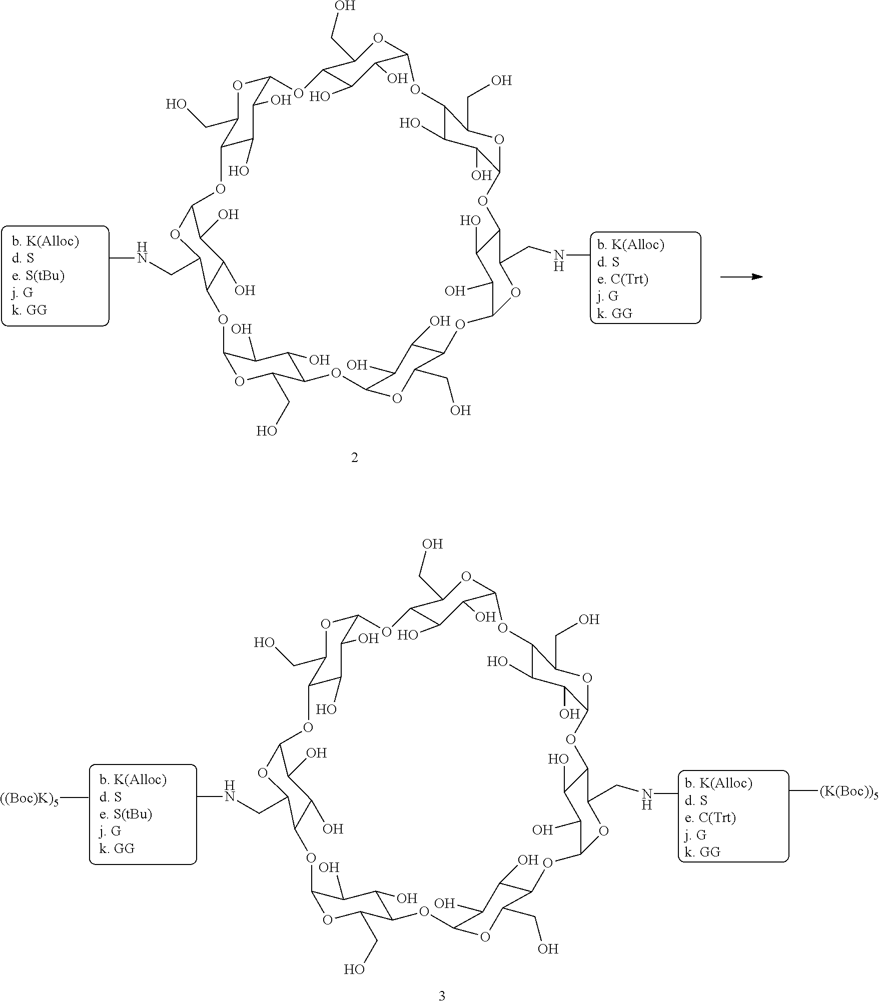

### Preparation of Compound 3b:

Compound 3b was prepared by using general procedure A between cyclodextrin 2b and Fmoc-(K(Boc))₅-OH (SEQ ID NO: 2). Fmoc group was removed using general procedure B to give compound 3b. MS m/z calcd. for C₁₇₂H₃₀₄N₂₆O₆₉ 3838, found 1920 ((M+2H)/2).

### Preparation of Compound 3d:

Compound 3d was prepared by using general procedure A between cyclodextrin 2d and Fmoc-(K(Boc))₅-OH (SEQ ID NO: 2). Fmoc group was removed using general procedure B to give compound 3d. MS m/z calcd. for C₁₅₈H₂₈₂N₂₄O₆₇ 3588, found 1795 ((M+2H)/2).

### Preparation of Compound 3e:

Compound 3e was prepared by using general procedure A between cyclodextrin 2e and Fmoc-(K(Boc))₅-OH (SEQ ID NO: 2). Fmoc group was removed using general procedure B to give compound 3e. MS m/z calcd. for C₁₈₁H₃₀₄N₂₄O₆₆S 3902, found 1952 ((M+2H)/2).

### Preparation of Compound 3j:

Compound 3j was prepared by using general procedure A between cyclodextrin 2j and Fmoc-(K(Boc))₅-OH (SEQ ID NO: 2). Fmoc group was removed using general procedure B to give compound 3j. MS m/z calcd. for C₁₅₆H₂₇₈N₂₄O₆₅ 3528, found 3529 (M+H).

### Preparation of Compound 3k:

Compound 3k was prepared by using general procedure A between cyclodextrin 2k and Fmoc-(K(Boc))₅-OH (SEQ ID NO: 2). Fmoc group was removed using general procedure B to give compound 3k. MS m/z calcd. for C₁₆₀H₂₈₄N₂₆O₆₇ 3642, found 1822 ((M+2H)/2).

### Example 4

### Preparation of Compounds 4a-4e (4a-4e Peptide Sequences Disclosed as SEQ ID NOS 11-15, Respectively)

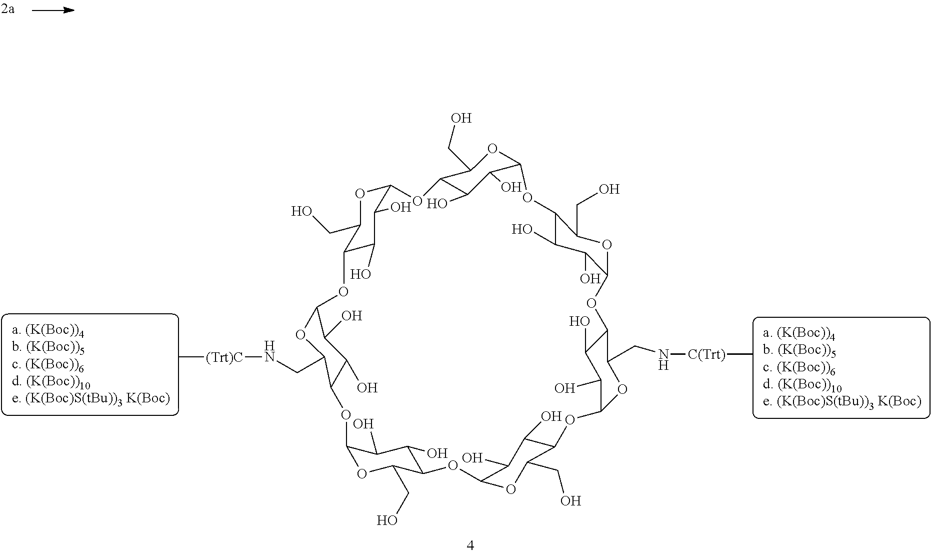

### Preparation of Compound 4a:

Compound 4a was prepared by using general procedure A between cyclodextrin 2a and Fmoc-(K(Boc))₄-OH (SEQ ID NO: 16). Fmoc group was removed using general procedure B and the mixture was purified by HPLC to give compound 4a. MS m/z calcd. for C₁₇₄H₂₇₀N₂₀O₅₉S₂ 3648, found 1825 ((M+2H)/2).

### Preparation of Compound 4b:

Compound 4b was prepared by using general procedure A between cyclodextrin 2a and Fmoc-(K(Boc))₅-OH (SEQ ID NO: 2). Fmoc group was removed using general procedure B and the mixture was purified by HPLC to give compound 4b. MS m/z calcd. for C₁₉₆H₃₁₀N₂₄O₆₅S₂ 4104, found 2053 ((M+2H)/2).

### Preparation of Compound 4c:

Compound 4c was prepared by using general procedure A between cyclodextrin 2a and Fmoc-(K(Boc))₆-OH (SEQ ID NO: 17). Fmoc group was removed using general procedure B and the mixture was purified by HPLC to give compound 4c. MS m/z calcd. for C₂₁₈H₃₅₀N₂₈O₇₁S₂ 4560, found 2281 ((M+2H)/2).

### Preparation of Compound 4d:

Compound 4d was prepared by using general procedure A between cyclodextrin 4b and Fmoc-(K(Boc))₅-OH (SEQ ID NO: 2). Fmoc group was removed using general procedure B and the mixture was purified by HPLC to give compound 4d. MS m/z calcd. for C₃₀₆H₅₁₀N₄₄O₉₅S₂ 6386, found 1598 ((M+4H)/4).

### Preparation of Compound 4e:

Compound 4e was prepared by using general procedure A between cyclodextrin 2a and Fmoc-(K(Boc)S(tBu))₃K(Boc)-OH (SEQ ID NO: 18). Fmoc group was removed using general procedure B and the mixture was purified by HPLC to give compound 4e. MS m/z calcd. for C₂₁₆H₃₄₈N₂₆O₇₁S₂ 4506, found 2254 ((M+2H)/2).

### Example 5

### Preparation of Compounds 6a-6o (Left-Hand Peptide Sequences 6a-6o Disclosed as SEQ ID NOS 19-25, 25, 27-29, 26, & 30-32, Respectively. Right-Hand Peptide Sequences 6a-6o Disclosed as SEQ ID NOS 19-29, 26, & 30-32, Respectively)

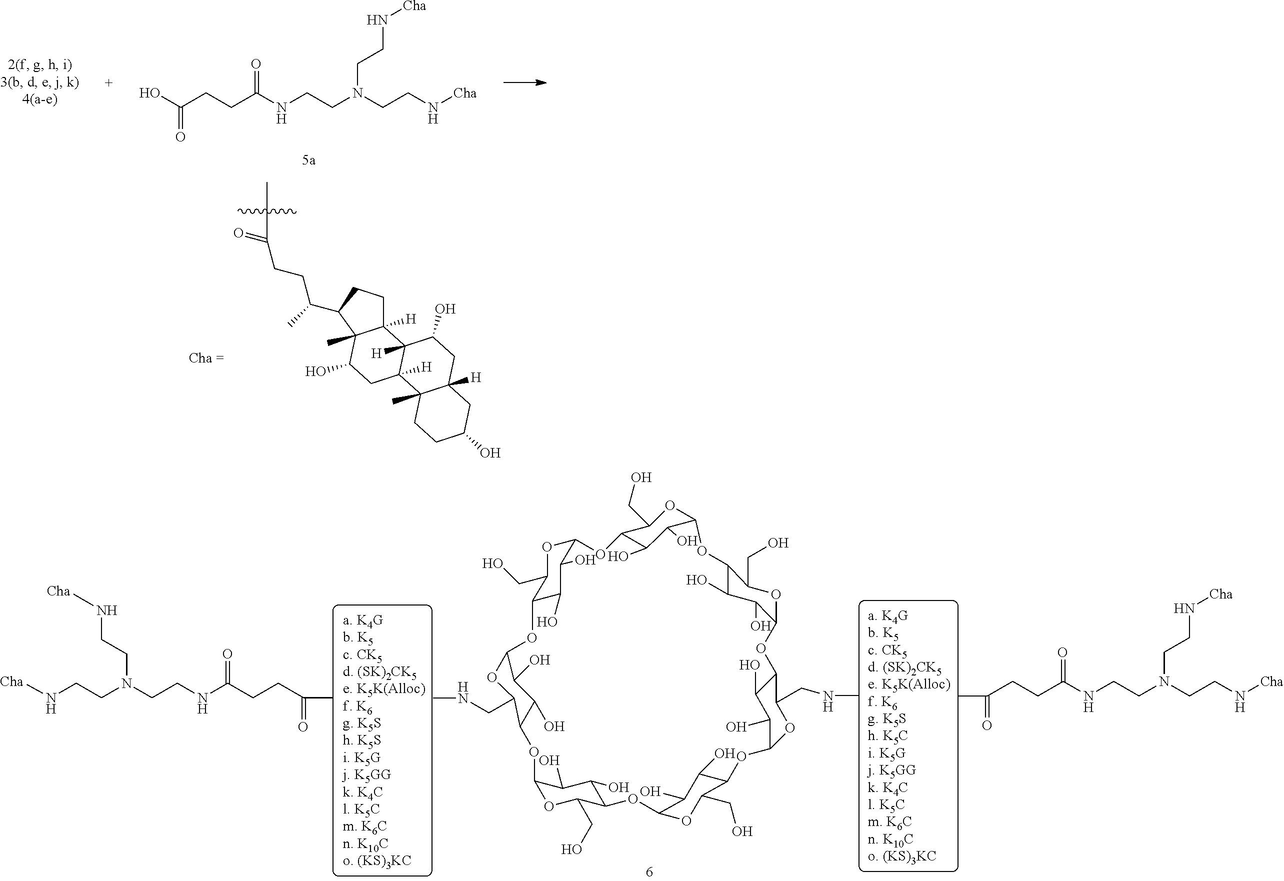

### Preparation of Compound 6a:

Compound 6a was prepared by using general procedure A between cyclodextrin 2f and acid 5. Boc was removed using general procedure E. The mixture was purified by HPLC to give compound 6a. MS m/z calcd. for C₂₁₀H₃₆₆N₂₈O₆₃ 4289, found 1431 ((M+3H)/3). ¹H-NMR (300 MHz, D₂O): δ 4.7-5.0 (m, 7H), 2.7-4.4 (m, 122H), 1.2-1.8 (m, 136H), 0.5-1.0 (m, 36H).

### Preparation of Compound 6b:

Compound 6b was prepared by using general procedure A between cyclodextrin 2g and acid 5. Boc was removed using general procedure E. The mixture was purified by HPLC to give compound 6b. MS m/z calcd. for C₂₁₈H₃₈₄N₃₀O₆₃ 4431, found 2218 ((M+2H)/2). ¹H-NMR (300 MHz, D₂O): δ 4.7-5.0 (m, 7H), 2.7-4.4 (m, 124H), 1.2-1.8 (m, 148H), 0.5-1.0 (m, 38H).

### Preparation of Compound 6c:

Compound 6c was prepared by using general procedure A between cyclodextrin 2 h and acid 5. Boc and Trt groups were removed using general procedure E. The mixture was purified by HPLC to give compound 6c. MS m/z calcd. for C₂₂₄H₃₉₄N₃₂O₆₅S₂ 4637, found 1547 ((M+3H)/3). ¹H-NMR (300 MHz, D₂O): δ 4.7-5.0 (m, 7H), 2.7-4.4 (m, 130H), 1.2-1.8 (m, 148H), 0.5-1.0 (m, 36H).

Preparation of compound 6d:

Compound 6d was prepared by using general procedure A between cyclodextrin 2i and acid 5. Boc and Trt groups were removed using general procedure E. The mixture was purified by HPLC to give compound 6d. MS m/z calcd. for C₂₆₀H₄₆₂N₄₄O₇₇S₂ 5497, found 1832 ((M+3H)/3). ¹H-NMR (300 MHz, D₂O): δ 4.7-5.0 (m, 7H), 2.7-4.4 (m, 154H), 1.2-1.8 (m, 172H), 0.5-1.0 (m, 36H).

### Preparation of Compound 6e:

Compound 6e was prepared by using general procedure A between cyclodextrin 3b and acid 5. Boc was removed using general procedure E. The mixture was purified by HPLC to give compound 6e. MS m/z calcd. for C₂₃₈H₄₁₆N₃₄O₆₉ 4855, found 1214 ((M+4H)/4). ¹H-NMR (300 MHz, D₂O): δ 5.0-6.0 (m, 6H), 4.7-5.0 (m, 7H), 2.7-4.4 (m, 134H), 1.2-1.8 (m, 160H), 0.5-1.0 (m, 36H).

### Preparation of Compound 6f:

Compound 6f was prepared by using general procedure D to remove Alloc group from 6e. Boc was removed using general procedure E. The mixture was purified by HPLC to give compound 6f. MS m/z calcd. for C₂₃₀H₄₀₈N₃₄O₆₅ 4687, found 1563 ((M+3H)/3). ¹H-NMR (300 MHz, D₂O): δ 4.7-5.0 (m, 7H), 2.7-4.4 (m, 130H), 1.2-1.8 (m, 160H), 0.5-1.0 (m, 36H).

### Preparation of Compound 6g:

Compound 6g was prepared by using general procedure A between cyclodextrin 3d and acid 5. Boc was removed using general procedure E. The mixture was purified by HPLC to give compound 6g. MS m/z calcd. for C₂₂₄H₃₉₄N₃₂O₆₇ 4605, found 1536 ((M+3H)/3). ¹H-NMR (300 MHz, D₂O): δ 4.7-5.0 (m, 7H), 2.7-4.4 (m, 130H), 1.2-1.8 (m, 148H), 0.5-1.0 (m, 36H).

### Preparation of Compound 6 h:

Compound 6 h was prepared by using general procedure A between cyclodextrin 3e and acid 5. Boc, tBu and Trt groups were removed using general procedure E. The mixture was purified by HPLC to give compound 6 h. MS m/z calcd. for C₂₂₄H₃₉₄N₃₂O₆₆S 4621, found 2311 ((M+2H)/2). ¹H-NMR (300 MHz, D₂O): δ 4.7-5.0 (m, 7H), 2.7-4.4 (m, 130H), 1.2-1.8 (m, 148H), 0.5-1.0 (m, 36H).

### Preparation of Compound 6i:

Compound 6i was prepared by using general procedure A between cyclodextrin 3j and acid 5. Boc was removed using general procedure E. The mixture was purified by HPLC to give compound 6i. MS m/z calcd. for C₂₂₂H₃₉₀N₃₂O₆₅ 4545, found 1516 ((M+3H)/3). ¹H-NMR (300 MHz, D₂O): δ 4.7-5.0 (m, 7H), 2.7-4.4 (m, 128H), 1.2-1.8 (m, 148H), 0.5-1.0 (m, 36H).

### Preparation of Compound 6j:

Compound 6j was prepared by using general procedure A between cyclodextrin 3k and acid 5. Boc was removed using general procedure E. The mixture was purified by HPLC to give compound 6j. MS m/z calcd. for C₂₂₆H₃₉₆N₃₄O₆₇ 4659, found 1165 ((M+4H)/4). ¹H-NMR (300 MHz, D₂O): δ 4.7-5.0 (m, 7H), 2.7-4.4 (m, 132H), 1.2-1.8 (m, 148H), 0.5-1.0 (m, 36H).

### Preparation of Compound 6k:

Compound 6k was prepared by using general procedure A between cyclodextrin 4a and acid 5. Boc and Trt groups were removed using general procedure E. The mixture was purified by HPLC to give compound 6k. MS m/z calcd. for C₂₁₂H₃₇₀N₂₈O₆₃S₂ 4381, found 2192 ((M+2H)/2). ¹H-NMR (300 MHz, D₂O): δ 4.7-5.0 (m, 7H), 2.7-4.4 (m, 124H), 1.2-1.8 (m, 136H), 0.5-1.0 (m, 36H).

### Preparation of Compound 61:

Compound 61 was prepared by using general procedure A between cyclodextrin 4b and acid 5. Boc and Trt groups were removed using general procedure E. The mixture was purified by HPLC to give compound 61. MS m/z calcd. for C₂₂₄H₃₉₄N₃₂O₆₅S₂ 4636, found 2318 ((M+2H)/2). ¹H-NMR (300 MHz, D₂O): δ 4.7-5.0 (m, 7H), 2.7-4.4 (m, 130H), 1.2-1.8 (m, 148H), 0.5-1.0 (m, 36H).

### Preparation of Compound 6m:

Compound 6m was prepared by using general procedure A between cyclodextrin 4c and acid 5. Boc and Trt groups were removed using general procedure E. The mixture was purified by HPLC to give compound 6m. MS m/z calcd. for C₂₃₆H₄₁₈N₃₆O₆₇S₂ 4893, found 1632 ((M+3H)/3). ¹H-NMR (300 MHz, D₂O): δ 4.7-5.0 (m, 7H), 2.7-4.4 (m, 136H), 1.2-1.8 (m, 160H), 0.5-1.0 (m, 36H).

### Preparation of Compound 6n:

Compound 6n was prepared by using general procedure A between cyclodextrin 4d and acid 5. Boc and Trt groups were removed using general procedure E. The mixture was purified by HPLC to give compound 6n. MS m/z calcd. for C₂₈₄H₅₁₄N₅₂O₇₅S₂ 5918, found 1974 ((M+3H)/3). ¹H-NMR (300 MHz, D₂O): δ 4.7-5.0 (m, 7H), 2.7-4.4 (m, 160H), 1.2-1.8 (m, 208H), 0.5-1.0 (m, 36H).

### Preparation of Compound 6o:

Compound 6o was prepared by using general procedure A between cyclodextrin 4e and acid 5. Boc and Trt groups were removed using general procedure E. The mixture was purified by HPLC to give compound 6o. MS m/z calcd. for C₂₃₀H₄₀₀N₃₄O₇₅S₂ 4903, found 982 ((M+5H)/5). ¹H-NMR (300 MHz, D₂O): δ 4.7-5.0 (m, 7H), 2.7-4.4 (m, 142H), 1.2-1.8 (m, 136H), 0.5-1.0 (m, 36H).

### Example 6

### Preparation of Compounds 7b-7e (“K₅C” Disclosed as SEQ ID NO: 26)

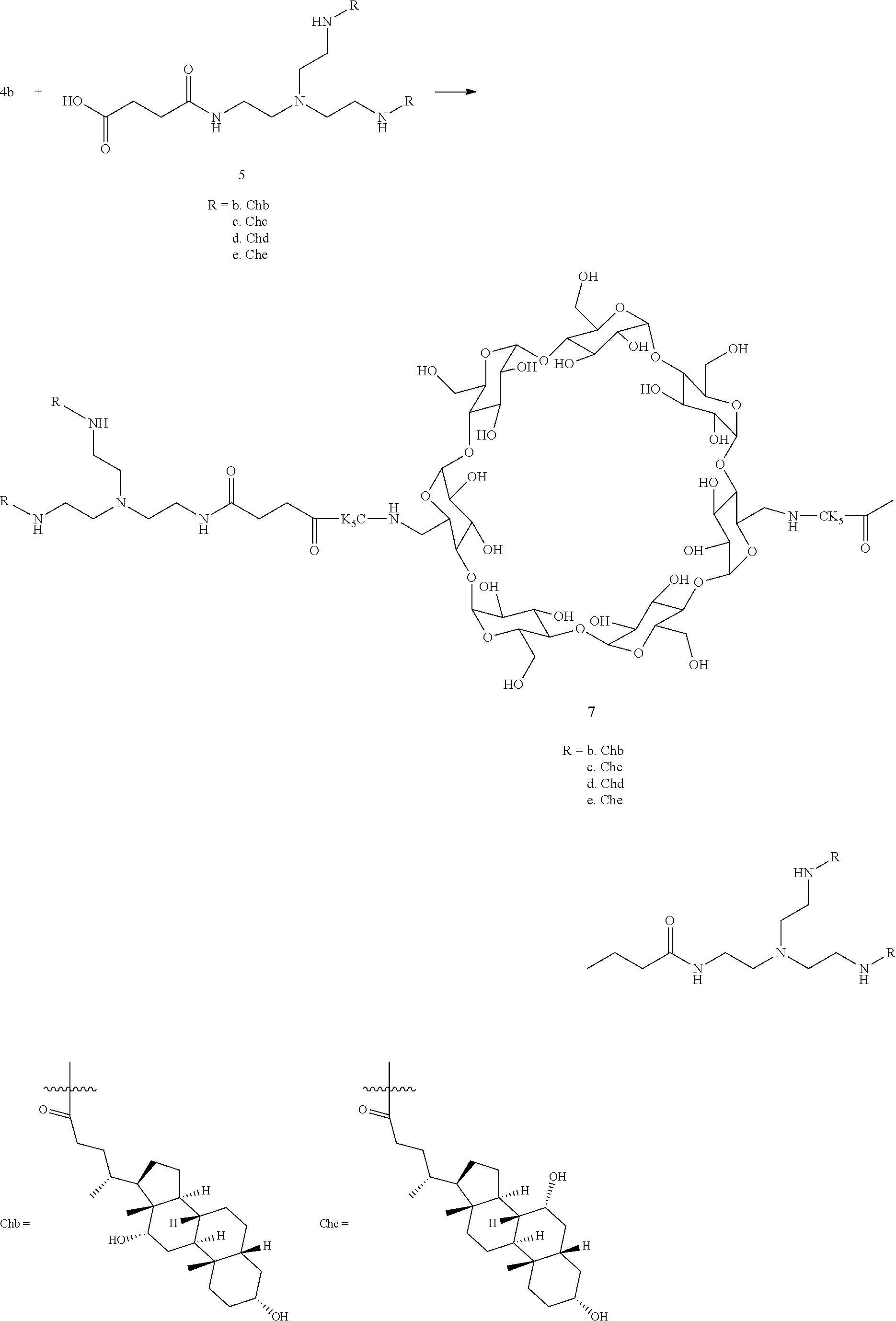

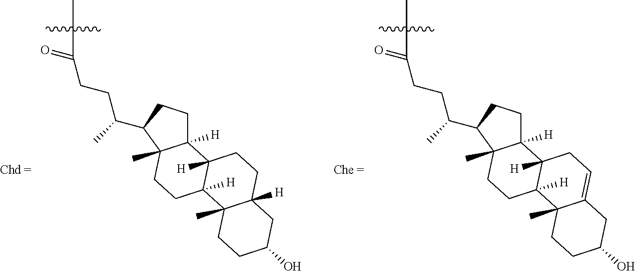

### Preparation of Compound 7b:

Compound 7b was prepared by using general procedure A between cyclodextrin 4b and acid 5b. Boc and Trt groups were removed using general procedure E. The mixture was purified by HPLC to give compound 7b. MS m/z calcd. for C₂₂₄H₃₉₄N₃₂O₆₁S₂ 4573, found 1144 ((M+4H)/4). ¹H-NMR (300 MHz, D₂O): δ 4.7-5.0 (m, 7H), 2.7-4.4 (m, 126H), 1.2-1.8 (m, 156H), 0.5-1.0 (m, 36H).

### Preparation of Compound 7c:

Compound 7c was prepared by using general procedure A between cyclodextrin 4b and acid 5c. Boc and Trt groups were removed using general procedure E. The mixture was purified by HPLC to give compound 7c. MS m/z calcd. for C₂₂₄H₃₉₄N₃₂O₆₁S₂ 4573, found 2288 ((M+2H)/2). ¹H-NMR (300 MHz, D₂O): δ 4.7-5.0 (m, 7H), 2.7-4.4 (m, 126H), 1.2-1.8 (m, 156H), 0.5-1.0 (m, 36H).

### Preparation of Compound 7d:

Compound 7d was prepared by using general procedure A between cyclodextrin 4b and acid 5d. Boc and Trt groups were removed using general procedure E. The mixture was purified by HPLC to give compound 7d. MS m/z calcd. for C₂₂₄H₃₉₄N₃₂O₅₇S₂ 4509, found 1128 ((M+4H)/4). ¹H-NMR (300 MHz, D₂O): δ 4.7-5.0 (m, 7H), 2.7-4.4 (m, 122H), 1.2-1.8 (m, 164H), 0.5-1.0 (m, 36H).

### Preparation of Compound 7e:

Compound 7e was prepared by using general procedure A between cyclodextrin 4b and acid 5e. Boc and Trt groups were removed using general procedure E. The mixture was purified by HPLC to give compound 7e. MS m/z calcd. for C₂₂₄H₃₈₆N₃₂O₅₇S₂ 4501, found 1126 ((M+4H)/4). ¹H-NMR (300 MHz, D₂O): δ 4.7-5.5 (m, 11H), 2.7-4.4 (m, 122H), 1.2-1.8 (m, 152H), 0.5-1.0 (m, 36H).

### Example 7

### Preparation of Compounds 8a-8d (8a-8d Peptide Sequences Disclosed as SEQ ID NOS 25, 26, 26, and 26, Respectively)

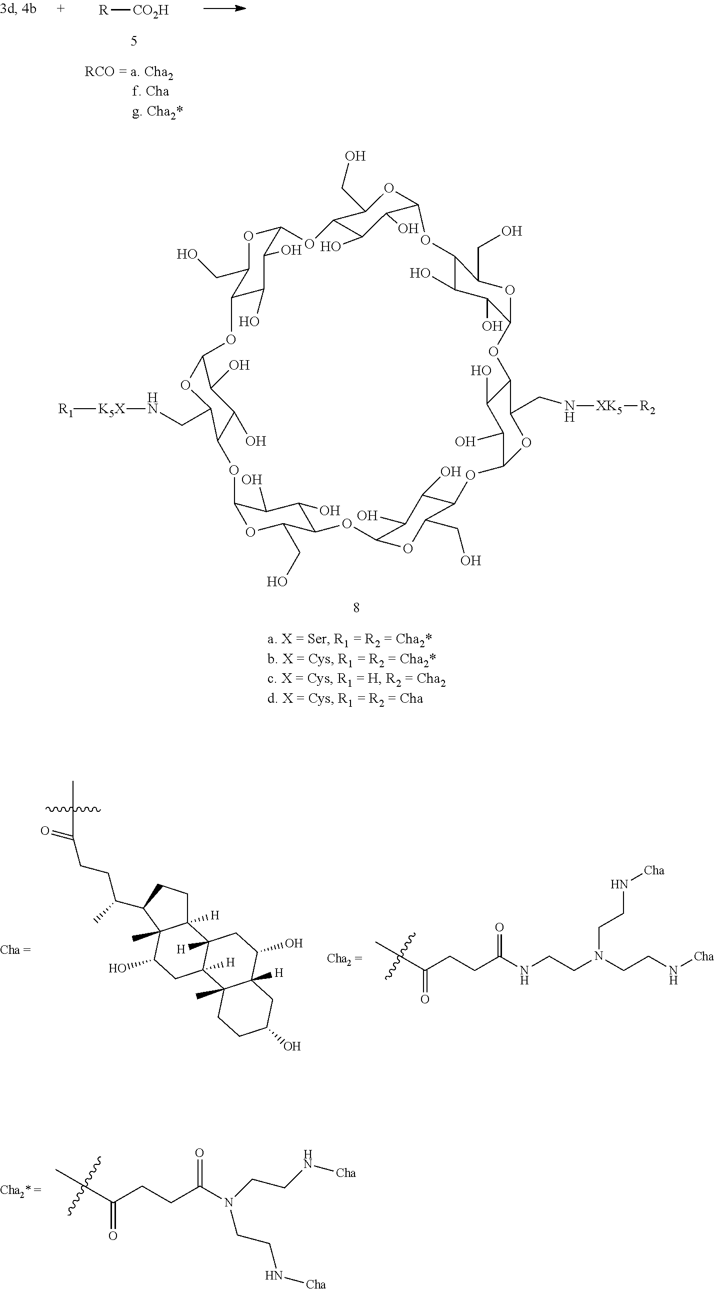

### Preparation of Compound 8a:

Compound 8a was prepared by using general procedure A between cyclodextrin 3d and acid 5g. Boc was removed using general procedure E. The mixture was purified by HPLC to give compound 8a. MS m/z calcd. for C₂₂₀H₃₈₄N₃₀O₆₇ 4519, found 1131 ((M+4H)/4). ¹H-NMR (300 MHz, D₂O): δ 4.7-5.0 (m, 7H), 2.7-4.4 (m, 114H), 1.2-1.8 (m, 148H), 0.5-1.0 (m, 36H).

### Preparation of Compound 8b:

Compound 8b was prepared by using general procedure A between cyclodextrin 4b and acid 5g. Boc and Trt groups were removed using general procedure E. The mixture was purified by HPLC to give compound 8b. MS m/z calcd. for C₂₂₀H₃₈₄N₃₀O₆₅S₂ 4551, found 1518 ((M+3H)/3). ¹H-NMR (300 MHz, D₂O): δ 4.7-5.0 (m, 7H), 2.7-4.4 (m, 114H), 1.2-1.8 (m, 148H), 0.5-1.0 (m, 36H).

### Preparation of Compound 8c:

Compound 8c was prepared by using general procedure A between cyclodextrin 4b and acid 5a, but only 1 eq of acid 5a was used. Boc and Trt groups were removed using general procedure E. The mixture was purified by HPLC to give compound 8c. MS m/z calcd. for C₁₆₆H₂₉₈N₂₈O₅₅S₂ 3628, found 1815 ((M+2H)/2). ¹H-NMR (300 MHz, D₂O): δ 4.7-5.0 (m, 7H), 2.7-4.4 (m, 104H), 1.2-1.8 (m, 104H), 0.5-1.0 (m, 18H).

### Preparation of Compound 8d:

Compound 8d was prepared by using general procedure A between cyclodextrin 4b and acid 5f. Boc and Trt groups were removed using general procedure E. The mixture was purified by HPLC to give compound 8d. MS m/z calcd. for C₁₅₆H₂₇₈N₂₄O₅₃S₂ 3400, found 1701 ((M+2H)/2). ¹H-NMR (300 MHz, D₂O): δ 4.7-5.0 (m, 7H), 2.7-4.4 (m, 88H), 1.2-1.8 (m, 104H), 0.5-1.0 (m, 18H).

### Example 8

### Preparation of Compounds 10a-10e (10a-10e Peptide Sequences Disclosed as SEQ ID NOS 20, 20, 20, 127, and 20, Respectively)

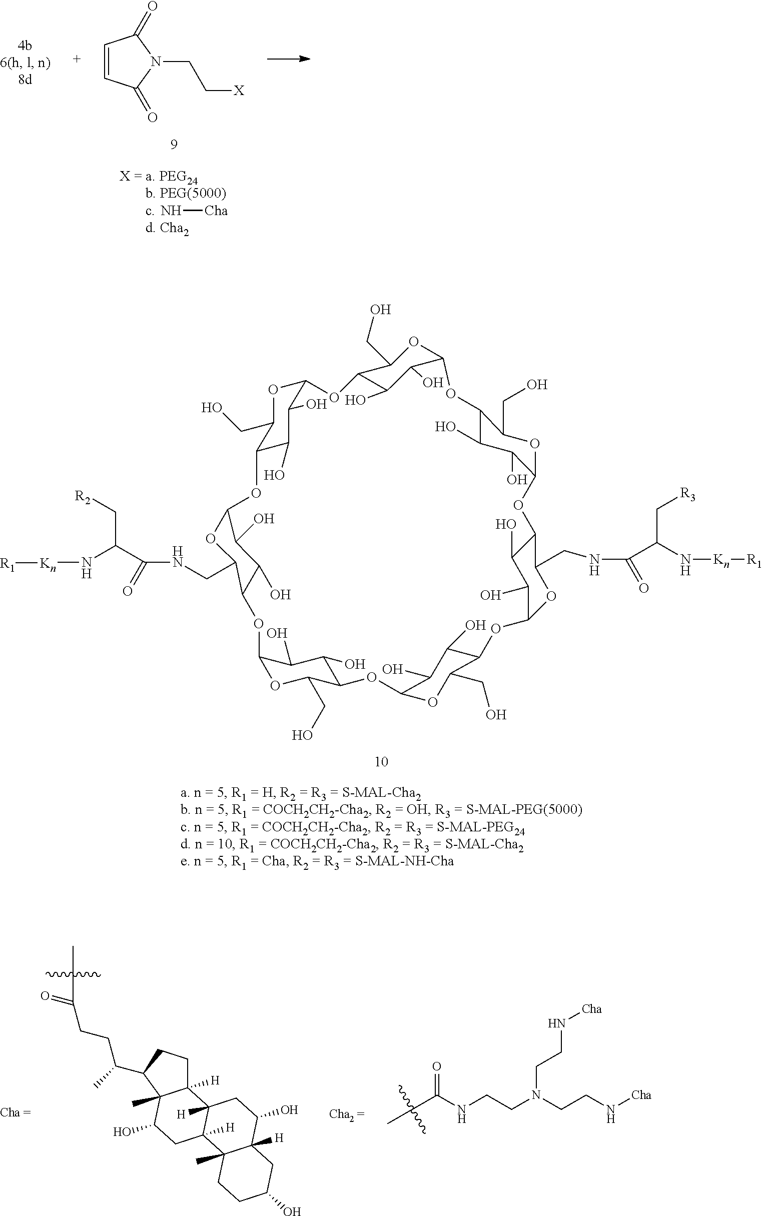

Preparation of Compound 10a:

Compound 10a was prepared by first removing Boc and Trt groups from compound 4b (1eq.) using general procedure C. The crude residue was washed two times with ether and then dissolved in DMF. Then MAL-Cha₂ (9d, 3eq.) was added. The pH of the reaction mixture was adjusted to 6 with DIEA and the mixture was stirred for 24 h under nitrogen. Then the mixture was evaporated and purified by HPLC to give compound 10a. MS m/z calcd. for C₂₃₀H₄₀₀N₃₄O₆₇S₂ 4774, found 2388 ((M+2H)/2). ¹H-NMR (300 MHz, D₂O): δ 4.7-5.0 (m, 7H), 2.7-4.4 (m, 136H), 1.2-1.8 (m, 148H), 0.5-1.0 (m, 36H).

### Preparation of Compound 10b:

Compound 6h (1eq.) was dissolved in MeOH and then MAL-PEG(5000) (9b, 1eq) was added. The pH of the reaction mixture was adjusted to 7 with DIEA and the mixture was stirred for 24 h under nitrogen. Then the mixture was evaporated and purified by HPLC to give compound 10b. MS m/z calcd. for C₄₅₈H₈₅₅N₃₃O₁₈₃S 9779, found 1088 ((M+9H)/9)/ ¹H-NMR (300 MHz, D₂O): δ 4.7-5.0 (m, 7H), 2.7-4.4 (m, 137H), 3.60 (s, 455H), 1.2-1.8 (m, 148H), 0.5-1.0 (m, 36H).

### Preparation of Compound 10c:

Compound 61 (1eq.) was dissolved in H₂O/dioxane (1:1) and then MAL-PEG₂₄ (9a, 2.4eq) was added. The pH of the reaction mixture was adjusted to 6 with K₂CO₃ and the mixture was stirred for 24 h under nitrogen. Then the mixture was evaporated and purified by HPLC to give compound 10c. MS m/z calcd. for C₃₃₆H₆₀₆N₃₆O₁₁₉S₂ 7114, found 2372 ((M+3H)/3). ¹H-NMR (300 MHz, D₂O): δ 4.7-5.0 (m, 7H), 2.7-4.4 (m, 148H), 3.30 (s, 194H), 1.2-1.8 (m, 148H), 0.5-1.0 (m, 36H).

### Preparation of Compound 10d:

Compound 6n (1eq.) was dissolved in 0.5 mL 0.1M sodium phosphate/10 mM EDTA buffer (pH 7.2) and 1 mL MeOH under nitrogen. Then MAL-Cha₂ (9d, 2.4eq) was added and the mixture was stirred for 24 h under nitrogen. Then the mixture was evaporated and purified by HPLC to give compound 10d. MS m/z calcd. for C₄₀₆H₇₁₂N₆₂O₉₇S₂ 8073, found 1616 ((M+5H)/5). ¹H-NMR (300 MHz, D₂O): δ 4.7-5.0 (m, 7H), 2.7-4.4 (m, 218H), 1.2-1.8 (m, 296H), 0.5-1.0 (m, 72H).

### Preparation of Compound 10e:

Compound 8d (1eq.) was dissolved in 1 mL of DMF and then MAL-NH-Cha (9c, 2.4eq) was added. The pH of the reaction mixture was adjusted to 7 with DIEA and the mixture was stirred for 30 min under nitrogen. Then the mixture was evaporated and purified by HPLC to give compound 10e. MS m/z calcd. for C₂₁₆H₃₇₀N₂₈O₆₅S₂ 4461, found 2232 ((M+2H)/3). ¹H-NMR (300 MHz, D₂O): δ 4.7-5.0 (m, 7H), 2.7-4.4 (m, 112H), 1.2-1.8 (m, 148H), 0.5-1.0 (m, 36H).

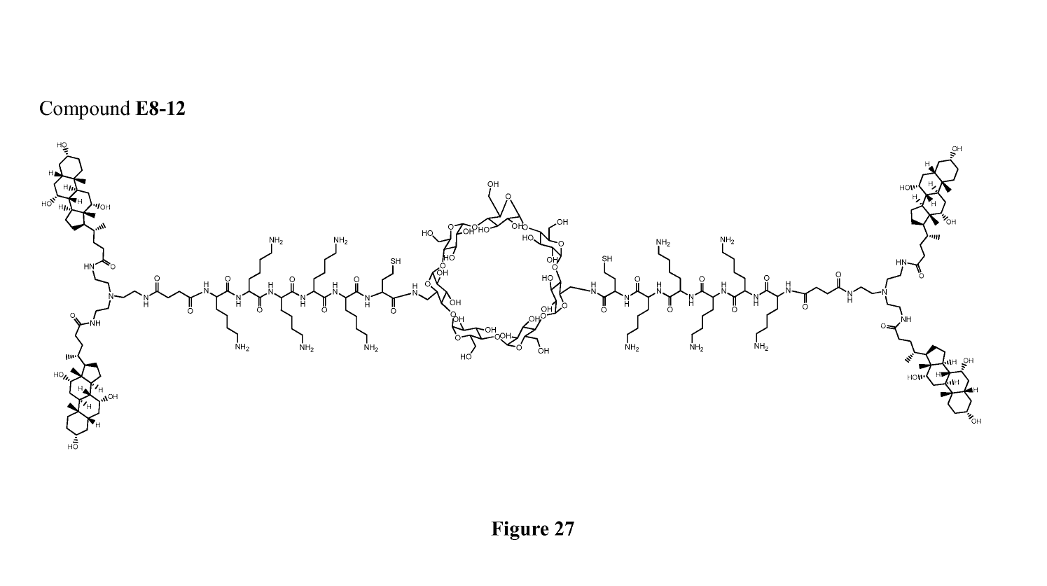

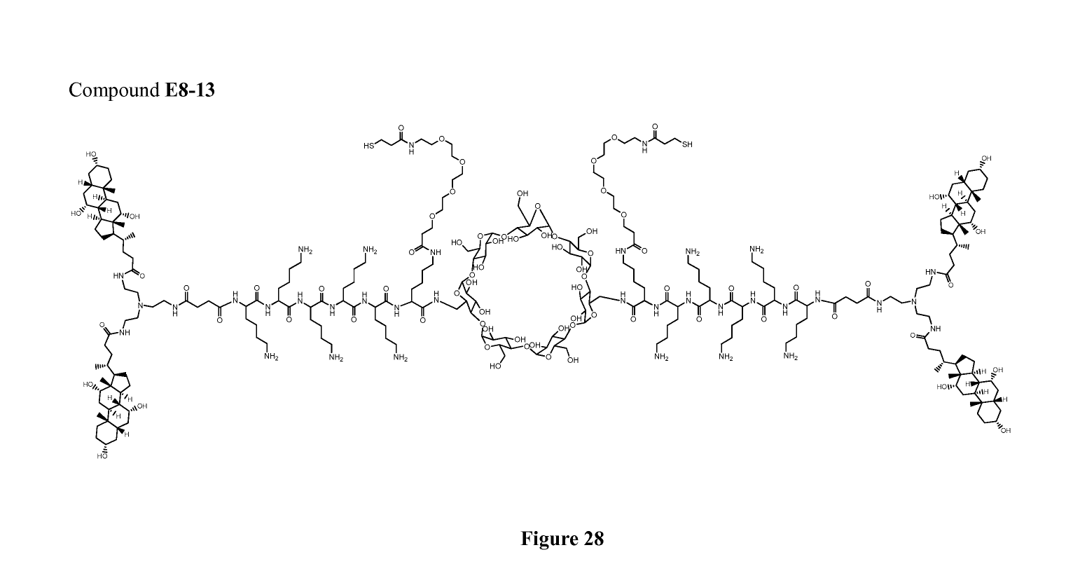

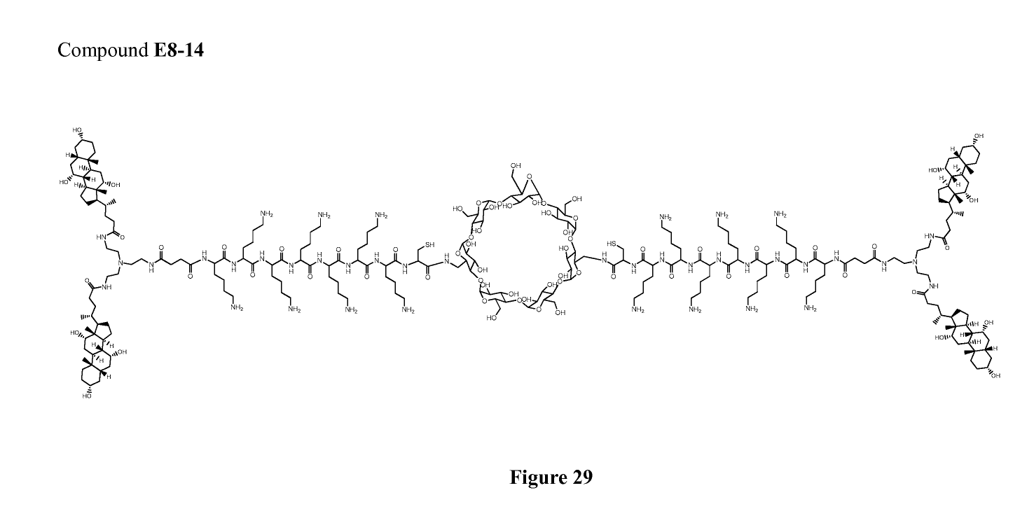

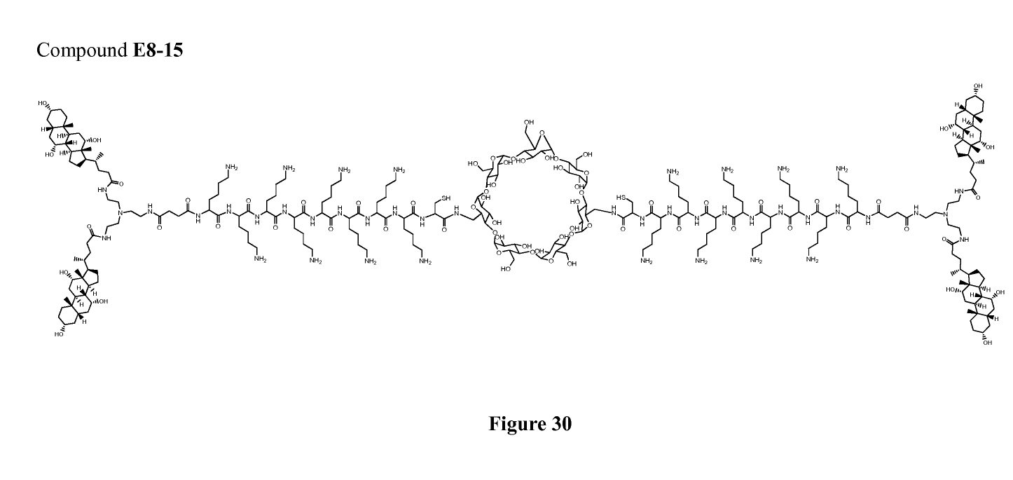

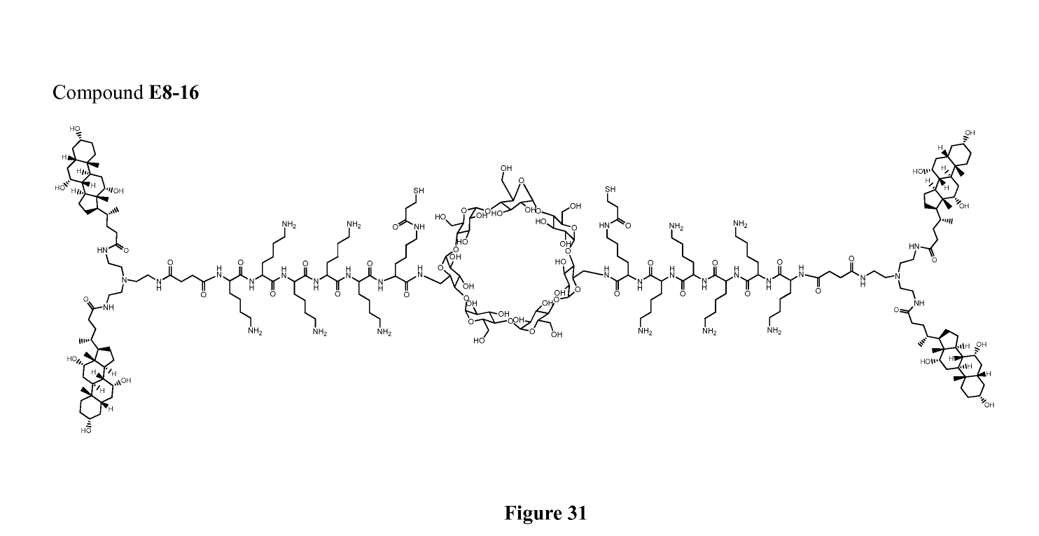

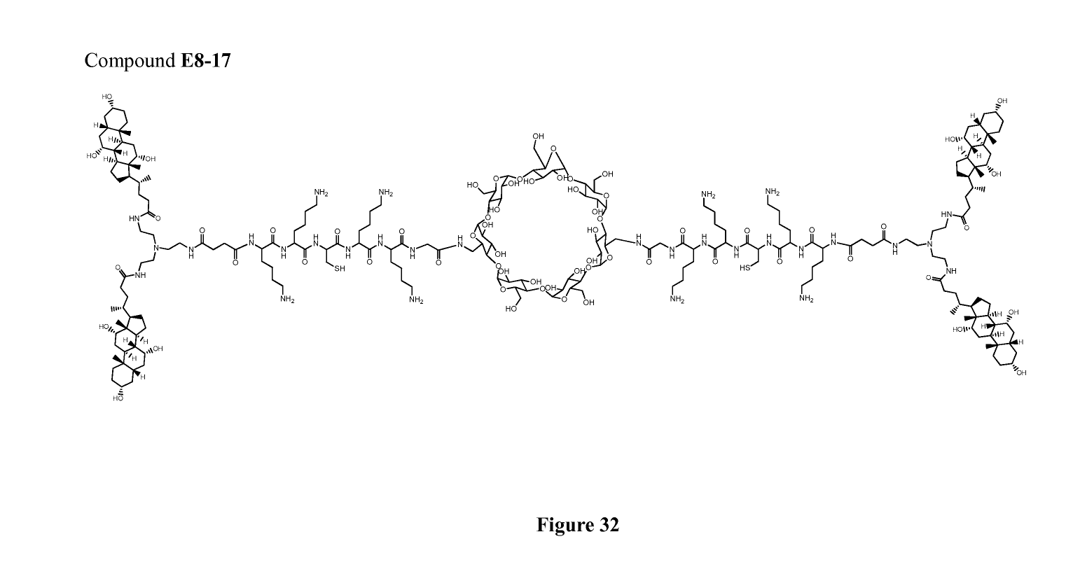

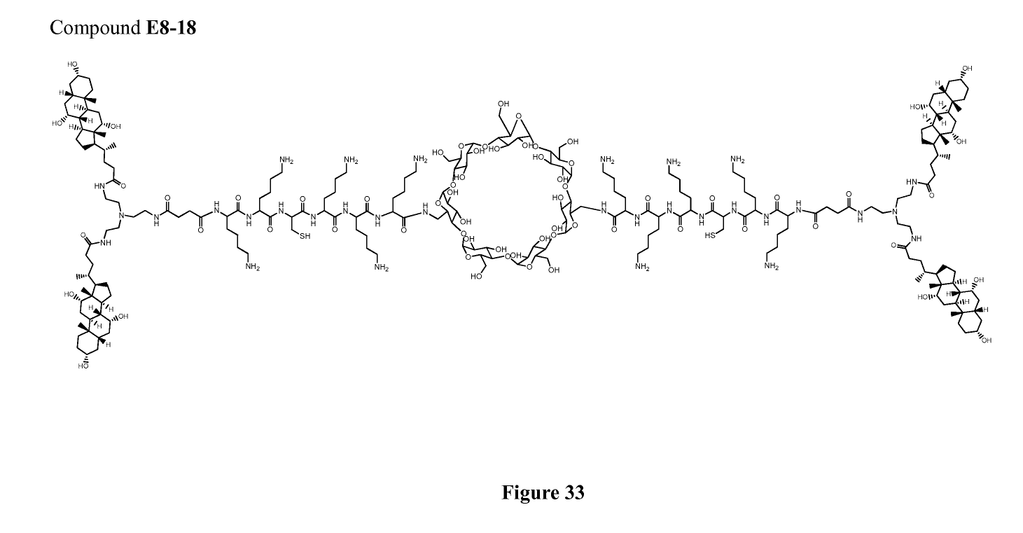

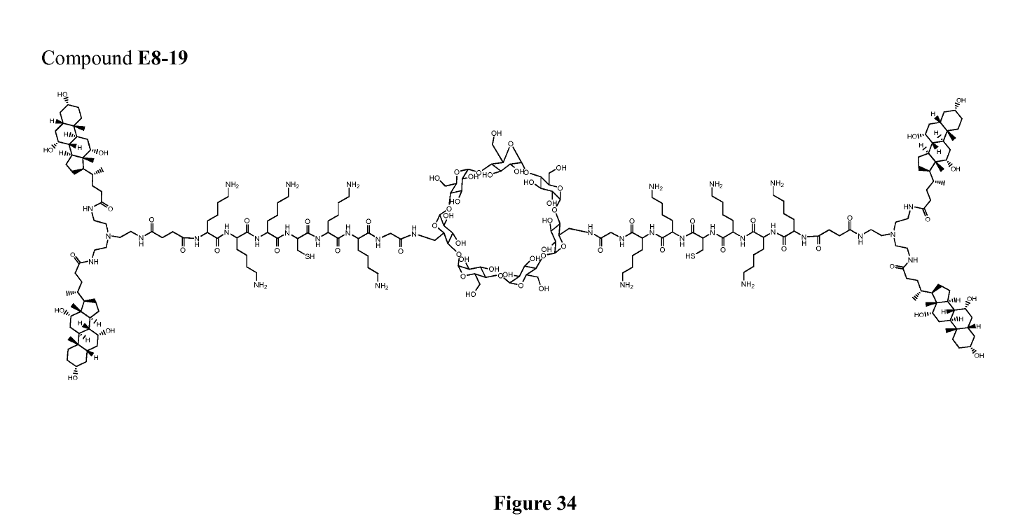

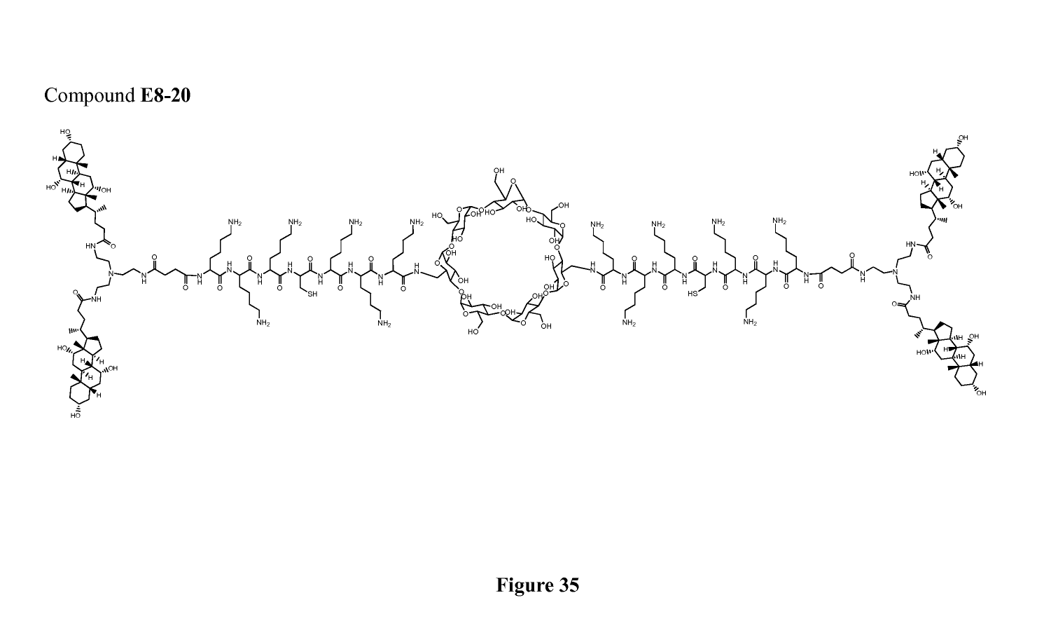

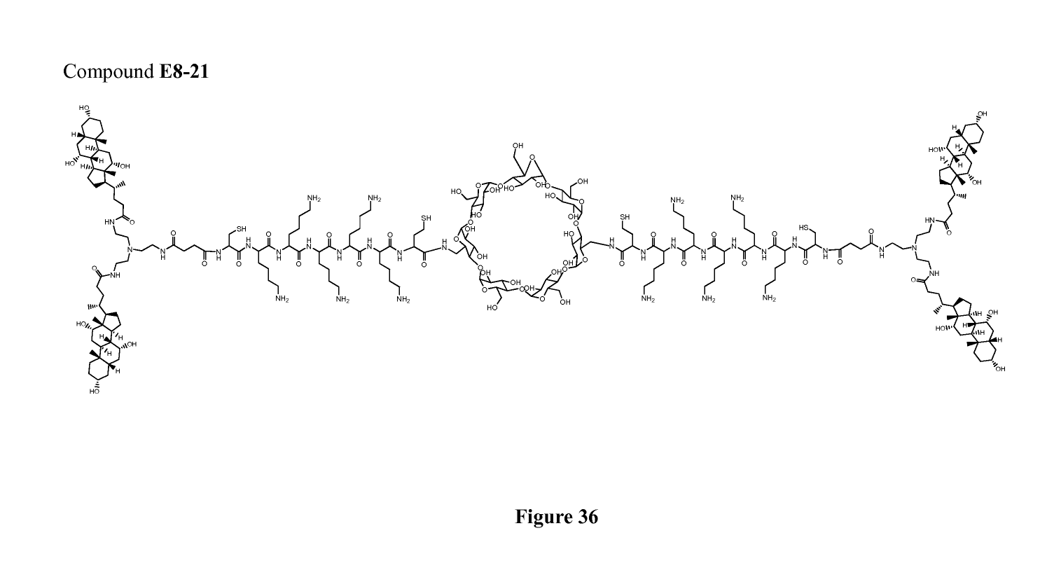

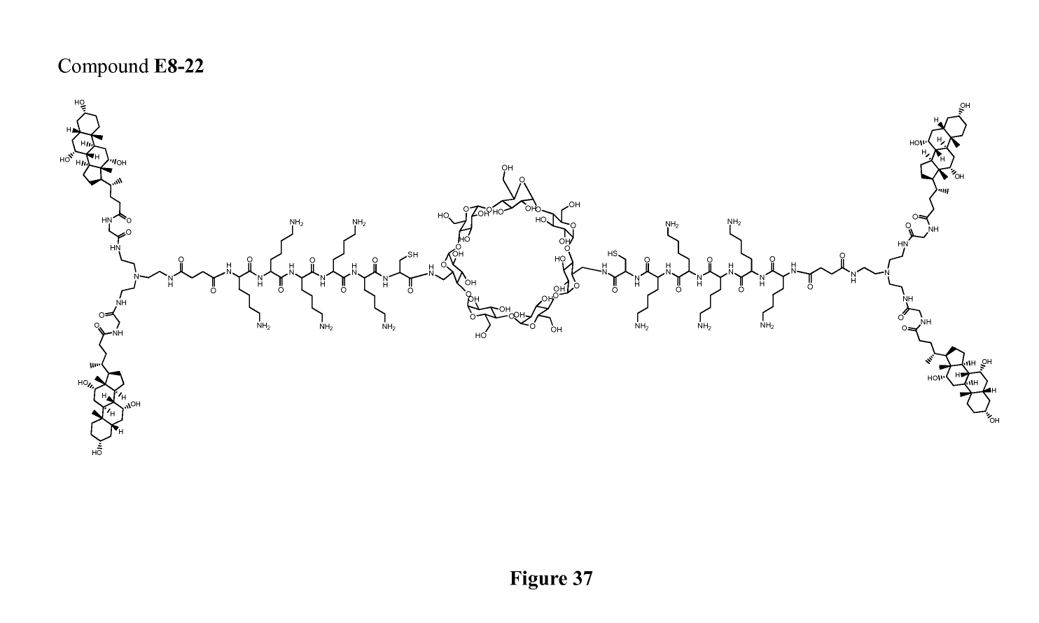

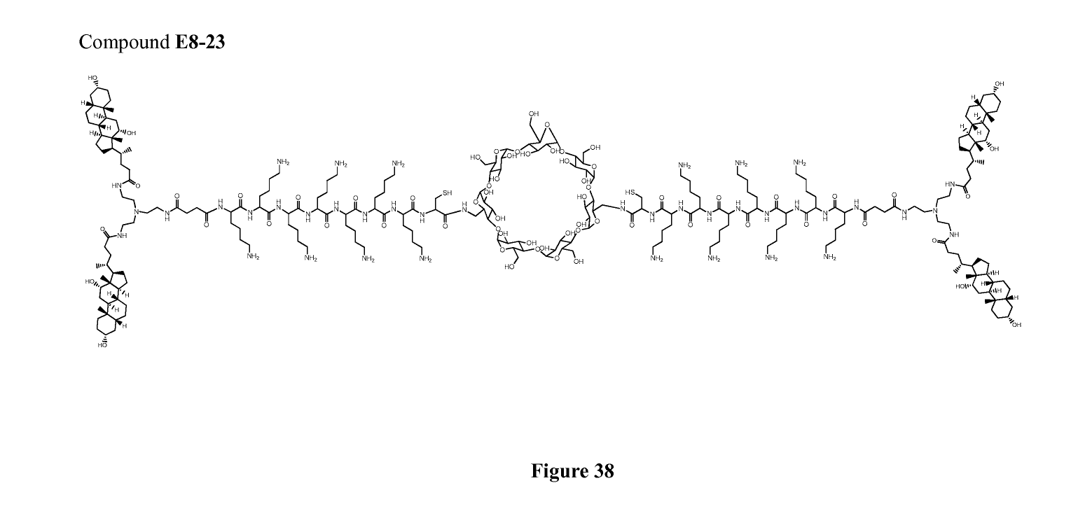

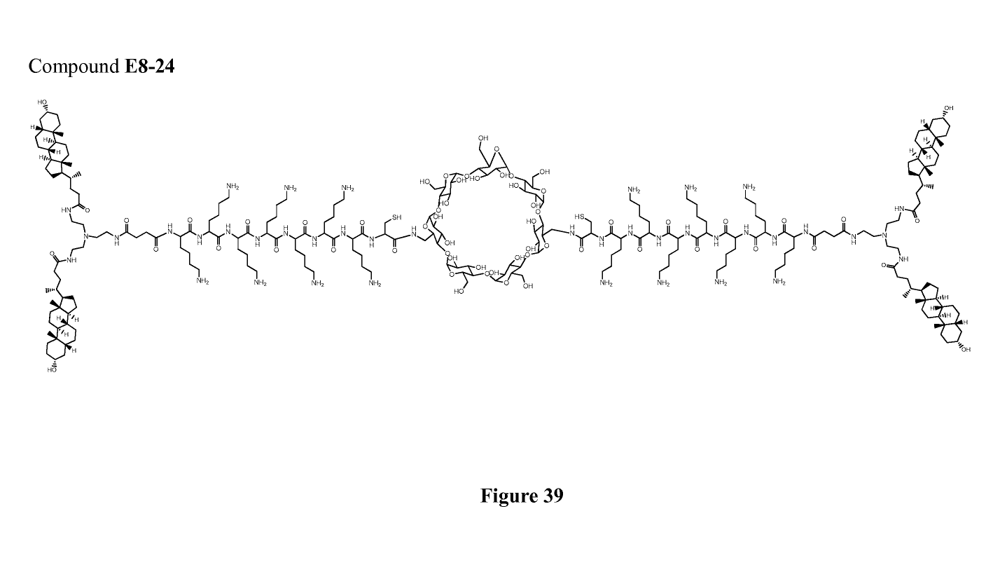

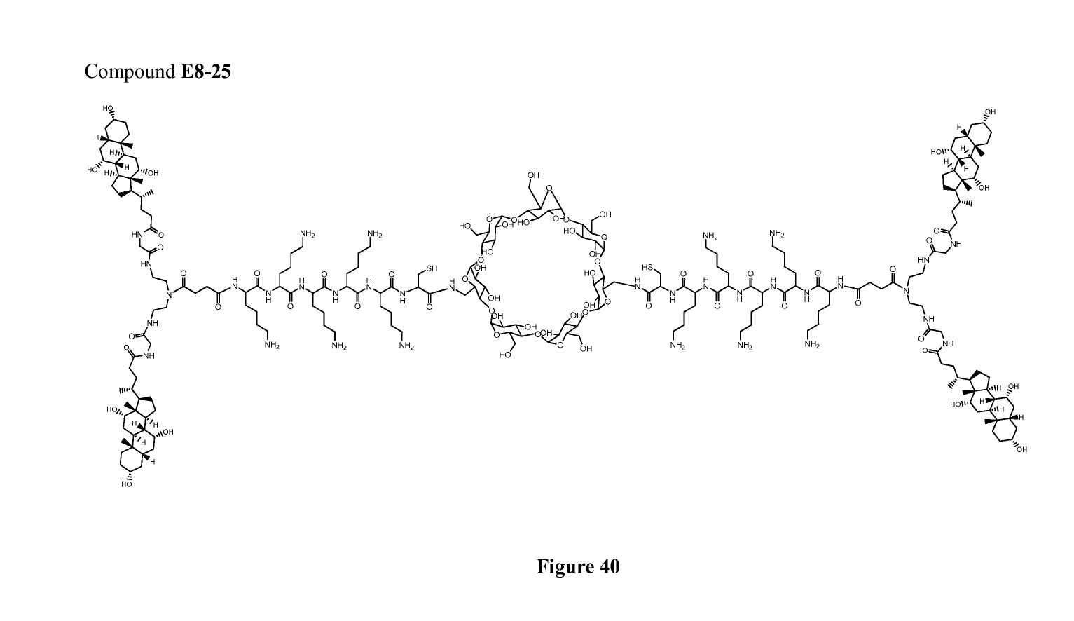

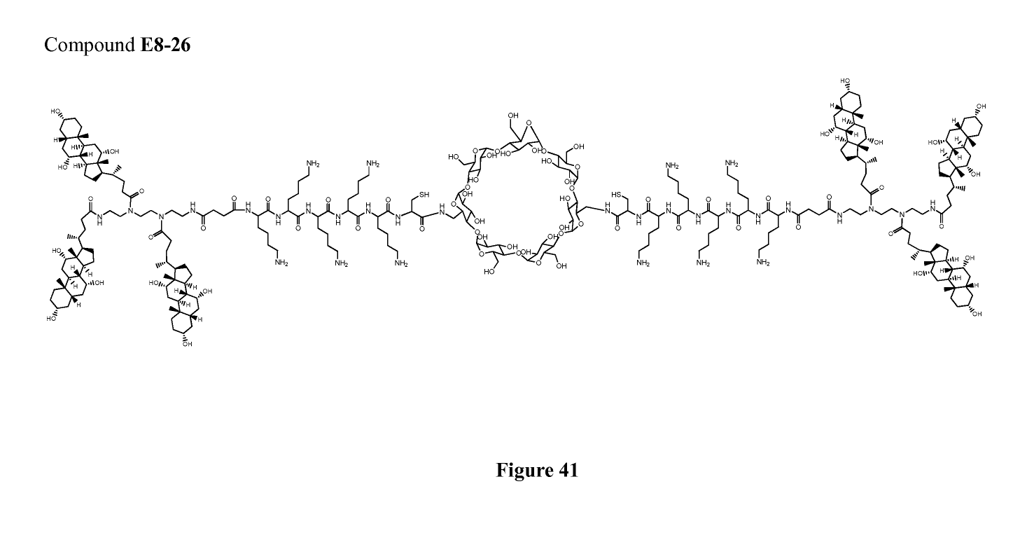

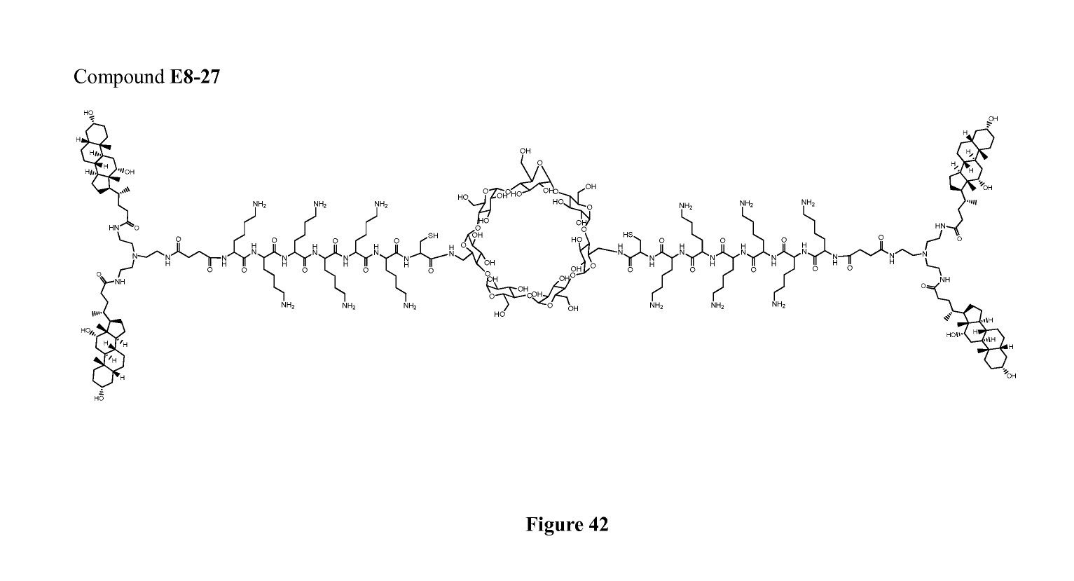

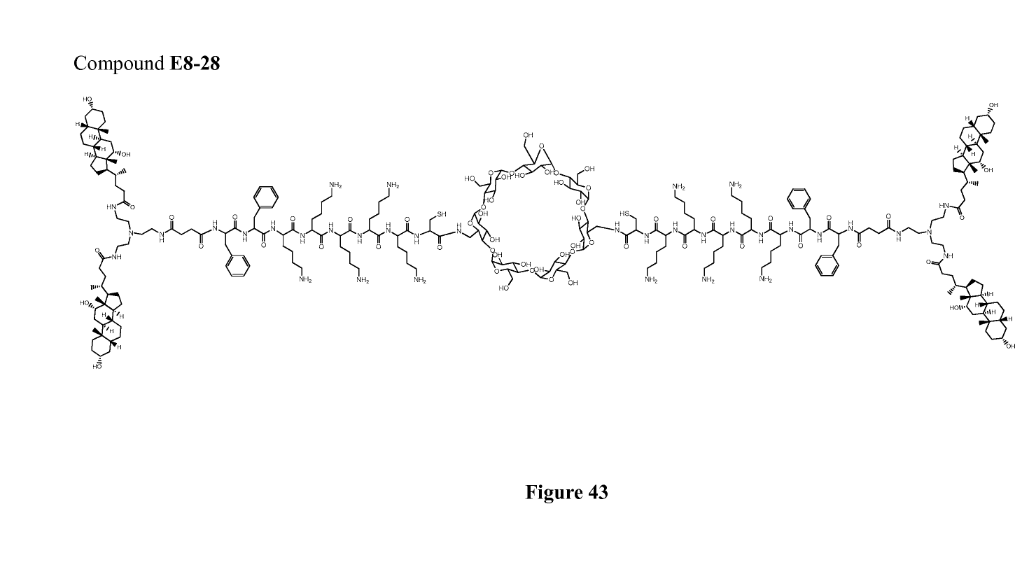

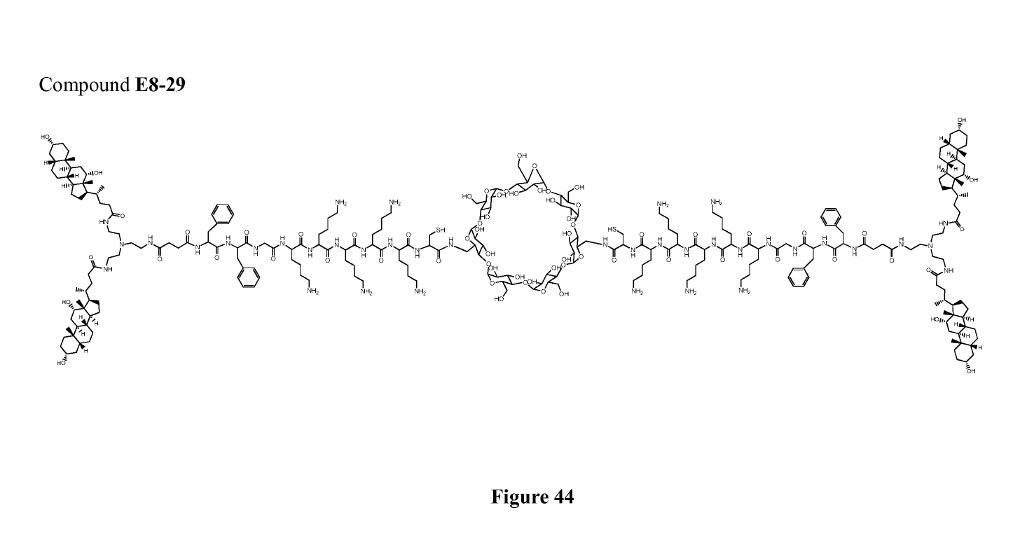

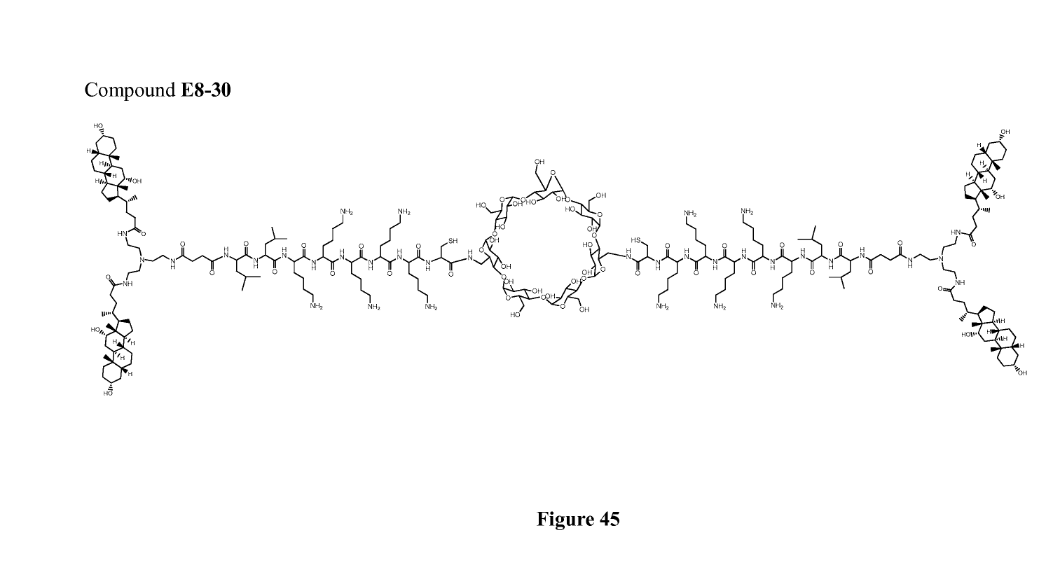

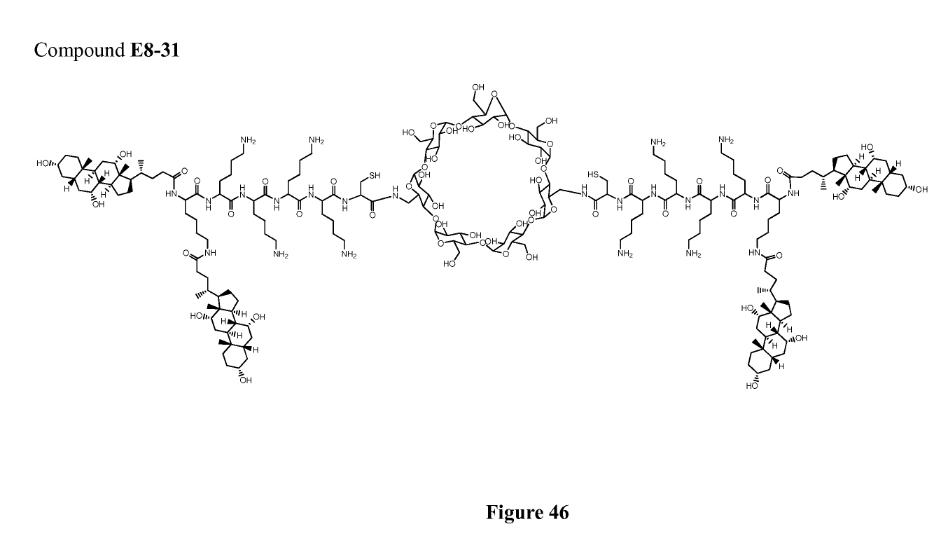

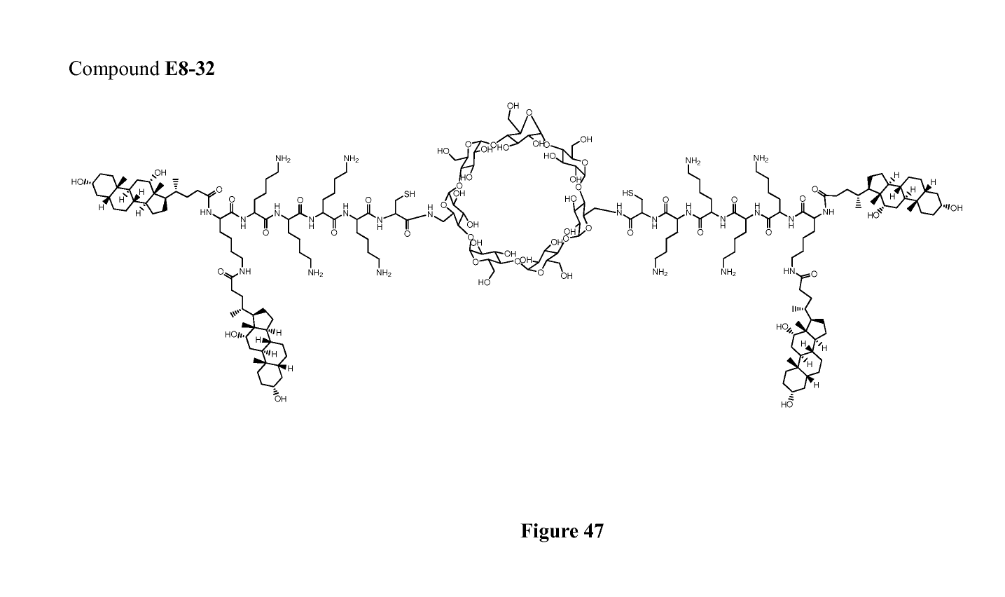

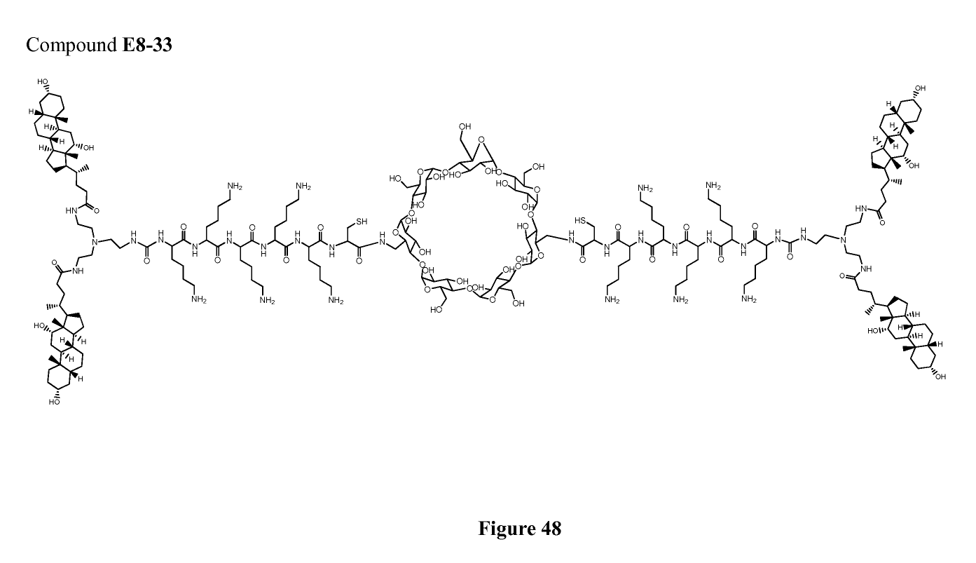

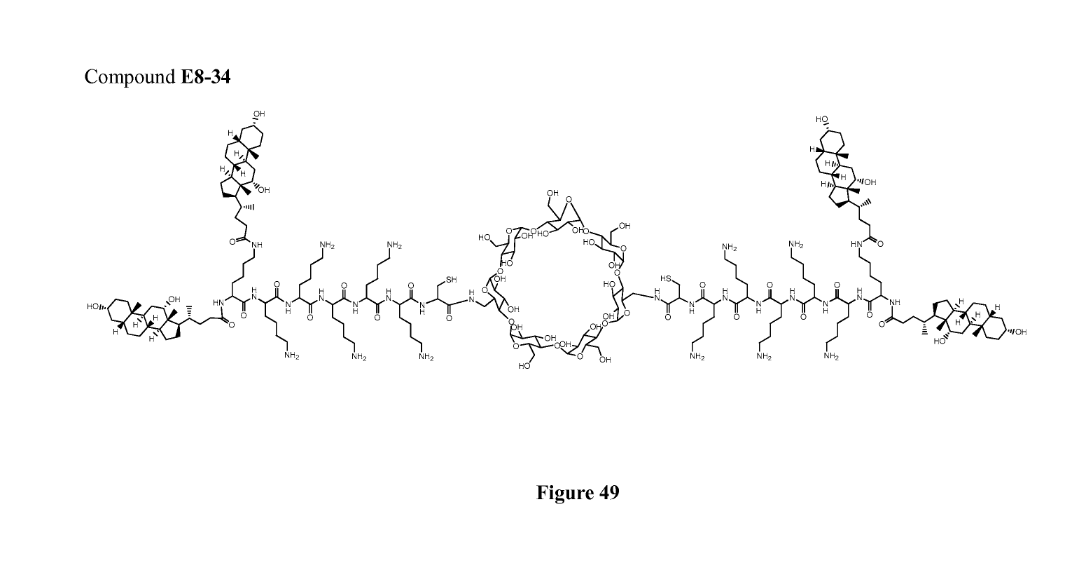

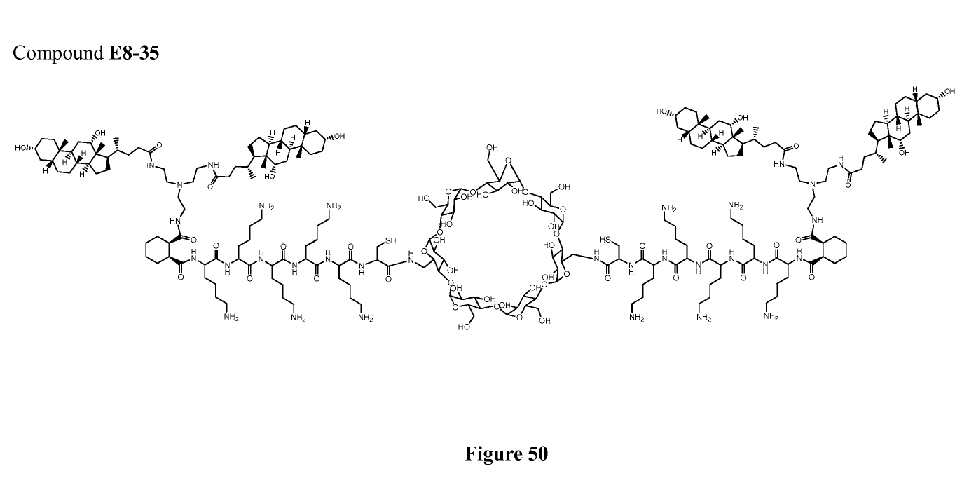

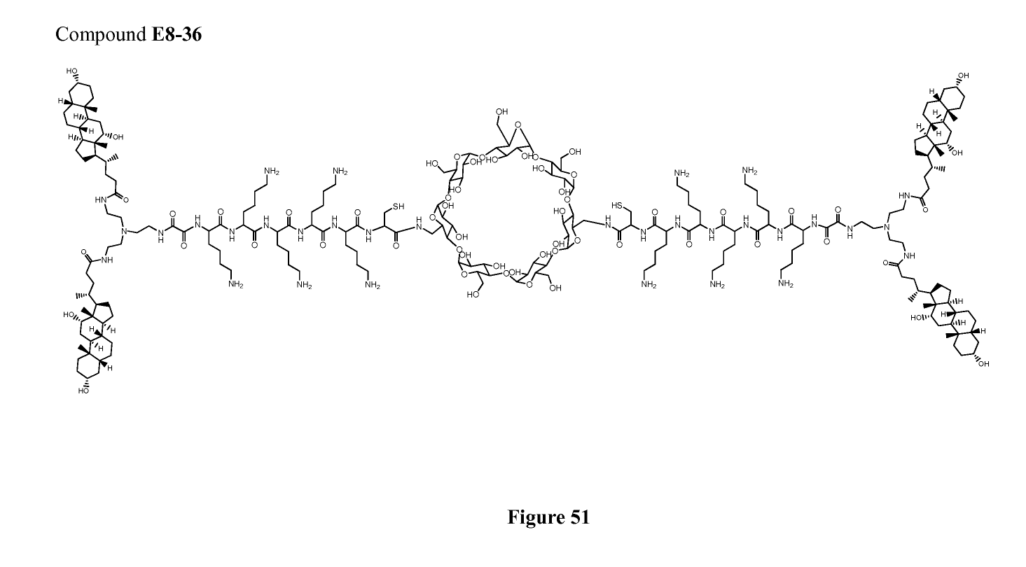

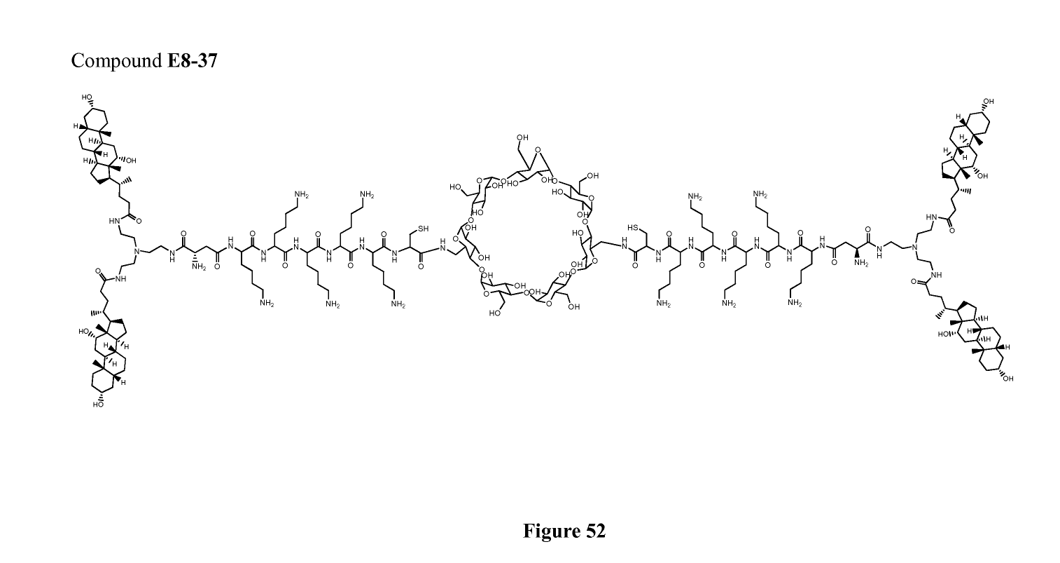

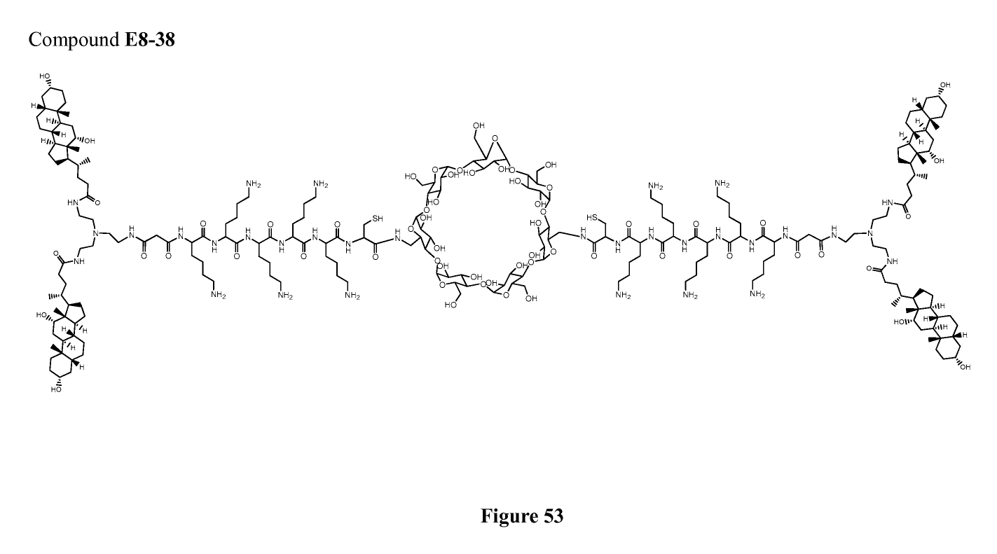

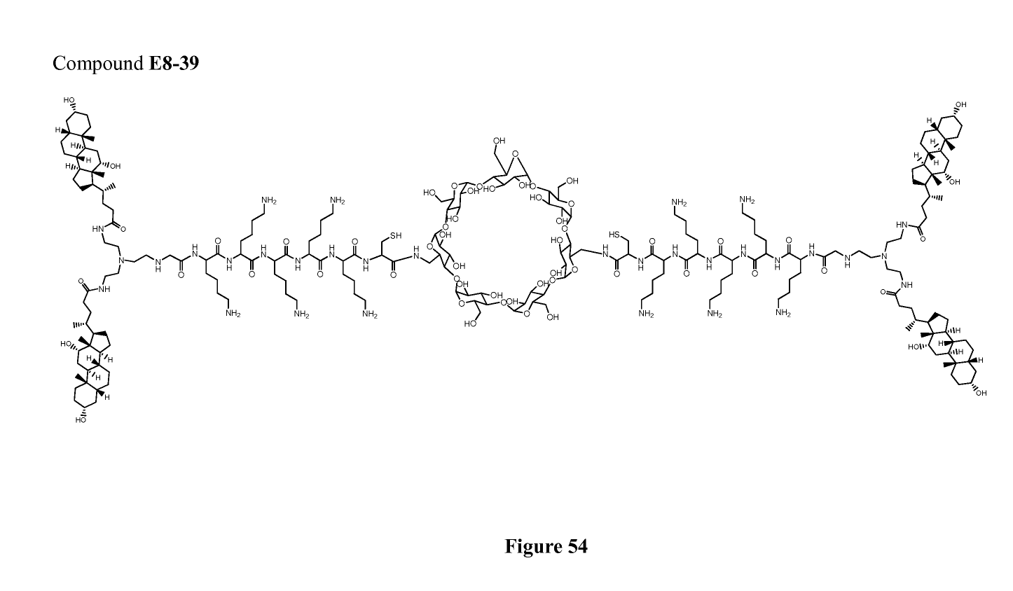

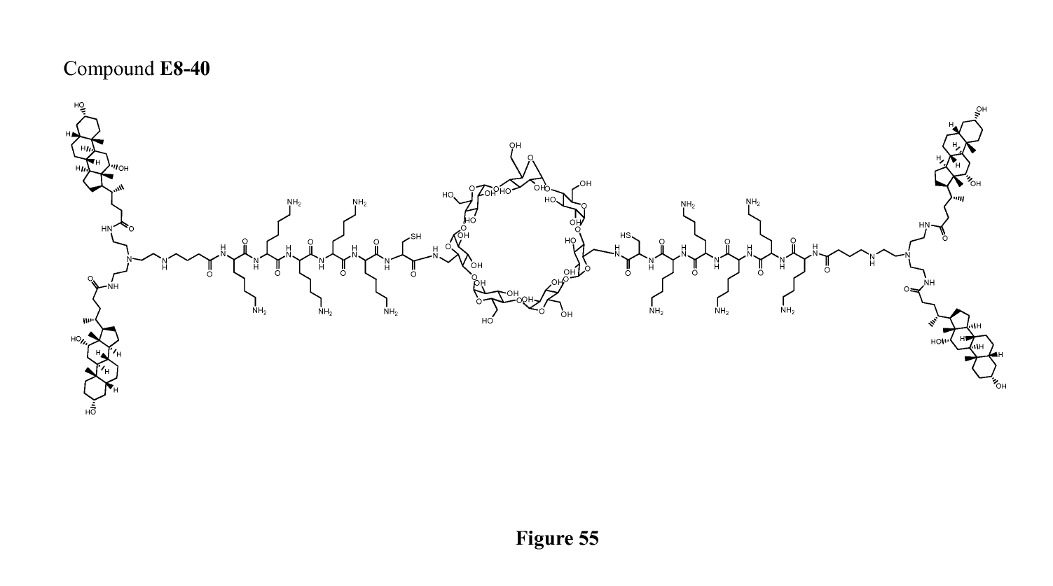

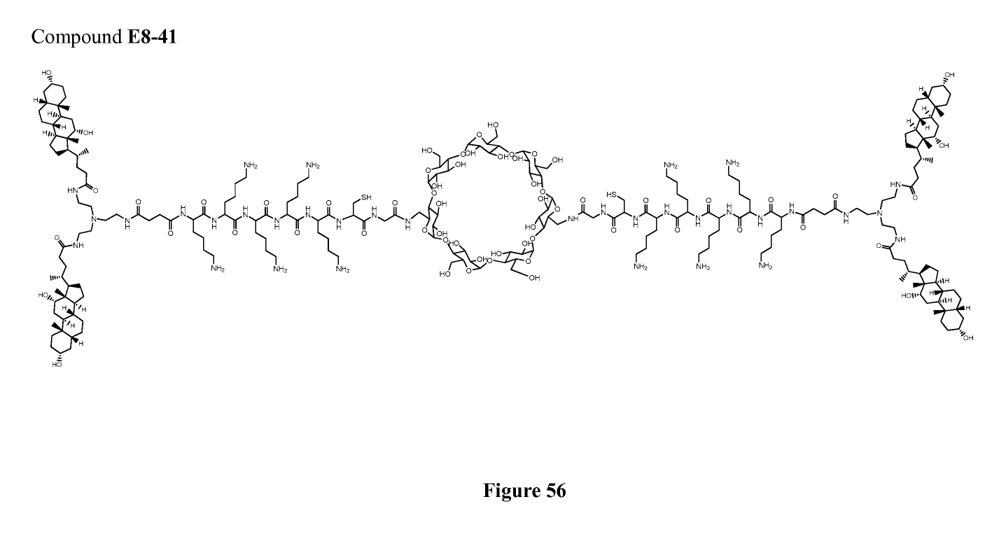

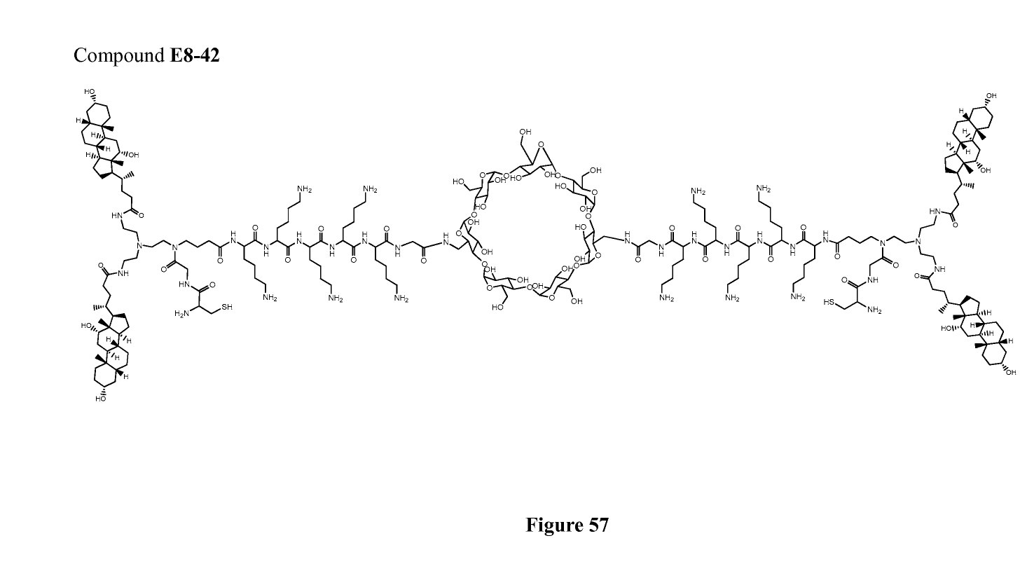

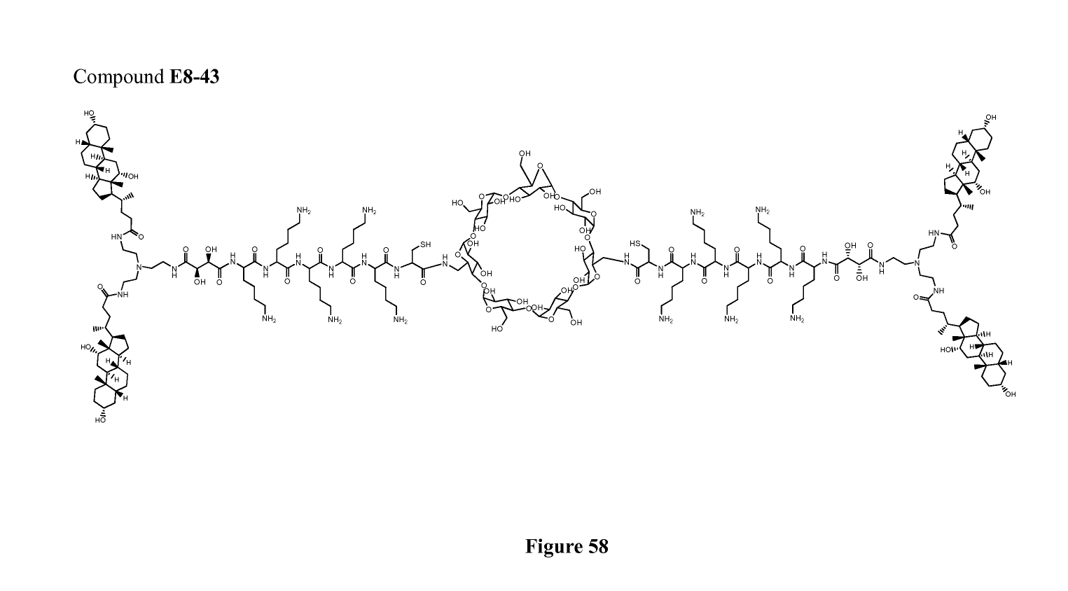

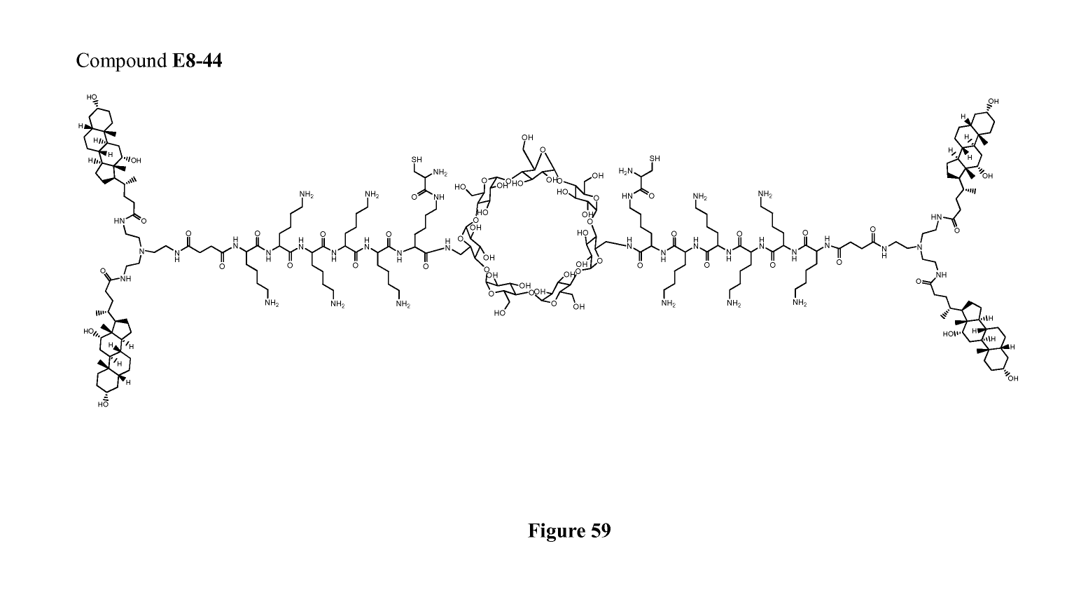

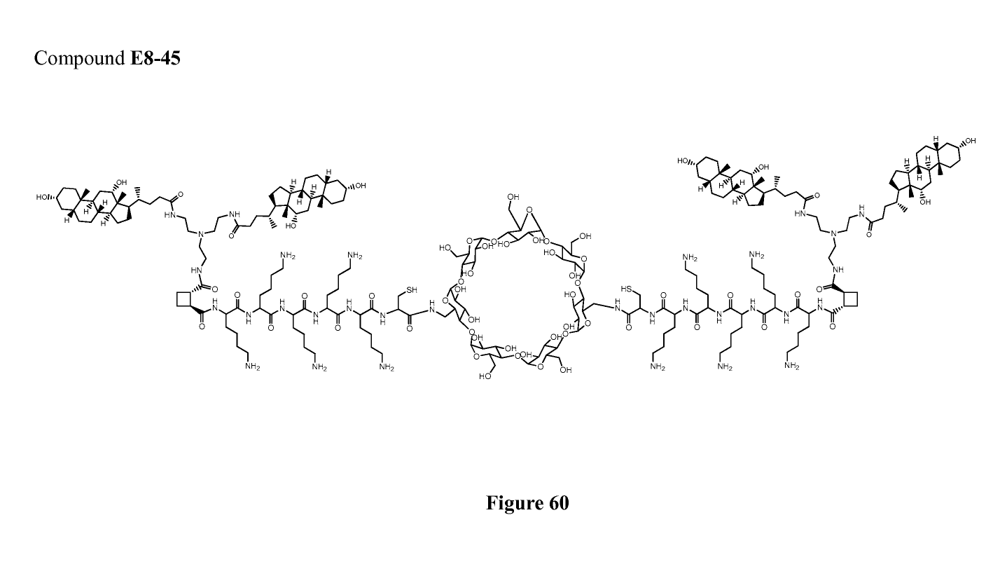

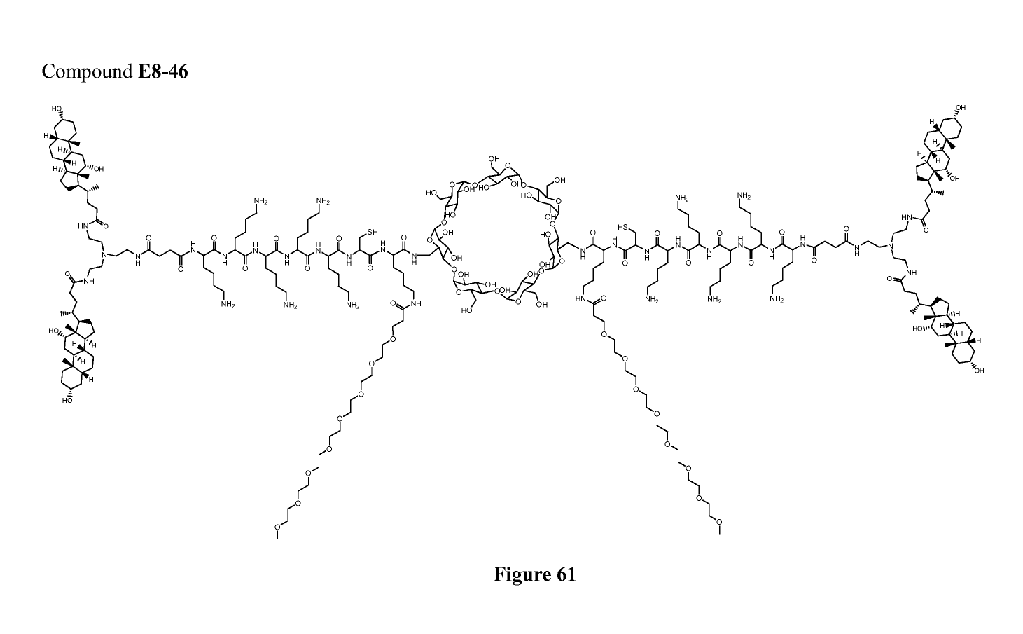

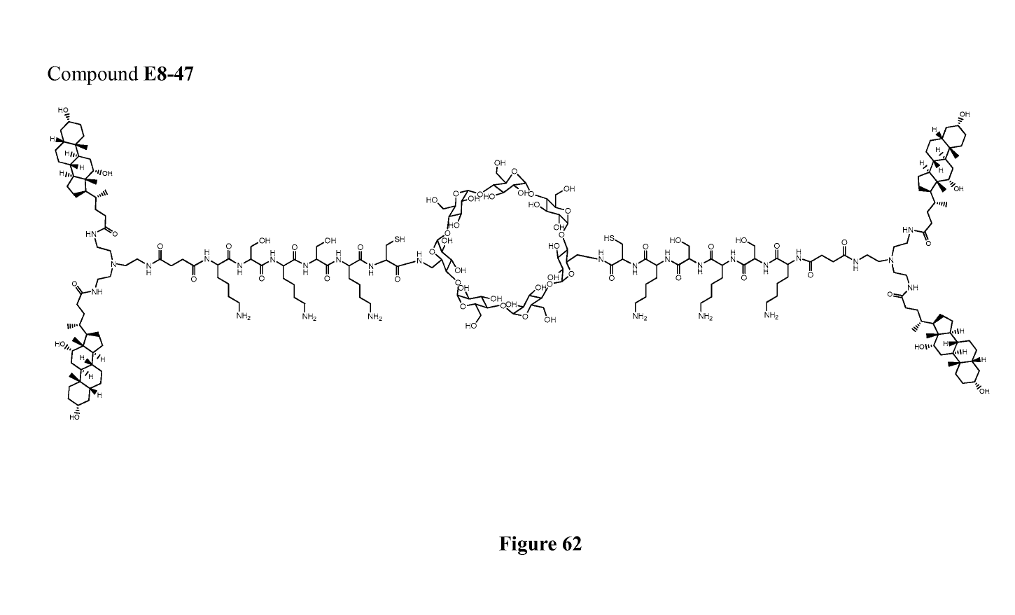

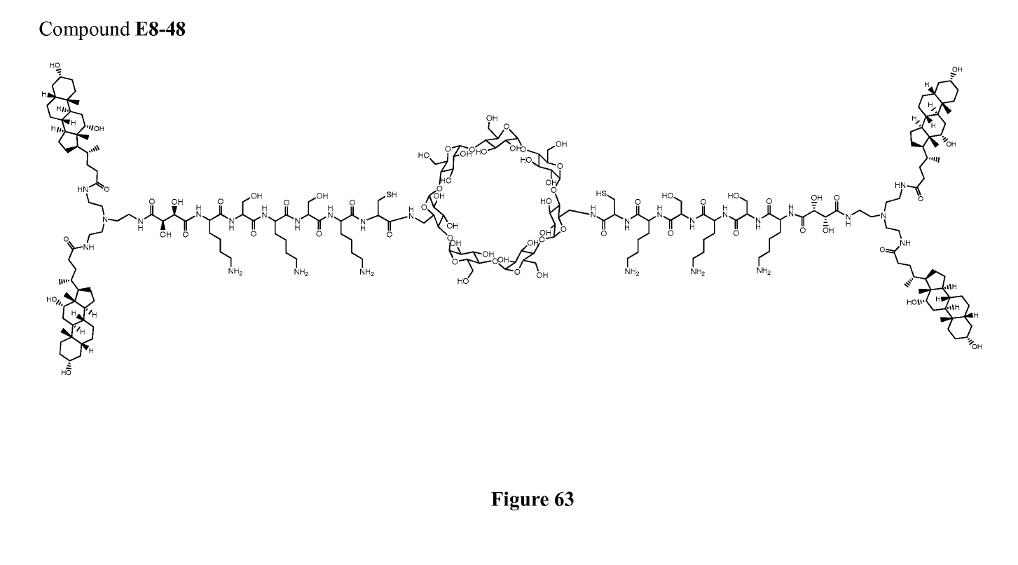

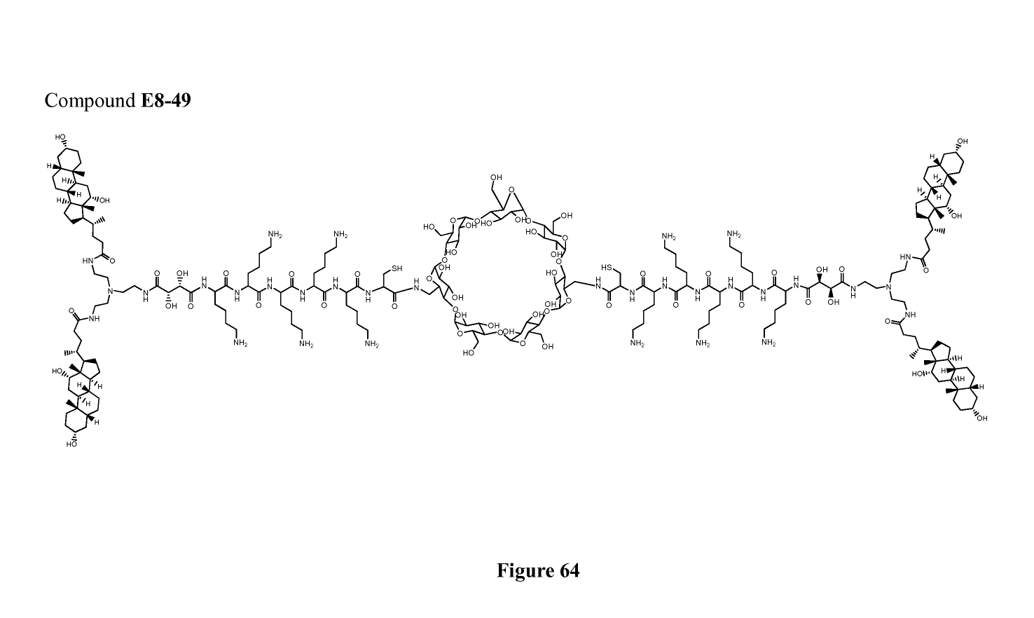

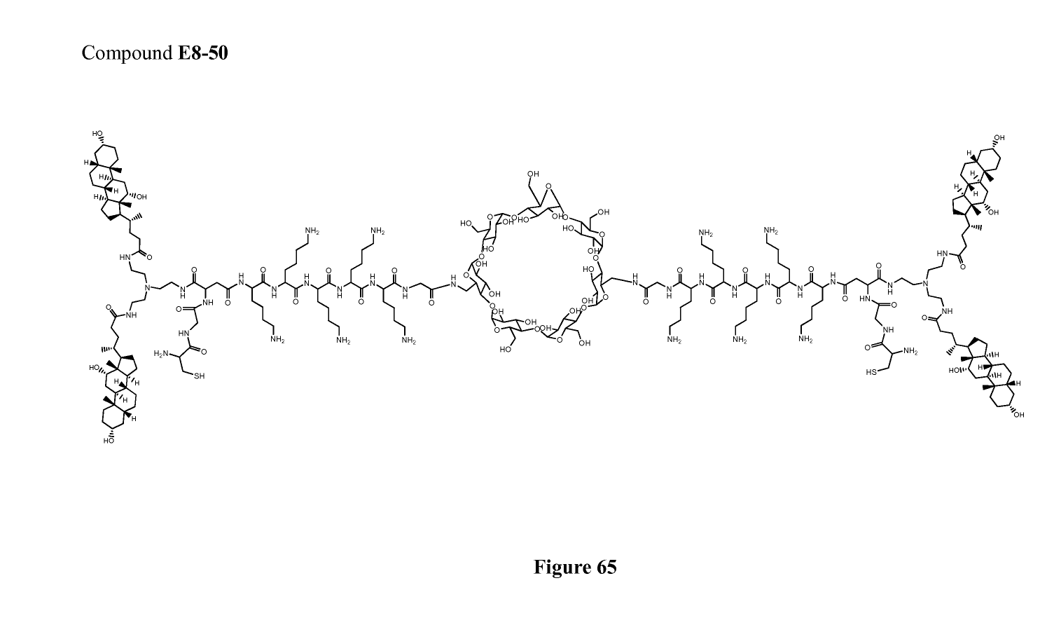

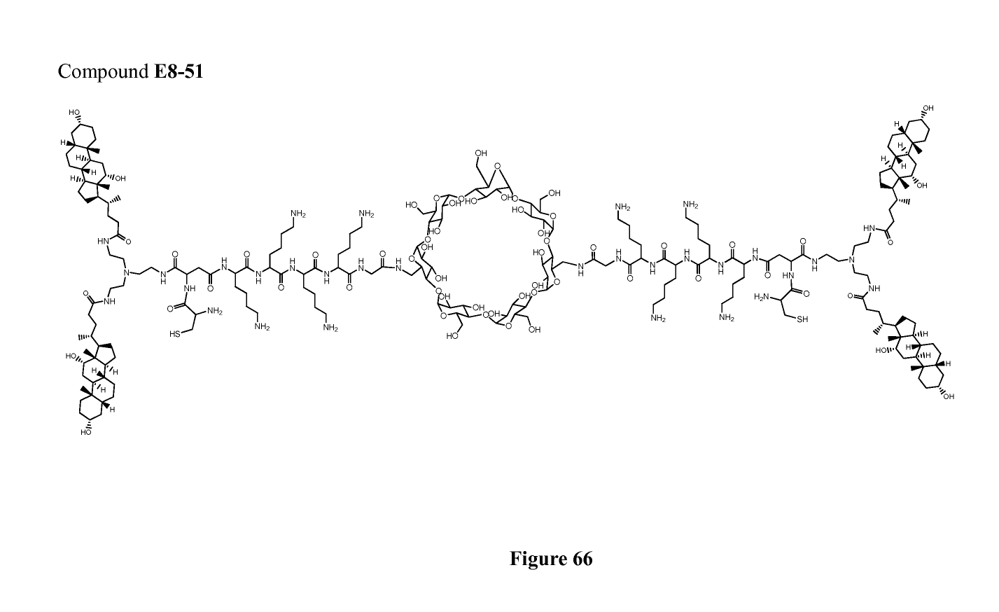

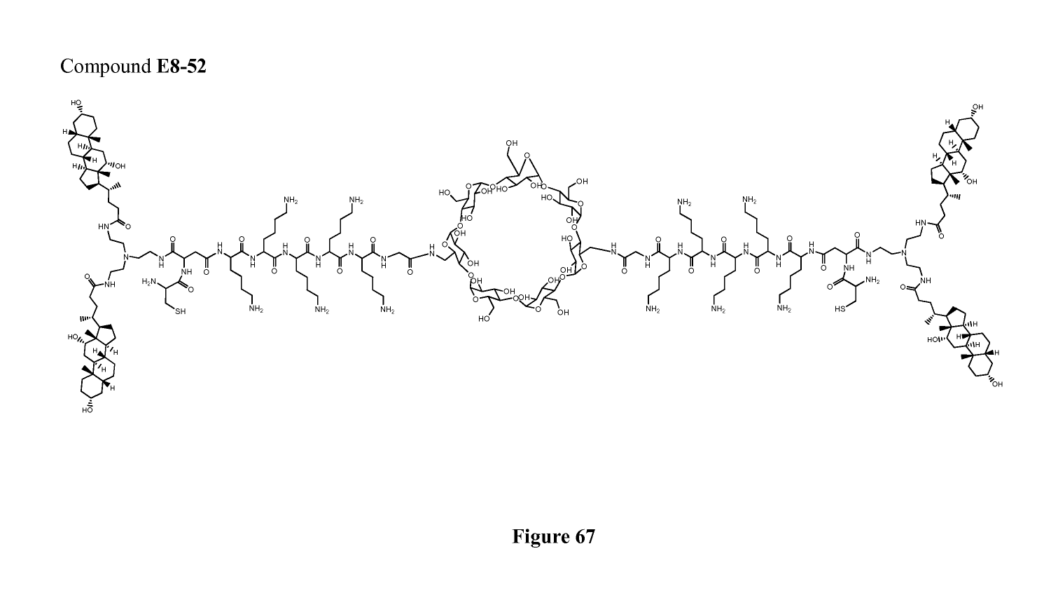

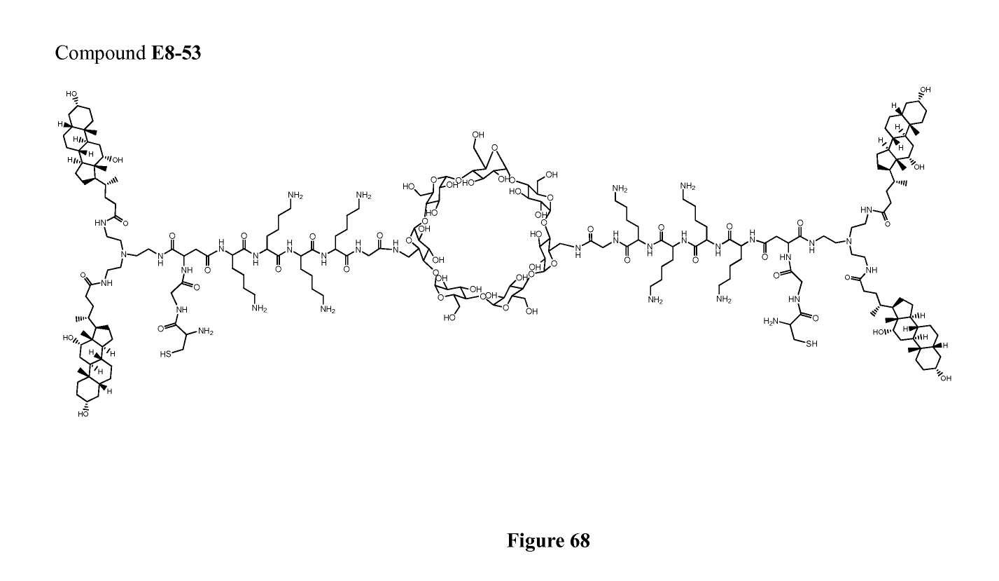

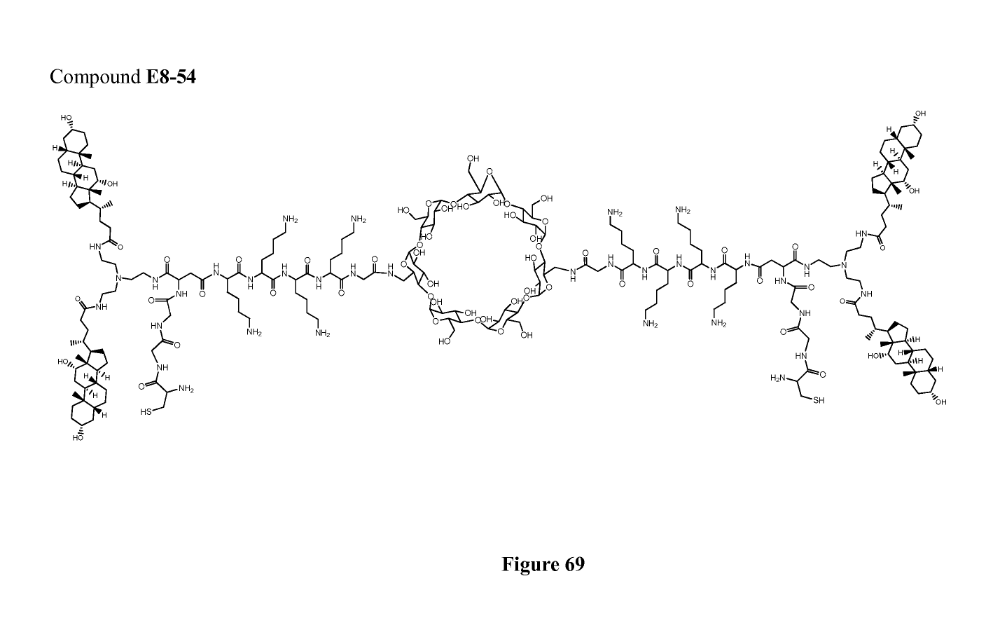

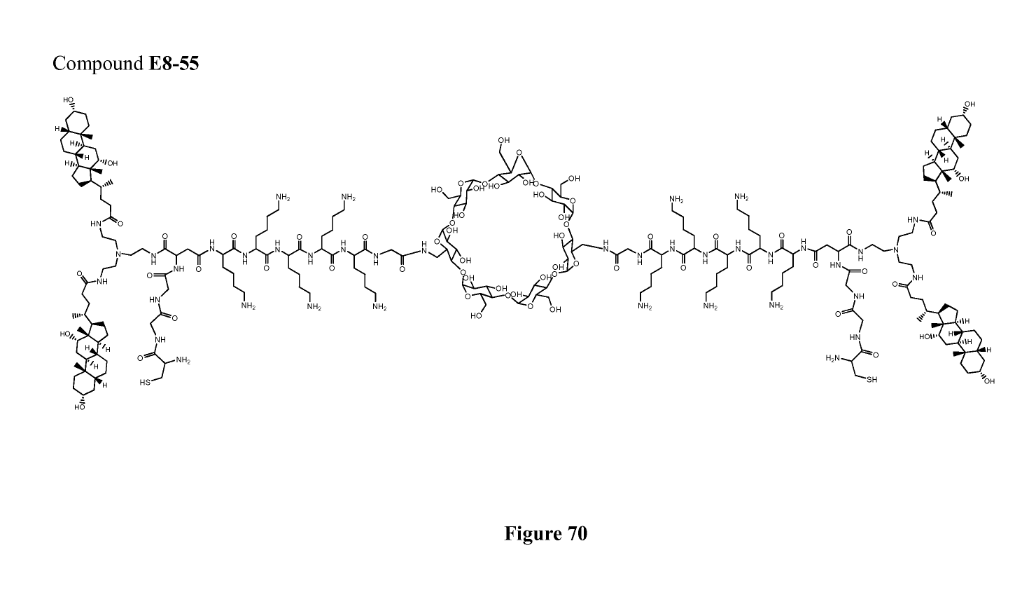

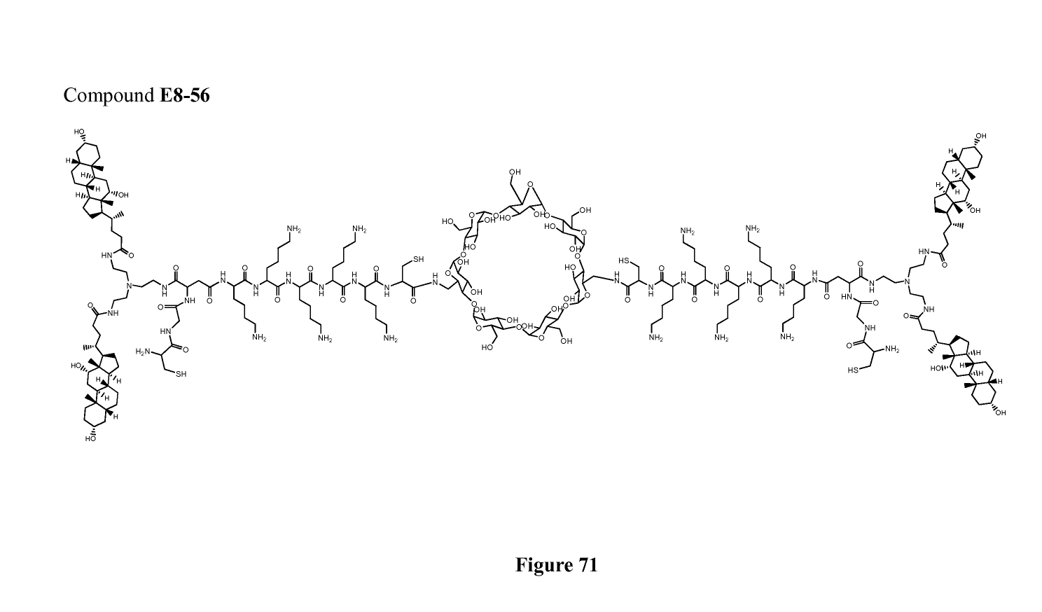

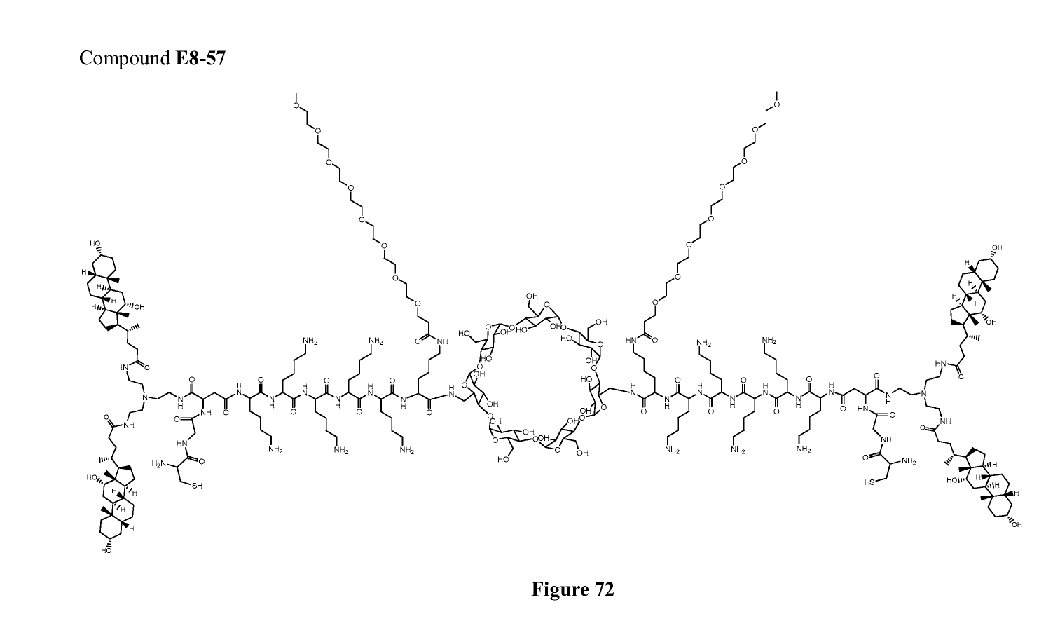

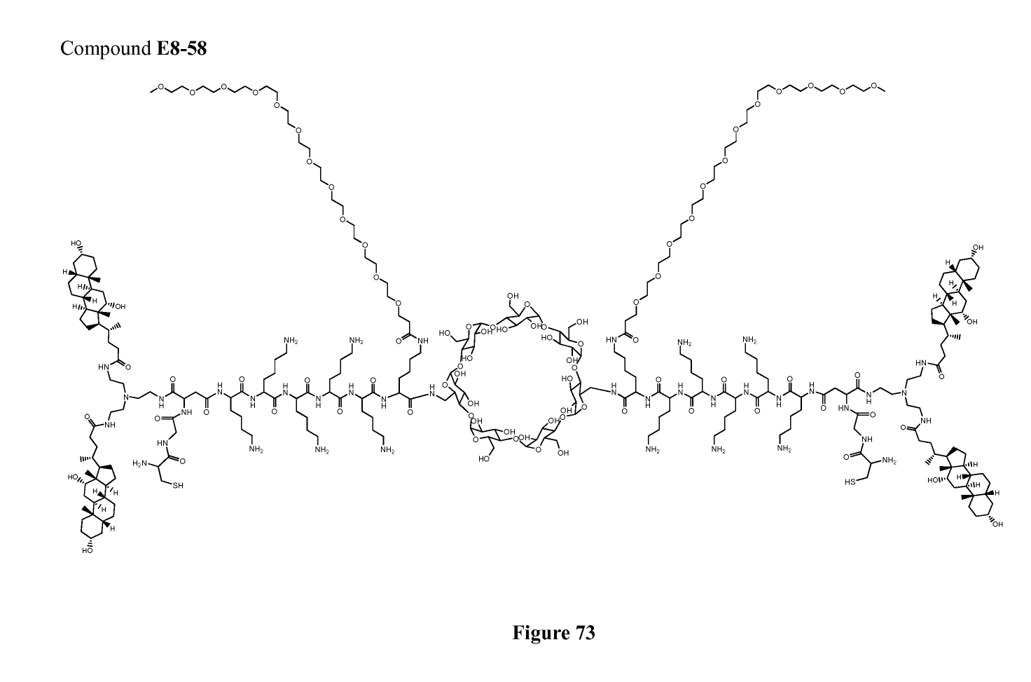

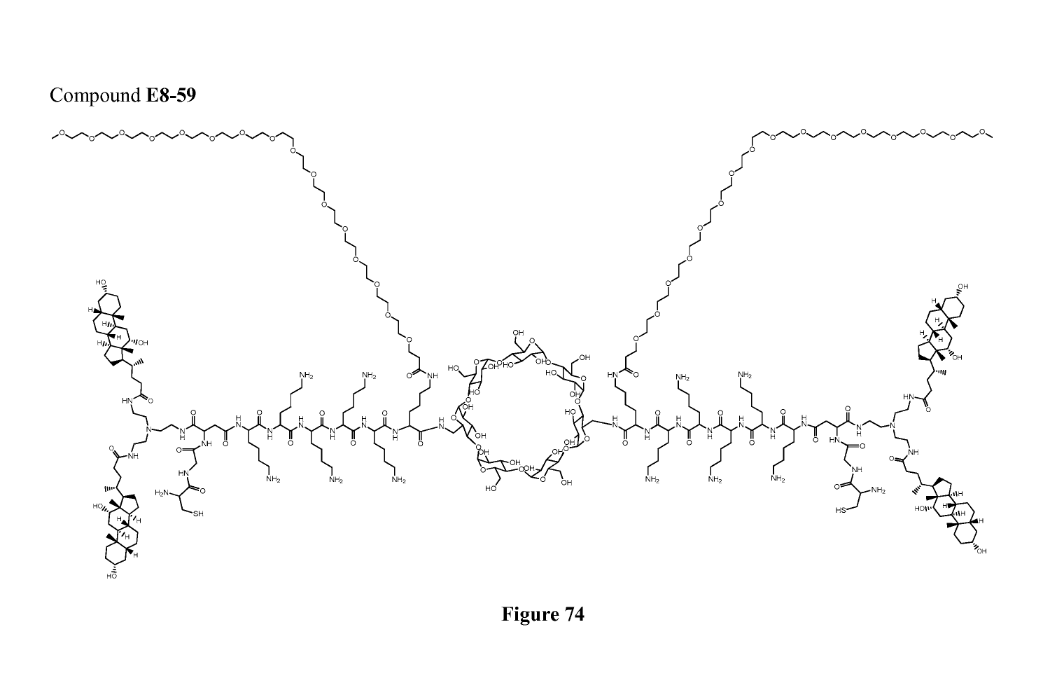

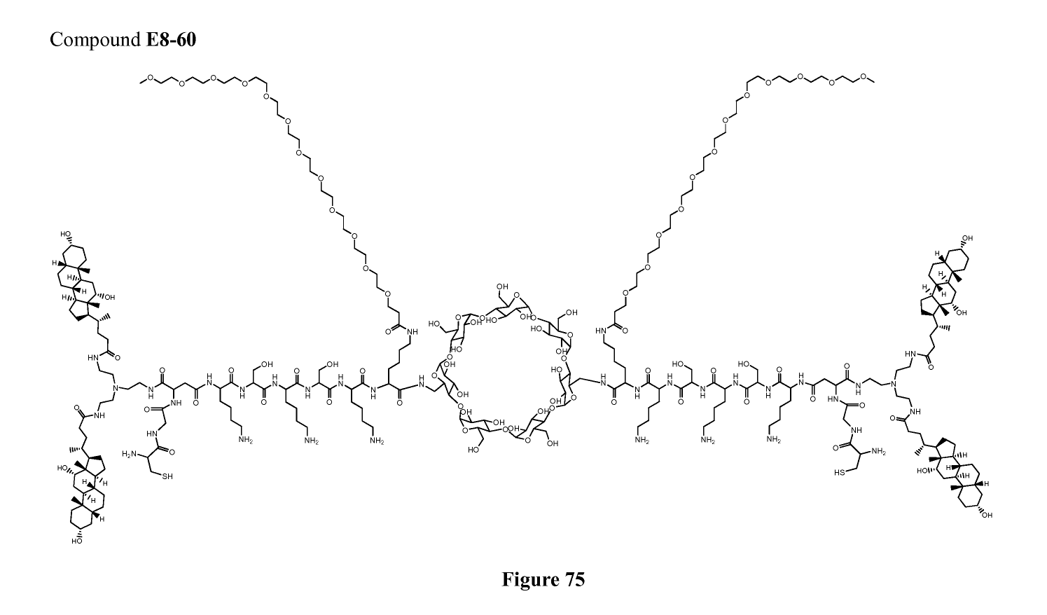

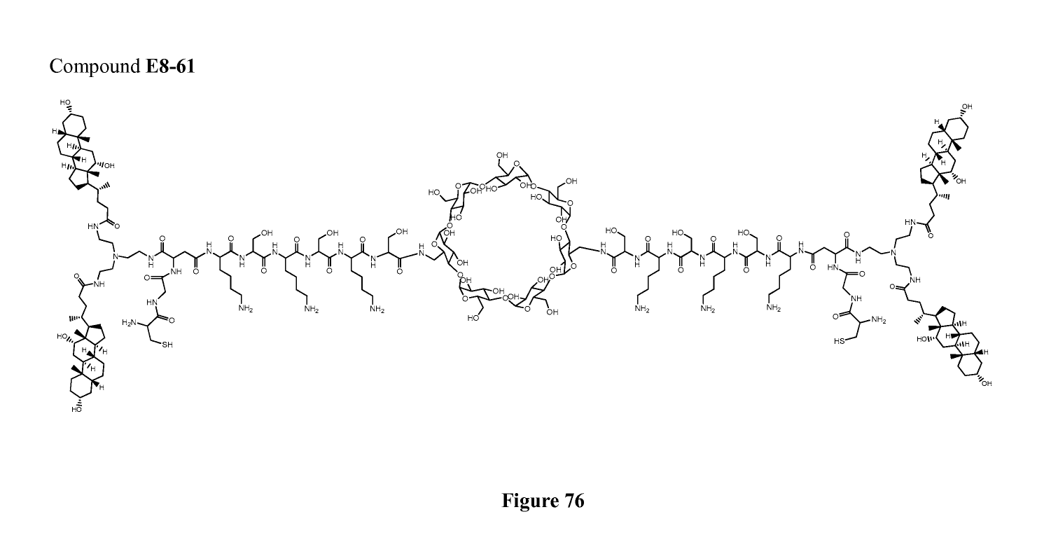

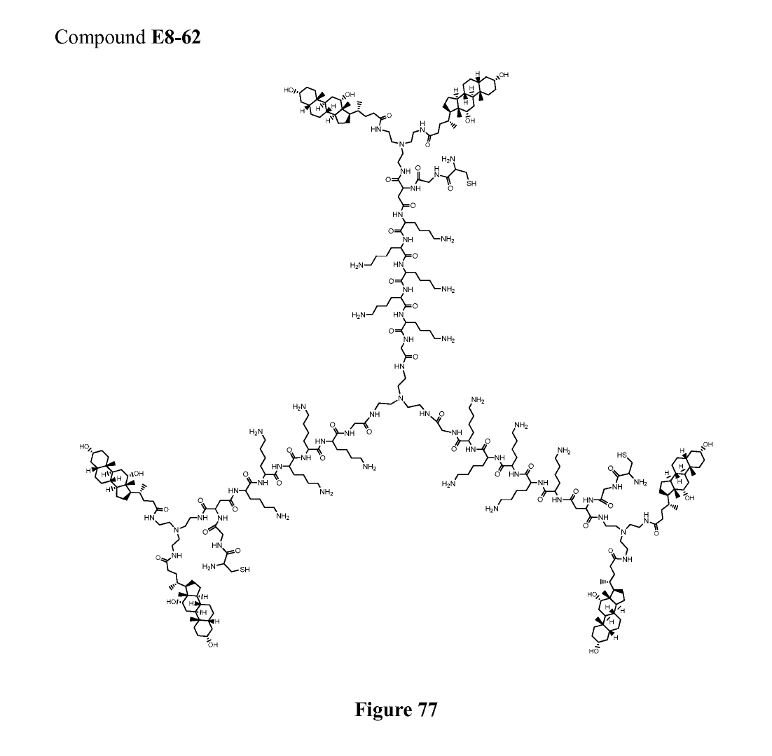

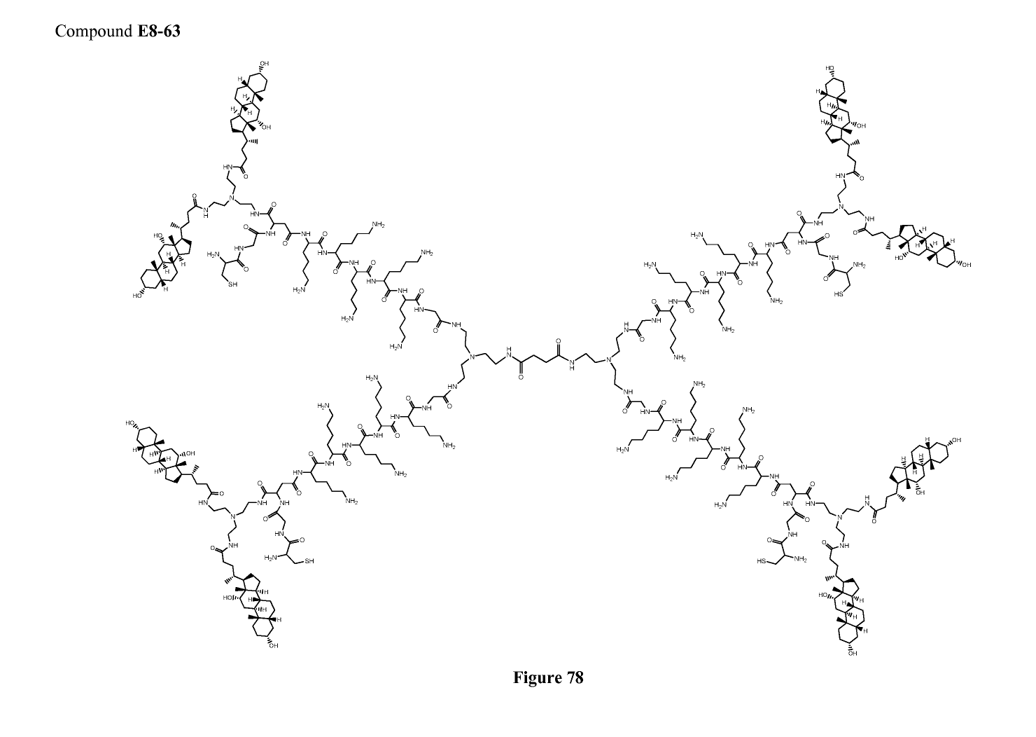

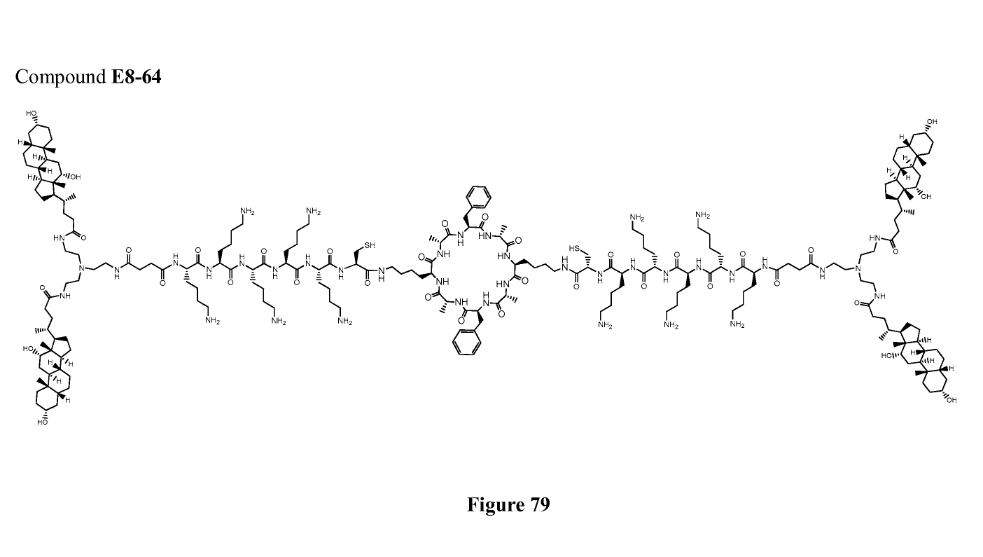

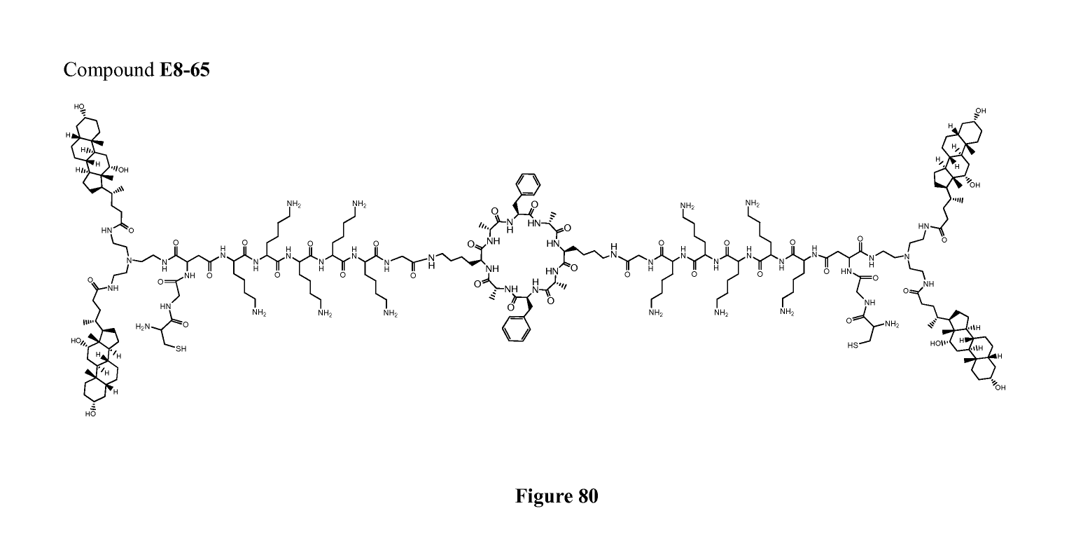

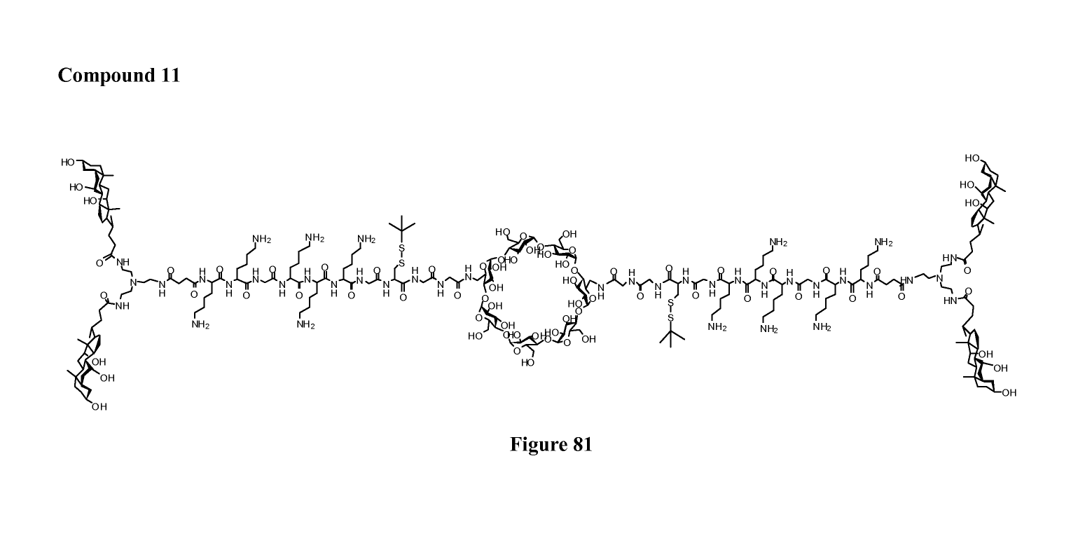

Additional compounds E8-11-E8-66 were prepared consistent with the procedures set forth herein (see FIGS. 27-81 for the structures thereof).

### Example 9

### Additional General Synthetic Procedures

I . . . for HATU Mediated Amide Bond Formation (A):

To a fully protected peptide/amino acid (C-terminus free, 1.1 eq with respect to amine) in anhydrous DMF was added HATU (1 eq with respect to acid) and DIEA (2 eq with respect to acid) and the mixture was stirred at room temperature for 5 minutes. The mixture was then added to a solution of amine in DMF and the reaction mixture was stirred at room temperature till the completion of the reaction (monitored by LC/MS). The solvent was removed under reduced pressure and the residue was triturated with saturated aqueous NaHCO₃. The precipitated solid was collected by filtration, washed with water twice and dried in vacuo. The crude product can be purified by reverse phase HPLC to give final pure product. The average yield after purification is between 20 to 60%.

### II . . . for DIC/HOBt Mediated Amide Bond Formation (B):

To a stirred solution of carboxylic acid (1.1 eq), amine and HOBt (1.1 eq) in anhydrous DMF was added DIC (1.1 eq) and the reaction mixture was stirred at room temperature for 24-48 h. Upon completion (monitored by LC/MS), the solvent was removed under reduced pressure and the residue was triturated with saturated aqueous NaHCO₃. The precipitated solid was collected by filtration, washed with water twice and dried in vacuo. The crude product can be purified by reverse phase HPLC to give final pure product. The average yield after purification is between 20 to 60%.

III . . . for Removal of Acid Sensitive Protecting Groups (Boc, Trt, Pbf) (C):

The acid sensitive protecting groups containing compound was dissolved in cleavage cocktail (TFA/TES/water, 95/2.5/2.5, v/v/v for compounds without Cys and TFA/EDT/TES/water, 90/2.5/5/2.5, v/v/v/v for S containing compound) and the mixture was stirred at room temperature for 2 h. The solution was then concentrated under reduced pressure and the residue was washed twice with cold ether. Purification was carried out on reverse phase HPLC if necessary.

### IV . . . for Removal of Fmoc Group (D):

The Fmoc containing compound was dissolved in 5% piperidine in DMF. The mixture was stirred at room temperature for 30 min. The solvents were removed under reduced pressure and the residue was washed with ether twice. The crude product can be used directly without further purification.

V . . . for Removal of S-tBu from Cys (E):

The Cys (S-tBu) containing compound was dissolved in sodium phosphate buffer (50 mM, pH 7.25) in the presence of EDTA (10 mM). MeOH can be added to aid dissolution. TCEP (100 eq per S-StBu) was added to the solution and the pH was adjusted by adding 1M aq. NaOH to 7-7.5. The mixture was stirred at room temperature under N₂ for 1 h and purified by reverse phase HPLC. The desired fractions were pooled and lyophilized.

### VI . . . for Removal of Cbz (Z) Group (F):

The Cbz containing compound was dissolved in MeOH/Dioxane (1/1, v/v) and ammonium formate (50 eq per Cbz group) was added. The mixture was purged by N₂ and catalytic amount of Pd in charcoal was added. The reaction was stirred at room temperature for 24 h. The catalyst was removed by filtration and the filtrate was concentrated and lyophilized to give the crude product which was purified by reverse phase HPLC.

VII . . . for Removal of Boc Group Using HCl Solution (G):

The Boc containing compound was dissolved in 4 N HCl in MeOH/H₂O (2/1, v/v). The reaction mixture was stirred at room temperature for 2 h before it was concentrated under reduced pressure. The residue can be purified by reverse phase HPLC if necessary.

### Example 10

### Preparation of Oligopeptide-Cyclodextrin Conjugates 11-27 and 31

A series of oligopeptide-cyclodextrin conjugates having the following structure were prepared as described herein:

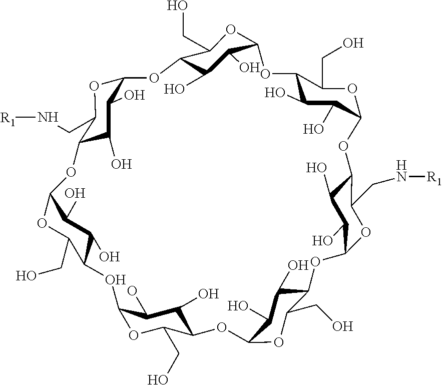

| | |
|---|---|
| 11. R1 = GGC(StBu)GKKKGKK-X1 | (SEQ ID NO: 33) |
| 12. R1 = GGCGKKKGKK-X1 | (SEQ ID NO: 34) |
| 13. R1 = GK(X1)GKKKK | (SEQ ID NO: 35) |
| 14. R1 = GGK(Ch1)GK(Ch1)GOOO | (SEQ ID NO: 36) |
| 15. R1 = GGK(Ch2)GK(Ch2)GOOO | (SEQ ID NO: 37) |
| 16. R1 = GGK(Ch1)GK(Ch1)GOOOO | (SEQ ID NO: 38) |
| 17. R1 = GGK(Ch1)GK(Ch1)GR | (SEQ ID NO: 39) |
| 18. R1 = GGK(X2)GKKKK | (SEQ ID NO: 40) |
| 19. R1 = GGK(X2)RRRR | (SEQ ID NO: 41) |
| 20. R1 = GGK(Ch2)GK(Ch2)GRR | (SEQ ID NO: 42) |
| 21. R1 = GGK(Ch1)GK(Ch1)GRR | (SEQ ID NO: 43) |
| 22. R1 = GGK(Ch2)GK(Ch2)GRRR | (SEQ ID NO: 44) |
| 23. R1 = GGK(Ch1)GK(Ch1)GRRR | (SEQ ID NO: 45) |
| 24. R1 = GGK(Ch2)GK(Ch2)GKKKK | (SEQ ID NO: 46) |
| 25. R1 = GGK(Ch1)GK(Ch1)GKKKK | (SEQ ID NO: 47) |
| 26. R1 = COOOOOX3 | |
| 27. R1 = COOOOOX1 | |
| 31. R1 = GKKKKK(Ch1)GK(Ch1)GC | (SEQ ID NO: 48) |

G = Glycine;
C = Cysteine;
K = Lysine;
O = Ornithine;
R = Arginine;

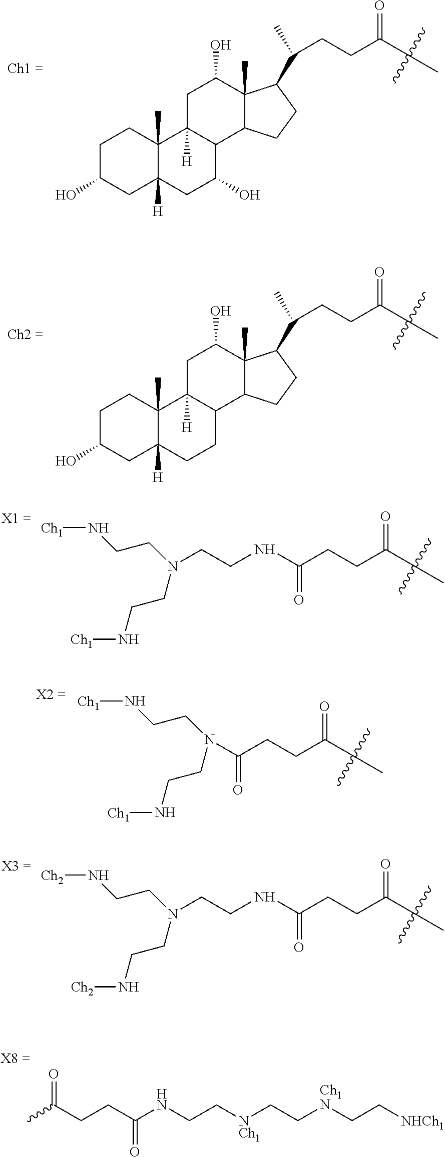

### Example 10-1

### Synthesis of Compound 11

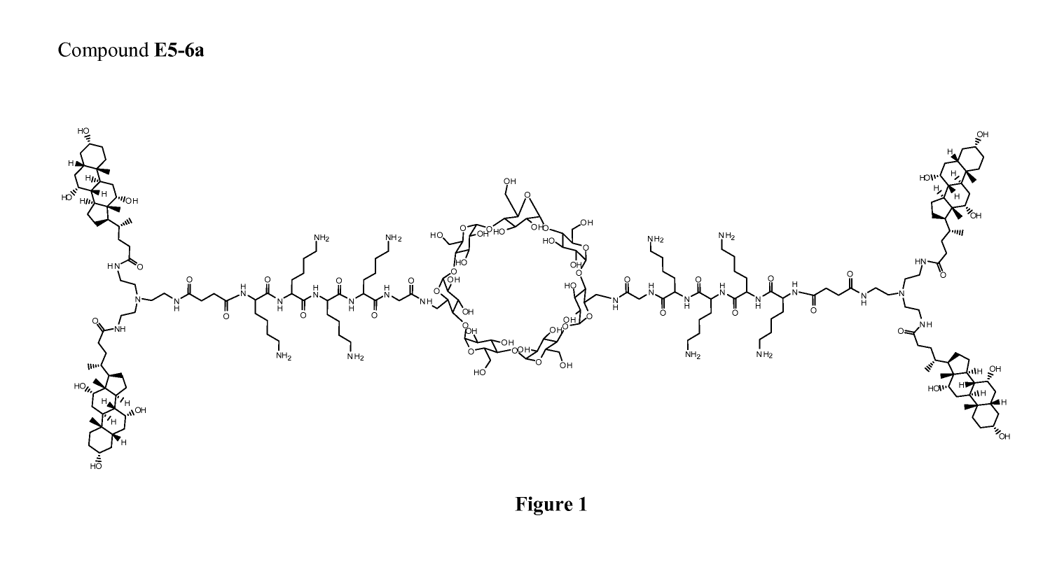

Compound 11 (see FIG. 1) was synthesized using the general procedures described above as follows: HATU mediated amide bond formation between 6^{A},6^{D}-dideoxy-diamino-β-cyclodextrin and Fmoc-GG-OH (General procedure A); Fmoc removal (General procedure D); HATU mediated amide bond formation between 6^{A},6^{D}-dideoxy-6^{A},6^{D}di-(Gly-Gly-amino)-β-cyclodextrin and Fmoc-Cys(StBu)-OH (General procedure A); Fmoc removal (General procedure D); HATU mediated amide bond formation between 6^{A},6^{D}-dideoxy-6^{A},6^{D}-di-(Cys(StBu)-Gly-Gly-amino)-β-cyclodextrin and peptide Fmoc-K(Boc)K(Boc)GK(Boc)K(Boc)K(Boc)G-OH (SEQ ID NO: 49) (General procedure A); Fmoc removal (General procedure D); DIC/HOBt mediated amide bond formation between the resulting cyclodextrin-peptide conjugate from the previous step and X1-OH (General procedure B); and final deprotection (General procedure C, no thiol scavenger). The final compound was purified by reverse phase HPLC to give compound 1 as a white powder after lyophilization. ¹H-NMR (300 MHz, D₂O) δ 5.07-4.92 (m, 7H), 4.35-3.35 (m, 82H), 3.05-2.85 (m, 20H), 2.65-0.60 (m, 246H); MS m/z calcd. for C₂₄₈H₄₃₄N₄₀O₇₃S₄ 5269.0, found 2636.8 ([M+2]⁺⁺/2).

### Example 10-2

### Synthesis of Compound 12

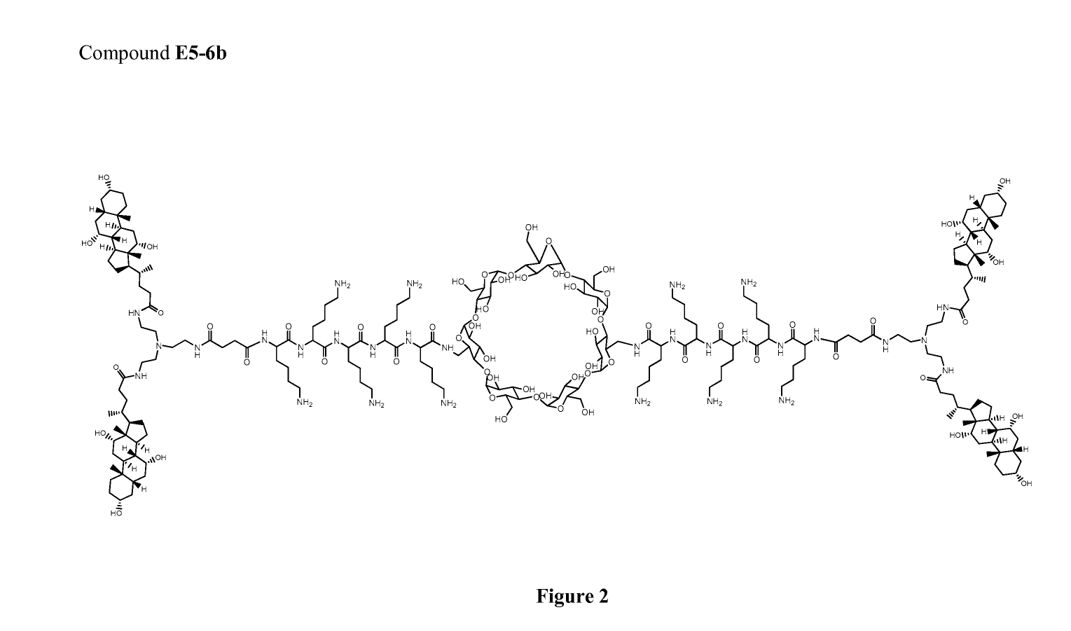

The S-tBu group on compound 11 was removed according to the procedure described above to yield compound 12 (see FIG. 2) as a white powder ¹H-NMR (300 MHz, D₂O) δ 5.07-4.92 (m, 7H), 4.35-3.30 (m, 82H), 3.05-2.85 (m, 20H), 2.65-0.60 (m, 228H); MS m/z calcd. for C₂₄₈H₄₁₈N₄₀O₇₃S₂ 5093.0, found 1699.8 ([M+3]⁺⁺⁻/3).

### Example 10-3

### Synthesis of Compound 13

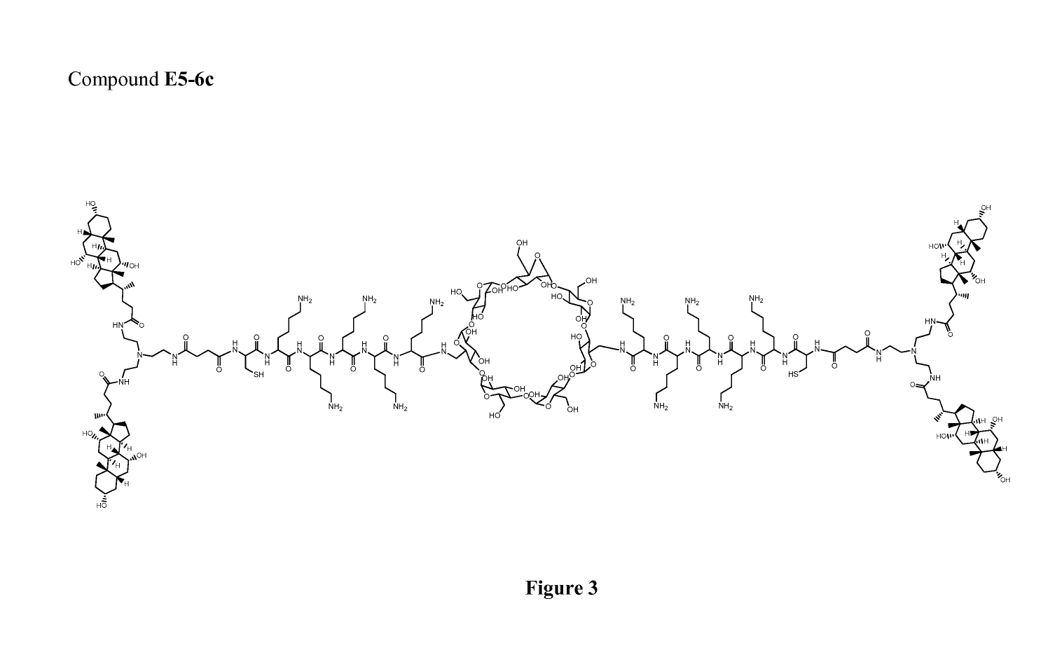

Compound 13 (see FIG. 3) was synthesized using the general procedures described above as follows: HATU mediated amide bond formation between 6^{A},6^{D}-dideoxy-diamino-β-cyclodextrin and Fmoc-K(Z)G-OH (General procedure A); Fmoc removal (General procedure D); HATU mediated amide bond formation between 6^{A},6^{D}-dideoxy-6^{A},6^{D}-di-(Lys(Z)-Gly-amino)-β-cyclodextrin and peptide Boc-K(Boc)K(Boc)K(Boc)K(Boc)G-OH (SEQ ID NO: 1) (General procedure A); Cbz removal (General procedure F); DIC/HOBt mediated amide bond formation between the resulting cyclodextrin-peptide conjugate from the previous step and X1-OH (General procedure B); and final deprotection (General procedure C). The final compound was purified by reverse phase HPLC to give compound 13 as a white powder after lyophilization. ¹H-NMR (300 MHz, D₂O) δ 4.95-4.80 (m, 7H), 4.35-3.25 (m, 72H), 3.10-2.95 (m, 4H), 2.90-2.80 (m, 16H), 2.45-0.45 (m, 224 H); MS m/z calcd. for C₂₂₆H₃₉₆N₃₄O₆₇ 4658.9, found 1555.8 ([M+3]⁺⁺⁺/3).

### Example 10-4

### Synthesis of Compound 14

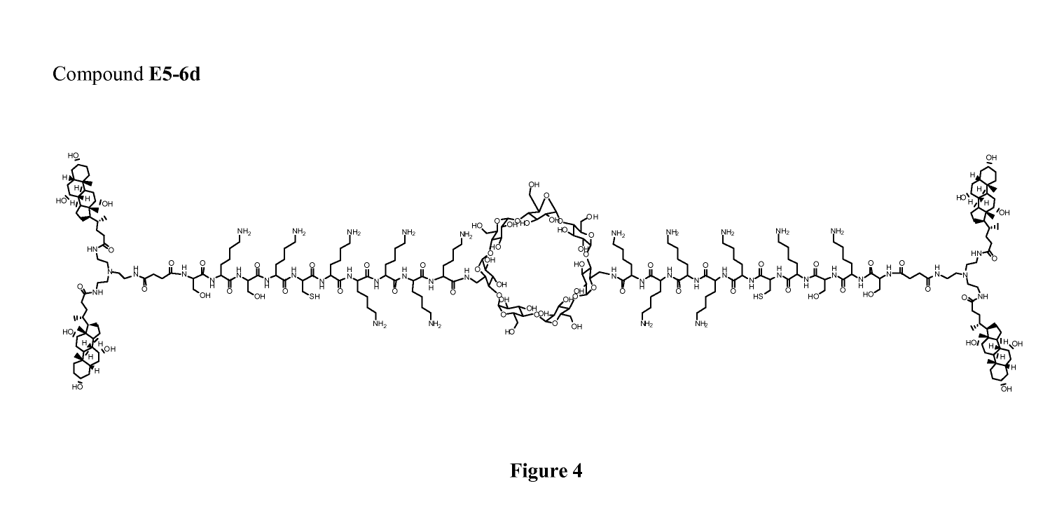

Compound 14 (see FIG. 4) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocO(Boc)O(Boc)O(Boc)GK(Z)GK(Z)-OH (SEQ ID NO: 50) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 14 as a white powder after lyophilization. ¹H-NMR (300 MHz, D₂O) δ 5.10-4.95 (m, 7H), 4.35-3.35 (m, 80H), 3.25-2.85 (m, 20H), 2.45-0.45 (m, 180H); MS m/z calcd. for C₂₀₈H₃₅₆N₃₀O₆₇ 4346.5, found 2175.4 ([M+2]⁻⁺/2).

### Example 10-5

### Synthesis of Compound 15

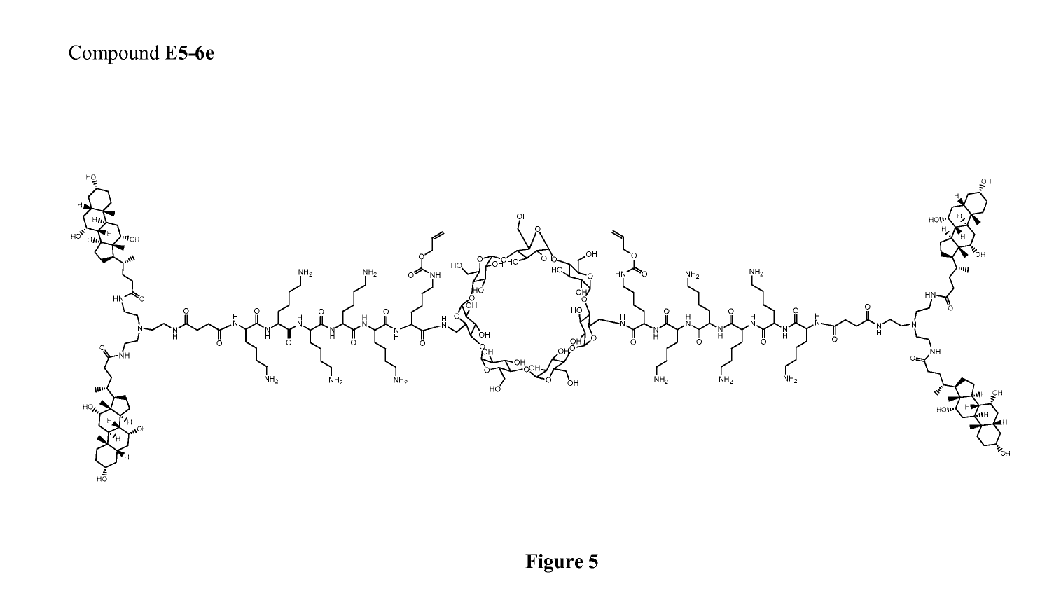

Compound 15 (see FIG. 5) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocO(Boc)O(Boc)O(Boc)GK(Z)GK(Z)-OH (SEQ ID NO: 50) (General procedure A); Cbz removal (General procedure F); DIC/HOBt mediated amide bond formation between the resulting cyclodextrin-peptide conjugate from the previous step and Ch2-OH (General procedure B); and final deprotection (General procedure C). The final compound was purified by reverse phase HPLC to give compound 15 as a white powder after lyophilization. ¹H-NMR (300 MHz, D₂O) δ 5.10-4.95 (m, 7H), 4.35-3.30 (m, 76H), 3.25-2.85 (m, 20H), 2.40-0.60 (m, 188H); MS m/z calcd. for C₂₀₈H₃₅₆N₃₀O₆₃ 4282.6, found 2143.2 ([M+2]⁻⁺/2).

### Example 10-6

### Synthesis of Compound 16

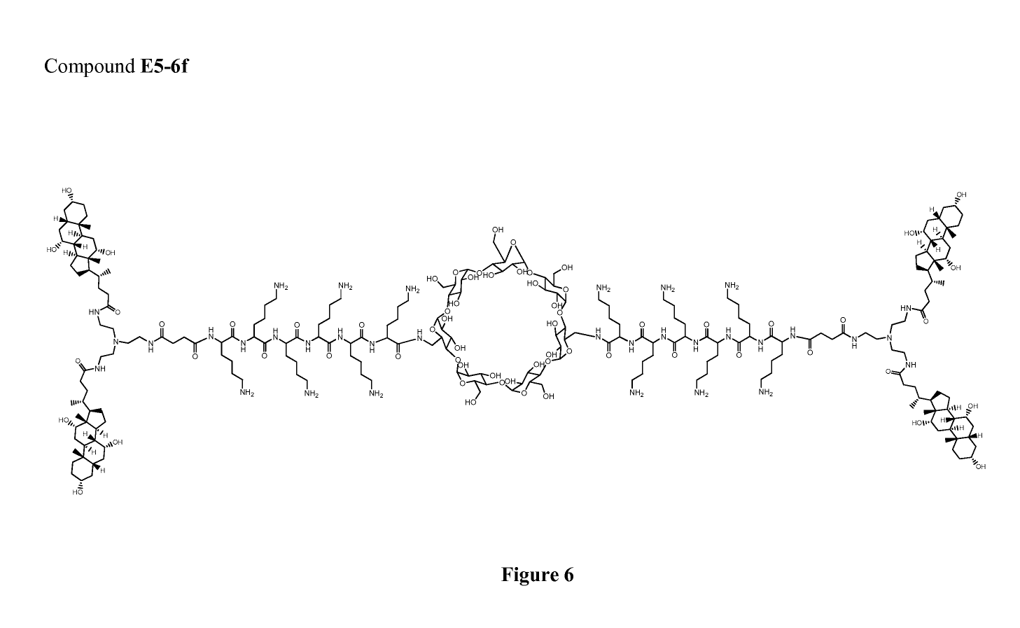

Compound 16 (see FIG. 6) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocO(Boc)O(Boc)O(Boc)O(Boc)GK(Z)GK(Z)-OH (SEQ ID NO: 51) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 16 as a white powder after lyophilization. ¹H-NMR (300 MHz, D₂O) δ 5.10-4.95 (m, 7H), 4.40-3.30 (m, 82H), 3.25-2.95 (m, 24H), 2.35-0.60 (m, 188H); MS m/z calcd. for C₂₁₈H₃₇₆N₃₄O₆₉ 4574.7, found 2289.4 ([M+2]⁺⁺/2).

### Example 10-7

### Synthesis of Compound 17

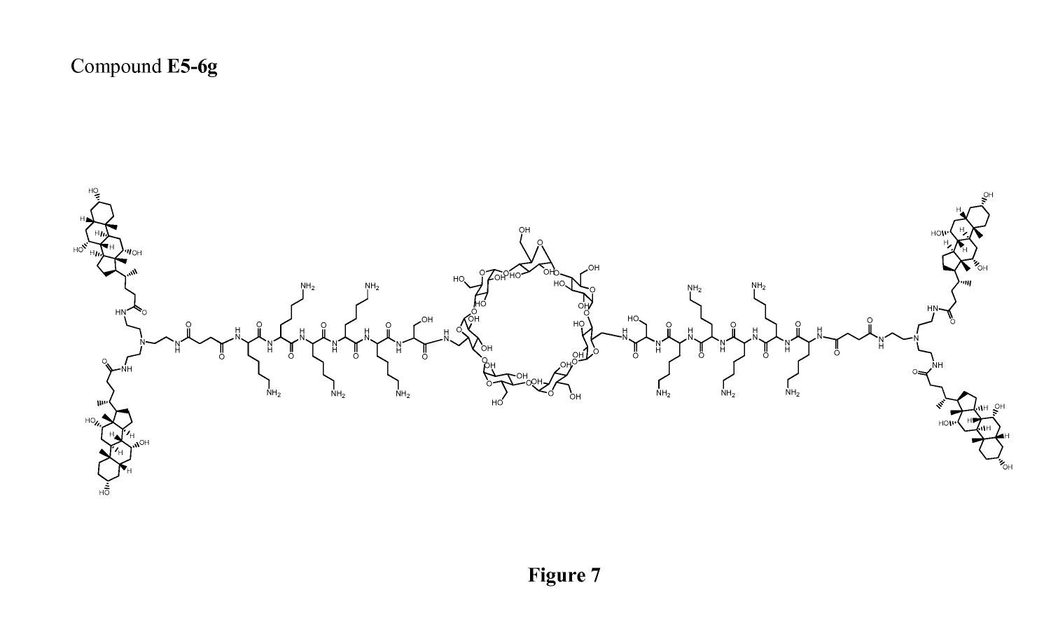

Compound 17 (see FIG. 7) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocR(Pbf)GK(Z)GK(Z)-OH (SEQ ID NO: 52) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 17 as a white powder after lyophilization. ¹H-NMR (300 MHz, D₂O) δ 5.10-4.95 (m, 7H), 4.40-3.35 (m, 76H), 3.20-3.00 (m, 12H), 2.35-0.55 (m, 164H); MS m/z calcd. for C₁₉₀H₃₂₀N₂₆O₆₃ 3974.3, found 1988.4 ([M+2]⁺⁺/2).

### Example 10-8

### Synthesis of Compound 18

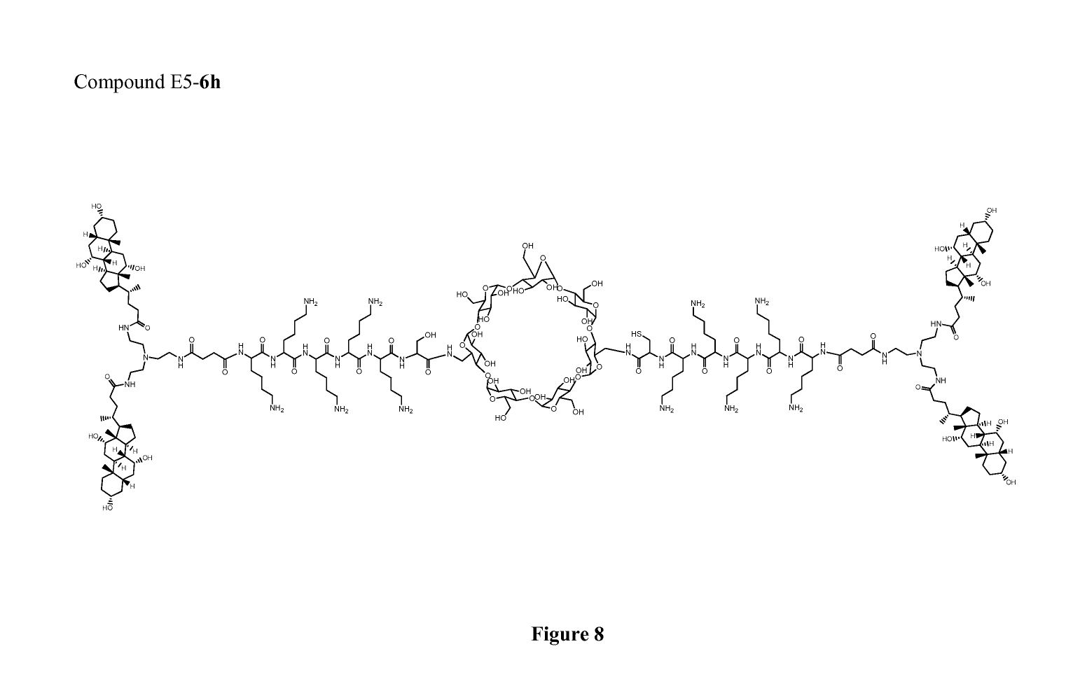

Compound 18 (see FIG. 8) was synthesized using the general procedures described above as follows: HATU mediated amide bond formation between 6^{A},6^{D}-dideoxy-diamino-β-cyclodextrin and Fmoc-K(Z)GG-OH (General procedure A); Fmoc removal (General procedure D); HATU mediated amide bond formation between 6^{A},6^{D}-dideoxy-6^{A},6^{D}-di-(Lys(Z)-Gly-Gly-amino)-β-cyclodextrin and peptide Boc-K(Boc)K(Boc)K(Boc)K(Boc)G-OH (SEQ ID NO: 1) (General procedure A); Cbz removal (General procedure F); DIC/HOBt mediated amide bond formation between the resulting cyclodextrin-peptide conjugate from the previous step and X2-OH (General procedure B); and final deprotection (General procedure C). The final compound was purified by reverse phase HPLC to give compound 18 as a white powder after lyophilization. ¹H-NMR (300 MHz, D₂O) δ 5.10-4.80 (m, 7H), 4.25-3.25 (m, 76H), 3.10-2.95 (m, 4H), 2.90-2.80 (m, 16H), 2.60-0.60 (m, 216H); MS m/z calcd. for C₂₂₆H₃₉₂N₃₄O₆₉ 4686.8, found 1564.4 ([M+3]⁺⁺⁺/3).

### Example 10-9

### Synthesis of Compound 19

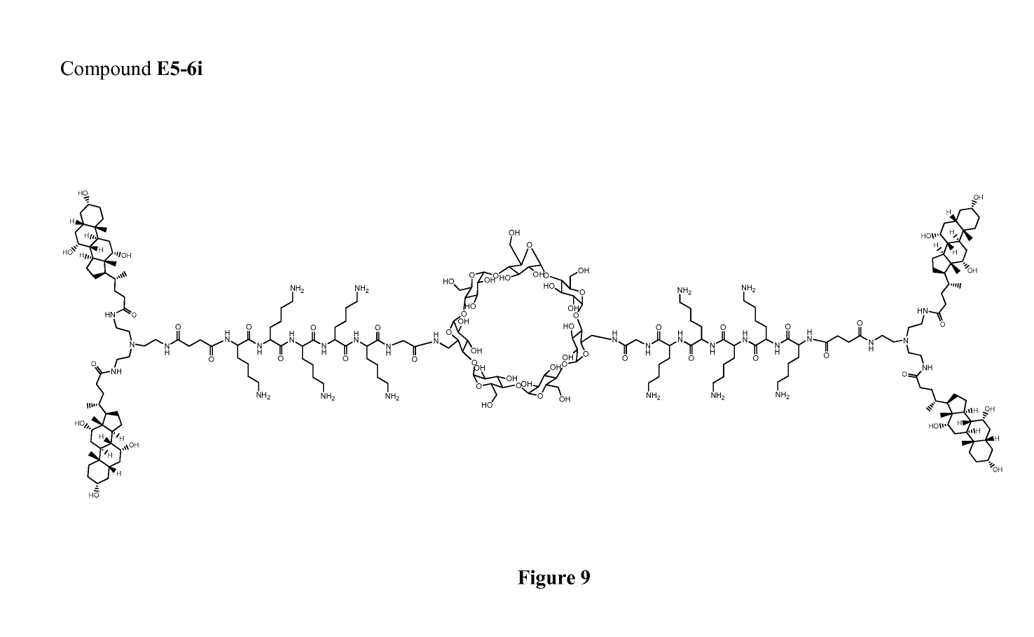

Compound 19 (see FIG. 9) was synthesized using the general procedures described above as follows: HATU mediated amide bond formation between 6^{A},6^{D}-dideoxy-diamino-β-cyclodextrin and Fmoc-K(Z)GG-OH (General procedure A); Fmoc removal (General procedure D); HATU mediated amide bond formation between 6^{A},6^{D}-dideoxy-6^{A},6^{D}-di-(Lys(Z)-Gly-Gly-amino)-β-cyclodextrin and peptide Boc-R(Pbf)R(Pbf) R(Pbf)R(Pbf)-OH (SEQ ID NO: 53) (General procedure A); Cbz removal (General procedure F); DIC/HOBt mediated amide bond formation between the resulting cyclodextrin-peptide conjugate from the previous step and X2-OH (General procedure B); and final deprotection (General procedure C). The final compound was purified by reverse phase HPLC to give compound 19 as a white powder after lyophilization. ¹H-NMR (300 MHz, D₂O) δ 5.05-4.95 (m, 7H), 4.35-3.05 (m, 92H), 2.70-0.55 (m, 200H); MS m/z calcd. for C₂₂₂H₃₈₆N₄₈O₆₇ 4796.8, found 1601.8 ([M+3]⁺⁺⁺/3).

### Example 10-10

### Synthesis of Compound 20

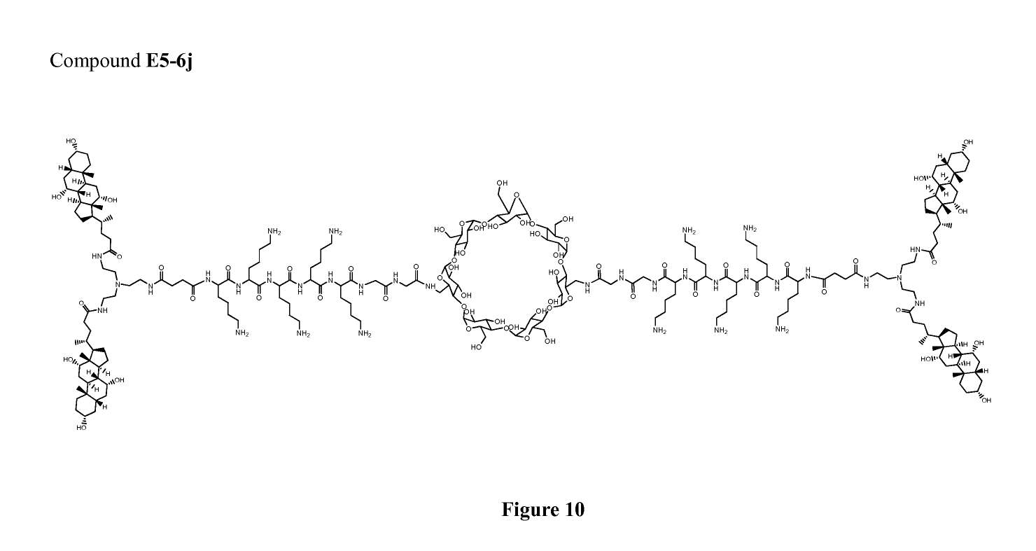

Compound 20 (see FIG. 10) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocR(Pbf)R(Pbf)GK(Z)GK(Z)-OH (SEQ ID NO: 54) (General procedure A); Cbz removal (General procedure F); DIC/HOBt mediated amide bond formation between the resulting cyclodextrin-peptide conjugate from the previous step and Ch2-OH (General procedure B); and final deprotection (General procedure C). The final compound was purified by reverse phase HPLC to give compound 20 as a white powder after lyophilization. ¹H-NMR (300 MHz, D₂O) δ 5.10-4.90 (m, 7H), 4.40-3.45 (m, 74H), 3.20-3.00 (m, 16H), 2.30-0.55 (m, 180H); MS m/z calcd. for C₂₀₂H₃₄₄N₃₄O₆₁ 4222.5, found 1409.8 ([M+3]⁻⁺⁺/3).

### Example 10-11

### Synthesis of Compound 21

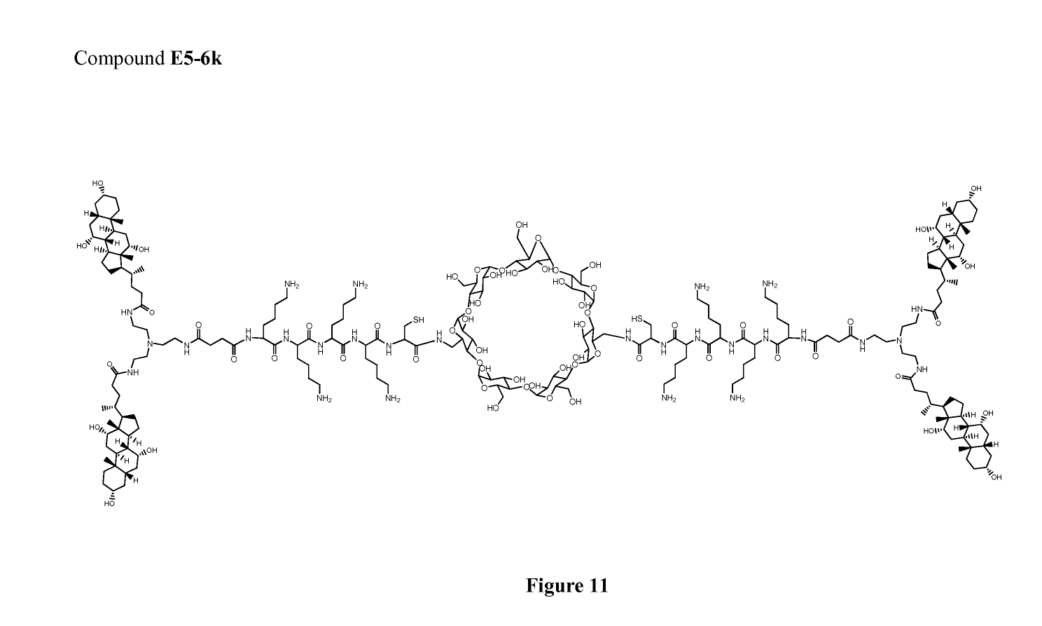

Compound 21 (see FIG. 11) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocR(Pbf)R(Pbf)GK(Z)GK(Z)-OH (SEQ ID NO: 54) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 21 as a white powder after lyophilization. ¹H-NMR (300 MHz, D₂O) δ 5.10-4.90 (m, 7H), 4.40-3.40 (m, 78H), 3.20-3.00 (m, 16H), 2.25-0.60 (m, 172H); MS m/z calcd. for C₂₀₂H₃₄₄N₃₄O₆₅ 4286.5, found 1430.6 ([M+3]⁻⁺⁺/3).

### Example 10-12

### Synthesis of Compound 22

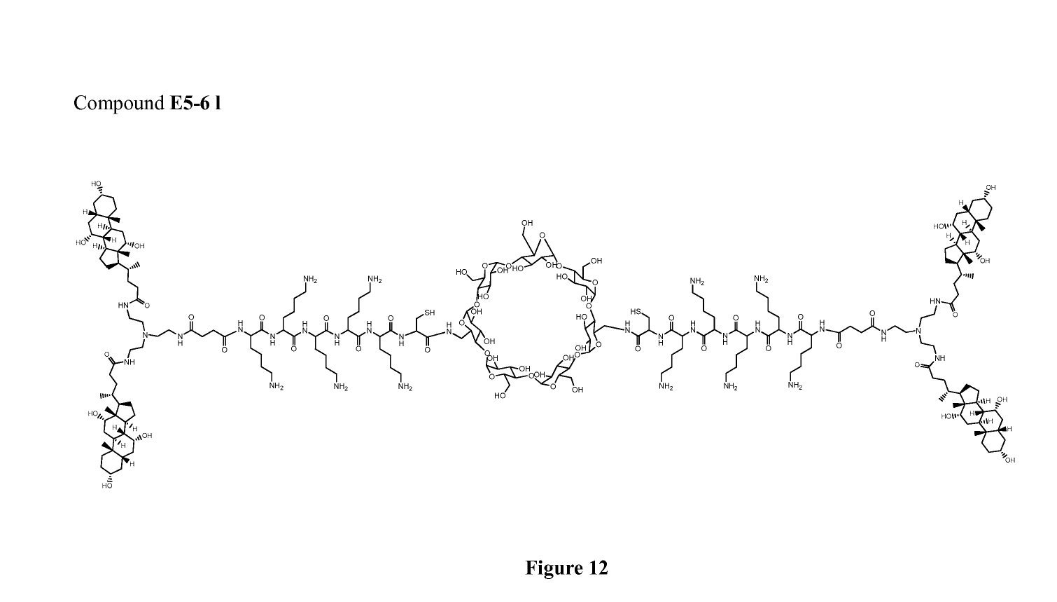

Compound 22 (see FIG. 12) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocR(Pbf)R(Pbf)R(Pbf)GK(Z)GK(Z)-OH (SEQ ID NO: 55) (General procedure A); Cbz removal (General procedure F); DIC/HOBt mediated amide bond formation between the resulting cyclodextrin-peptide conjugate from the previous step and Ch2-OH (General procedure B); and final deprotection (General procedure C). The final compound was purified by reverse phase HPLC to give compound 22 as a white powder after lyophilization. ¹H-NMR (300 MHz, D₂O) δ 5.05-4.95 (m, 7H), 4.35-3.30 (m, 76H), 3.20-3.00 (m, 20H), 2.25-0.60 (m, 188H); MS m/z calcd. for C₂₁₄H₃₆₈N₄₂O₆₃ 4534.7, found 1513.6 ([M+3]⁺⁺⁺/3).

### Example 10-13

### Synthesis of Compound 23

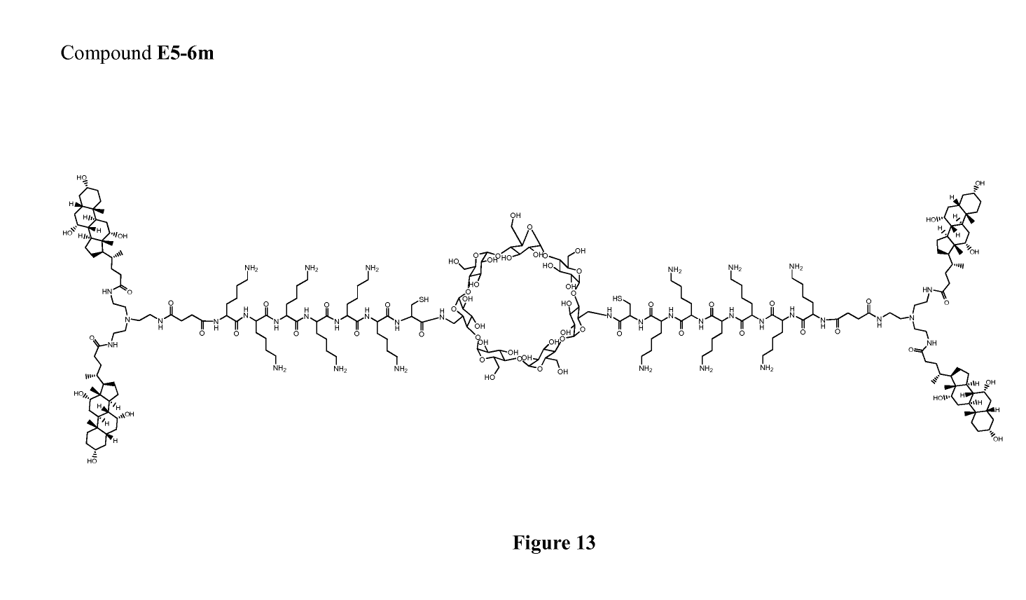

Compound 23 (see FIG. 13) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocR(Pbf)R(Pbf)R(Pbf)GK(Z)GK(Z)-OH (SEQ ID NO: 55) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 23 as a white powder after lyophilization. ¹H-NMR (300 MHz, D₂O) δ 5.05-4.95 (m, 7H), 4.35-3.30 (m, 80H), 3.20-3.00 (m, 20H), 2.25-0.55 (m, 180H); MS m/z calcd. for C₂₁₄H₃₆₈N₄₂O₆₇ 4598.7, found 1535.4 ([M+3]⁺⁺⁺/3).

### Example 10-14

### Synthesis of Compound 24

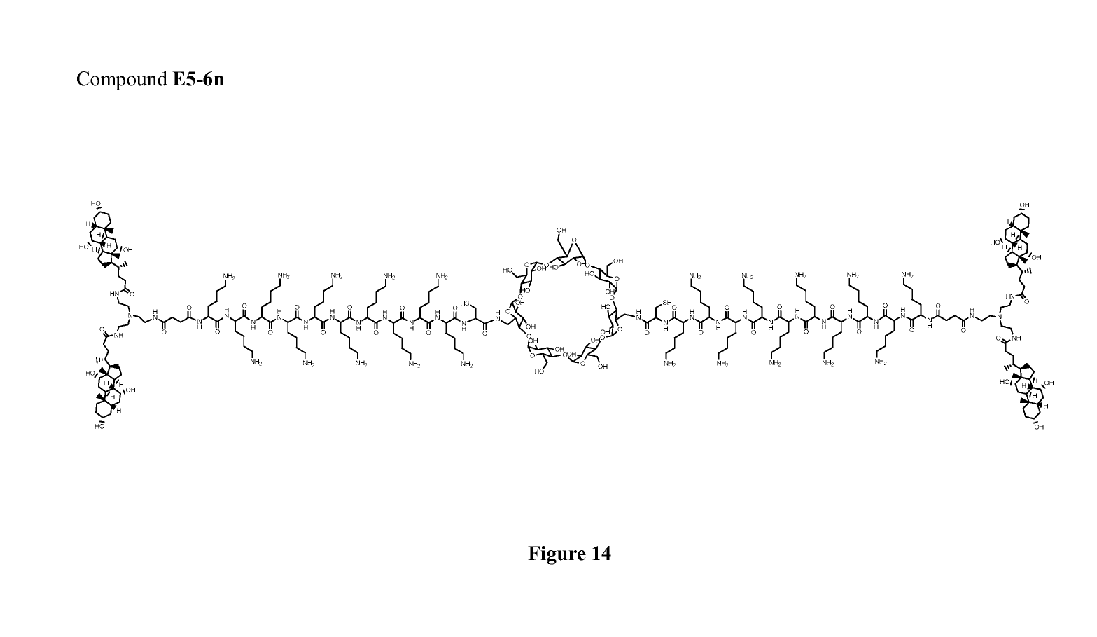

Compound 24 (see FIG. 14) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocK(Boc)K(Boc)K(Boc)K(Boc)GK(Z)GK(Z)-OH (SEQ ID NO: 56) (General procedure A); Cbz removal (General procedure F); DIC/HOBt mediated amide bond formation between the resulting cyclodextrin-peptide conjugate from the previous step and Ch2-OH (General procedure B); and final deprotection (General procedure C). The final compound was purified by reverse phase HPLC to give compound 24 as a white powder after lyophilization. ¹H-NMR (300 MHz, D₂O) δ 5.05-4.95 (m, 7H), 4.35-3.35 (m, 78H), 3.20-2.90 (m, 24H), 2.25-0.55 (m, 212H); MS m/z calcd. for C₂₂₆H₃₉₂N₃₄O₆₅ 4622.8, found 1543.8 ([M+3]⁺⁺⁺/3).

### Example 10-15

### Synthesis of Compound 25

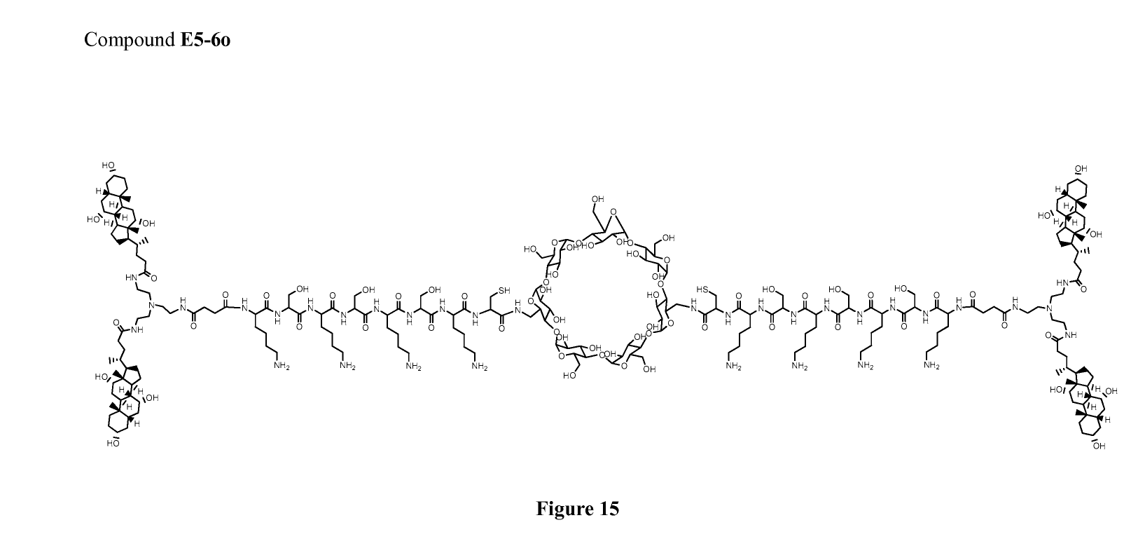

Compound 25 (see FIG. 15) was synthesized using the general procedures described above as follows: HATU mediated amide bond formation between 6^{A},6^{D}-dideoxy-6^{A},6^{D}-di-(Gly-Gly-amino)-β-cyclodextrin and peptide BocK(Boc)K(Boc)K(Boc)K(Boc)GK(Z)GK(Z)-OH (SEQ ID NO: 56) (General procedure A); Cbz removal (General procedure F); DIC/HOBt mediated amide bond formation between the resulting cyclodextrin-peptide conjugate from the previous step and Ch1-OH (General procedure B); and final deprotection (General procedure C). The final compound was purified by reverse phase HPLC to give compound 25 as a white powder after lyophilization. ¹H-NMR (300 MHz, D₂O) δ 5.05-4.95 (m, 7H), 4.35-3.35 (m, 82H), 3.25-2.90 (m, 24H), 2.25-0.55 (m, 204H); MS m/z calcd. for C₂₂₆H₃₉₂N₃₄O₆₉ 4686.8, found 1564.3 ([M+3]⁺⁺⁺/3).

### Example 10-16

### Synthesis of Compound 26

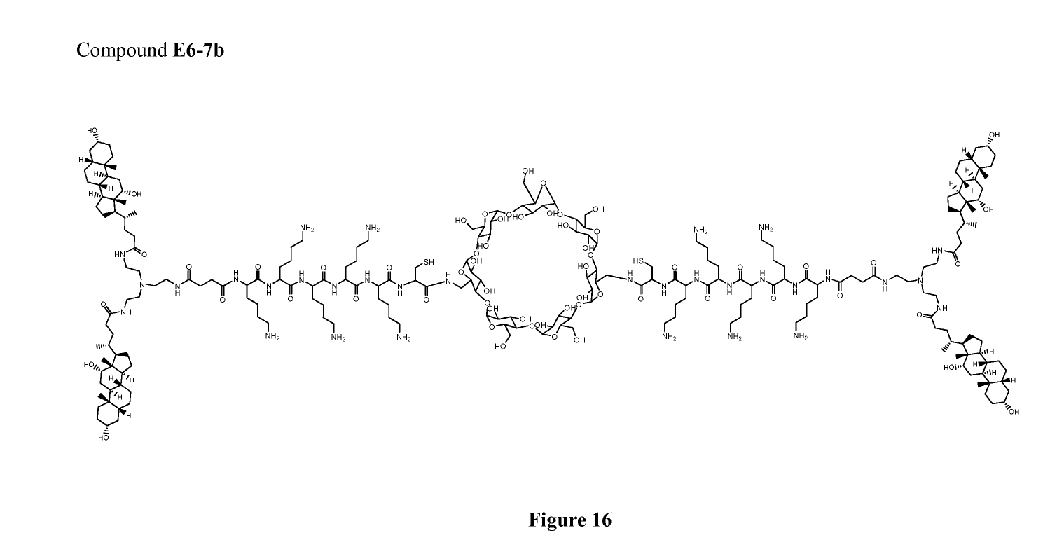

Compound 26 (see FIG. 16) was synthesized using the general procedures described above as follows: HATU mediated amide bond formation between 6^{A},6^{D}-dideoxy-diamino-β-cyclodextrin and Fmoc-Cys(StBu)-OH (General procedure A); Fmoc removal (General procedure D); HATU mediated amide bond formation between 6^{A},6^{D}-dideoxy-6^{A},6^{D}-di-(Cys(StBu)-amino)-β-cyclodextrin and peptide Fmoc-O(Boc)O(Boc)O(Boc)O(Boc)O(Boc)-OH (General procedure A); Fmoc removal (General procedure D); DIC/HOBt mediated amide bond formation between the resulting cyclodextrin-peptide conjugate from the previous step and X3-OH (General procedure B); Boc deprotection (General procedure C, no thiol scavenger) and S-tBu removal (General procedure E). The final compound was purified by reverse phase HPLC to give compound 26 as a white powder after lyophilization. ¹H-NMR (300 MHz, D₂O) δ 5.10-4.90 (m, 7H), 4.40-4.20 (m, 12H), 4.05-3.25 (m, 50H), 3.05-2.90 (m, 20H), 2.65-0.60 (m, 216H); MS m/z calcd. for C₂₁₄H₃₇₄N₃₂O₆₁S₂ 4432.7, found 1479.6 ([M+3]⁺⁺⁺/3).

### Example 10-17

### Synthesis of Compound 27

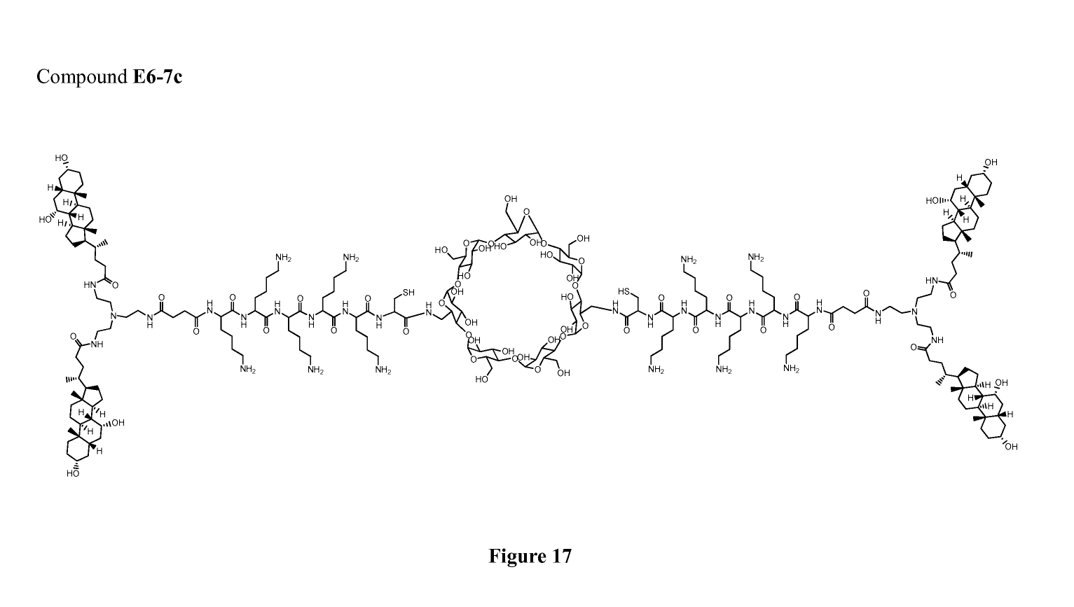

Compound 27 (see FIG. 17) was synthesized using the general procedures described above as follows: HATU mediated amide bond formation between 6^{A},6^{D}-dideoxy-diamino-β-cyclodextrin and Fmoc-Cys(StBu)-OH (General procedure A); Fmoc removal (General procedure D); HATU mediated amide bond formation between 6^{A},6^{D}-dideoxy-6^{A},6^{D}-di-(Cys(StBu)-amino)-β-cyclodextrin and peptide Fmoc-O(Boc)O(Boc)O(Boc)O(Boc)O(Boc)-OH (General procedure A); Fmoc removal (General procedure D); DIC/HOBt mediated amide bond formation between the resulting cyclodextrin-peptide conjugate from the previous step and X1-OH (General procedure B); Boc deprotection (General procedure C, no thiol scavenger) and S-tBu removal (General procedure E). The final compound was purified by reverse phase HPLC to give compound 27 as a white powder after lyophilization. ¹H-NMR (300 MHz, D₂O) δ 5.10-4.95 (m, 7H), 4.40-4.20 (m, 12H), 4.05-3.25 (m, 54H), 3.05-2.85 (m, 20H), 2.60-0.55 (m, 208H), MS m/z calcd. for C₂₁₄H₃₇₄N₃₂O₆₅S₂ 4496.6, found 1501.8 ([M+3]⁺⁺⁺/3).

### Example 10-18

### Synthesis of Compound 31

Compound 31 was synthesized using the general procedures described above as follows: DIC/HOBt mediated amide bond formation between 6^{A},6^{D}-dideoxy-diamino-β-cyclodextrin and Fmoc-K(Boc)K(Boc)K(Boc)K(Boc)G-OH (SEQ ID NO: 1) (General procedure B); Fmoc removal (General procedure D); DIC/HOBt mediated amide bond formation between 6^{A},6^{D}-dideoxy-6^{A},6^{D}-di-(K(Boc)K(Boc)K(Boc)K(Boc)G (SEQ ID NO: 1)-amino)-β-cyclodextrin and compound Boc-C(StBu)GK(Ch2)GK(Ch2)-OH (SEQ ID NO: 57) (General procedure B); Boc removal (General procedure G); purification by RP-HPLC and final reduction (General procedure E). The final compound was purified by reverse phase HPLC to give compound 31 as a white powder after lyophilization. MS m/z calcd. for C₂₂₈H₃₉₆N₃₄O₆₅S₂ 4716.8, found 1573.6 ([M+3]⁺⁺/3).

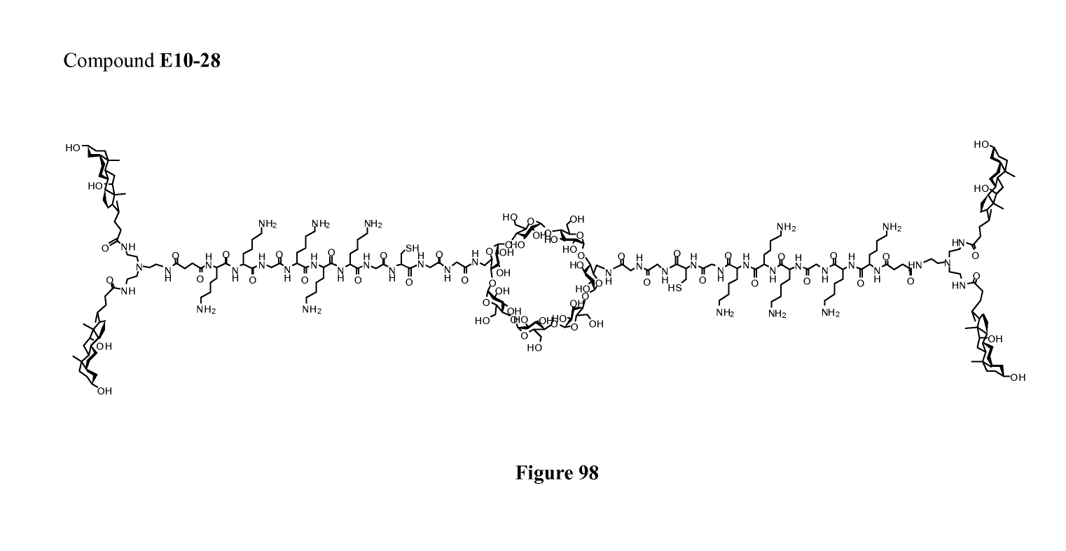

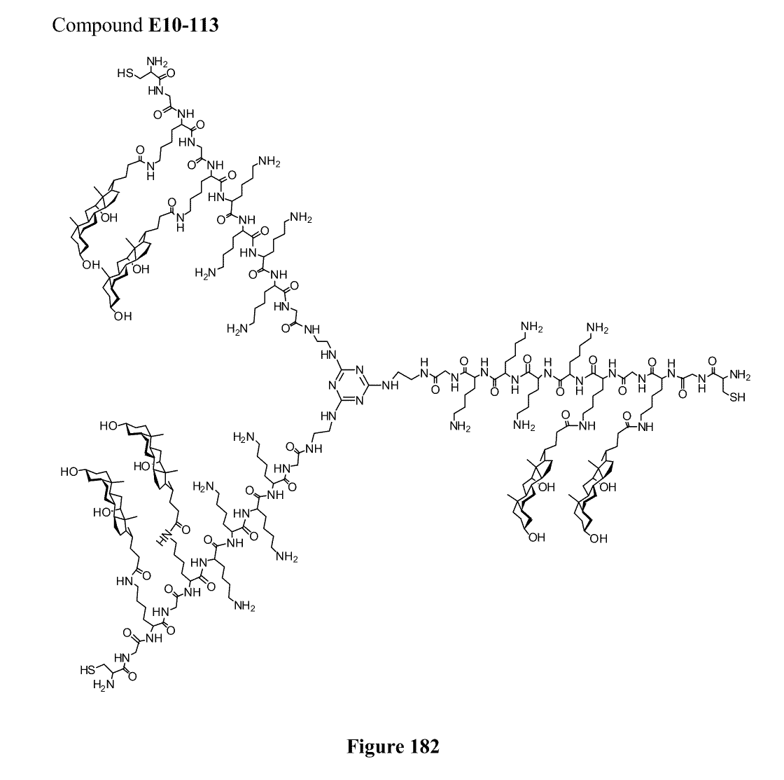

Additional compounds E10-28, E10-29, E10-30 and E10-32-E10-113 were prepared consistent with the procedures set forth herein (see FIGS. 98-182 for the structures thereof).

### Example 11

### Synthesis of Oligopeptide-Cyclodextrin Conjugates 2-50

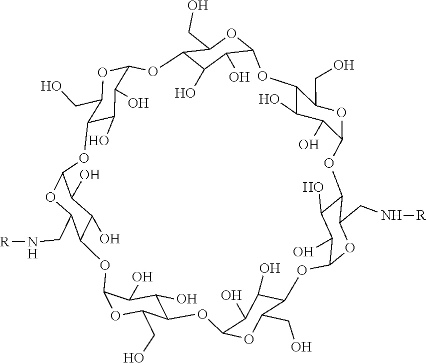

1: R = H
(SEQ ID NO: 58)
2: R = K(Ch1)GKKKKGKKKK
(SEQ ID NO: 59)
3: R = K(Ch4)GKKKKGKKKK
(SEQ ID NO: 60)
4: R = GGKKKKKKKK-Ch1
(SEQ ID NO: 61)
5: R = GGKKKKKKKK-Ch4
(SEQ ID NO: 62)
6: R = GGKKKKKKKK-Ch2
(SEQ ID NO: 63)
7: R = K(Ch1)K(Ch1)KKKKKK
(SEQ ID NO: 64)
8: R = K(Ch1)K(Ch1)KKKKKKKK
(SEQ ID NO: 65)
9: R = GK(Ch1)GK(Ch1)
(SEQ ID NO: 66)
10: R = GK(Ch1)GK(Ch1)G
(SEQ ID NO: 67)
11: R = GK(Ch1)GK(Ch1)GGKKK
(SEQ ID NO: 68)
12: R = GK(Ch1)GK(Ch1)KHCC
(SEQ ID NO: 69)
13: R = GK(Ch1)GK(Ch1)GKKKK
(SEQ ID NO: 70)
14: R = GK(Ch1)GK(Ch1)GKKKKK
(SEQ ID NO: 71)
15: R = GK(Ch1)GK(Ch1)GKKKKKK
(SEQ ID NO: 72)
16: R = GK(Ch1)GK(Ch1)KKKKKC
(SEQ ID NOS 65 & 73,
respectively,in order of appearance)
17: R = GK(Ch1)GK(Ch1)-L1-CKKKKKKKK
(SEQ ID NOS 65 & 74,
respectively, in order of appearance)
18: R = GK(Ch1)GK(Ch1)-L1-CKKKKKKKKK
(SEQ ID NOS 65 & 75,
respectively, in order of appearance)
19: R = GK(Ch1)GK(Ch1)-L1-CKKKGKKKGKKKGKKK
(SEQ ID NO: 65)
20: R = GK(Ch1)GK(Ch1)-L1-cyclo(C-df-RGH)
(SEQ ID NO: 76)
21: R = GK(Ch2)GK(Ch2)GKKKK
(SEQ ID NO: 77)
22: R = GK(Ch2)GK(Ch2)GKKKKK
(SEQ ID NO: 78)
23: R = GK(Ch2)GK(Ch2)GKKKKKK
(SEQ ID NO: 79)
24: R = GK(Ch2)GK(Ch2)KKKKKC
(SEQ ID NO: 80)
25: R = GK(Ch3)GK(Ch3)GKKK
(SEQ ID NO: 81)
26: R = GK(Ch3)GK(Ch3)GKKKK
(SEQ ID NO: 82)
27: R = GK(Ch3)GK(Ch3)GKKKKK
(SEQ ID NO: 83)
28: R = GK(Ch4)GK(Ch4)GKKK
(SEQ ID NO: 84)
29: R = GK(Ch4)GK(Ch4)GKKKK
(SEQ ID NO: 85)
30: R = GK(Ch4)GK(Ch4)GKKKKK
(SEQ ID NO: 86)
31: R = GK(Ch4)GK(Ch4)GKKKKKK
(SEQ ID NO: 87)
32: R = GK(Ch4)GK(Ch4)KKKKKC
(SEQ ID NO: 88)
33: R = GK(Ch5)GK(Ch5)GKKKK
(SEQ ID NO: 89)
34: R = GK(Ch1)GKGKGK
(SEQ ID NO: 90)
35: R = GK(Ch1)GKKKK
(SEQ ID NO: 91)
36: R = GK(Ch2)GK(Ch2)GKGKGK
(SEQ ID NO: 92)
37: R = GK(Ch2)GK(Ch2)GKKGKK
38: R = GK(Palm)
(SEQ ID NO: 93)
39: R = GK(Palm)GKKKK
(SEQ ID NO: 94)
40: R = GK(Palm) GK(Palm)GKKKK
(SEQ ID NO: 95)
41: R = GK(Palm) GK(Palm)GKKGKK (26-44)
42: R = GK(Palm)SPERM
(SEQ ID NO: 96)
43: R = GK(Palm) GK(Palm)SPERM

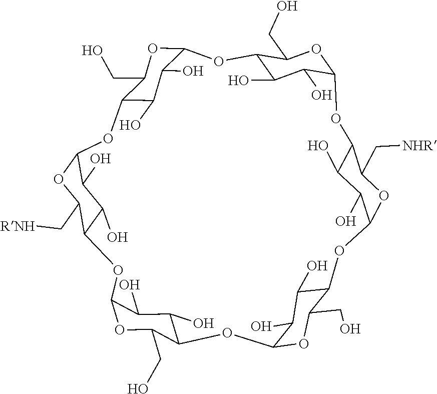

| | |
|---|---|
| 44: R' = H | |
| 45: R' = GKKKK(Ch2)GK(Ch2)GC | (SEQ ID NO: 97) |
| 46: R' = GKKKKK(Ch2)GK(Ch2)GC | (SEQ ID NO: 98) |
| 47: R' = CKKKKKK(Ch2)GK(Ch2)GC | (SEQ ID NO: 99) |
| 48: R' = CKKKKK(Ch2)GK(Ch2)G | (SEQ ID NO: 100) |
| 49: R' = CKKKKK(Ch2)GK(Ch2)GC | (SEQ ID NO: 101) |
| 50: R' = CKKKKKCh6 | (SEQ ID NO: 102) |

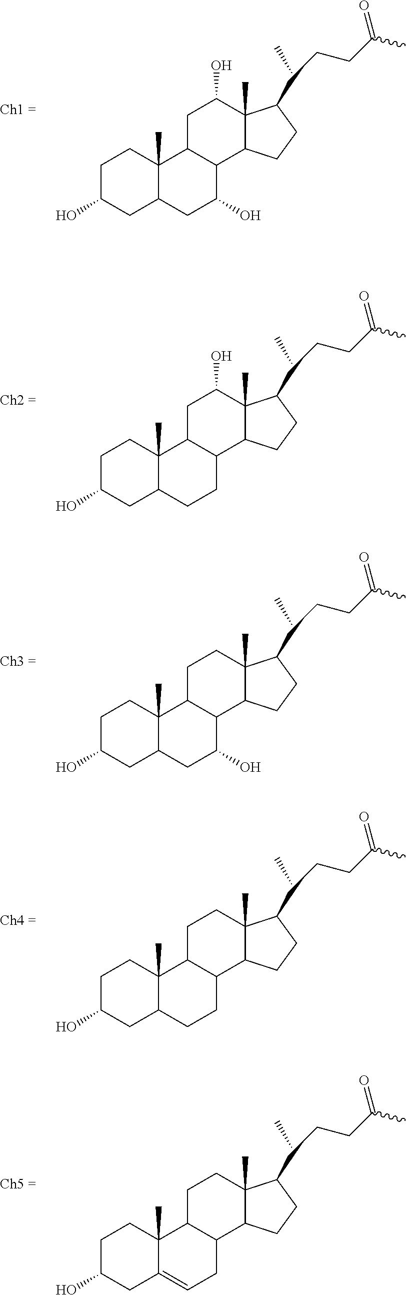

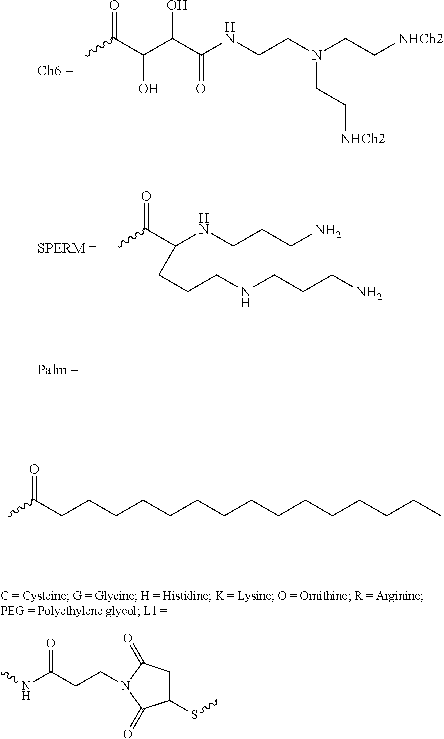

### Example 11-1

### General Procedure A: the Formation of Peptide Bond

To a solution of 1,4-diamino-β-cyclodextrin (1, 1 eq) or its derivative and C-terminus oligopeptide building block or simple amino acid with all amino group protected by t-butyl carbamate (Boc) or 9-fluorenylmethyl carbamate (Fmoc) (2.2 eq) in anhydrous DMF at room temperature was added coupling agents (DIC or TBTU or HATU and HOBt) (2.2 eq) and diisopropylamine (DIPEA) (2.2 eq). The resulting solution was stirred at ambient temperature until completion (monitored by HPLC). The solution was concentrated under reduced pressure. The residue was washed with water and ethyl acetate. The compound was further purified by preparative HPLC if necessary. Refer to the general procedure in Example 7-1 if DCC was used as the coupling agent.

### Example 11-2

### General Procedure B: Deprotection of Fmoc Protected Amino Group

The Fmoc protected amino compound was dissolved in 20% piperidine/DMF. The resulting solution was stirred at room temperature for 0.5-1 hour until the protecting group was completely removed (monitored by HPLC). The solvent was removed under reduced pressure and the residue was mixed with water to form a slurry. The resulting slurry was filtered, and the filtrate was washed with ethyl acetate and dried to give the desired product. The product was used to the next step without further purification.

### Example 11-3

### General Procedure C: Deprotection of Boc Protected Amino Group

The Boc protected amino compound was dissolved in methylene chloride-trifluoroacetic acid solution (1:3) or MeOH/Con HCl (5/2). The resulting solution was stirred at room temperature for 0.5-1 hour until completion. The solvent was then evaporated under reduced pressure to give a TFA or HCl salt. If necessary, the TFA salt can be converted to a HCl salt by dissolving the compound in 1 M HCl methanol solution and then evaporated to dryness two times. The overall yields from coupling to the final product were from 5% to 90%. The products were further purified by preparative HPLC, if needed.

### Example 11-4

### General Procedure D for Coupling with Cholic Acid

The same procedure in Example 11-1 was used to couple with alkylcarboxylic acids or NHS activated esters in the presence of DIPEA (2.2 eq) in DMF.

### Example 11-5

### General Procedure E: Coupling with Cross Linking Reagent

The oligopeptide-cyclodextrin with free amino groups at the end of each peptide (1 eq) was dissolved in DMF, after the cross linking reagent (NHS-R-MAL) (2.5 eq) and DIPEA (2.5 eq) were added to the reaction solution, the resulting reaction mixture was stirred at room temperature until completion of the reaction (monitored by HPLC). The reaction solution was concentrated under reduced pressure and the residue was washed with water and ethyl acetate. The crude product was used without further purification.

### Example 11-6

### General Procedure F: Reaction Between Maleinmide Group and Thiol Group

The oligopeptide-cyclodextrin with maleinmide group (1 eq) was dissolved in a mixed solvent of methanol-1 M Tris buffer (pH 7.2) (ratio 4:1). The solution was degassed and the peptide with a free thiol group (2.5 eq) was added to the solution. After the reaction was complete (monitored by HPLC), the solvent was removed and the residue was purified by preparative HPLC to give product.

### Example 11-7

### General Procedure G: Deprotection of Alloc Protected Amino Group

Oligopeptide-cyclodextrin with an Alloc protected amino group (1 eq) was dissolved in DMF at room temperature. After the solution was degassed, Pd(Ph₃)₄ (2.05 eq) and Me₂NH/BH₃ (2.05 eq) were added to the solution. The mixture was stirred at room temperatures under positive nitrogen pressure overnight. After adding MeOH, the resulting mixture was filtered and the solid was washed with H₂O, NaHCO₃ and NH₄Cl solution and dried to provide the desired product with 50-90% yields.

### Example 11-8

### General Procedure H: Deprotection of Triyl Protected Thiol and Histidine Group

The trityl protected thiol and histidine protected compound was dissolved in trifluoroacetic acid/EDT/ solution. The resulting solution was stirred at room temperature for 0.5-3 hour until the protection group completely removed. The solvent was then evaporated under reduced pressure to give a TFA salt. If necessary, the TFA salt can be converted to a HCl salt by dissolving the compound in 1M HCl solution and then evaporated to dryness two times. The overall yields from coupling to the final product were from 5% to 90%. The products were further purified by preparative HPLC, if needed.

### Example 11-9

### Preparation of Compound 2

Compound 2 was synthesized using the general procedures described above as follows: coupled 1 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (SEQ ID NO: 1) (procedure A); Fmoc deprotection (procedure B); Boc deprotection (procedure C); further coupled with Boc-Lys(Boc)-Lys(Boc)-Lys(Boc)-Lys(Boc)-Gly-OH (SEQ ID NO: 1) (procedure A); Alloc deprotection (procedure G); further coupled with cholic acid (procedure D); Boc deprotection (procedure C). Compound 2 was isolated using preparative HPLC and converted to the HCl salt. ¹H NMR (300 MHz, D₂O): δ 0.5-2.5 (m, 174 H), 2.80-2.95 (m, 32H), 3.15-3.30 (m, 4H); 3.40-4.85 (m, 74H), 4.95 (br, 7H); MS (MALDI) m/z calcd. for C₂₀₆H₃₇₆N₄₂O₆₃ 4448, found 4449.

### Example 11-10

### Preparation of Compound 3

Compound 3 was synthesized using the general procedures described above as follows: coupled 1 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (SEQ ID NO: 1) (procedure A); Fmoc deprotection (procedure B); Boc deprotection (procedure C); further coupled with Boc-Lys(Boc)-Lys(Boc)-Lys(Boc)-Lys(Boc)-Gly-OH (SEQ ID NO: 1) (procedure A); Alloc deprotection (procedure G); further coupled with lithocholic acid (procedure D); Boc deprotection (procedure C). Compound 3 was isolated using preparative HPLC and converted to the HCl salt. ¹HNMR (300 MHz, D₂O): δ 0.5-2.5 (m, 182 H), 2.80-2.95 (m, 32H), 3.15-3.30 (m, 4H); 3.40-4.85 (m, 7H), 4.95 (br, 7H); MS (MALDI) m/z calcd. for C₂₀₆H₃₇₆N₄₂O₅₉ 4385, found 4384.

### Example 11-11

### Preparation of Compound 4

Compound 4 was synthesized using the general procedures described above as follows: coupled 1 with Fmoc-Gly-Gly-OH (procedure A); Fmoc deprotection (Procedure B); further coupled with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (SEQ ID NO: 2) (procedure A); Fmoc deprotection (procedure B); further coupled with cholic acid (procedure D); Boc deprotection (procedure C). Compound 4 was isolated using preparative HPLC and converted to the HCl salt. ¹HNMR (300 MHz, D₂O): δ 0.5-2.5 (m, 162H), 2.85-3.0 (m, 32H), 3.25-4.35 (m, 72H), 4.95-5.10 (br, 7H). MS (MALDI) m/z calcd. for C₁₉₄H₃₅₂N₃₈O₆₁ 4192, found 4215.

### Example 11-12

### Preparation of Compound 5

Compound 5 was synthesized using the general procedures described above as follows: coupled 1 with Fmoc-Gly-Gly-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (SEQ ID NO: 2) (procedure A); Fmoc deprotection (procedure B); further coupled with lithocholic acid (procedure D); Boc deprotection (procedure C). Compound 5 was isolated using preparative HPLC and converted to the HCl salt. ¹HNMR (300 MHz, D₂O): δ 0.5-2.5 (m, 170H), 2.85-3.0 (m, 32H), 3.25-4.35 (m, 68H), 5.05-5.15 (br, 7H). MS (MALDI) m/z calcd. for C₁₉₄H₃₅₂N₃₈O₅₇ 4128, found 4129.

### Example 11-13

### Preparation of Compound 6

Compound 6 was synthesized using the general procedures described above as follows: coupled 1 with Fmoc-Gly-Gly-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (SEQ ID NO: 2) (procedure A); Fmoc deprotection (procedure B); further coupled with deoxycholic acid (procedure D); Boc deprotection (procedure C). Compound 6 was isolated using preparative HPLC and converted to the HCl salt. ¹HNMR (300 MHz, D₂O): δ 0.5-2.5 (m, 166H), 2.90-3.05 (m, 32H), 3.25-4.35 (m, 70H), 4.95-5.10 (br, 7H). MS (MALDI) m/z calcd. for C₁₉₄H₃₅₂N₃₈O₅₉ 4160, found 4182.

### Example 11-14

### Preparation of Compound 7

Compound 7 was synthesized using the general procedures described above as follows: coupled 1 with Fmoc-Lys(Alloc)-OH (procedure A); Fmoc deprotection (procedure B); further coupled with Fmoc-Lys(Alloc)-OH (procedure A); Fmoc deprotection (procedure B); further coupled with Boc-Lys(Boc)-Lys(Boc)-Lys(Boc)-Lys(Boc)-Lys(Boc)-Lys(Boc)-OH (SEQ ID NO: 17) (procedure A); Alloc deprotection (procedure G); further coupled with cholic acid (procedure D); Boc deprotection (procedure C). Compound 7 was isolated using preparative HPLC and converted to the HCl salt. ¹HNMR (300 MHz, D₂O): δ 0.5-2.5 (m, 228H), 2.90-3.05 (m, 20H), 3.10-3.20 (m, 8H), 3.25-4.35 (m, 70H), 4.95-5.10 (br, 7H). MS (MALDI) m/z calcd. for C₂₃₄H₄₁₆N₃₄O₆₅ 4745, found 4769.

### Example 11-15

### Preparation of Compound 8

Compound 8 was synthesized using the general procedures described above as follows: coupled 1 with Fmoc-Lys(Alloc)-OH (procedure A); Fmoc deprotection (procedure B); further coupled with Fmoc-Lys(Alloc)-OH (procedure A); Fmoc deprotection (procedure B); further coupled with Boc-Lys(Boc)-Lys(Boc)-Lys(Boc)-Lys(Boc)-Lys(Boc)-Lys(Boc)-Lys(Boc)-Lys(Boc)-OH (SEQ ID NO: 103) (procedure A); Alloc deprotection (procedure G); further coupled with cholic acid (procedure D); Boc deprotection (procedure C). Compound 8 was isolated using preparative HPLC and converted to the HCl salt. ¹HNMR (300 MHz, D₂O): δ 0.5-2.5 (m, 252H), 2.90-3.05 (m, 32H), 3.10-3.20 (m, 8H), 3.25-4.35 (m, 72H), 4.95-5.10 (br, 7H). MS (MSD trap) m/z calcd. for C₂₅₈H₄₆₄N₄₂O₆₉ 5258, found 876 (M⁶⁺).

### Example 11-16

### Preparation of Compound 9

Compound 9 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with cholic acid (procedure D); Fmoc deprotection (procedure B). Compound 9 was isolated using preparative HPLC and converted to the HCl salt. ¹HNMR (300 MHz, D₂O): δ 0.5-2.5 (m, 156H), 3.05-3.15 (m, 8H), 3.25-4.35 (m, 66H), 4.95-5.10 (br, 7H). MS (MALDI) m/z calcd. for C₁₇₀H₂₈₄N₁₄O₅₇ 3436, found 3457.

### Example 11-17

### Preparation of Compound 10

Compound 10 was synthesized using the general procedures described above as follows: coupled 9 with Fmoc-Gly-OH (procedure A); Fmoc deprotection (procedure B). Compound 10 was isolated using preparative HPLC and converted to the HCl salt. MS (MSD trap) m/z calcd. for C₁₇₄H₂₉₀N₁₆O₅₉ 3549, found 1184.5(M³⁺).

### Example 11-18

### Preparation of Compound 11

Compound 11 was synthesized using the general procedures described above as follows: coupled 9 with Boc-Lys(Boc)-Lys(Boc)-Lys(Boc)-Gly-Gly-OH (SEQ ID NO: 105) (procedure A); Boc deprotection (procedure C). Compound 11 was isolated using preparative HPLC and converted to the HCl salt. ¹HNMR (300 MHz, D₂O): δ 0.5-2.5 (m, 192H), 2.90-3.05 (m, 20H), 3.25-4.35 (m, 80H), 5.0-5.10 (br, 7H); MS (MSD trap) m/z calcd. for C₂₁₄H₃₆₈N₃₀O₆₇ 4433, found 1107 (M⁴⁺).

### Example 11-19

### Preparation of Compound 12

Compound 12 is synthesized using the general procedures described above as follows: couple 9 with Fmoc-Cys(Trt)-Cys(Trt)-His(Trt)-Lys(Boc)-OH (SEQ ID NO: 106) (procedure A); Fmoc deprotection (procedure B); Boc and trityl deprotection (procedure H). Compound 12 is isolated using preparative HPLC and converted to the HCl salt.

### Example 11-20

### Preparation of Compound 13

Compound 13 was synthesized using the general procedures described above as follows: coupled 9 with Boc-Lys(Boc)-Lys(Boc)-Lys(Boc)-Lys(Boc)-Gly-OH (SEQ ID NO: 1) (procedure A); Boc deprotection (procedure C). Compound 13 was isolated using preparative HPLC and converted to the HCl salt. ¹HNMR (300 MHz, D₂O): δ 0.5-2.5 (m, 204H), 2.90-3.05 (m, 16H), 3.10-3.20 (m, 8H, covered by a solvent peak), 3.25-4.35 (m, 78H), 4.95-5.10 (br, 7H); MS (MALDI) m/z calcd. for C₂₂₂H₃₈₆N₃₂O₆₇ 4575, found 4599.

### Example 11-21

### Preparation of Compound 14

Compound 14 was synthesized using the general procedures described above as follows: coupled 9 with Boc-Lys(Boc)-Lys(Boc)-Lys(Boc)-Lys(Boc)-Lys(Boc)-Gly-OH (SEQ ID NO: 9) (procedure A); Boc deprotection (procedure C). Compound 14 was isolated using preparative HPLC and converted to the HCl salt. MS (MSD trap) m/z calcd. for C₂₃₄H₄₁₀N₃₆O₆₉ 4830, found 1109 (M⁴⁺).

### Example 11-22.

### Preparation of Compound 15

Compound 15 was synthesized using the general procedures described above as follows: coupled 9 with Boc-Lys(Boc)-Lys(Boc)-Lys(Boc)-Lys(Boc)-Lys(Boc)-Lys(Boc)-Gly-OH (SEQ ID NO: 107) (procedure A); Boc deprotection (procedure C). Compound 15 was isolated using preparative HPLC and converted to the HCl salt. MS (MSD trap) m/z calcd. for C₂₄₆H₄₃₄N₄₀O₇₁ 5088, found 1088(M⁵⁺).

### Example 11-23

### Preparation of compound 16

Compound 16 was synthesized using the general procedures described above as follows: coupled 9 with Fmoc-Cys(Trt)-Lys(Boc)-Lys(Boc)-Lys(Boc)-Lys(Boc)-Lys(Boc)-Gly-OH (SEQ ID NO: 108) (procedure A); Fmoc deprotection (procedure B); Boc and trityl deprotection (procedure H). Compound 16 was isolated using preparative HPLC and converted to the HCl salt. MS (MSD trap) m/z calcd. for C₂₃₆H₄₁₄N₃₆O₆₉S₂ 4923, found 1232 (M⁴⁺).

### Example 11-24

### Preparation of Compound 17

Compound 17 was synthesized using the general procedures described above as follows: coupled 9 with NHS-3-maleimideoproppionate (procedure F); coupled with Cys-Lys-Lys-Lys-Lys-Lys-Lys-Lys-Lys-OH (SEQ ID NO: 109) (procedure F). Compound 17 was isolated using preparative HPLC and converted to the HCl salt. ¹HNMR (300 MHz, D₂O): δ 0.5-2.5 (m, 264H), 2.90-3.05 (m, 32H), 3.10-3.25(m, 8H); 3.25-4.35 (m, 80H), 5.0-5.10 (br, 7H); MS (MALDI) m/z calcd. for C₂₈₆H₅₀₀N₅₀O₈₃S2 6031, found 6057.

### Example 11-25

### Preparation of Compound 18

Compound 18 was synthesized using the general procedures described above as follows: coupled 9 with NHS-3-maleimideoproppionate (procedure F); coupled with Cys-Lys-Lys-Lys-Lys-Lys-Lys-Lys-Lys-Lys-OH (SEQ ID NO: 110) (procedure F). Compound 18 was isolated using preparative HPLC and converted to the HCl salt. ¹HNMR (300 MHz, D₂O): δ 0.5-2.5 (m, 276H), 2.90-3.05 (m, 36H), 3.10-3.25 (m, 8H); 3.25-4.35 (m, 82H), 5.0-5.10 (br, 7H); MS (MSD trap) m/z calcd. for C₂₉₈H₅₂₄N₅₄O₈₅S₂ 6288, found 690 (M⁹⁺).

### Example 11-26

### Preparation of Compound 19

Compound 19 was synthesized using the general procedures described above as follows: coupled 9 with NHS-3-maleimideoproppionate (procedure F); coupled with Cys-Lys-Lys-Lys-Gly-Lys-Lys-Lys-Gly-Lys-Lys-Lys-Gly-Lys-Lys-Lys-OH (SEQ ID NO: 111) (procedure F). Compound 19 was isolated using preparative HPLC and converted to the HCl salt. ¹HNMR (300 MHz, D₂O): δ 0.5-2.5 (m, 308H), 2.90-3.05 (m, 48H), 3.10-3.25 (m, 8H); 3.25-4.35 (m, 114H), 5.0-5.10 (br, 7H); MS (MSD trap) m/z calcd. for C₃₄₆H₆₁₄N₇₂O₉₇S₂ 7397, found 3720 (M²⁺+Na).

### Example 11-27

### Preparation of Compound 20

Compound 20 was synthesized using the general procedures described above as follows: coupled 9 with NHS-3-maleimideoproppionate (procedure F); coupled with cyclo(C-df-RGD) (procedure F). Compound 20 was isolated using preparative HPLC and converted to the HCl salt. ¹HNMR (300 MHz, CD₃OD): δ 0.5-2.5 (m, 176H), 3.10-25(m, 12H); 3.25-4.53 (m, 92H), 5.0-5.10 (br, 7H), 7.2 (m, 10H); MS (MAS Trap) m/z calcd. for C₂₃₂H₃₆₂N₃₂O₇₇S₂ 4896, found 2448 (M²⁺).

### Example 11-28

### Preparation of Compound 21

Compound 21 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B); further coupled with Boc-Lys(Boc)-Lys(Boc)-Lys(Boc)-Lys(Boc)-Gly-OH (SEQ ID NO: 1) (procedure A); Boc deprotection (procedure C). Compound 21 was isolated using preparative HPLC and converted to the HCl salt. ¹HNMR (300 MHz, D₂O): δ 0.5-2.5 (m, 212H), 2.90-3.05 (m, 16H), 3.10-3.20 (m, 8H), 3.25-4.35 (m, 74H), 4.95-5.10 (br, 7H), MS (MSD trap) m/z calcd. for C₂₂₂H₃₈₆N₃₂O₆₃ 4510, found 1129 (M⁴⁺).

### Example 11-29

### Preparation of Compound 22

Compound 22 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B). further coupled with Boc-Lys(Boc)-Lys(Boc)-Lys(Boc)-Lys(Boc)-Lys(Boc)-Gly-OH (SEQ ID NO: 9) (procedure A); Boc deprotection (procedure C). Compound 22 was isolated using preparative HPLC and converted to the HCl salt. MS (MSD trap) m/z calcd. for C₂₃₄H₄₁₀N₃₆O₆₅ 4767, found 1193 (M⁴⁺).

### Example 11-30

### Preparation of Compound 23

Compound 23 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B). further coupled with Boc-Lys(Boc)-Lys(Boc)-Lys(Boc)-Lys(Boc)-Lys(Boc)-Lys(Boc)-Gly-OH (SEQ ID NO: 107) (procedure A); Boc deprotection (procedure C). Compound 23 was isolated using preparative HPLC and converted to the HCl salt. MS (MSD trap) m/z calcd. for C₂₄₆H₄₃₄N₄₀O₆₇ 5024, found 1257 (M⁴⁺).

### Example 11-31

### Preparation of Compound 24

Compound 24 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B). further coupled with Fmoc-Cys(Trt)-Lys(Boc)-Lys(Boc)-Lys(Boc)-Lys(Boc)-Lys(Boc)-OH (SEQ ID NO: 3) (procedure A); Boc and trityl deprotection (procedure H). Compound 24 was isolated using preparative HPLC and converted to the HCl salt. MS (MSD trap) m/z calcd. for C₂₃₆H₄₁₄N₃₆O₆₅S₂ 4860, found 972 (M⁵⁺).

### Example 11-32

### Preparation of Compound 25

Compound 25 is synthesized using the general procedures described above as follows: couple 1 with Fmoc-Lys(Boc)-Gly-Lys(Boc)-Gly-OH (SEQ ID NO: 104) (procedure A); Boc deprotection (procedure C); further couple with chenodeoxycholic acid (procedure D); Fmoc deprotection (procedure B); further couple with Boc-Lys(Boc)-Lys(Boc)-Lys(Boc)-Gly-OH (SEQ ID NO: 112) (procedure A); Boc deprotection (procedure C). Compound 24 is isolated using preparative HPLC and converted to the HCl salt.

### Example 11-33

### Preparation of Compound 26

Compound 26 is synthesized using the general procedures described above as follows: couple 1 with Fmoc-Lys(Boc)-Gly-Lys(Boc)-Gly-OH (SEQ ID NO: 104) (procedure A); Boc deprotection (procedure C); further couple with chenodeoxycholic acid (procedure D); Fmoc deprotection (procedure B); further couple with Boc-Lys(Boc)-Lys(Boc)-Lys(Boc)-Lys(Boc)-Gly-OH (SEQ ID NO: 1) (procedure A); Boc deprotection (procedure C). Compound 26 is isolated using preparative HPLC and converted to the HCl salt.

### Example 11-34

### Preparation of Compound 27

Compound 27 is synthesized using the general procedures described above as follows: couple 1 with Fmoc-Lys(Boc)-Gly-Lys(Boc)-Gly-OH (SEQ ID NO: 104) (procedure A); Boc deprotection (procedure C); further couple with chenodeoxycholic acid (procedure D); Fmoc deprotection (procedure B); further couple with Boc-Lys(Boc)-Lys(Boc)-Lys(Boc)-Lys(Boc)-Lys(Boc)-Gly-OH (SEQ ID NO: 9) (procedure A); Boc deprotection (procedure C). Compound 27 is isolated using preparative HPLC and converted to the HCl salt.

### Example 11-35

### Preparation of Compound 28

Compound 28 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with lithocholic acid (procedure D); Fmoc deprotection (procedure B); further coupled with Boc-Lys(Boc)-Lys(Boc)-Lys(Boc)-Gly-OH (SEQ ID NO: 112) (procedure A); Boc deprotection (procedure C). Compound 28 was isolated using preparative HPLC and converted to the HCl salt. MS (MSD trap) m/z calcd. for C₂₁₀H₃₆₂N₂₈O₅₇ 4191, found 1049 (M⁴⁺).

### Example 11-36

### Preparation of Compound 29

Compound 29 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with lithocholic acid (procedure D); Fmoc deprotection (procedure B); further coupled with Boc-Lys(Boc)-Lys(Boc)-Lys(Boc)-Lys(Boc)-Gly-OH (SEQ ID NO: 1) (procedure A); Boc deprotection (procedure C). Compound 29 was isolated using preparative HPLC and converted to the HCl salt. MS (MAS Trap) m/z calcd. for C₂₂₂H₃₈₆N₃₂O₅₉ 4447, found 1113 (M⁴⁺).

### Example 11-37

### Preparation of Compound 30

Compound 30 was synthesized using the general procedures described above as follows: couple 1 with Fmoc-Lys(Boc)-Gly-Lys(Boc)-Gly-OH (SEQ ID NO: 104) (procedure A); Boc deprotection (procedure C); further couple with lithocholic acid (procedure D); Fmoc deprotection (procedure B); further couple with Boc-Lys(Boc)-Lys(Boc)-Lys(Boc)-Lys(Boc)-Lys(Boc)-Gly-OH (SEQ ID NO: 9) (procedure A); Boc deprotection (procedure C). Compound 30 is isolated using preparative HPLC and converted to the HCl salt.

### Example 11-38

### Preparation of Compound 31

Compound 31 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with lithocholic acid (procedure D); Fmoc deprotection (procedure B); further coupled with Boc-Lys(Boc)-Lys(Boc)-Lys(Boc)-Lys(Boc)-Lys(Boc)-Lys(Boc)-Gly-OH (SEQ ID NO: 107) (procedure A); Boc deprotection (procedure C). Compound 31 was isolated using preparative HPLC and converted to the HCl salt. MS (MSD trap) m/z calcd. for C₂₄₆H₄₃₄N₄₀O₆₃ 4960, found 1241 (M⁴⁺).

### Example 11-39

### Preparation of Compound 32

Compound 32 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with lithocholic acid (procedure D); Fmoc deprotection (procedure B); further coupled with Fmoc-Cys(Trt)-Lys(Boc)-Lys(Boc)-Lys(Boc)-Lys(Boc)-Lys(Boc)-OH (SEQ ID NO: 3) (procedure A); Boc and trityl deprotection (procedure H). Compound 32 was isolated using preparative HPLC and converted to the HCl salt. MS (MSD trap) m/z calcd. for C₂₃₆H₄₁₄N₃₆O₆₁S₂ 4796, found 1199 (M⁴⁺).

### Example 11-40

### Preparation of Compound 33

Compound 33 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with 3β-hydroxy-Δ⁵-cholenic acid (procedure D); Fmoc deprotection (procedure B); further coupled with Boc-Lys(Boc)-Lys(Boc)-Lys(Boc)-Lys(Boc)-Gly-OH (SEQ ID NO: 1) (procedure A); Boc deprotection (procedure C). Compound 33 was isolated using preparative HPLC and converted to the HCl salt. MS (MSD trap) m/z calcd. for C₂₂₂H₃₇₈N₃₂O₅₉ 4439, found 1109 (M⁴⁺).

### Example 11-41

### Preparation of Compound 34

Compound 34 was synthesized using the general procedures described above as follows: coupled 1 with Fmoc-Lys(Boc)-Gly-OH (procedure A); Boc deprotection (procedure C); further coupled with cholic acid (procedure D); Fmoc deprotection (procedure B); further coupled with Boc-Lys(Boc)-Gly-Lys(Boc)-Gly-Lys(Boc)-Gly-OH (SEQ ID NO: 113) (procedure A); Boc deprotection (procedure C). Compound 34 was isolated using preparative HPLC and converted to the HCl salt. MS (MSD trap) m/z calcd. C₁₅₄H₂₆₈N₂₈O₅₇ 3394, Found 1133.4 (M³⁺).

### Example 11-42

### Preparation of Compound 35

Compound 35 was synthesized using the general procedures described above as follows: coupled 1 with Fmoc-Lys(Boc-Gly-OH (procedure A); Boc deprotection (procedure C); further coupled with cholic acid (procedure D); Fmoc deprotection (procedure B); further coupled with Boc-Lys(Boc)-Lys(Boc)-Lys(Boc)-Lys(Boc)-Gly-OH (SEQ ID NO: 1) (procedure A); Boc deprotection (procedure C). Compound 35 was isolated using preparative HPLC and converted to the HCl salt. MS (MSD trap) m/z calcd. C₁₅₈H₂₈₀N₂₆O₅₅ 3424, Found 1144.4 (M³⁺).

### Example 11-43

### Preparation of Compound 36

Compound 36 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B); further coupled with Boc-Lys(Boc)-Gly-Lys(Boc)-Gly-Lys(Boc)-Gly-OH (SEQ ID NO: 113) (procedure A); Boc deprotection (procedure C). Compound 36 was isolated using preparative HPLC and converted to the HCl salt. MS (MSD trap) m/z calcd. C₂₁₈H₃₇₄N₃₂O₆₅ 4482, Found 1122.4 (M⁴⁺).

### Example 11-44

### Preparation of Compound 37

Compound 37 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with deoxycholic acid (procedure D); Fmoc deprotection (procedure B); further coupled with Boc-Lys(Boc)-Lys(Boc)-Gly-Lys(Boc)-Lys(Boc)-Gly-OH (SEQ ID NO: 114) (procedure A); Boc deprotection (procedure C). Compound 37 was isolated using preparative HPLC and converted to the HCl salt. MS (MSD trap) m/z calcd. C₂₂₆H₃₉₂N₃₄O₆₅ 4624, Found 1158 (MH⁴⁺).

### Example 11-45

### Preparation of Compound 38

Compound 38 was synthesized using the general procedures described above as follows: coupled 1 with Fmoc-Gly-OH (procedure A); Boc deprotection (procedure C); Further coupled with palmitic acid (procedure D); Fmoc deprotection (procedure B). Compound 38 was isolated using preparative HPLC and converted to the HCl salt. MS (MSD trap) m/z calcd. C₉₀H₁₆₂N₈O₃₉ 1980, Found 1981.2 (MH⁺).

### Example 11-46

### Preparation of Compound 39

Compound 39 was synthesized using the general procedures described above as follows: coupled 1 with Fmoc-Gly-OH (procedure A); Boc deprotection (procedure C); Further coupled with palmitic acid (procedure D); Fmoc deprotection (procedure B). Further coupled with Boc-Lys(Boc)-Lys(Boc)-Lys(Boc)-Lys(Boc)-Gly-OH (SEQ ID NO: 1) (procedure A); Boc Deprotection (procedure C). Compound 39 was isolated using preparative HPLC and converted to the HCl salt. MS (MSD trap) m/z calcd. C₁₄₂H₂₆₄N₂₆O₄₉ 3118.9, Found 1041.5 (MH³⁺).

### Example 11-47

### Preparation of Compound 40

Compound 40 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with palmitic acid (procedure D); Fmoc deprotection (procedure B). Further coupled with Boc-Lys(Boc)-Lys(Boc)-Lys(Boc)-Lys(Boc)-Gly-OH (SEQ ID NO: 1) (procedure A); Boc Deprotection (procedure C). Compound 40 was isolated using preparative HPLC and converted to the HCl salt. MS (MSD trap) m/z calcd. C₁₉₀H₃₅₄N₃₂O₅₅ 3965.6, Found 1324.0 (MH³⁺).

### Example 11-48

### Preparation of Compound 41

Compound 41 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with palmitic acid (procedure D); Fmoc deprotection (procedure B). Further coupled with Boc-Lys(Boc)-Lys(Boc)-Gly-Lys(Boc)-Lys(Boc)-Gly-OH (SEQ ID NO: 114) (procedure A); Boc Deprotection (procedure C). Compound 41 was isolated using preparative HPLC and converted to the HCl salt. MS (MSD trap) m/z calcd. C₁₉₄H₃₆₀N₃₄O₅₇ 4080.0, Found 1361.8 (MH³⁺).

### Example 11-49

### Preparation of Compound 42

Compound 42 was synthesized using the general procedures described above as follows: coupled 1 with Fmoc-Lys(Boc)-Gly-OH (procedure A); Boc deprotection (procedure C); further coupled with palmitic acid (procedure D); Fmoc deprotection (procedure B). Further coupled with BocNH(CH₂)₃N(Boc)CH₂CH₂CH (COOH)NH)Boc)CH₂)₃)NHBoc (procedure A); Boc Deprotection (procedure C). Compound 42 was isolated using preparative HPLC and converted to the HCl salt. MS (MSD trap) m/z calcd. C₁₁₂H₂₁₀N₁₆O₄₁ 2436.5, Found 1220.0 (MH²⁺).

### Example 11-50

### Preparation of Compound 43

Compound 43 was synthesized using the general procedures described above as follows: coupled 1 with Fmoc-Lys(Boc)-Gly-Lys(Boc)-Gly-OH (SEQ ID NO: 104) (procedure A); Boc deprotection (procedure C); further coupled with palmitic acid (procedure D); Fmoc deprotection (procedure B). Further coupled with BocNH(CH₂)₃N(Boc) CH₂CH₂CH(COOH)NH)Boc)CH₂)₃)NHBoc (procedure A); Boc Deprotection (procedure C). Compound 43 was isolated using preparative HPLC and converted to the HCl salt. MS (MSD trap) m/z calcd. C₁₆₀H₃₀₀N₂₂O₄₇ 3284.18, Found 1644.0 (MH²⁺).

### Example 11-51

### Preparation of Compound 45

Compound 45 was synthesized using the general procedures described above as follows: coupled 44 with Fmoc-Lys(Boc)-Lys(Boc)-Lys(Boc)-Gly-OH (SEQ ID NO: 112) (procedure A); Fmoc deprotection (procedure B). Further coupled with Boc-Cys(trityl)-Gly-Lys(Ch2)-Gly-Lys(Ch2)-OH (SEQ ID NO: 115) (procedure A); Boc and Trityl Deprotection (procedure G) and a subsequent hydrolysis of the trifluoroacetate on Ch2 with 5 M HCl Methanol/Conc hydrochloric acid (2/1). Compound 45 was isolated using preparative HPLC and converted to the HCl salt. MS (MSD trap) m/z calcd. C₂₁₀H₃₆₂N₃₄O₆₀S₂ 4298.58, Found 1433.20 (MH³⁺).

### Example 11-52

### Preparation of Compound 46

Compound 46 was synthesized using the general procedures described above as follows: coupled 44 with Fmoc-Lys(Boc)-Lys(Boc)-Lys(Boc)-Lys(Boc)-Gly-OH (SEQ ID NO: 1) (procedure A); Fmoc deprotection (procedure B). Further coupled with Boc-Cys(trityl)-Gly-Lys(Ch2)-Gly-Lys(Ch2)-OH (SEQ ID NO: 115) (procedure A); Boc and Trityl Deprotection (procedure G) and a subsequent hydrolysis of the trifluoroacetate on Ch2 with 5 M HCl Methanol/Conc hydrochloric acid (2/1). Compound 46 was isolated using preparative HPLC and converted to the HCl salt. MS (MSD trap) m/z calcd. C₂₂₂H₃₈₆N₃₀O₅₈S₂ 4554.77, Found 1520.10 (MH³⁺).

### Example 11-53

### Preparation of Compound 47

Compound 47 was synthesized using the general procedures described above as follows: coupled 44 with Fmoc-Cys(Trt)-OH; Fmoc Deprotection (procedure B); Further coupled with Fmoc-Lys(Boc)-Lys(Boc)-Lys(Boc)-Lys(Boc)-Lys(Boc)-OH (SEQ ID NO: 2) (procedure A); Fmoc deprotection (procedure B). Further coupled with Boc-Cys(trityl)-Gly-Lys(Ch2)-Gly-Lys(Ch2)-OH (SEQ ID NO: 115) (procedure A); Boc and Trityl Deprotection (procedure G) and a subsequent hydrolysis of the trifluoroacetate on Ch2 with 5 M HCl Methanol/Conc hydrochloric acid (2/1). Compound 47 was isolated using preparative HPLC and converted to the HCl salt. MS (MSD trap) m/z calcd. C₂₃₆H₄₁₄N₃₈O₆₂S₄ 4902.94, Found 1635.90 (MH³⁺).

### Example 11-54

### Preparation of Compound 48

Compound 48 was synthesized using the general procedures described above as follows: coupled 44 with Fmoc-Cys(Trt)-OH; Fmoc Deprotection (procedure B); Further coupled with Fmoc-Lys(Boc)-Lys(Boc)-Lys(Boc)-Lys(Boc)-OH (SEQ ID NO: 16) (procedure A); Fmoc deprotection (procedure B). Further coupled with Boc-Gly-Lys(Ch2)-Gly-Lys(Ch2)-OH (SEQ ID NO: 116) (procedure A); Boc and Trityl Deprotection (procedure G) and a subsequent hydrolysis of the trifluoroacetate on Ch2 with 5 M HCl Methanol /Conc hydrochloric acid (2/1). Compound 48 was isolated using preparative HPLC and converted to the HCl salt. MS (MSD trap) m/z calcd. C₂₁₈H₃₈₀N₃₂O₅₈S₂ 4440.73, Found 1481,80 (MH³⁺).

### Example 11-55

### Preparation of Compound 49

Compound 49 was synthesized using the general procedures described above as follows: coupled 44 with Fmoc-Cys(Trt)-OH; Fmoc Deprotection (procedure B); Further coupled with Fmoc-Lys(Boc)-Lys(Boc)-Lys(Boc)-Lys(Boc)-OH (SEQ ID NO: 16) (procedure A); Fmoc deprotection (procedure B). Further coupled with Boc-Cys(trityl)-Gly-Lys(Ch2)-Gly-Lys(Ch2)-OH (SEQ ID NO: 115) (procedure A); Boc and Trityl Deprotection (procedure G) and a subsequent hydrolysis of the trifluoroacetate on Ch2 with 5 M HCl Methanol/Conc hydrochloric acid (2/1). Compound 49 was isolated using preparative HPLC and converted to the HCl salt. MS (MSD trap) m/z calcd. C₂₂₄H₃₉₀N₃₄O₆₀S₄ 4646.75, Found 1549.5 (MH^{3⇄}).

### Example 11-56

### Preparation of Compound 50

Compound 50 was synthesized using the general procedures described above as follows: coupled 44 with Fmoc-Cys(Trt)-OH; Fmoc Deprotection (procedure B); Further coupled with Fmoc-Lys(Boc)-Lys(Boc)-Lys(Boc)-Lys(Boc)-OH (SEQ ID NO: 16) (procedure A); Fmoc deprotection (procedure B). Further coupled with Ch6-OH (procedure A); Boc and Trityl Deprotection (procedure G) and a subsequent hydrolysis of the trifluoroacetate on Ch2 with 5 M HCl Methanol/Conc hydrochloric acid (2/1). Compound 50 was isolated using preparative HPLC and converted to the HCl salt. MS (MSD trap) m/z calcd. C₂₁₈H₃₈₄N₃₂O₆₀S₂ 4476.75, Found 1493.3 (MH³⁺).

### Example 12

### Knock-Down Activities of Exemplary Compounds of the Invention

Luciferase knockdown assays are indicative of treatments that are capable of modulating gene expression. Thus, a test compound that is capable of depressing gene expression in a cell is a prime candidate for further clinical studies. To evaluate the potential efficacy of invention compounds, luciferase knockdown assays of numerous compounds of the invention were carried out substantially as previously described in the literature (see, for example, Journal of Biomolecular Screening, Vol. 12, No. 4, 546-559, 2007; and Nucleic Acids Research, Vol. 31, No. 11, 2717-2724, 2003).

Specifically, Human Embryonic Kidney cells (HEK-293) were obtained from the American Type Culture Collection (Mannasas, Va.) and grown in DMEM medium supplemented with 10% fetal bovine serum. Luciferase expressing clones of HEK-293 were generated by transfection with the luciferase mammalian expression vector pGL4 (Promega Corp., Madison, Wis.) and drug selection on 500 μg/ml of neomycin. The neomycin selected pool was then single cell cloned by limiting dilution. Luciferase expression of individual clones was determined using the Steady Glo assay kit (Promega Corporation). A high expression clone (clone #11) was selected for use in knockdown assays.

The siRNA sequences:

| | |
|---|---|
| CCUACGCCGAGUACUUCGATT (sense) | (SEQ ID NO: 117) |
| and | |
| UCGAAGUACUCGGCGUAGGTT (antisense) | (SEQ ID NO: 118) |

encoding siRNA knockdown sequence for luciferase mRNA were purchase from Integrated DNA Technologies (San Diego, Calif.). The siRNAs were annealed at 65 degrees for 5 minutes and allowed to cool to room temperature to form 19 bp duplexes with 2 bp overhangs. Control siRNAs using scrambled luciferase knockdown sequence were also obtained from Integrated DNA Technologies for use as a negative control.

For knock down assays, HEK 293-luciferase clone #11 cells were plated at a density of 5000 cells per well in 96 well white assay plates with clear bottoms (Corning Costar) in 100 μl growth medium per well. For positive control wells, 25 pmol per well of luciferase knockdown siRNA was complexed with lipofectamine 2000 (Invitrogen Corp., San Diego, Calif.) as per manufacturers' recommendations. Negative control wells received equal amounts of scrambled sequence complexed with lipofectamine 2000. Test wells received 25 pmols luciferase knockdown siRNA or scrambled siRNA complexed with 125 pmols of test compound diluted in 50 μA of phosphate buffered saline. These complexes were incubated with cells for 4 hours at 37° C. After 4 hours, 100 μl per well of DMEM growth medium supplemented with 10% fetal bovine serum was added to each well to yield a final test volume of 150 μl per well. After a 72 h incubation of HEK-luciferase cells with test complexes in a 5% CO₂, 37° C. incubator, luciferase expression was measured in a plate luminometer (Molecular Devices M5) using the steady glo luciferase assay kit as per manufacturers' recommendations. Percent knockdown was calculated by comparing the luciferase expression of the test compound complexed with the luciferase knockdown sequence versus the luciferase expression of the test compound complexed with the scrambled knockdown sequence. Knockdown activity of compounds were scored based on the following activity scale:

− 0-14% reduction of luciferase mRNA expression;

+ 15-40% reduction of luciferase mRNA expression; and

++ >40% reduction of luciferase mRNA expression.

The results of knock-down experiments employing a number of exemplary compounds according to the invention are summarized in the following table:

| Example | Compound No. | Relative Activity |
|---|---|---|
| 5 | 6a | + |
| 5 | 6b | − |
| 5 | 6c | ++ |
| 5 | 6d | ++ |
| 5 | 6e | − |
| 5 | 6f | − |
| 5 | 6g | − |
| 5 | 6h | + |
| 5 | 6i | ++ |
| 5 | 6j | + |
| 5 | 6k | + |
| 5 | 6l | ++ |
| 5 | 6m | ++ |
| 5 | 6n | − |
| 5 | 6o | − |
| 6 | 7b | ++ |
| 6 | 7c | − |
| 6 | 7d | − |
| 6 | 7e | − |
| 7 | 8a | − |
| 7 | 8b | + |
| 8 | 10a | ++ |
| 8 | 10c | − |
| 8 | 10d | ++ |
| 8 | 11 | − |
| 8 | 12 | − |
| 8 | 13 | − |
| 8 | 14 | + |
| 8 | 15 | + |
| 8 | 16 | − |
| 8 | 17 | ++ |
| 8 | 18 | + |
| 8 | 19 | + |
| 8 | 20 | ++ |
| 8 | 21 | − |
| 8 | 22 | − |
| 8 | 23 | ++ |
| 8 | 24 | − |
| 8 | 25 | + |
| 8 | 26 | + |
| 8 | 27 | ++ |
| 8 | 28 | ++ |
| 8 | 29 | ++ |
| 8 | 30 | + |
| 8 | 31 | + |
| 8 | 32 | + |
| 8 | 33 | + |
| 8 | 34 | ++ |
| 8 | 35 | ++ |
| 8 | 36 | ++ |
| 8 | 37 | ++ |
| 8 | 38 | ++ |
| 8 | 39 | ++ |
| 8 | 40 | ++ |
| 8 | 41 | ++ |
| 8 | 42 | ++ |
| 8 | 43 | ++ |
| 8 | 44 | ++ |
| 8 | 45 | ++ |
| 8 | 46 | ++ |
| 8 | 47 | ++ |
| 8 | 48 | ++ |
| 8 | 49 | ++ |
| 8 | 50 | ++ |
| 8 | 51 | ++ |
| 8 | 52 | ++ |
| 8 | 53 | ++ |
| 8 | 54 | ++ |
| 8 | 55 | ++ |
| 8 | 56 | ++ |
| 8 | 57 | ++ |
| 8 | 58 | ++ |
| 8 | 59 | + |
| 8 | 60 | ++ |
| 8 | 61 | ++ |
| 8 | 62 | ++ |
| 8 | 63 | ++ |
| 8 | 64 | + |
| 8 | 65 | ++ |
| 8 | 66 | ++ |
| 10 | 11 | − |
| 10 | 12 | ++ |
| 10 | 13 | ++ |
| 10 | 14 | − |
| 10 | 15 | − |
| 10 | 16 | − |
| 10 | 17 | − |
| 10 | 18 | − |
| 10 | 19 | − |
| 10 | 20 | − |
| 10 | 21 | ++ |
| 10 | 22 | ++ |
| 10 | 23 | − |
| 10 | 24 | + |
| 10 | 25 | + |
| 10 | 26 | ++ |
| 10 | 27 | ++ |
| 10 | 28 | − |
| 10 | 29 | − |
| 10 | 30 | − |
| 10 | 31 | ++ |
| 10 | 32 | ++ |
| 10 | 33 | + |
| 10 | 34 | ++ |
| 10 | 35 | ++ |
| 10 | 36 | ++ |
| 10 | 37 | ++ |
| 10 | 38 | + |
| 10 | 39 | ++ |
| 10 | 40 | + |
| 10 | 41 | ++ |
| 10 | 42 | ++ |
| 10 | 43 | − |
| 10 | 44 | ++ |
| 10 | 45 | ++ |
| 10 | 46 | ++ |
| 10 | 47 | ++ |
| 10 | 48 | ++ |
| 10 | 49 | ++ |
| 10 | 50 | + |
| 10 | 51 | ++ |
| 10 | 52 | ++ |
| 10 | 53 | ++ |
| 10 | 54 | ++ |
| 10 | 55 | ++ |
| 10 | 56 | ++ |
| 10 | 57 | ++ |
| 10 | 58 | ++ |
| 10 | 59 | ++ |
| 10 | 60 | ++ |
| 10 | 61 | ++ |
| 10 | 62 | ++ |
| 10 | 63 | ++ |
| 10 | 64 | ++ |
| 10 | 65 | ++ |
| 10 | 66 | ++ |
| 10 | 67 | ++ |
| 10 | 68 | ++ |
| 10 | 69 | ++ |
| 10 | 70 | ++ |
| 10 | 71 | ++ |
| 10 | 72 | ++ |
| 10 | 72 | ++ |
| 10 | 73 | ++ |
| 10 | 74 | ++ |
| 10 | 75 | ++ |
| 10 | 76 | ++ |
| 10 | 77 | ++ |
| 10 | 78 | ++ |
| 10 | 79 | ++ |
| 10 | 80 | ++ |
| 10 | 81 | ++ |
| 10 | 82 | + |
| 10 | 83 | ++ |
| 10 | 84 | ++ |
| 10 | 85 | ++ |
| 10 | 86 | ++ |
| 10 | 87 | ++ |
| 10 | 88 | + |
| 10 | 89 | + |
| 10 | 90 | + |
| 10 | 91 | + |
| 10 | 92 | ++ |
| 10 | 93 | − |
| 10 | 94 | − |
| 10 | 95 | ++ |
| 10 | 96 | ++ |
| 10 | 97 | ++ |
| 10 | 98 | ++ |
| 10 | 99 | ++ |
| 10 | 100 | + |
| 10 | 101 | ++ |
| 10 | 102 | ++ |
| 10 | 103 | ++ |
| 10 | 104 | ++ |
| 10 | 105 | ++ |
| 10 | 106 | ++ |
| 10 | 107 | ++ |
| 10 | 108 | ++ |
| 10 | 109 | + |
| 10 | 110 | + |
| 10 | 111 | ++ |
| 10 | 112 | ++ |
| 10 | 113 | ++ |
| 11 | 7 | − |
| 11 | 8 | − |
| 11 | 11 | ++ |
| 11 | 12 | + |
| 11 | 13 | ++ |
| 11 | 14 | − |
| 11 | 15 | − |
| 11 | 16 | + |
| 11 | 17 | − |
| 11 | 18 | − |
| 11 | 19 | − |
| 11 | 20 | + |
| 11 | 21 | ++ |
| 11 | 22 | + |
| 11 | 23 | − |
| 11 | 24 | + |
| 11 | 28 | − |
| 11 | 29 | − |
| 11 | 30 | − |
| 11 | 31 | − |
| 11 | 32 | − |
| 11 | 33 | − |
| 11 | 36 | − |
| 11 | 37 | − |
| 11 | 45 | ++ |
| 11 | 46 | ++ |
| 11 | 47 | ++ |
| 11 | 48 | ++ |
| 11 | 49 | + |
| 11 | 50 | ++ |

### Example 13

### Knock-Down Activities of Control Compounds

Luciferase knockdown assays as described above were carried out with several control compounds. Results therewith are summarized in the following table. The compounds tested had either the core structure:

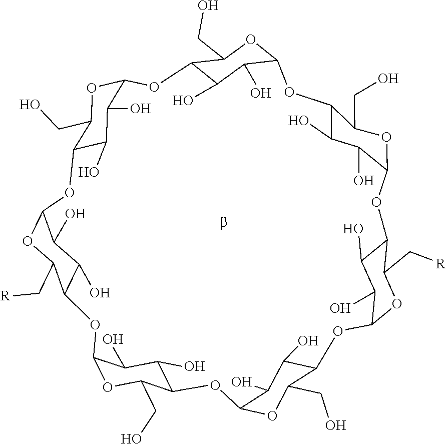

and the positively charged arms, R, were:

both CKKKKK-Ch1 (SEQ ID NO: 119),

one arm CKKKKK (SEQ ID NO: 120), second arm CKKKKK-X1 (SEQ ID NO: 121),

both CKKKKK (SEQ ID NO: 120),

both CKKKKK-X23 (SEQ ID NO: 122), wherein X23 is as set forth below,

both CKKKKK-X24 (SEQ ID NO: 123), wherein X24 is as set forth below,

both GGOOOOOC,

both GKKKKK(Ch2)GC (SEQ ID NO: 124)

both GKKKKK(Ch2)G (SEQ ID NO: 125), or

the core structure:

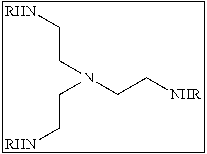

and the following positively charged arm—GKKKKGKGC (SEQ ID NO: 126), wherein

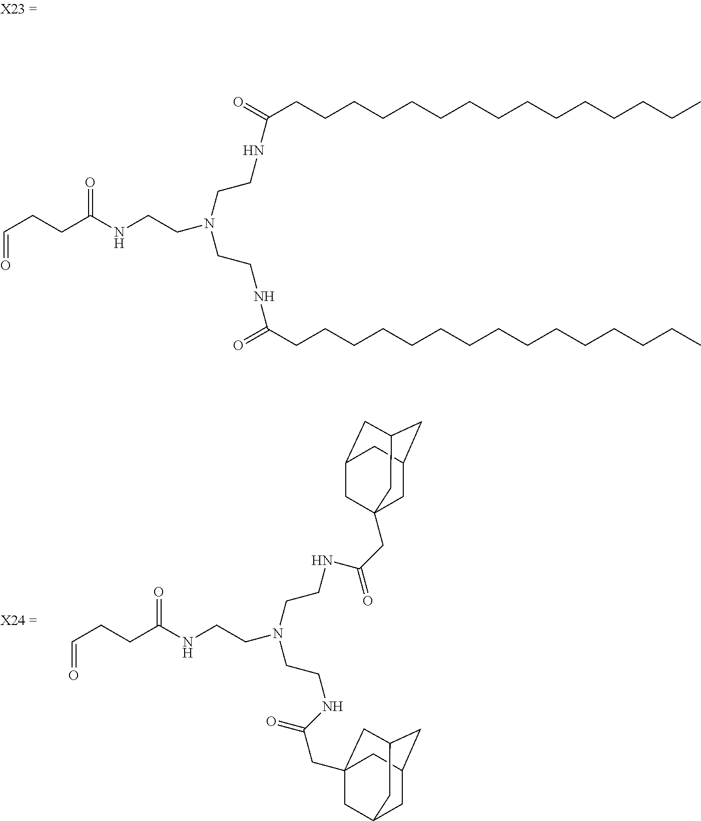

None of the preceding control compounds displayed any knock-down activity in the assays described in the previous example.

As can be seen by review of the above data, numerous variations of compounds according to the present invention are highly effective to knock-down the activity in a model system employing luciferase expressing clones.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope. Changes therein and other uses will occur to those skilled in the art, which are encompassed within the spirit of the invention, are defined by the scope of the claims.

Definitions provided herein are not intended to be limiting from the meaning commonly understood by one of skill in the art unless indicated otherwise.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A molecular entity comprising:
an amphiphilic core having
at least two positively charged arms covalently attached thereto, and
a plurality of bile acid moieties covalently attached to said positively charged arms.

2. The molecular entity of claim 1 wherein said positively charged arms are symmetrically substituted with said plurality of bile acid moieties.

3. The molecular entity of claim 1 wherein each positively charged arm comprises two bile acid moieties thereon.

4. The molecular entity of claim 1 having the Formula I:)
A(-B)ₙ (I)
wherein:
A is an amphiphilic core,
each B is independently a positively charged arm having a plurality of positive charges thereon, and a plurality of bile acid moieties covalently attached thereto, and
n is an integer from 2 to 7.

5. The molecular entity of claim 4 wherein n is an integer from 2 to 4.

6. The molecular entity of claim 1, wherein said amphiphilic core comprises at least two attachment sites separated by a distance in the range of about 5-35 Angstroms for linkage of said arms to said core.

7. The molecular entity of claim 1, wherein said amphiphilic core is an atom, a linearly extended structure, a branched structure, a cyclic structure, a macrocyclic structure, or a cyclic peptide.

8. The molecular entity of claim 1, wherein said amphiphilic core is a cyclic peptide or one of the following structures: 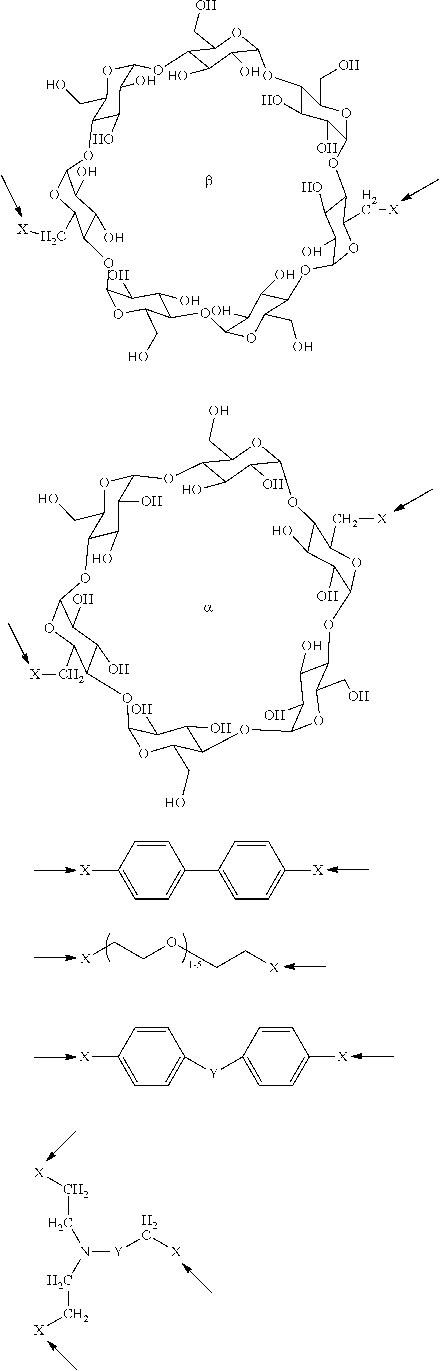 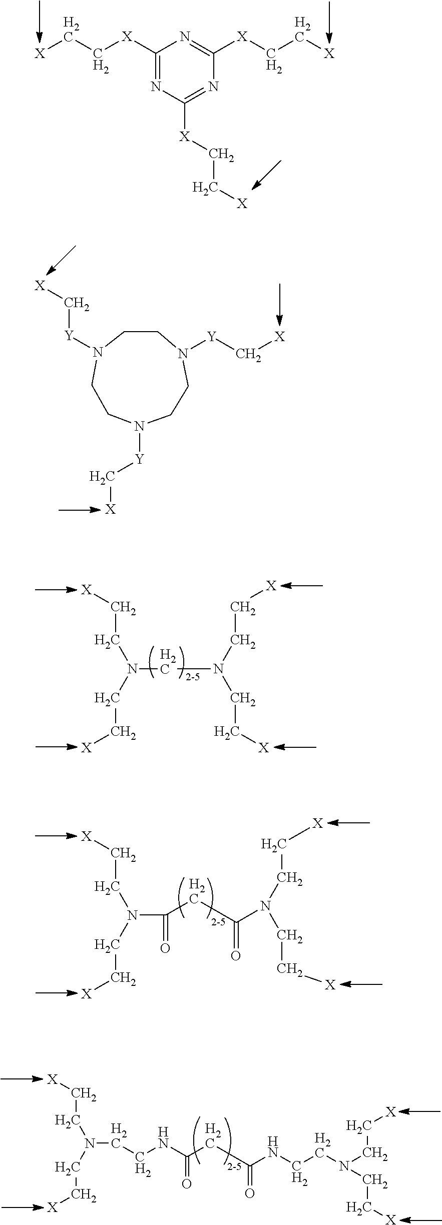
wherein:
→ identifies the atom through which the positively charged arm is attached to the amphiphilic core;
X═NH, O, S, CH₂NH, C(═O), SO₂, SO₂NH or NHC(═O); and
Y═CH₂, O, or C(═O).

9. The molecular entity of claim 1, wherein said positively charged arm has the Formula (II): 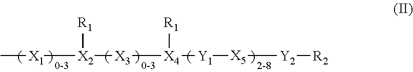
wherein:
each occurrence of X₁, X₂, X₃ and X₄ is independently a monomer unit;
X₅ is a monomer unit having at least one positive charge;
Y₁ is a short spacer;
Y₂ is an extended spacer;
each R₁ is independently selected from the group consisting of bile acids; and
R₂ is H, an amine or a polyethyleneglycol polymer (PEG) optionally linked to a fusogenic moiety, a targeting moiety, or a cell membrane active moiety.

10. The molecular entity of claim 9, wherein said positively charged arm has the Formula (IIa):

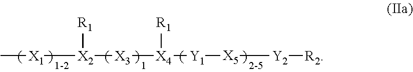

11. The molecular entity of claim 1, wherein said positively charged arm has the Formula (III): 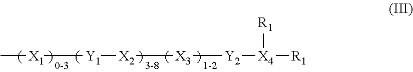
wherein:
each occurrence of X₁ and X₃ is independently a monomer unit;
X₂ is a monomer unit having at least one positive charge;
X₄ is a monomer unit having two sites of attachment;
Y₁ is a short spacer;
Y₂ is an extended spacer; and
each R₁ is independently a bile acid.

12. The molecular entity of claim 11, wherein said positively charged arm has the Formula (IIIa):

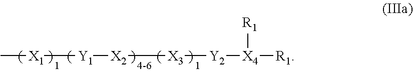

13. The molecular entity of claim 1, wherein said positively charged arm has Formula (IV): 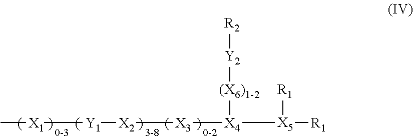
wherein:
each occurrence of X₁, X₃, and X₆ is independently a monomer unit;
X₂ is a monomer unit having at least one positive charge;
X₄ is a monomer having a site for attachment;
X₅ is a monomer unit having two sites of attachment;
Y₁ is a short spacer;
Y₂ is an extended spacer;
each R₁ is independently a bile acid; and
each R₂ is independently selected from the group consisting of H, an amine and a polyethyleneglycol polymer (PEG) optionally linked to a fusogenic moiety, a targeting moiety, or a cell membrane active moiety.

14. The molecular entity of claim 13, wherein said positively charged arm has the Formula (IVa):

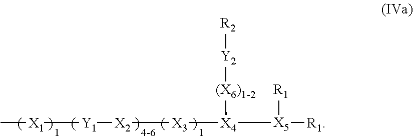

15. The molecular entity of claim 1, wherein said positively charged arm has Formula (V): 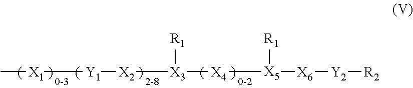
wherein:
each occurrence of X₁, X₃, X₄, X₅ and X₆ is independently a monomer unit;
X₂ is a monomer unit having at least one positive charge;
Y₁ is a short spacer;
Y₂ is an extended spacer;
each R₁ is independently a bile acid; and
R₂ is H, an amine, or a polyethyleneglycol polymer (PEG) optionally linked to a fusogenic moiety, a targeting moiety, or a cell membrane active subunit.

16. The molecular entity of claim 15, wherein said positively charged arm has the Formula (Va):

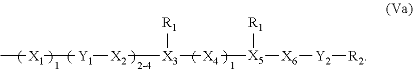

17. The molecular entity of claim 1, wherein said bile acids are selected from the group consisting of cholic acid, chenodeoxycholic acid, glycocholic acid, taurocholic acid, deoxycholic acid, and lithocholic acid.

18. The molecular entity of claim 1, wherein said plurality of lipophilic moieties are covalently attached to said charged arms via ether, thioether, disulfide, amine, imine, amidine, keto, ester, amide, imide, carboxamide, urea, linkage.

19. The molecular entity of claim 1, wherein said plurality of lipophilic moieties are covalently attached to said charged arms via an amide linkage.

20. The molecular entity of claim 1, wherein said entity binds, stabilizes and/or facilitates cellular delivery of negatively charged entities.

21. The molecular entity of claim 1, wherein said positively charged arms comprise a plurality of residues selected from amines, guanidines, amidines, N-containing heterocycles, or combinations thereof.

22. The molecular entity of claim 1, wherein one or both of said positively charged arms further comprise neutral and/or polar functional groups.

23. The molecular entity of claim 9, wherein said monomer units X₁, X₂, X₃, X₄, X₅ and X₆ are independently selected from compounds having the general Formula (VI): 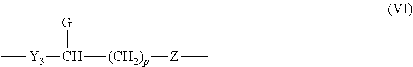
wherein:
G is hydrogen, lower alkyl or functionalized lower alkyl having any alpha-amino acid side chain, or a cationically or an anionically functionalized side chain thereon;
Y₃ is a covalent bond, O, NR¹, C(O), S or SO₂,
Z is a covalent bond, O, NR¹R², C(O), NR¹C(O), C(O)NR¹, S or SO₂,
R¹ and R² are independently a bond, hydrogen, lower alkyl or heteroatom-substituted lower alkyl; and
p is 0, 1, 2, 3, 4, 5 or 6.

24. The molecular entity of claim 9, wherein short spacer, Y₁, is selected from the group consisting of a bond, or a monomer unit having the general Formula (VI), with the proviso that said monomer unit does not have a cationically charged side chain.

25. The molecular entity of claim 9, wherein extended spacer, Y₂, is selected from the group consisting of a bond, a monomer unit having the general Formula (VI), with the proviso that said monomer unit does not have a cationically charged side chain, an hydroxylated alkyl chain, a multi-hydroxylated alkyl chain (i.e., an open-chain carbohydrate), a polyethylene glycol (PEG), and combinations of any two or more thereof.

26. The molecular entity of claim 1, wherein said molecular entity is pegylated or partially pegylated.

27. The molecular entity of claim 1, further comprising a bio-recognition molecule.

28. The molecular entity of claim 1, further comprising one or more thiol groups (SH) thereon.

29. A composition comprising oligomeric or polymeric molecular entities prepared by interacting the thiol group(s) of a plurality of molecular entities of claim 28 with one another under conditions suitable to form stable disulfide bonds.

30. A composition comprising an aggregation of a plurality of molecular entities of claim 1, wherein said aggregation comprises particles of between about 10 nanometers up to about 500 nanometers in size.

31. A composition comprising:
a pharmaceutical excipient,
an entity bearing an overall negative charge, and
a molecular entity of claim 1, or a pharmaceutically acceptable ester, salt, or hydrate thereof.

32. The composition of claim 31, wherein said entity bearing an overall negative charge is a double-stranded or hairpin nucleic acid.

33. The composition of claim 31, wherein said entity bearing an overall negative charge is selected from the group consisting of single-stranded DNA, double-stranded DNA, single-stranded RNA, double-stranded RNA and oligonucleotide comprising non-natural monomers.

34. The composition of claim 31, wherein said entity bearing an overall negative charge is a single-stranded RNA.

35. The composition of claim 34, wherein said single-stranded RNA is mRNA or miRNA.

36. The composition of claim 31, wherein said entity bearing an overall negative charge is a double-stranded RNA.

37. The composition of claim 36, wherein said double-stranded RNA is siRNA or a chemically modified form thereof.

38. A complex comprising a molecular entity of claim 1, associated with a charged entity.

39. The complex of claim 38 wherein said core is alpha, beta or gamma cyclodextrin.

40. The complex of claim 38, wherein the charge ratio of said molecular entity to said charged entity ranges from 1:12 to 12:1.

41. The complex of claim 40 wherein said charge ratio ranges from 1:1 to 8:1.

42. A composition comprising:
a pharmaceutical excipient, and
a complex of claim 38, or a pharmaceutically acceptable ester, salt, or hydrate thereof.

43. A method for reducing the susceptibility of a double-stranded or hairpin nucleic acid to digestion by enzymatic nuclease, said method comprising contacting said nucleic acid with a molecular entity of claim 1.

44. A method for reducing the susceptibility of a double-stranded or hairpin nucleic acid to hydrolysis of the phosphodiester backbone, said method comprising contacting said nucleic acid with a molecular entity of claim 1.

45. A method for delivering a negatively charged entity to a cell, said method comprising:
a) binding non-covalently a molecular entity of claim 1 to said negatively charged entity to form a complex; and
b) contacting said cell with said complex; wherein said negatively charged entity is taken up by said cell.

46. A method for delivering a negatively charged entity to a cell, said method comprising contacting said cell with a complex prepared by binding non-covalently a molecular entity of claim 1 to said negatively charged entity, wherein said negatively charged entity is taken up by said cell.

47. A method for stabilizing a negatively charged entity in vivo, said method comprising contacting said negatively charged entity with a molecular entity of claim 1.

48. A method for causing knock-down of a gene in a cell, said method comprising contacting said cell with a composition according to claim 37.


SEQUENCE LISTING (127 sequences)

SEQ ID NO: 1 - protein, 5 aa, Artificial Sequence
  Description of Artificial Sequence Synthetic peptide
KKKKG

SEQ ID NO: 2 - protein, 5 aa, Artificial Sequence
  Description of Artificial Sequence Synthetic peptide
KKKKK

SEQ ID NO: 3 - protein, 6 aa, Artificial Sequence
  Description of Artificial Sequence Synthetic peptide
CKKKKK

SEQ ID NO: 4 - protein, 10 aa, Artificial Sequence
  Description of Artificial Sequence Synthetic peptide
SKSKCKKKKK

SEQ ID NO: 5 - protein, 4 aa, Artificial Sequence
  Description of Artificial Sequence Synthetic peptide
SKSK

SEQ ID NO: 6 - protein, 6 aa, Artificial Sequence
  Description of Artificial Sequence Synthetic peptide
KKKKKK

SEQ ID NO: 7 - protein, 6 aa, Artificial Sequence
  Description of Artificial Sequence Synthetic peptide
KKKKKS

SEQ ID NO: 8 - protein, 6 aa, Artificial Sequence
  Description of Artificial Sequence Synthetic peptide
KKKKKS

SEQ ID NO: 9 - protein, 6 aa, Artificial Sequence
  Description of Artificial Sequence Synthetic peptide
KKKKKG

SEQ ID NO: 10 - protein, 7 aa, Artificial Sequence
  Description of Artificial Sequence Synthetic peptide
KKKKKGG

SEQ ID NO: 11 - protein, 5 aa, Artificial Sequence
  Description of Artificial Sequence Synthetic peptide
KKKKC

SEQ ID NO: 12 - protein, 6 aa, Artificial Sequence
  Description of Artificial Sequence Synthetic peptide
KKKKKC

SEQ ID NO: 13 - protein, 7 aa, Artificial Sequence
  Description of Artificial Sequence Synthetic peptide
KKKKKKC

SEQ ID NO: 14 - protein, 11 aa, Artificial Sequence
  Description of Artificial Sequence Synthetic peptide
KKKKKKKKKKC

SEQ ID NO: 15 - protein, 8 aa, Artificial Sequence
  Description of Artificial Sequence Synthetic peptide
KSKSKSKC

SEQ ID NO: 16 - protein, 4 aa, Artificial Sequence
  Description of Artificial Sequence Synthetic peptide
KKKK

SEQ ID NO: 17 - protein, 6 aa, Artificial Sequence
  Description of Artificial Sequence Synthetic peptide
KKKKKK

SEQ ID NO: 18 - protein, 7 aa, Artificial Sequence
  Description of Artificial Sequence Synthetic peptide
KSKSKSK

SEQ ID NO: 19 - protein, 5 aa, Artificial Sequence
  Description of Artificial Sequence Synthetic peptide
KKKKG

SEQ ID NO: 20 - protein, 5 aa, Artificial Sequence
  Description of Artificial Sequence Synthetic peptide
KKKKK

SEQ ID NO: 21 - protein, 6 aa, Artificial Sequence
  Description of Artificial Sequence Synthetic peptide
CKKKKK

SEQ ID NO: 22 - protein, 10 aa, Artificial Sequence
  Description of Artificial Sequence Synthetic peptide
SKSKCKKKKK

SEQ ID NO: 23 - protein, 6 aa, Artificial Sequence
  Description of Artificial Sequence Synthetic peptide
KKKKKK

SEQ ID NO: 24 - protein, 6 aa, Artificial Sequence
  Description of Artificial Sequence Synthetic peptide
KKKKKK

SEQ ID NO: 25 - protein, 6 aa, Artificial Sequence
  Description of Artificial Sequence Synthetic peptide
KKKKKS

SEQ ID NO: 26 - protein, 6 aa, Artificial Sequence
  Description of Artificial Sequence Synthetic peptide
KKKKKC

SEQ ID NO: 27 - protein, 6 aa, Artificial Sequence
  Description of Artificial Sequence Synthetic peptide
KKKKKG

SEQ ID NO: 28 - protein, 7 aa, Artificial Sequence
  Description of Artificial Sequence Synthetic peptide
KKKKKGG

SEQ ID NO: 29 - protein, 5 aa, Artificial Sequence
  Description of Artificial Sequence Synthetic peptide
KKKKC

SEQ ID NO: 30 - protein, 7 aa, Artificial Sequence
  Description of Artificial Sequence Synthetic peptide
KKKKKKC

SEQ ID NO: 31 - protein, 11 aa, Artificial Sequence
  Description of Artificial Sequence Synthetic peptide
KKKKKKKKKKC

SEQ ID NO: 32 - protein, 8 aa, Artificial Sequence
  Description of Artificial Sequence Synthetic peptide
KSKSKSKC

SEQ ID NO: 33 - protein, 10 aa, Artificial Sequence
  Description of Artificial Sequence Synthetic peptide
GGCGKKKGKK

SEQ ID NO: 34 - protein, 10 aa, Artificial Sequence
  Description of Artificial Sequence Synthetic peptide
GGCGKKKGKK

SEQ ID NO: 35 - protein, 7 aa, Artificial Sequence
  Description of Artificial Sequence Synthetic peptide
GKGKKKK

SEQ ID NO: 36 - protein, 9 aa, Artificial Sequence
  Description of Artificial Sequence Synthetic peptide
GGKGKGXXX

SEQ ID NO: 37 - protein, 9 aa, Artificial Sequence
  Description of Artificial Sequence Synthetic peptide
GGKGKGXXX

SEQ ID NO: 38 - protein, 10 aa, Artificial Sequence
  Description of Artificial Sequence Synthetic peptide
GGKGKGXXXX

SEQ ID NO: 39 - protein, 7 aa, Artificial Sequence
  Description of Artificial Sequence Synthetic peptide
GGKGKGR

SEQ ID NO: 40 - protein, 8 aa, Artificial Sequence
  Description of Artificial Sequence Synthetic peptide
GGKGKKKK

SEQ ID NO: 41 - protein, 7 aa, Artificial Sequence
  Description of Artificial Sequence Synthetic peptide
GGKRRRR

SEQ ID NO: 42 - protein, 8 aa, Artificial Sequence
  Description of Artificial Sequence Synthetic peptide
GGKGKGRR

SEQ ID NO: 43 - protein, 8 aa, Artificial Sequence
  Description of Artificial Sequence Synthetic peptide
GGKGKGRR

SEQ ID NO: 44 - protein, 9 aa, Artificial Sequence
  Description of Artificial Sequence Synthetic peptide
GGKGKGRRR

SEQ ID NO: 45 - protein, 9 aa, Artificial Sequence
  Description of Artificial Sequence Synthetic peptide
GGKGKGRRR

SEQ ID NO: 46 - protein, 10 aa, Artificial Sequence
  Description of Artificial Sequence Synthetic peptide
GGKGKGKKKK

SEQ ID NO: 47 - protein, 10 aa, Artificial Sequence
  Description of Artificial Sequence Synthetic peptide
GGKGKGKKKK

SEQ ID NO: 48 - protein, 10 aa, Artificial Sequence
  Description of Artificial Sequence Synthetic peptide
GKKKKKGKGC

SEQ ID NO: 49 - protein, 7 aa, Artificial Sequence
  Description of Artificial Sequence Synthetic peptide
KKGKKKG

SEQ ID NO: 50 - protein, 7 aa, Artificial Sequence
  Description of Artificial Sequence Synthetic peptide
XXXGKGK

SEQ ID NO: 51 - protein, 8 aa, Artificial Sequence
  Description of Artificial Sequence Synthetic peptide
XXXXGKGK

SEQ ID NO: 52 - protein, 5 aa, Artificial Sequence
  Description of Artificial Sequence Synthetic peptide
RGKGK

SEQ ID NO: 53 - protein, 4 aa, Artificial Sequence
  Description of Artificial Sequence Synthetic peptide
RRRR

SEQ ID NO: 54 - protein, 6 aa, Artificial Sequence
  Description of Artificial Sequence Synthetic peptide
RRGKGK

SEQ ID NO: 55 - protein, 7 aa, Artificial Sequence
  Description of Artificial Sequence Synthetic peptide
RRRGKGK

SEQ ID NO: 56 - protein, 8 aa, Artificial Sequence
  Description of Artificial Sequence Synthetic peptide
KKKKGKGK

SEQ ID NO: 57 - protein, 5 aa, Artificial Sequence
  Description of Artificial Sequence Synthetic peptide
CGKGK

SEQ ID NO: 58 - protein, 11 aa, Artificial Sequence
  Description of Artificial Sequence Synthetic peptide
KGKKKKGKKKK

SEQ ID NO: 59 - protein, 11 aa, Artificial Sequence
  Description of Artificial Sequence Synthetic peptide
KGKKKKGKKKK

SEQ ID NO: 60 - protein, 10 aa, Artificial Sequence
  Description of Artificial Sequence Synthetic peptide
GGKKKKKKKK

SEQ ID NO: 61 - protein, 10 aa, Artificial Sequence
  Description of Artificial Sequence Synthetic peptide
GGKKKKKKKK

SEQ ID NO: 62 - protein, 10 aa, Artificial Sequence
  Description of Artificial Sequence Synthetic peptide
GGKKKKKKKK

SEQ ID NO: 63 - protein, 8 aa, Artificial Sequence
  Description of Artificial Sequence Synthetic peptide
KKKKKKKK

SEQ ID NO: 64 - protein, 10 aa, Artificial Sequence
  Description of Artificial Sequence Synthetic peptide
KKKKKKKKKK

SEQ ID NO: 65 - protein, 4 aa, Artificial Sequence
  Description of Artificial Sequence Synthetic peptide
GKGK

SEQ ID NO: 66 - protein, 5 aa, Artificial Sequence
  Description of Artificial Sequence Synthetic peptide
GKGKG

SEQ ID NO: 67 - protein, 9 aa, Artificial Sequence
  Description of Artificial Sequence Synthetic peptide
GKGKGGKKK

SEQ ID NO: 68 - protein, 8 aa, Artificial Sequence
  Description of Artificial Sequence Synthetic peptide
GKGKKHCC

SEQ ID NO: 69 - protein, 9 aa, Artificial Sequence
  Description of Artificial Sequence Synthetic peptide
GKGKGKKKK

SEQ ID NO: 70 - protein, 10 aa, Artificial Sequence
  Description of Artificial Sequence Synthetic peptide
GKGKGKKKKK

SEQ ID NO: 71 - protein, 11 aa, Artificial Sequence
  Description of Artificial Sequence Synthetic peptide
GKGKGKKKKKK

SEQ ID NO: 72 - protein, 10 aa, Artificial Sequence
  Description of Artificial Sequence Synthetic peptide
GKGKKKKKKC

SEQ ID NO: 73 - protein, 9 aa, Artificial Sequence
  Description of Artificial Sequence Synthetic peptide
CKKKKKKKK

SEQ ID NO: 74 - protein, 10 aa, Artificial Sequence
  Description of Artificial Sequence Synthetic peptide
CKKKKKKKKK

SEQ ID NO: 75 - protein, 16 aa, Artificial Sequence
  Description of Artificial Sequence Synthetic peptide
CKKKGKKKGKKKGKKK

SEQ ID NO: 76 - protein, 9 aa, Artificial Sequence
  Description of Artificial Sequence Synthetic peptide
GKGKGKKKK

SEQ ID NO: 77 - protein, 10 aa, Artificial Sequence
  Description of Artificial Sequence Synthetic peptide
GKGKGKKKKK

SEQ ID NO: 78 - protein, 11 aa, Artificial Sequence
  Description of Artificial Sequence Synthetic peptide
GKGKGKKKKKK

SEQ ID NO: 79 - protein, 10 aa, Artificial Sequence
  Description of Artificial Sequence Synthetic peptide
GKGKKKKKKC

SEQ ID NO: 80 - protein, 8 aa, Artificial Sequence
  Description of Artificial Sequence Synthetic peptide
GKGKGKKK

SEQ ID NO: 81 - protein, 9 aa, Artificial Sequence
  Description of Artificial Sequence Synthetic peptide
GKGKGKKKK

SEQ ID NO: 82 - protein, 10 aa, Artificial Sequence
  Description of Artificial Sequence Synthetic peptide
GKGKGKKKKK

SEQ ID NO: 83 - protein, 8 aa, Artificial Sequence
  Description of Artificial Sequence Synthetic peptide
GKGKGKKK

SEQ ID NO: 84 - protein, 9 aa, Artificial Sequence
  Description of Artificial Sequence Synthetic peptide
GKGKGKKKK

SEQ ID NO: 85 - protein, 10 aa, Artificial Sequence
  Description of Artificial Sequence Synthetic peptide
GKGKGKKKKK

SEQ ID NO: 86 - protein, 11 aa, Artificial Sequence
  Description of Artificial Sequence Synthetic peptide
GKGKGKKKKKK

SEQ ID NO: 87 - protein, 10 aa, Artificial Sequence
  Description of Artificial Sequence Synthetic peptide
GKGKKKKKKC

SEQ ID NO: 88 - protein, 9 aa, Artificial Sequence
  Description of Artificial Sequence Synthetic peptide
GKGKGKKKK

SEQ ID NO: 89 - protein, 8 aa, Artificial Sequence
  Description of Artificial Sequence Synthetic peptide
GKGKGKGK

SEQ ID NO: 90 - protein, 7 aa, Artificial Sequence
  Description of Artificial Sequence Synthetic peptide
GKGKKKK

SEQ ID NO: 91 - protein, 10 aa, Artificial Sequence
  Description of Artificial Sequence Synthetic peptide
GKGKGKGKGK

SEQ ID NO: 92 - protein, 10 aa, Artificial Sequence
  Description of Artificial Sequence Synthetic peptide
GKGKGKKGKK

SEQ ID NO: 93 - protein, 7 aa, Artificial Sequence
  Description of Artificial Sequence Synthetic peptide
GKGKKKK

SEQ ID NO: 94 - protein, 9 aa, Artificial Sequence
  Description of Artificial Sequence Synthetic peptide
GKGKGKKKK

SEQ ID NO: 95 - protein, 53 aa, Artificial Sequence
  Description of Artificial Sequence Synthetic polypeptide
GKGKGKKGKKKKKKKKKKKKKKKKKKKKKKKKKKKKKKKKKKKKKKKKKKKKK

SEQ ID NO: 96 - protein, 4 aa, Artificial Sequence
  Description of Artificial Sequence Synthetic peptide
GKGK

SEQ ID NO: 97 - protein, 9 aa, Artificial Sequence
  Description of Artificial Sequence Synthetic peptide
GKKKKGKGC

SEQ ID NO: 98 - protein, 10 aa, Artificial Sequence
  Description of Artificial Sequence Synthetic peptide
GKKKKKGKGC

SEQ ID NO: 99 - protein, 11 aa, Artificial Sequence
  Description of Artificial Sequence Synthetic peptide
CKKKKKKGKGC

SEQ ID NO: 100 - protein, 9 aa, Artificial Sequence
  Description of Artificial Sequence Synthetic peptide
CKKKKKGKG

SEQ ID NO: 101 - protein, 10 aa, Artificial Sequence
  Description of Artificial Sequence Synthetic peptide
CKKKKKGKGC

SEQ ID NO: 102 - protein, 6 aa, Artificial Sequence
  Description of Artificial Sequence Synthetic peptide
CKKKKK

SEQ ID NO: 103 - protein, 8 aa, Artificial Sequence
  Description of Artificial Sequence Synthetic peptide
KKKKKKKK

SEQ ID NO: 104 - protein, 4 aa, Artificial Sequence
  Description of Artificial Sequence Synthetic peptide
KGKG

SEQ ID NO: 105 - protein, 5 aa, Artificial Sequence
  Description of Artificial Sequence Synthetic peptide
KKKGG

SEQ ID NO: 106 - protein, 4 aa, Artificial Sequence
  Description of Artificial Sequence Synthetic peptide
CCHK

SEQ ID NO: 107 - protein, 7 aa, Artificial Sequence
  Description of Artificial Sequence Synthetic peptide
KKKKKKG

SEQ ID NO: 108 - protein, 7 aa, Artificial Sequence
  Description of Artificial Sequence Synthetic peptide
CKKKKKG

SEQ ID NO: 109 - protein, 9 aa, Artificial Sequence
  Description of Artificial Sequence Synthetic peptide
CKKKKKKKK

SEQ ID NO: 110 - protein, 10 aa, Artificial Sequence
  Description of Artificial Sequence Synthetic peptide
CKKKKKKKKK

SEQ ID NO: 111 - protein, 16 aa, Artificial Sequence
  Description of Artificial Sequence Synthetic peptide
CKKKGKKKGKKKGKKK

SEQ ID NO: 112 - protein, 4 aa, Artificial Sequence
  Description of Artificial Sequence Synthetic peptide
KKKG

SEQ ID NO: 113 - protein, 6 aa, Artificial Sequence
  Description of Artificial Sequence Synthetic peptide
KGKGKG

SEQ ID NO: 114 - protein, 6 aa, Artificial Sequence
  Description of Artificial Sequence Synthetic peptide
KKGKKG

SEQ ID NO: 115 - protein, 5 aa, Artificial Sequence
  Description of Artificial Sequence Synthetic peptide
CGKGK

SEQ ID NO: 116 - protein, 4 aa, Artificial Sequence
  Description of Artificial Sequence Synthetic peptide
GKGK

SEQ ID NO: 117 - DNA, 21 bp, Artificial Sequence
  Description of Artificial Sequence Synthetic oligonucleotide
ccuacgccgaguacuucgatt

SEQ ID NO: 118 - DNA, 21 bp, Artificial Sequence
  Description of Artificial Sequence Synthetic oligonucleotide
ucgaaguacucggcguaggtt

SEQ ID NO: 119 - protein, 6 aa, Artificial Sequence
  Description of Artificial Sequence Synthetic peptide
CKKKKK

SEQ ID NO: 120 - protein, 6 aa, Artificial Sequence
  Description of Artificial Sequence Synthetic peptide
CKKKKK

SEQ ID NO: 121 - protein, 6 aa, Artificial Sequence
  Description of Artificial Sequence Synthetic peptide
CKKKKK

SEQ ID NO: 122 - protein, 6 aa, Artificial Sequence
  Description of Artificial Sequence Synthetic peptide
CKKKKK

SEQ ID NO: 123 - protein, 6 aa, Artificial Sequence
  Description of Artificial Sequence Synthetic peptide
CKKKKK

SEQ ID NO: 124 - protein, 8 aa, Artificial Sequence
  Description of Artificial Sequence Synthetic peptide
GKKKKKGC

SEQ ID NO: 125 - protein, 7 aa, Artificial Sequence
  Description of Artificial Sequence Synthetic peptide
GKKKKKG

SEQ ID NO: 126 - protein, 9 aa, Artificial Sequence
  Description of Artificial Sequence Synthetic peptide
GKKKKGKGC

SEQ ID NO: 127 - protein, 10 aa, Artificial Sequence
  Description of Artificial Sequence Synthetic peptide
KKKKKKKKKK